(19) United States
(12) Patent Application Publication
(10) Pub. No.: US 2026/0209808 A1
(43) Pub. Date: Jul. 23, 2026

(54) Methods for the Production of Tryptophan Derivatives

(71) Applicant: Natural MedTech Pty Ltd, Sherbrooke, Victoria (AU)

(72) Inventors: Simon Colin KESSLER, Sherbrooke (AU); Yit-Heng CHOOI, Sherbrooke (AU); Mark Heinrich Wolfgang HESTERMANN, Sherbrooke (AU); Luke Andrew MCFARLANE, Sherbrooke (AU)

(73) Assignee: Natural MedTech Pty Ltd, Sherbrooke, Victoria (AU)

(21) Appl. No.: 19/140,659

(22) PCT Filed: Dec. 20, 2023
(86) PCT No.: PCT/AU2023/051347

§ 371 Date: Jun. 18, 2025

(30) Foreign Application Priority Data
Dec. 20, 2022 (AU) 2022903920
Aug. 2, 2023 (AU) 2023902441

(51) Int. Cl. C12P 17/10; C07D 209/20; C07F 9/572; C12N 9/02; C12N 9/10; C12N 9/12; C12N 9/14; C12N 9/88

(52) U.S. Cl. CPC C12P 17/10; C07D 209/20; C07F 9/5728; C12N 9/0042; C12N 9/0083; C12N 9/1007; C12N 9/1205; C12N 9/14; C12N 9/88; C12Y 106/02004; C12Y 201/01049; C12Y 207/0102; C12Y 401/01028

(57) ABSTRACT

The present disclosure relates generally to the biosynthetic production of tryptophan derivatives, in particular tryptamine and tryptamine-derived alkaloids.

### RELATED APPLICATION DATA

The present application claims priority from Australian Patent Application No. 2022903920 filed on 20 Dec. 2022 entitled “Methods for the production of tryptophan derivatives” and Australian Patent Application No. 2023902441 filed on 2 Aug. 2023 entitled “Methods for the production of tryptophan derivatives”. The entire contents of these applications are hereby incorporated by reference.

### SEQUENCE LISTING

The present application is filed together with a Sequence Listing in electronic form. The entire contents of the Sequence Listing are hereby incorporated by reference.

### FIELD

The present disclosure relates generally to the biosynthetic production of tryptophan derivatives, in particular tryptamine and tryptamine-derived alkaloids.

### BACKGROUND

Secondary metabolite production by various fungi, microorganisms and plants have been an extremely important source of a variety of therapeutically significant pharmaceuticals. Many natural products with therapeutic properties, or with the potential to be used therapeutically, are synthesized from tryptophan and tryptamine, including for example psilocybin, psilocin, N,N-dimethyltryptamine (DMT), 5-methoxy-N,N-dimethyltryptamine, 5-hydroxy-N,N-dimethyltryptamine (bufotenin), serotonin (5-hydroxytryptamine), melatonin, communesins, ergotamines, harmala alkaloids and lysergamides. Psilocybin (4-phosphoryloxy-N,N-dimethyltryptamine or [3-(2-trimethylaminoethyl)-1H-indol-4-yl]dihydrogen phosphate) is an abundant tryptophan-derived alkaloid in a number of fungal species. Upon ingestion, psilocybin is dephosphorylated to produce psilocin, which is psychoactive.

Unlike a number of psychoactive substances, psilocybin and psilocin do not appear to be addictive. Psilocybin has been the subject of considerable interest for its potential use in the treatment of mental illness. For example, psilocybin is a promising candidate for the treatment of conditions such as depression (including treatment-resistant depression), anxiety disorders, personality disorders, obsessive compulsive disorder and substance dependency.

Accordingly, there is a need for efficient large-scale production of psilocybin. The psilocybin content of hallucinogenic mushrooms is too low to make extraction a viable source of the compound. Psilocybin is often produced by complex chemical synthesis. However this is expensive, time consuming and involves the use of harsh, toxic chemicals. Also, yields and purity of the synthesised compounds can be low.

The pathways for the production of psilocybin, and numerous other alkaloid derivatives of tryptophan and tryptamine have been elucidated and the genes responsible for the generation of intermediates in the pathways have been identified. This opens up the possibility of in vivo production of compounds such as psilocybin from suitable host cells, in particular genetically modified microorganisms replicating the natural biosynthetic pathways found in the natural fungal and plant sources of the compounds. There is a need in the art for improved methods to increase the efficiency of synthesis of tryptophan- and tryptamine-derived compounds.

### SUMMARY

The present disclosure is predicated on the inventors' surprising identification that a class of tryptophan decarboxylases exemplified by fungal pyridoxal phosphate (PLP)-dependent tryptophan decarboxylases derived from members of the class Eurotiomycetes, for example the tryptophan decarboxylase CnsB from Penicillium expansum and the homologous tryptophan decarboxylase (AsTDC1) from Aspergillus steynii, when expressed in cells such as Saccharomyces cerevisiae, can significantly increase the production of tryptamine in comparison to that achieved in S. cerevisiae expressing the PsiD enzyme. Additionally, it was found that tryptophan decarboxylase (AcTDC) from Aspergillus caelatus can significantly increase the production of tryptamine in comparison to that achieved with a PsiD enzyme.

The inventors also found that PLP-dependent tryptophan decarboxylases, for example those derived from flowering plants, such as CrTDC, can significantly increase the production of tryptamine in comparison to that achieved with a PsiD enzyme.

Based on the foregoing, the present disclosure provides a cell capable of producing at least one tryptophan derivative comprising at least one exogenous polynucleotide encoding a PLP-dependent tryptophan decarboxylase, wherein the level of production of the at least one tryptophan derivative in the cell is increased relative to the level of production of a second tryptophan derivative in an equivalent cell lacking the exogenous polynucleotide or comprising a second polynucleotide encoding a second tryptophan decarboxylase, and wherein the second polynucleotide is not the same as the exogenous polynucleotide.

The present disclosure also provides a cell capable of producing at least one tryptophan derivative comprising at least one exogenous polynucleotide encoding a pyridoxal phosphate (PLP)-dependent tryptophan decarboxylase, wherein the level of production of the at least one tryptophan derivative in the cell is increased relative to the level of production of a second tryptophan derivative in an equivalent cell lacking the exogenous polynucleotide or comprising a second polynucleotide encoding a second tryptophan decarboxylase, and wherein the second polynucleotide is not the same as the exogenous polynucleotide and wherein the PLP-dependent tryptophan decarboxylase comprises one or more or all of:
a) a first amino acid sequence of GX₁X₂X₃X₄X₅X₆X₇X₈X₉X₁₀X₁₁X₁₂X₁₃X₁₄X₁₅X₁₆X₁₇R wherein:
   X₁, X₂, X₃, X₄, X₈, X₉, X₁₀, X₁₁, X₁₂, X₁₃, X₁₄, X₁₅, and X₁₆ are any amino acid residue,
   X₅ and X₆ are an amino acid residue selected from the group consisting of glycine, alanine, serine and threonine,
   X₇ is an amino acid residue selected from the group consisting of serine and threonine, and
   X₁₇ is an amino acid residue selected from the group consisting of alanine and glycine;
b) a second amino acid sequence of X₁X₂X₃X₄X₅X₆HX₇X₈X₉X₁₀KX₁₁X₁₂X₁₃X₁₄X₁₅X₁₆ wherein:
   X₁ is any amino acid residue selected from the group consisting of tyrosine, leucine, phenylalanine, valine, cysteine and tryptophan,
   X₂, X₄, X₅, X₆, X₇, X₈, X₉, X₁₀, X₁₁, X₁₂, X₁₃, X₁₄ and X₁₅ are any amino acid residue,
   X₃ is any amino acid residue selected from the group consisting of serine and threonine, and
   X₁₆ is an amino acid residue selected from the group consisting of glycine, alanine, serine and threonine;
c) a third amino acid sequence of DX₁X₂X₃X₄X₅X₆X₇X₈X₉X₁₀X₁₁X₁₂X₁₃X₁₄GX₁₅X₁₆X₁₇X₁₈ X₁₉X₂₀X₂₁D wherein:
   X₁, X₂, X₃, X₄, X₅, X₆, X₈, X₉, X₁₀, X₁₁, X₁₂, X₁₃, X₁₄, X₁₅, X₁₇, X₁₈, X₁₉ and X₂₁ are any amino acid residue, and
   X₇ is an amino acid residue selected from the group consisting of proline and asparagine,
   X₁₆ is an amino acid residue selected from the group consisting of threonine, proline and valine, and
   X₂₀ is 1, 2, 3, 4, 5, or 6 amino acid residues and is any amino acid residue;
d) a fourth amino acid sequence of X₁X₂HX₃X₄X₅X₆X₇X₈X₉X₁₀ wherein:
   X₁ is an amino acid residue selected from the group consisting of tryptophan and phenylalanine,
   X₂, X₃, X₆, X₉ and X₁₀ are any amino acid residue,
   X₄ is an amino acid residue selected from the group consisting of aspartic acid and glutamic acid,
   X₅ is an amino acid residue selected from the group consisting of glycine, alanine and serine,
   X₇ is an amino acid residue selected from the group consisting of phenylalanine, tyrosine and tryptophan, and
   X₈ is an amino acid residue selected from the group consisting of glycine, alanine, serine and threonine; and
e) a fifth amino acid sequence of X₁X₂X₃X₄X₅X₆X₇HKX₈X₉X₁₀X₁₁X₁₂X₁₃X₁₄X₁₅X₁₆ wherein:
   X₁ is an amino acid residue selected from the group consisting of aspartic acid, glutamic acid, histidine and asparagine,
   X₂ is an amino acid residue selected from the group consisting of serine and threonine,
   X₃, X₄, X₅, X₇, X₁₀, X₁₁, X₁₂, X₁₃ and X₁₄ are any amino acid residue,
   X₆ is an amino acid residue selected from the group consisting of aspartic acid, glutamic acid, asparagine, serine and threonine,
   X₈ is an amino acid residue selected from the group consisting of tryptophan, phenylalanine, tyrosine, leucine, methionine and cysteine,
   X₉ is an amino acid residue selected from the group consisting of leucine, isoleucine, valine, methionine and phenylalanine,
   X₁₅ is an amino acid residue selected from the group consisting of cysteine, alanine and serine, and
   X₁₆ is an amino acid residue selected from the group consisting of glycine, serine, alanine and threonine.

In some embodiments, the cell comprises the first amino acid sequence of
GX₁X₂X₃X₄X₅X₆X₇X₈X₉X₁₀X₁₁X₁₂X₁₃X₁₄X₁₅X₁₆X₁₇R wherein:
   X₁, X₂, X₃, X₈, X₉, X₁₂, X₁₃, X₁₄, X₁₅, and X₁₆ are any amino acid residue,
   X₄ is an amino acid residue selected from the group consisting of serine, threonine and asparagine,
   X₅ and X₆ are an amino acid residue selected from the group consisting of glycine, alanine, serine and threonine,
   X₇ is an amino acid residue selected from the group consisting of serine and threonine,
   X₁₀ is an amino acid residue selected from the group consisting of asparagine, isoleucine and valine,
   X₁₁ is an amino acid residue selected from the group consisting of leucine, isoleucine, methionine and valine, and
   X₁₇ is an amino acid residue selected from the group consisting of alanine and glycine.

In some embodiments, the cell comprises the first amino acid sequence of GX₁X₂X₃X₄X₅X₆X₇X₈X₉X₁₀X₁₁X₁₂X₁₃X₁₄X₁₅X₁₆X₁₇R wherein:
X₁, X₂, X₃, X₈, X₁₂, X₁₃, X₁₄, X₁₅, and X₁₆ are any amino acid residue,
X₄ is an amino acid residue selected from the group consisting of serine, threonine and asparagine,
X₅ and X₆ are an amino acid residue selected from the group consisting of glycine and threonine,
X₇ is an amino acid residue selected from the group consisting of serine and threonine,
X₉ is an amino acid residue selected from the group consisting of alanine, glycine, cysteine and serine,
X₁₀ is an amino acid residue selected from the group consisting of asparagine, isoleucine and valine,
X₁₁ is an amino acid residue selected from the group consisting of leucine, methionine and valine, and
X₁₇ is an amino acid residue selected from the group consisting of alanine and glycine.

In some embodiments, the cell comprises the second amino acid sequence of X₁X₂X₃X₄X₅X₆HX₇X₈X₉X₁₀KX₁₁X₁₂X₁₃X₁₄X₁₅X₁₆ wherein:
X₁ is any amino acid residue selected from the group consisting of tyrosine, leucine, phenylalanine, valine, cysteine and tryptophan,
X₂, X₄, X₅, X₆, X₇, X₉, X₁₀, X₁₁, X₁₂, X₁₃, X₁₄ and X₁₅ are any amino acid residue,
X₃ is any amino acid residue selected from the group consisting of serine and threonine,
X₈ is any amino acid residue selected from the group consisting of serine and methionine, and
X₁₆ is an amino acid residue selected from the group consisting of glycine, alanine, serine and threonine.

In some embodiments, the cell comprises the second amino acid sequence of X₁X₂X₃X₄X₅X₆HX₇X₈X₉X₁₀KX₁₁X₁₂X₁₃X₁₄X₁₅X₁₆ wherein:
X₁ is any amino acid residue selected from the group consisting of tyrosine, leucine, phenylalanine, valine, cysteine and tryptophan,
X₂, X₄, X₇, X₉, X₁₀, X₁₁, X₁₂, X₁₃, X₁₄ and X₁₅ are any amino acid residue,
X₃ is any amino acid residue selected from the group consisting of serine and threonine,
X₅ is any amino acid residue selected from the group consisting of glutamine and glutamic acid,
X₆ is any amino acid residue selected from the group consisting of glycine, alanine, serine and threonine,
X₈ is the amino acid residue serine, and
X₁₆ is the amino acid residue glycine.

In some embodiments, the cell comprises the third amino acid sequence of DX₁X₂X₃X₄X₅X₆X₇X₈X₉X₁₀X₁₁X₁₂X₁₃X₁₄GX₁₅X₁₆X₁₇X₁₈X₁₉X₂₀X₂₁D wherein:
X₁, X₂, X₃, X₅, X₆, X₉, X₁₀, X₁₁, X₁₅, X₁₇ and X₁₈ are any amino acid residue,
X₄ is an amino acid residue selected from the group consisting of glycine, alanine, serine, aspartic acid and asparagine,
X₇ is an amino acid residue selected from the group consisting of proline and asparagine
X₈ is an amino acid residue selected from the group consisting of phenylalanine, isoleucine, leucine, threonine, tyrosine and tryptophan,
X₁₂ is an amino acid residue selected from the group consisting of alanine and glycine,
X₁₃ is an amino acid residue selected from the group consisting of serine, threonine and asparagine,
X₁₄ is an amino acid residue selected from the group consisting of cysteine, alanine, serine, threonine, leucine, glycine and valine, and
X₁₆ is an amino acid residue selected from the group consisting of threonine, proline and valine,
X₁₉ is an amino acid residue selected from the group consisting of glycine, alanine, serine and threonine,
X₂₀ is 1, 2, 3, 4, 5 or 6 amino acid residues and is any amino acid residue, and
X₂₁ is an amino acid residue selected from the group consisting of isoleucine, leucine, valine and threonine.

In some embodiments, the cell comprises the third amino acid sequence of DX₁X₂X₃X₄X₅X₆X₇X₈X₉X₁₀X₁₁X₁₂X₁₃X₁₄GX₁₅X₁₆X₁₇X₁₈X₁₉X₂₀X₂₁D wherein:
X₁, X₂, X₃, X₅, X₆, X₉, X₁₁, X₁₅, X₁₈ and X₂₀ are any amino acid residue,
X₄ is an amino acid residue selected from the group consisting of glycine, alanine, serine, aspartic acid and asparagine,
X₇ is the amino acid residue proline,
X₈ is an amino acid residue selected from the group consisting of phenylalanine, threonine and tyrosine,
X₁₀ is an amino acid residue selected from the group consisting of isoleucine, leucine, valine, alanine and glycine,
X₁₂ is an amino acid residue selected from the group consisting of alanine and glycine,
X₁₃ is an amino acid residue selected from the group consisting of serine, threonine and asparagine,
X₁₄ is an amino acid residue selected from the group consisting of cysteine, serine, threonine, leucine, glycine and valine,
X₁₆ is an amino acid residue selected from the group consisting of threonine, proline and valine,
X₁₇ is an amino acid residue selected from the group consisting of asparagine, lysine, arginine, aspartic acid, serine and valine,
X₁₉ is an amino acid residue selected from the group consisting of glycine, alanine, serine and threonine, and
X₂₁ is an amino acid residue selected from the group consisting of isoleucine, leucine, valine and threonine.

In some embodiments, the cell comprises the fourth amino acid sequence of X₁X₂HX₃X₄X₅X₆X₇X₈X₉X₁₀ wherein:
X₁ is an amino acid residue selected from the group consisting of tryptophan and phenylalanine,
X₂, X₃, X₆ and X₁₀ are any amino acid residue,
X₄ is an amino acid residue selected from the group consisting of aspartic acid and glutamic acid,
X₅ is an amino acid residue selected from the group consisting of glycine, alanine and serine,
X₇ is an amino acid residue selected from the group consisting of phenylalanine, tyrosine and tryptophan,
X₈ is an amino acid residue selected from the group consisting of glycine, alanine, serine and threonine, and
X₉ is an amino acid residue selected from the group consisting of alanine, glycine, leucine, serine and threonine.

In some embodiments, the cell comprises the fourth amino acid sequence of X₁X₂HX₃X₄X₅X₆X₇X₈X₉X₁₀ wherein:
X₁ is an amino acid residue selected from the group consisting of tryptophan and phenylalanine,
X₂ and X₃ are any amino acid residue,
X₄ is the amino acid residue aspartic acid,
X₅ is an amino acid residue selected from the group consisting of glycine, alanine and serine,
X₆ is an amino acid residue selected from the group consisting of alanine, valine and serine,
X₇ is an amino acid residue selected from the group consisting of phenylalanine, tyrosine and tryptophan,
X₈ is an amino acid residue selected from the group consisting of glycine, alanine, serine and threonine,
X₉ is an amino acid residue selected from the group consisting of alanine, glycine, leucine, serine and threonine, and
X₁₀ is an amino acid residue selected from the group consisting of serine, alanine, phenylalanine and threonine.

In some embodiments, the cell comprises the fifth amino acid sequence of X₁X₂X₃X₄X₅X₆X₇HKX₈X₉X₁₀X₁₁X₁₂X₁₃X₁₄X₁₅X₁₆ wherein:
X₁ is an amino acid residue selected from the group consisting of aspartic acid, glutamic acid, histidine and asparagine,
X₂ is an amino acid residue selected from the group consisting of serine and threonine,
X₃, X₄, X₅, X₇, X₁₀, X₁₂ and X₁₄ are any amino acid residue,
X₆ is an amino acid residue selected from the group consisting of aspartic acid, glutamic acid, asparagine, serine and threonine,
X₈ is an amino acid residue selected from the group consisting of tryptophan, phenylalanine, tyrosine and cysteine,
X₉ is an amino acid residue selected from the group consisting of leucine, isoleucine, valine, methionine and phenylalanine,
X₁₁ is an amino acid residue selected from the group consisting of glutamine, valine, threonine and alanine,
X₁₃ is an amino acid residue selected from the group consisting of tyrosine, phenylalanine, leucine, isoleucine and valine,
X₁₅ is an amino acid residue selected from the group consisting of cysteine, alanine and serine, and
X₁₆ is an amino acid residue selected from the group consisting of glycine, serine, alanine and threonine.

In some embodiments, the cell comprises the fifth amino acid sequence of X₁X₂X₃X₄X₅X₆X₇HKX₈X₉X₁₀X₁₁X₁₂X₁₃X₁₄X₁₅X₁₆ wherein:
X₁ is an amino acid residue selected from the group consisting of aspartic acid, glutamic acid, histidine and asparagine,
X₂ is an amino acid residue selected from the group consisting of serine and threonine,
X₃, X₄ and X₁₄ are any amino acid residue,
X₅ is the amino acid residue tryptophan,
X₆ is an amino acid residue aspartic acid,
X₇ is an amino acid residue selected from the group consisting of alanine and glycine,
X₈ is the amino acid residue tryptophan,
X₉ is the amino acid residue leucine,
X₁₀ is an amino acid residue selected from the group consisting of phenylalanine, leucine, glycine, asparagine and methionine,
X₁₁ is the amino acid residue glutamine,
X₁₂ is an amino acid residue selected from the group consisting of threonine, serine, leucine, isoleucine, valine and alanine,
X₁₃ is an amino acid residue selected from the group consisting of tyrosine, phenylalanine, leucine, isoleucine and valine,
X₁₅ is an amino acid residue selected from the group consisting of cysteine and serine, and
X₁₆ is an amino acid residue selected from the group consisting of glycine and serine.

In some embodiments, the exogenous polynucleotide comprises in order from 5′ to 3′ the first amino acid sequence, the second amino acid sequence, the third amino acid sequence, the fourth amino acid sequence and the fifth amino acid sequence.

In some embodiments, there are about 2 to about 50 amino acids between the first and the second amino acid sequence. In some embodiments, there are about 10 to about 35 amino acids between the first and the second amino acid sequence. In some embodiments, there are about 12 to about 25 amino acids between the first and the second amino acid sequence.

In some embodiments, there are about 2 to about 50 amino acids between the second and the third amino acid sequence. In some embodiments, there are about 20 to about 40 amino acids between the second and the third amino acid sequence. In some embodiments, there are about 25 to about 35 amino acids between the second and the third amino acid sequence.

In some embodiments, there are about 5 to about 50 amino acids between the third and the fourth amino acid sequence. In some embodiments, there are about 10 to about 25 amino acids between the third and the fourth amino acid sequence. In some embodiments, there are about 12 to about 22 amino acids between the third and the fourth amino acid sequence.

In some embodiments, there are about 5 to about 50 amino acids between the fourth and the fifth amino acid sequence. In some embodiments, there are about 10 to about 30 amino acids between the fourth and the fifth amino acid sequence. In some embodiments, there are about 15 to about 20 amino acids between the fourth and the fifth amino acid sequence.

The present disclosure also provides a cell comprising an exogenous gene and capable of producing at least one tryptophan derivative, wherein the exogenous gene encodes a fungal pyridoxal phosphate (PLP)-dependent tryptophan decarboxylase derived from the class Eurotiomycetes, or a PLP-dependent tryptophan decarboxylase at least about 80% identical to, or comprising one or more conservative amino acid substitutions with respect to, said fungal PLP-dependent tryptophan decarboxylase, and wherein the level of production of the at least one tryptophan derivative in the cell is increased relative to the level of production of the at least one tryptophan derivative in an equivalent cell that does not possess the exogenous gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.

The present disclosure also provides a cell comprising an exogenous gene and capable of producing at least one tryptophan derivative, wherein the exogenous gene encodes a PLP-dependent tryptophan decarboxylase (CnsB) having an amino acid sequence set forth in SEQ ID NO: 1, SEQ ID NO: 3 or SEQ ID NO: 5, or a sequence having at least about 70% sequence identity thereto, and wherein the level of production of the at least one tryptophan derivative in the cell is increased relative to the level of production of the at least one tryptophan derivative in an equivalent cell that does not possess the exogenous gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.

In an embodiment, the tryptophan decarboxylase comprises the amino acid sequence set forth in SEQ ID NO: 1, or a sequence at least or about 70%, 75%, 80%, 85%, 90%, 95% or 99% identical thereto.

In an embodiment, the cell comprises more than one copy of the exogenous gene. For example, the cell comprises two copies of the exogenous gene.

The exogenous gene may comprise the nucleic acid sequence of SEQ ID NO: 2, SEQ ID NO: 4 or SEQ ID NO: 6, or a sequence having at least about 75% sequence identity thereto. Optionally, the exogenous gene is operably linked to a heterologous promoter. In an exemplary embodiment, the cell comprises two copies of said exogenous gene.

The present disclosure also provides a cell comprising an exogenous gene and capable of producing at least one tryptophan derivative, wherein the exogenous gene encodes a PLP-dependent tryptophan decarboxylase having an amino acid sequence set forth in SEQ ID NO: 7, or a sequence having at least about 70% sequence identity thereto, and wherein the level of production of the at least one tryptophan derivative in the cell is increased relative to the level of production of the at least one tryptophan derivative in an equivalent cell that does not possess the exogenous gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.

The present disclosure also provides a cell capable of producing at least one tryptophan derivative comprising at least one exogenous polynucleotide encoding an amino acid sequence set forth in any one of SEQ ID NOs:1, 3, 5, 7, 29 and 48 or a sequence having at least about 70% sequence identity thereto, wherein the level of production of the at least one tryptophan derivative in the cell is increased relative to the level of production of a second tryptophan derivative in an equivalent cell lacking the exogenous polynucleotide or comprising a second polynucleotide encoding a second tryptophan decarboxylase, and wherein the second polynucleotide is not the same as the exogenous polynucleotide.

In an embodiment, the exogenous polynucleotide encodes an amino acid sequence set forth in SEQ ID NO: 1, or a sequence at least or about 70%, 75%, 80%, 85%, 90%, 95% or 99% identical thereto.

In an embodiment, the exogenous polynucleotide encodes an amino acid sequence set forth in SEQ ID NO: 3, or a sequence at least or about 70%, 75%, 80%, 85%, 90%, 95% or 99% identical thereto.

In an embodiment, the exogenous polynucleotide encodes an amino acid sequence set forth in SEQ ID NO: 5, or a sequence at least or about 70%, 75%, 80%, 85%, 90%, 95% or 99% identical thereto.

In an embodiment, the exogenous polynucleotide encodes an amino acid sequence set forth in SEQ ID NO: 7, or a sequence at least or about 70%, 75%, 80%, 85%, 90%, 95% or 99% identical thereto.

In an embodiment, the exogenous polynucleotide encodes an amino acid sequence set forth in SEQ ID NO: 29, or a sequence at least or about 70%, 75%, 80%, 85%, 90%, 95% or 99% identical thereto.

In an embodiment, the exogenous polynucleotide encodes an amino acid sequence set forth in SEQ ID NO: 48, or a sequence at least or about 70%, 75%, 80%, 85%, 90%, 95% or 99% identical thereto.

In one embodiment, the encoded polypeptide is produced using ancestral sequence reconstruction (e.g., as substantially described in Scossa and Fernie, Computational and Structural Biotechnology Journal, 19: 1579-1594, 2021) or by sequence optimization (e.g., as substantially described in Fox and Huisman, Trends in Biotechnology, 26: 132-138, 2008 or Chakrababarti et al., Biophysics and Computational Biology, 102: 12035-12040, 2005).

In one embodiment, the encoded polypeptide is produced using directed evolution (e.g., as substantially described in Cobb et al., AIChE J. 2013 May; 59(5): 1432-1440).

In some embodiments, the exogenous polynucleotide comprises the nucleic acid sequence of any one of SEQ ID NOs: 2, 4, 6, 8, 23, 30, 31, 47, and 49-51 or a sequence having at least about 75% sequence identity thereto.

In some embodiments, the exogenous polynucleotide is operably linked to a heterologous promoter.

In an embodiment, the cell comprises two or more copies of said exogenous polynucleotide. For example, the cell comprises two, or three, or four, or five, or six, or seven copies of said exogenous polynucleotide.

In some embodiments, the exogenous polynucleotide is derived from the class Eurotiomycetes. In other embodiments, the exogenous polynucleotide is derived from the class Dicotyledons. In other embodiments, the exogenous polynucleotide is derived from the phylum Ascomycota. In other embodiments, the exogenous polynucleotide is derived from the phylum Basidiomycota.

The skilled person will understand that a polynucleotide as used in the disclosure need not comprise the same sequence as occurs in nature or from which it is originally derived. For example, a sequence may be codon optimized for enhanced expression in a cell or organism in which the encoded polypeptide is to be expressed.

The exogenous gene may comprise the nucleic acid sequence of SEQ ID NO: 8, or a sequence having at least about 75% sequence identity thereto. Optionally, the exogenous gene is operably linked to a heterologous promoter. In an exemplary embodiment, the cell comprises two copies of said exogenous gene.

In some embodiments, the level of production of the at least one tryptophan derivative in the cell is increased relative to the level of production of the at least one tryptophan derivative in an equivalent cell that does not possess the exogenous gene and that comprises a non-PLP-dependent tryptophan decarboxylase. Optionally, the non-PLP-dependent tryptophan decarboxylase is PsiD. In an embodiment, the PsiD tryptophan decarboxylase comprises the amino acid sequence set forth in SEQ ID NO: 9, and optionally the at least tryptophan derivative is tryptamine or psilocybin.

In an embodiment, the cell may further comprise a nucleic acid sequence encoding an additional tryptophan decarboxylase. The additional tryptophan decarboxylase may be PsiD comprising the amino acid sequence set forth in SEQ ID NO: 9 or a sequence having at least about 70% sequence identity thereto. The additional tryptophan decarboxylase may be CrTDC comprising the amino acid sequence set forth in SEQ ID NO: 29 or a sequence having at least about 70% sequence identity thereto.

In an embodiment, the equivalent cell comprises a polynucleotide encoding a second tryptophan decarboxylase. In some embodiments, the second tryptophan decarboxylase is a PLP-independent tryptophan decarboxylase. For example, the PLP-independent tryptophan decarboxylase is a PsiD tryptophan decarboxylase.

In an embodiment, the cell further comprises at least one polynucleotide encoding an additional tryptophan decarboxylase. For example, the cell further comprises at least two, three, four, five, six, or seven polynucleotides encoding an additional tryptophan decarboxylase.

In some embodiments, the additional tryptophan decarboxylase is CnsBv1. In an embodiment, the CnsBv1 tryptophan decarboxylase comprises the amino acid sequence set forth in SEQ ID NO: 1 or a sequence having at least about 70% sequence identity thereto.

In some embodiments, the additional tryptophan decarboxylase is CnsBv2. In an embodiment, the CnsBv2 tryptophan decarboxylase comprises the amino acid sequence set forth in SEQ ID NO: 3 or a sequence having at least about 70% sequence identity thereto.

In some embodiments, the additional tryptophan decarboxylase is ΔCnsBv2. In an embodiment, the ΔCnsBv2 tryptophan decarboxylase comprises the amino acid sequence set forth in SEQ ID NO: 5 or a sequence having at least about 70% sequence identity thereto.

In some embodiments, the additional tryptophan decarboxylase is asTDC1. In an embodiment, the asTDC1 tryptophan decarboxylase comprises the amino acid sequence set forth in SEQ ID NO: 7 or a sequence having at least about 70% sequence identity thereto.

In some embodiments, the additional tryptophan decarboxylase is PsiD. In an embodiment, the PsiD tryptophan decarboxylase comprises the amino acid sequence set forth in SEQ ID NO: 9 or a sequence having at least about 70% sequence identity thereto.

In other embodiments, the additional tryptophan decarboxylase is CrTDC. In an embodiment, the CrTDC tryptophan decarboxylase comprises the amino acid sequence set forth in SEQ ID NO: 29 or a sequence having at least about 70% sequence identity thereto.

In some embodiments, the additional tryptophan decarboxylase is AcTDC. In an embodiment, the AcTDC tryptophan decarboxylase comprises the amino acid sequence set forth in SEQ ID NO: 48 or a sequence having at least about 70% sequence identity thereto.

In an embodiment, the cell may further comprise a nucleic acid sequence encoding a tryptamine 4-monooxygenase comprising the amino acid sequence set forth in SEQ ID NO: 11 or a sequence having at least about 70% sequence identity thereto. The tryptamine 4-monooxygenase may be PsiH. In an exemplary embodiment, the cell comprises two or more copies of a gene encoding PsiH.

In an embodiment, the cell may further comprise a polynucleotide encoding a tryptamine 4-monooxygenase. In such embodiments, the tryptamine 4-monooxygenase is PsiH. In some embodiments, the PsiH comprises the amino acid sequence set forth in SEQ ID NO: 11 or a sequence having at least about 70% sequence identity thereto. In an embodiment, the cell comprises two or more copies of a polynucleotide encoding PsiH. In some embodiments, the cell comprises at least two, three, four, five, six, or seven polynucleotides encoding PsiH.

In an embodiment, the cell may further comprise a nucleic acid sequence encoding a 4-hydroxytryptamine kinase comprising the amino acid sequence set forth in SEQ ID NO: 13 or a sequence having at least about 70% sequence identity thereto. The 4-hydroxytryptamine kinase may be PsiK. In an exemplary embodiment, the cell comprises two or more copies of a gene encoding PsiK.

In an embodiment, the cell may further comprise a polynucleotide encoding a 4-hydroxytryptamine kinase. In such embodiments, the 4-hydroxytryptamine kinase is PsiK. In some embodiments, the PsiK comprises the amino acid sequence set forth in SEQ ID NO: 13 or a sequence having at least about 70% sequence identity thereto. In an embodiment, the cell comprises two or more copies of a polynucleotide encoding PsiK. In some embodiments, the cell comprises at least two, three, four, five, six, or seven polynucleotides encoding PsiK.

In an embodiment, the cell may further comprise a nucleic acid sequence encoding a methyl transferase comprising the amino acid sequence set forth in SEQ ID NO: 15 or a sequence having at least about 70% sequence identity thereto. The methyl transferase may be PsiM. In an exemplary embodiment, the cell comprises two or more copies of a gene encoding PsiM.

In an embodiment, the cell may further comprise a polynucleotide encoding a methyl transferase. In such embodiments, the methyl transferase is PsiM. In some embodiments, the PsiM comprises the amino acid sequence set forth in SEQ ID NO: 15 or a sequence having at least about 70% sequence identity thereto. In an embodiment, the cell comprises two or more copies of a polynucleotide encoding PsiM. In some embodiments, the cell comprises at least two, three, four, five, six, or seven polynucleotides encoding PsiM.

In other embodiments, the methyl transferase is RmNMT. In some embodiments, the RmNMT comprises the amino acid sequence set forth in SEQ ID NO: 42 or a sequence having at least about 70% sequence identity thereto. In an embodiment, the cell comprises two or more copies of a polynucleotide encoding RmNMT. In some embodiments, the cell comprises at least two, three, four, five, six, or seven polynucleotides encoding RmNMT.

In an embodiment, the cell may further comprise a nucleic acid sequence encoding a cytochrome P450 reductase comprising the amino acid sequence set forth in SEQ ID NO: 17 or a sequence having at least about 70% sequence identity thereto. The cytochrome P450 reductase may be PcCpr. In an exemplary embodiment, the cell comprises two or more copies of a gene encoding PcCpr.

In an embodiment, the cell may further comprise a polynucleotide encoding a cytochrome P450 reductase. In such embodiments, the cytochrome P450 reductase is PcCpr. In some embodiments, the PcCpr comprises the amino acid sequence set forth in SEQ ID NO: 17 or a sequence having at least about 70% sequence identity thereto. In an embodiment, the cell comprises two or more copies of a polynucleotide encoding PcCpr. In some embodiments, the cell comprises at least two, three, four, five, six, or seven polynucleotides encoding PcCpr.

In other embodiments, the cytochrome P450 reductase is OsCPR. In some embodiments, the OsCPR comprises the amino acid sequence set forth in SEQ ID NO: 39 or a sequence having at least about 70% sequence identity thereto. In an embodiment, the cell comprises two or more copies of a polynucleotide encoding OsCPR. In some embodiments, the cell comprises at least two, three, four, five, six, or seven polynucleotides encoding OsCPR.

In an embodiment, the cell may further comprise one or more, optionally at least two, copies of a gene encoding an adenosylhomocysteinase. The adenosylhomocysteinase may be Sahl. In an embodiment, the cell further comprises one or more, optionally at least two, copies of a gene encoding an adenosine kinase. The adenosine kinase may be Ado1.

In an embodiment, the cell may further comprise at least one copy of a polynucleotide encoding an adenosylhomocysteinase. In some embodiments, the adenosylhomocysteinase is Sahl. In some embodiments, the Sahl comprises the amino acid sequence set forth in SEQ ID NO: 19 or a sequence having at least about 70% sequence identity thereto. In some embodiments, the cell comprises at least two, three, four, five, six, or seven polynucleotides encoding Sah1.

In an embodiment, the cell may further comprise at least one copy of a polynucleotide encoding an adenosine kinase. In some embodiments, the adenosine kinase is Ado1. In some embodiments, the Ado1 comprises the amino acid sequence set forth in SEQ ID NO: 21 or a sequence having at least about 70% sequence identity thereto. In some embodiments, the cell comprises at least two, three, four, five, six, or seven polynucleotides encoding Ado1.

In an embodiment, the cell may further comprise at least one copy of a polynucleotide encoding a tryptamine 5-hydroxylase. In some embodiments, the tryptamine 5-hydroxylase is OsT5H. In some embodiments, the OsT5H comprises the amino acid sequence set forth in SEQ ID NO: 36 or a sequence having at least about 70% sequence identity thereto. In some embodiments, the cell comprises at least two, three, four, five, six, or seven polynucleotides encoding OsT5H.

In an embodiment, the cell is a yeast cell. In an embodiment, the yeast cell is a Saccharomyces sp. cell, for example a S. cerevisiae cell. In an embodiment, the yeast cell is a Yarrowia sp. cell.

In an embodiment, the cell is a bacterial cell. In an embodiment, the bacterial cell is a Escherichia sp. For example, a E. coli. In an embodiment, the bacterial cell is a Corynebacterium sp. For example, a C. glutamicum.

In an embodiment, the cell is a fungal cell. In an embodiment, the fungal cell is a Aspergillus sp.

In an embodiment, the at least one tryptophan derivative may be tryptamine or a tryptamine-derived alkaloid. In an exemplary embodiment, the tryptophan derivative is tryptamine. In another embodiment, the tryptamine-derived alkaloid is psilocybin. In another embodiment, the tryptophan derivative is serotonin. In another embodiment, the tryptophan derivative is bufotenine (5-hydroxy-N,N-dimethyltryptamine). In another embodiment, the tryptophan derivative is N,N-dimethyltryptamine. In another embodiment, the tryptophan derivative is 5-methoxy-N,N-dimethyltryptamine. In another embodiment, the tryptophan derivative is N-methyltryptamine. In another embodiment, the tryptophan derivative is N-acetylserotonin. In another embodiment, the tryptophan derivative is melatonin.

The present disclosure also provides a cell capable of producing at least one tryptophan derivative, comprising:
(a) one or more copies of a gene encoding a fungal PLP-dependent tryptophan decarboxylase derived from the class Eurotiomycetes, or a PLP-dependent tryptophan decarboxylase at least about 80% identical to, or comprising one or more conservative amino acid substitutions with respect to, said fungal PLP-dependent tryptophan decarboxylase;
(b) one or more copies of a gene encoding a PsiH tryptamine 4-monooxygenase;
(c) one or more copies of a gene encoding a PsiK hydroxytryptamine kinase; and
(d) one or more copies of a gene encoding a PsiM methyl transferase.

The present disclosure also provides a cell capable of producing at least one tryptophan derivative, comprising:
(a) one or more copies of a gene encoding a PLP-dependent tryptophan decarboxylase (CnsB) having an amino acid sequence set forth in SEQ ID NO: 1, SEQ ID NO: 3 or SEQ ID NO: 5, or a sequence having at least about 70% sequence identity thereto;
(b) one or more copies of a gene encoding a PsiH tryptamine 4-monooxygenase;
(c) one or more copies of a gene encoding a PsiK hydroxytryptamine kinase; and
(d) one or more copies of a gene encoding a PsiM methyl transferase.

The present disclosure also provides a cell capable of producing at least one tryptophan derivative, comprising:
(a) one or more copies of a gene encoding a PLP-dependent tryptophan decarboxylase having an amino acid sequence set forth in SEQ ID NO: 7, or a sequence having at least about 70% sequence identity thereto;
(b) one or more copies of a gene encoding a PsiH tryptamine 4-monooxygenase;
(c) one or more copies of a gene encoding a PsiK hydroxytryptamine kinase; and
(d) one or more copies of a gene encoding a PsiM methyl transferase.

The present disclosure also provides a cell capable of producing at least one tryptophan derivative, comprising:
(a) at least one exogenous polynucleotide encoding a PLP-dependent tryptophan decarboxylase as described herein;
(b) at least one exogenous polynucleotide encoding a PsiH tryptamine 4-monooxygenase;
(c) at least one exogenous polynucleotide encoding a PsiK hydroxytryptamine kinase; and
(d) at least one exogenous polynucleotide encoding a PsiM methyl transferase.

The present disclosure also provides a cell capable of producing at least one tryptophan derivative, comprising:
(a) at least one exogenous polynucleotide encoding a PLP-dependent tryptophan decarboxylase having an amino acid sequence set forth in any one of SEQ ID NOs: 1, 3, 5, 7, 29 and 48, or a sequence having at least about 70% sequence identity thereto;
(b) at least one exogenous polynucleotide encoding a PsiH tryptamine 4-monooxygenase;
(c) at least one exogenous polynucleotide encoding a PsiK hydroxytryptamine kinase; and
(d) one or more copies of an exogenous polynucleotide encoding a PsiM methyl transferase.

The present disclosure also provides a cell capable of producing at least one tryptophan derivative, comprising:
(a) at least one exogenous polynucleotide encoding a PLP-dependent tryptophan decarboxylase described herein;
(b) one or more copies of an exogenous polynucleotide encoding a OsT5H tryptamine 5-hydroxylase;
(c) one or more copies of an exogenous polynucleotide encoding a OsCPR cytochrome P450 reductase; and
(d) one or more copies of an exogenous polynucleotide encoding a RmNMT methyl transferase.

The present disclosure also provides a cell capable of producing at least one tryptophan derivative, comprising:
(a) at least one exogenous polynucleotide encoding a PLP-dependent tryptophan decarboxylase having an amino acid sequence set forth in SEQ ID NO: 7;
(b) at least one exogenous polynucleotide encoding a OsT5H tryptamine 5-hydroxylase;
(c) at least one exogenous polynucleotide encoding a OsCPR cytochrome P450 reductase; and
(d) at least one exogenous polynucleotide encoding a RmNMT methyl transferase.

In some embodiments, the cell is capable of a higher level of production of at least one tryptophan derivative relative to the level of production of the at least one tryptophan derivative in an equivalent cell that does not possess (a), and that comprises a non-PLP-dependent tryptophan decarboxylase, optionally wherein the non-PLP-dependent tryptophan decarboxylase is PsiD. Optionally the tryptophan derivative is tryptamine or psilocybin. Optionally, the tryptophan derivative is serotonin, bufotenine (5-hydroxy-N,N-dimethyltryptamine), N,N-dimethyltryptamine, 5-methoxy-N,N-dimethyltryptamine, N-methyltryptamine, N-acetylserotonin or melatonin.

In some embodiments, the cell is capable of an increased level of production of the at least one tryptophan derivative relative to the level of production of a second tryptophan derivative in an equivalent cell comprising a second polynucleotide encoding a second tryptophan decarboxylase, wherein the second polynucleotide is not the same as (a). In some embodiments, the second polynucleotide encodes a PsiD tryptophan decarboxylase.

The cell may further comprise one or more copies of a gene encoding a cytochrome P450 reductase, optionally a PcCpr cytochrome P450 reductase. The cell may further comprise one or more copies of a gene encoding an adenosylhomocysteinase, optionally an Sah1 adenosylhomocysteinase. The cell may further comprise one or more copies of a gene encoding an adenosine kinase, optionally an Ado1 adenosine kinase.

The cell may further comprise one or more copies of a gene encoding an additional tryptophan decarboxylase, optionally a PsiD and/or CrTDC tryptophan decarboxylase.

Those skilled in the art will appreciate that all embodiments of the cells described herein relate any of the cells described herein.

The present disclosure also provides a nucleic acid construct or vector comprising a nucleic acid sequence encoding a fungal PLP-dependent tryptophan decarboxylase derived from the class Eurotiomycetes, or a PLP-dependent tryptophan decarboxylase at least about 80% identical to, or comprising one or more conservative amino acid substitutions with respect to, said fungal PLP-dependent tryptophan decarboxylase, optionally wherein said nucleic acid sequence is operably linked to a heterologous promoter, and wherein the tryptophan decarboxylase encoded by said gene is capable of a higher level of production of at least one tryptophan derivative in a cell relative to the level of production of the at least one tryptophan derivative in an equivalent cell that does not possess said gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.

The present disclosure also provides a nucleic acid construct or vector comprising the nucleic acid sequence of SEQ ID NO: 2, SEQ ID NO: 4, SEQ ID NO: 6 or SEQ ID NO: 23, or a sequence having at least about 75% sequence identity thereto, optionally operably linked to a heterologous promoter, and wherein the tryptophan decarboxylase encoded by said gene is capable of a higher level of production of at least one tryptophan derivative in a cell relative to the level of production of the at least one tryptophan derivative in an equivalent cell that does not possess said gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.

The present disclosure also provides a nucleic acid construct or vector comprising the nucleic acid sequence of SEQ ID NO: 8, or a sequence having at least about 75% sequence identity thereto, optionally operably linked to a heterologous promoter, and wherein the tryptophan decarboxylase encoded by said gene is capable of a higher level of production of at least one tryptophan derivative in a cell relative to the level of production of the at least one tryptophan derivative in an equivalent cell that does not possess said gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.

The present disclosure also provides a vector comprising a polynucleotide encoding a PLP-dependent tryptophan decarboxylase as described herein, optionally wherein the polynucleotide is operably linked to a heterologous promoter capable of directing expression of the polynucleotide. In some embodiments, the polynucleotide is capable of an increased level of production of the at least one tryptophan derivative in a cell relative to the level of production of at least one tryptophan derivative in an equivalent cell lacking the polynucleotide or comprising a second polynucleotide encoding a second tryptophan decarboxylase, and wherein the second polynucleotide is not the same as the polynucleotide.

The present disclosure also provides a vector comprising a polynucleotide encoding a PLP-dependent tryptophan decarboxylase having an amino acid sequence set forth in any one of SEQ ID NOs: 1, 3, 5 and 7, optionally wherein the polynucleotide is operably linked to a heterologous promoter capable of directing expression of the polynucleotide in a cell that does not naturally express said polynucleotide. In some embodiments, the polynucleotide is capable of an increased level of production of at least one tryptophan derivative in a cell relative to the level of production of at least one tryptophan derivative in an equivalent cell lacking the polynucleotide or comprising a second polynucleotide encoding a second tryptophan decarboxylase, and wherein the second polynucleotide is not the same as the polynucleotide.

The present disclosure also provides a vector comprising a polynucleotide encoding a PLP-dependent tryptophan decarboxylase having an amino acid sequence set forth in any one of SEQ ID NOs: 29 and 48, optionally wherein the polynucleotide is operably linked to a heterologous promoter capable of directing expression of the polynucleotide in a cell that does not naturally express said polynucleotide. In some embodiments, the polynucleotide is capable of an increased level of production of at least one tryptophan derivative in a cell relative to the level of production of at least one tryptophan derivative in an equivalent cell lacking the polynucleotide or comprising a second polynucleotide encoding a second tryptophan decarboxylase, and wherein the second polynucleotide is not the same as the polynucleotide.

In an embodiment, the non-PLP-dependent tryptophan decarboxylase is PsiD, optionally comprising the amino acid sequence set forth in SEQ ID NO: 9. Optionally the at least tryptophan derivative is tryptamine or psilocybin.

The present disclosure also provides a host cell comprising a nucleic acid construct or vector of the disclosure.

The present disclosure also provides a method for producing at least one tryptophan derivative, comprising culturing a cell of the present disclosure under conditions suitable for the production of the at least one tryptophan derivative.

The method may further comprise extracting the at least one tryptophan derivative from the cell and/or the medium in which the cell is cultured. The at least one tryptophan derivative may be tryptamine or a tryptamine-derived alkaloid. In an exemplary embodiment, the tryptophan derivative is tryptamine. In another embodiment, the tryptamine-derived alkaloid is psilocybin.

The present disclosure also provides a method for producing at least one tryptophan derivative, the method comprising contacting tryptophan with at least one pyridoxal phosphate (PLP)-dependent tryptophan decarboxylase as described herein, under conditions suitable for the production of the at least one tryptophan derivative.

The present disclosure also provides a method for producing at least one tryptophan derivative, the method comprising contacting tryptophan with at least one pyridoxal phosphate (PLP)-dependent tryptophan decarboxylase comprising an amino acid sequence set forth in any one of SEQ ID NOs:1, 3, 5, 7, 29 and 48 or a sequence having at least about 70% sequence identity thereto, under conditions suitable for the production of the at least one tryptophan derivative.

In one example, the method is a cell-free method or is performed with a cell lysate, e.g., from a cell described herein.

In some embodiments, the method is performed in a bioreactor.

In some embodiments, the polypeptide and/or tryptophan is provided as within a cell lysate. In some embodiments, the polypeptide is provided as a purified and/or isolated polypeptide.

The present disclosure also provides a tryptophan derivative produced by a method described herein.

The present disclosure also provides a composition comprising a cell of the present disclosure, or a tryptophan derivative extracted therefrom.

The present disclosure also provides an isolated nucleotide sequence comprising a gene encoding a fungal PLP-dependent tryptophan decarboxylase derived from the class Eurotiomycetes, or a PLP-dependent tryptophan decarboxylase at least about 80% identical to, or comprising one or more conservative amino acid substitutions with respect to, said fungal PLP-dependent tryptophan decarboxylase, operably linked to a heterologous promoter capable of directing expression of the gene in a cell that does not naturally express said gene.

The present disclosure also provides an isolated nucleotide sequence comprising a gene encoding a tryptophan decarboxylase having an amino acid sequence set forth in SEQ ID NO: 1, SEQ ID NO: 3 or SEQ ID NO: 5, or a sequence having at least about 70% sequence identity thereto, operably linked to a heterologous promoter capable of directing expression of the gene in a cell that does not naturally express said gene.

The present disclosure also provides an isolated nucleotide sequence comprising a gene encoding a tryptophan decarboxylase having an amino acid sequence set forth in SEQ ID NO: 7, or a sequence having at least about 70% sequence identity thereto, operably linked to a heterologous promoter capable of directing expression of the gene in a cell that does not naturally express said gene.

The present disclosure also provides an isolated nucleotide sequence comprising a gene encoding a tryptophan decarboxylase having an amino acid sequence set forth in SEQ ID NO: 29 and 48, or a sequence having at least about 70% sequence identity thereto, operably linked to a heterologous promoter capable of directing expression of the gene in a cell that does not naturally express said gene.

The present disclosure also provides a method for treating or preventing a disease or disorder in a subject, comprising administering to the subject a composition of the present disclosure. Optionally the disease or disorder is a neurodevelopmental, neurological, neuromuscular, movement, psychiatric, or psychological disease, disorder or syndrome. In exemplary embodiments, the disease or disorder is selected from depression or a depressive disorder, an anxiety disorder, obsessive-compulsive disorder, a personality disorder, substance addiction or dependence, post-traumatic stress disorder, migraine and/or chronic headache.

The present disclosure also provides a composition of the present disclosure for use or administration as a nootropic or functional food.

The present disclosure also provides the use of a cell of the present disclosure, or a tryptophan derivative extracted therefrom, in the manufacture of a medicament for the treatment or prevention of a disease or disorder in a subject. Optionally the disease or disorder is a neurodevelopmental, neurological, neuromuscular, movement, psychiatric, or psychological disease, disorder or syndrome. In some embodiments, the medicament is provided as a nootropic. In other embodiments, the medicament is provided as a functional food.

### BRIEF DESCRIPTION OF THE DRAWINGS

Embodiments of the present disclosure are described herein, by way of non-limiting example only, with reference to the following drawings.

FIG. 1 shows the biosynthetic pathway for the production of psilocybin and psilocin from L-tryptophan.

FIG. 2. Pairwise Needleman-Wunsch alignment of the polypeptide sequences of: (A) CnsBv1 (SEQ ID NO: 1) and CnsBv2 (SEQ ID NO: 3); and (B) CnsBv1 (SEQ ID NO: 1) and ΔCnsBv2 (SEQ ID NO: 5). Conserved residues are shown in the line between the two sequences.

FIG. 3. Pairwise Needleman-Wunsch alignment of the polypeptide sequences of: (A) CnsBv1 (SEQ ID NO: 1) and PsiD (SEQ ID NO: 9); and (B) CnsBv2 (SEQ ID NO: 3) and PsiD (SEQ ID NO: 9). Conserved residues are shown in the line between the two sequences.

FIG. 4 is a pairwise Needleman-Wunsch alignment of the polypeptide sequences of: (A) CnsBv1 (SEQ ID NO: 1) and CrTDC (SEQ ID NO: 29); and (B) CnsBv2 (SEQ ID NO: 3) and CrTDC (SEQ ID NO: 29). Conserved residues are shown in the line between the two sequences identified.

FIG. 5 is a pairwise Needleman-Wunsch alignment of the polypeptide sequences of: (A) AsTDC1 (SEQ ID NO: 7) and CnsBv1 (SEQ ID NO: 1); (B) AsTDC1 (SEQ ID NO: 7) and CnsBv2 (SEQ ID NO: 3); (C) AsTDC1 (SEQ ID NO: 7) and PsiD (SEQ ID NO: 9); and (D) AsTDC1 (SEQ ID NO: 7) and CrTDC (SEQ ID NO: 29). Conserved residues are shown in the line between the two sequences identified.

FIG. 6 shows tryptamine yield (mg/L) from cells (A) and growth medium (B) of S. cerevisiae strains SC-cnsBv1 and SC-psiD measured after day 5 and day 6 of culture.

FIG. 7 shows tryptamine yield (mg/L) from S. cerevisiae cells SC-cnsBv1, SC-asTDC1 and SC-psiD measured after day 3, day 5 and day 6 of culture.

FIG. 8 shows combined psilocybin and psilocin yield (mg/L) from cells (A) and growth medium (B) of S. cerevisiae strains SC-D and SC-B measured after day 2 and day 3 of culture.

FIG. 9 shows combined psilocybin and psilocin yield (mg/L) from cells (A) and growth medium (B) of S. cerevisiae strains SC-DM and SC-BM measured after day 2 and day 3 of culture.

FIG. 10 shows combined psilocybin and psilocin yield (mg/L) from cells (A) and growth medium (B) of S. cerevisiae strains SC-DMAS and SC-BMAS measured after day 2 and day 3 of culture.

FIG. 11 shows combined psilocybin and psilocin yield (mg/L) from cells (A) and growth medium (B) of S. cerevisiae strains SC-DMASB and SC-BMASB measured after day 2 and day 3 of culture.

FIG. 12 shows combined psilocybin and psilocin yield (mg/L) from cells and growth medium of S. cerevisiae strains SC-D, SC-B, SC-DM, SC-BM, SC-DMAS, SC-BMAS, SC-DMASB and SC-BMASB measured after day 3 of culture.

FIG. 13 shows the biosynthetic pathway from tryptophan to N,N-dimethyltryptamine and bufotenine.

FIG. 14 shows relative compound abundance of metabolite extractions for strains S. cerevisiae strains (A) SC-rmNMT1, (B) SC-rmNMT2, (C) SC-rmNMT3, (D) SC-rmNMT4 and (E) SC-rmNMT5 measured after day 6 of culture. Shown are the average peak areas of extracted ion chromatograms from three biological replicates per strain for tryptamine, 5-OH-tryptophan, serotonin, N,N-dimethyltryptamine and bufotenine. The individual gene combinations of each strain are shown below their respective bar charts.

FIG. 15 shows tryptamine yield of E. coli strains EC-psiD, EC-asTDC1 and EC-ctrl measured after day 3 of culture.

FIG. 16 shows tryptamine yields of six strains of S. cerevisiae expressing different TDCs on day 3 of culturing.

### DETAILED DESCRIPTION

### Definitions

Unless defined otherwise, all technical and scientific terms used herein have the same meaning as is commonly understood by one of skill in the art to which the present disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e., at least one or more than one) unless the context clearly dictates otherwise. Thus, for example, reference to “a polypeptide” includes a single polypeptide, as well as two or more polypeptides.

As used herein, “and/or” refers to and encompasses any and all possible combinations of one or more of the associated listed items, as well as the lack of combinations when interpreted in the alternative (or).

In the context of this specification, the term “about” is understood to refer to a range of numbers that a person of skill in the art would consider equivalent to the recited value in the context of achieving the same function or result.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. By “consisting of” is meant including, and limited to, whatever follows the phrase “consisting of”. Thus, the phrase “consisting of” indicates that the listed elements are required or mandatory, and that no other elements may be present.

As used herein the term “exogenous” in the context of a gene or polynucleotide refers to the gene or polynucleotide when present in a cell that does not naturally comprise the gene or polynucleotide. The exogenous gene or polynucleotide may include a promoter and/or other regulatory genetic elements from its original source, or may be operably linked to one or more heterologous regulatory elements. Such heterologous regulatory elements may be endogenous to the ell or may themselves be exogenous.

The term “operably linked” as used herein refers to a functional relationship between two or more nucleic acid (e.g., DNA) segments. Typically, it refers to the functional relationship of transcriptional regulatory element (promoter) to a transcribed sequence, such as an exogenous gene as described herein. For example, a promoter is operably linked to a gene coding sequence, such as a polynucleotide defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance. For example, an intron in a 5′ UTR sequence or towards the 5′ end of a protein coding region can contain a transcriptional enhancer, providing an increased expression level, for example an FBAIN promoter region.

As used herein the term “pyridoxal phosphate (PLP)-dependent tryptophan decarboxylase” means a tryptophan decarboxylase that requires active site binding of pyridoxal 5′ phosphate (PLP) for catalytic activity, i.e., for catalysing the conversion of tryptophan to tryptamine.

As used herein the term “derived” in the context of a fungal pyridoxal phosphate (PLP)-dependent tryptophan decarboxylase derived from the class Eurotiomycetes includes PLP-dependent tryptophan decarboxylases obtained from fungal organisms belonging to the class Eurotiomycetes, and to variants of such PLP-dependent tryptophan decarboxylases. The PLP-dependent tryptophan decarboxylase may be a wild-type or native enzyme found in a Eurotiomycete, or a mutant, derivative or variant form thereof. The terms “wild-type”, “native” and “naturally occurring” are used interchangeably herein to refer to a gene or gene product that has the characteristics of that gene or gene product when isolated from a naturally occurring source. A wild type, native or naturally occurring gene or gene product (e.g., a protein or polypeptide) is that which is most frequently observed in a population and is thus arbitrarily designed the “normal” or “wild-type” form of the gene or gene product. As will be understood by the skilled person, the terms a “fungal PLP-dependent tryptophan decarboxylase”, a “fungal PLP-dependent tryptophan decarboxylase derived from the class Eurotiomycetes” and a “PLP-dependent tryptophan decarboxylase derived from the class Eurotiomycetes” are used interchangeably herein.

As would be understood by the skilled person, the terms “pyridoxal phosphate (PLP)-independent tryptophan decarboxylase” and “non-PLP-dependent tryptophan decarboxylase” can be used interchangeable and refers to a tryptophan decarboxylase that does not require active site binding of pyridoxal 5′ phosphate (PLP) for catalytic activity.

As used herein the terms “variant” and “variants” refer to substantially similar sequences. Variants may be naturally occurring or artificially generated. A variant of a fungal PLP-dependent tryptophan decarboxylase may share at least about 80%, 81%, 82%, 83%, 84%, 85%, 86%, 87%, 88%, 89%, 90%, 91%, 92%, 93%, 94%, 95%, 96%, 97%, 98% or 99% sequence identity with the fungal PLP-dependent tryptophan decarboxylase of which it is a variant, such as for example the decarboxylase comprising an amino acid sequence of any one of SEQ ID NOs: 1, 3, 5 or 7. Generally, variant polypeptides and proteins also possess qualitative biological activity in common, in particular catalytic activity. A variant of a fungal PLP-dependent tryptophan decarboxylase of the present disclosure may comprise one or more conservative amino acid substitutions with respect to the fungal PLP-dependent tryptophan decarboxylase.

A variant of a PLP-dependent tryptophan decarboxylase may share at least about 80%, 81%, 82%, 83%, 84%, 85%, 86%, 87%, 88%, 89%, 90%, 91%, 92%, 93%, 94%, 95%, 96%, 97%, 98% or 99% sequence identity with the PLP-dependent tryptophan decarboxylase of which it is a variant, such as for example the decarboxylase comprising an amino acid sequence of any one of SEQ ID NOs: 29 or 48. Generally, variant polypeptides and proteins also possess qualitative biological activity in common, in particular catalytic activity. A variant of a fungal PLP-dependent tryptophan decarboxylase of the present disclosure may comprise one or more conservative amino acid substitutions with respect to the PLP-dependent tryptophan decarboxylase.

A “conservative amino acid substitution” is one in which the amino acid residue is replaced with an amino acid residue having a similar side chain. Families of amino acid residues having similar side chains have been defined in the art. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartic acid with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity.

Suitable examples of amino acid changes to the tryptophan decarboxylases are set forth in US2023/0357744 and WO2021/248087. It will be appreciated that the scope of the present disclosure encompasses such amino acid changes.

In some embodiments, amino acid changes to the tryptophan decarboxylases described herein are produced by directed evolution (e.g., unnatural sequences produced by sequence optimisation).

The terms “protein” and “polypeptide” are used interchangeably herein. A polypeptide or protein that has a non-polypeptide moiety covalently or non-covalently associated therewith is still considered a “polypeptide”. Exemplary modifications include glycosylation and palmitoylation. Polypeptides and proteins may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (e.g., the succession of letters or three letter codes used as abbreviations for amino acid names) that biochemically characterizes a polypeptide. A polypeptide sequence presented herein is presented in an N-terminal to C-terminal direction unless otherwise indicated.

The term “polynucleotide” is used herein interchangeably with “nucleic acid”, “nucleic acid molecule” and “nucleotide sequence” and “gene” to indicate a polymer of nucleosides. Typically, a polynucleotide of present disclosure is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” or “nucleotide sequence” as used herein can refer to the polynucleotide material itself and/or to the sequence information (e.g., the succession of letters used as abbreviations for bases) that biochemically characterizes a specific nucleic acid. A polynucleotide sequence presented herein is presented in a 5′ to 3′ direction unless otherwise indicated.

The term “nucleic acid construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present disclosure will generally include the necessary elements to direct expression of a nucleic acid sequence of interest that is also contained in the construct, such as, for example, a target nucleic acid sequence or a modulator nucleic acid sequence. Such elements may include control elements such as a promoter that is operably linked to (so as to direct transcription of) the nucleic acid sequence of interest, and often also includes a polyadenylation sequence. Within certain embodiments of the disclosure, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the methods of the present disclosure, conventional compositions and methods for preparing and using constructs and host cells are well known to one skilled in the art, see for example, Molecular Cloning: A Laboratory Manual, 3^{rd} edition Volumes 1, 2, and 3. J. F. Sambrook, D. W. Russell, and N. Irwin, Cold Spring Harbor Laboratory Press, 2000.

As used herein the term “derivative”, in the context of a tryptophan derivative, means a compound that is produced biosynthetically, either directly or indirectly, from tryptophan in one or more enzymatically catalyzed steps. That is, the compound is part of the same biosynthetic pathway as tryptophan, and downstream of tryptophan in that biosynthetic pathway.

Similarly, the term “derived” in the context of a tryptamine-derived compound or alkaloid as described herein, refers to a compound that is produced biosynthetically, either directly or indirectly, from tryptamine in one or more enzymatically catalyzed steps. That is, the compound is part of the same biosynthetic pathway as tryptamine, and downstream of tryptamine in that biosynthetic pathway.

As used herein, a “vector” includes reference to both polynucleotide vectors and viral vectors, each of which are capable of delivering a transgene contained within the vector into a host cell. Vectors can be episomal, i.e., do not integrate into the genome of a host cell, or can integrate into the host cell genome. The vectors may also be replication competent or replication deficient. Exemplary polynucleotide vectors include, but are not limited to, plasmids, cosmids and transposons. Exemplary viral vectors include, for example, AAV, lentiviral, retroviral, adenoviral, herpes viral and hepatitis viral vectors.

As used herein, the term “operably linked” refers to functional linkage between a nucleic acid expression control sequence (such as a promoter, signal sequence, enhancer or array of transcription factor binding sites) and a coding sequence (e.g., of an exogenous gene as described herein) wherein the expression control sequence affects transcription and/or translation of the coding sequence.

The term “host cell” refers to a cell, such as a yeast cell, that has introduced into it an exogenous gene as described herein, such as in a vector or other polynucleotide. The term includes the progeny of the original cell into which the exogenous gene has been introduced. Thus, a “host cell” as used herein generally refers to a cell that has been transfected or transduced with exogenous DNA.

As used herein, “isolated” with reference to a polynucleotide or polypeptide means that the polynucleotide or polypeptide is substantially free of cellular material or other contaminating proteins from the cells from which the polynucleotide or polypeptide is derived, or substantially free from chemical precursors or other chemicals when chemically synthesized. As used herein, the term “purified” when used in connection with tryptophan derivatives disclosed herein extracted from cells or cell growth or culture medium, means that that the extracted tryptophan derivative has been subjected to one or more processing steps of increase the purity of the tryptophan derivative.

As used herein, the term “subject” includes any mammal, such as humans, non-human primates, livestock animals (e.g., sheep, pigs, cattle, horses, donkeys, goats), laboratory test animals (e.g., mice, rabbits, rats, guinea pigs, other rodents), companion animals (e.g., dogs, cats). In preferred embodiments, the subject is a human.

As used herein, the terms “treatment”, “treating”, and the like, refer to obtaining a desired pharmacologic and/or physiologic effect in a subject in need of treatment, that is, a subject who has a disease or disorder. By “treatment” is meant ameliorating or preventing one or more symptoms or effects (e.g., consequences) of a disease or disorder. Reference to “treatment”, “treat” or “treating” does not necessarily mean to reverse or prevent any or all symptoms or effects of a disease or disorder. For example, the subject may ultimately suffer one or more symptoms or effects, but the number and/or severity of the symptoms or effects is reduced and/or the quality of life is improved compared to prior to treatment.

As used herein, the term “nootropic” will be understood to refer to any natural, semi-synthetic, bio-synthetic, and synthetic neuroprotectant or cognitive enhancing compounds.

As used herein, the term “functional food” will be understood to refer to a consumable that can beneficially modulate one or more targeted functions in a subject (e.g., enhancing a physiological response and/or by reducing the risk of disease).

It will be appreciated that the above described terms and associated definitions are used for the purpose of explanation only and are not intended to be limiting.

Each embodiment described herein is to be applied mutatis mutandis to each and every embodiment unless specifically stated otherwise.

TABLE 1
Brief description of sequences described herein

| SEQ | |
|---|---|
| ID NO: | Description |
| 1 | CnsB polypeptide from Penicillium expansum (GenBank: |
| | A0A0A2IDH4.1) (herein “CnsBv1”) |
| 2 | cnsBv1 coding sequence (5′-3′) encoding cnsBv1 of SEQ ID |
| | NO: 1 (GenBank: JQFZ01000090) |
| 3 | CnsB polypeptide from Penicillium expansum (GenBank: |
| | AMQ76109.1) (herein “CnsBv2”) |
| 4 | cnsBv2 coding sequence (5′-3′) encoding CnsBv2 of SEQ ID |
| | NO: 3 (GenBank: KU932036.1) |
| 5 | CnsB polypeptide (28 aa truncation of SEQ ID NO: 3) (herein |
| | “ΔCnsBv2”) |
| 6 | ΔcnsBv2 coding sequence (5′-3′) encoding ΔCnsBv2 of SEQ |
| | ID NO: 5 |
| 7 | Tryptophan decarboxylase from Aspergillus steynii (GenBank: |
| | XP_024705640) (herein “AsTDC1”) |
| 8 | asTDC1 coding sequence (5′-3′) encoding asTDC1 of SEQ ID |
| | NO: 7 (GenBank:XM_024851485.1) |
| 9 | PsiD polypeptide (GenBank: ASU62239.1) |
| 10 | psiD coding sequence (5′-3′) encoding PsiD of SEQ ID NO: 9 |
| | (GenBank: KY984101.1) |
| 11 | PsiH polypeptide (GenBank: ASU62246.1) |
| 12 | psiH coding sequence (5′-3′) encoding PsiH of SEQ ID NO: |
| | 11 (GenBank: MF000993.1) |
| 13 | PsiK polypeptide (GenBank: ASU62237.1) |
| 14 | psiK coding sequence (5′-3′) encoding PsiK of SEQ ID NO: |
| | 13 (GenBank: KY984099.1) |
| 15 | PsiM polypeptide (GenBank: ASU62238.1) |
| 16 | psiM coding sequence (5′-3′) encoding SEQ PsiM of ID NO: |
| | 15 (GenBank: KY984100.1) |
| 17 | PcCpr polypeptide |
| 18 | Pccpr coding sequence (5′-3′) encoding PcCpr of SEQ ID NO: |
| | 17 From Milne et al, Metab Eng, 60, 25-36. |
| 19 | Sah1 polypeptide |
| | (GenBank: NP_010961.3) |
| 20 | SAH1 coding sequence (5′-3′) encoding Sah1 of SEQ ID NO: |
| | 19 (GenBank: NM_001178934.3) |
| 21 | Ado1 polypeptide |
| | (GenBank: NP_012639.1) |
| 22 | ADO1 coding sequence encoding Ado1 of SEQ ID NO: 21 |
| | (GenBank: NM_001181763.1) |
| 23 | cnsBv1 coding sequence (5′-3′) - codon optimized for |
| | expression in S. cerevisiae encoding CnsBv1 of SEQ ID NO: 1 |
| 24 | psiD coding sequence (5′-3′) - codon optimized for expression |
| | in S. cerevisiae encoding PsiD of SEQ ID NO: 9 |
| 25 | psiH coding sequence (5′-3′) - codon optimized for expression |
| | in S. cerevisiae encoding PsiH of SEQ ID NO: 11 |
| 26 | psiK coding sequence (5′-3′) - codon optimized for expression |
| | in S. cerevisiae encoding PsiK of SEQ ID NO: 13 |
| 27 | psiM coding sequence (5′-3′) - codon optimized for expression |
| | in S. cerevisiae encoding PsiM of SEQ ID NO: 15 |
| 28 | Pccpr coding sequence (5′-3′) - codon optimized for expression |
| | in S. cerevisiae encoding PcCpr of SEQ ID NO: 17 |
| 29 | CrTDC polypeptide (GenBank: AYA72254.1) |
| 30 | crTDC coding sequence (5′-3′) encoding CrTDC of SEQ ID |
| | NO: 29 (GenBank: MG748691.1) |
| 31 | asTDC coding sequence (5′-3′) - codon optimized for expression |
| | in Saccharomyces cerevisiae encoding AsTDC of SEQ ID NO: 7 |
| 32 | SAH1F primer |
| 33 | SAH1R primer |
| 34 | ADO1F primer |
| 35 | ADO1R primer |
| 36 | OsT5H polypeptide (GenBank: NP_001410381) |
| 37 | osT5H coding sequence (5′-3′) encoding OsT5H of SEQ ID NO: |
| | 36 (GenBank: NM_001423452) |
| 38 | osT5H, coding sequence (5′-3′), codon optimized, encoding |
| | OsT5H of SEQ ID NO: 36 |
| 39 | OsCPR polypeptide (GenBank: XP_015636692) |
| 40 | osCPR coding sequence (5′-3′) encoding |
| | OsCPR of SEQ ID NO: 38 (GenBank: XM_015781206) |
| 41 | osCPR, coding sequence (5′-3′), codon optimized, encoding |
| | OsCPR of SEQ ID NO: 38 |
| 42 | RmNMT polypeptide (GenBank: WKR38372) |
| 43 | rmNMT coding sequence (5′-3′) encoding RmNMT of SEQ ID |
| | NO: 42 (GenBank: OQ557631) |
| 44 | rmNMT, coding sequence (5′-3′), codon optimized, encoding |
| | RmNMT of SEQ ID NO: 42 |
| 45 | psiD, coding sequence (5′-3′), codon optimized for E. coli, |
| | encoding PsiD of SEQ ID NO: 9 |
| 46 | asTDC1, coding sequence (5′-3′), codon optimized for E. coli, |
| | encoding asTDC1 of SEQ ID NO: 7 |
| 47 | crTDC coding sequence (5′-3′), codon optimized, encoding |
| | CrTDC of SEQ ID NO: 29 |
| 48 | Tryptophan decarboxylase from Aspergillus caelatus (GenBank: |
| | XP_031931155) (herein “AcTDC”) |
| 49 | acTDC coding sequence (5′-3′) encoding AcTDC of SEQ ID |
| | NO: 48 (GenBank: XM_032069937) |
| 50 | acTDC coding sequence (5′-3′), codon optimized, encoding |
| | AcTDC of SEQ ID NO: 48 |
| 51 | cnsBv2 coding sequence (5′-3′), codon optimized, encoding |
| | CnsBv2 of SEQ ID NO: 3 |

### PLP-Dependent Tryptophan Decarboxylase-Expressing Cells

With the elucidation of the biosynthetic pathways for the production of secondary metabolites derived from tryptophan, and of the genes responsible for the production of various intermediates in such pathways, some attention has been devoted to the manipulation of these pathways in an effort to increase or improve the production of various tryptophan derivatives, including with the heterologous in vivo expression of one or more enzymes operative in the pathway in suitable host cells. Most attention to date has been focussed on the use of the PsiD, PsiH, PsiK and PsiM enzymes, in particular those derived from Psilocybe cubensis (e.g., PcPsiD, PcPsiH, PcPsiK and PcPsiM), in for the production of psilocybin.

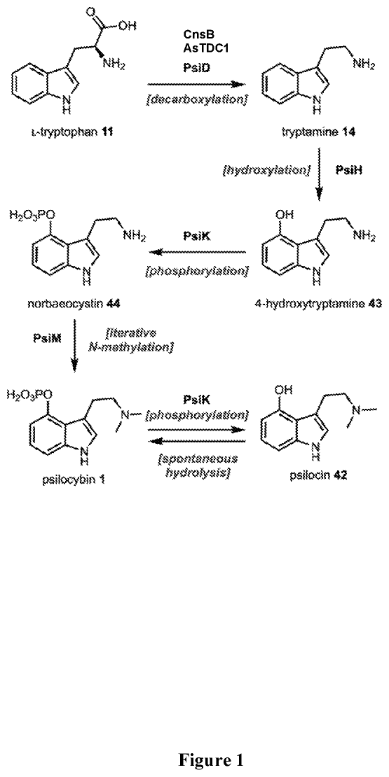

The biosynthetic pathway for the production of psilocybin from tryptophan, illustrating the roles of the PsiD, PsiH, PsiK and PsiM enzymes, is shown in FIG. 1. PsiD is a PLP-independent tryptophan decarboxylase that catalyses the decarboxylation of an aliphatic carboxylic acid converting L-trytophan to tryptamine. PsiH is a monooxygenase catalysing the oxidative hydroxylation of the phenyl ring of tryptamine to 4-hydroxytryptamine. PsiH belongs to a superfamily of heme-containing monooxygenases that typically require a cytochrome P450 reductase partner (CPR) for efficient catalysis. PsiK is a kinase that catalyses the phosphorylation of the phenolic oxygen of 4-hydroxytryptamine to norbaeocystin, as well as the later phosphorylation of psilocin to psilocybin. PsiM is a methyl transferase catalysing the alkylation of the primary amine in norbaeocystin to baecystin, and of the secondary amine of baecystin to become a tertiary amine of psilocybin. U.S. Pat. No. 11,441,164 describes methods for the production of psilocybin in recombinant organisms expressing genes, codon optimized for expression in the host organism, encoding PsiD, PsiH, PsiK and PsiM.

Penicillium expansum (phylum Ascomycota, subphylum Pezizomycotina, class Eurotiomycetes) is a blue mold fungus, a post-harvest pathogen of fruit such as apples and the causative agent of blue rot disease. Lin et al., 2015 (Angew Chem Int Ed Engl, 54: 3004-7) elucidated the genes of the biosynthetic pathway of communesins from L-tryptophan in P. expansum, which includes the cnsB decarboxylase genes described herein, encoding a PLP-dependent tryptophan decarboxylase. The amino acid sequences of CnsB polypeptides derived from P. expansum are in Uniprot Accession No. A0A0A2IDH4, set forth herein in SEQ ID NO: 1 (and termed herein ‘CnsBv1’) and in GenBank Accession No. AMQ76109.1, is set forth herein in SEQ ID NO: 3 (and termed herein ‘CnsBv2’). The nucleotide sequences of the cnsB genes encoding CnsBv1 and CnsB2 are set forth in SEQ ID NO: 2 and SEQ ID NO: 4, respectively.

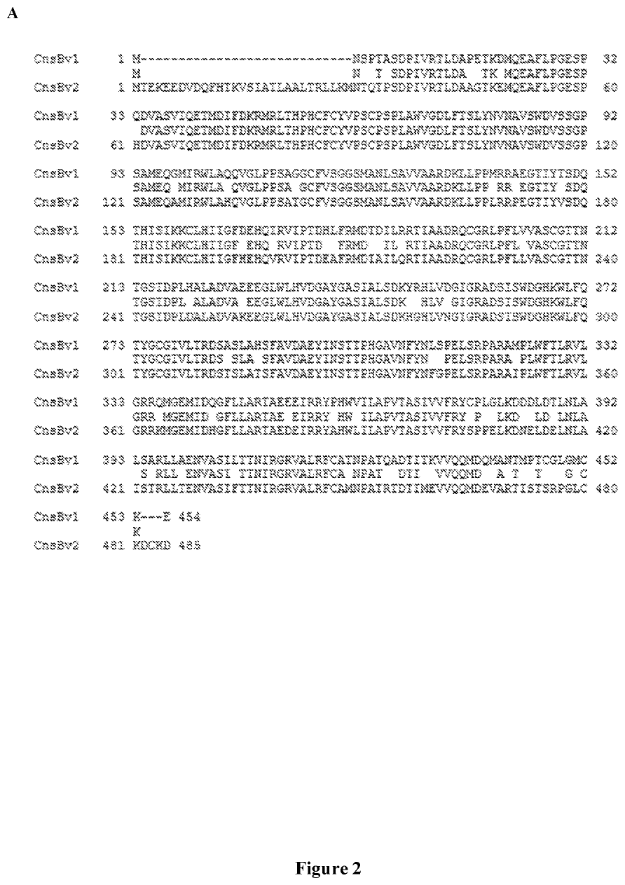

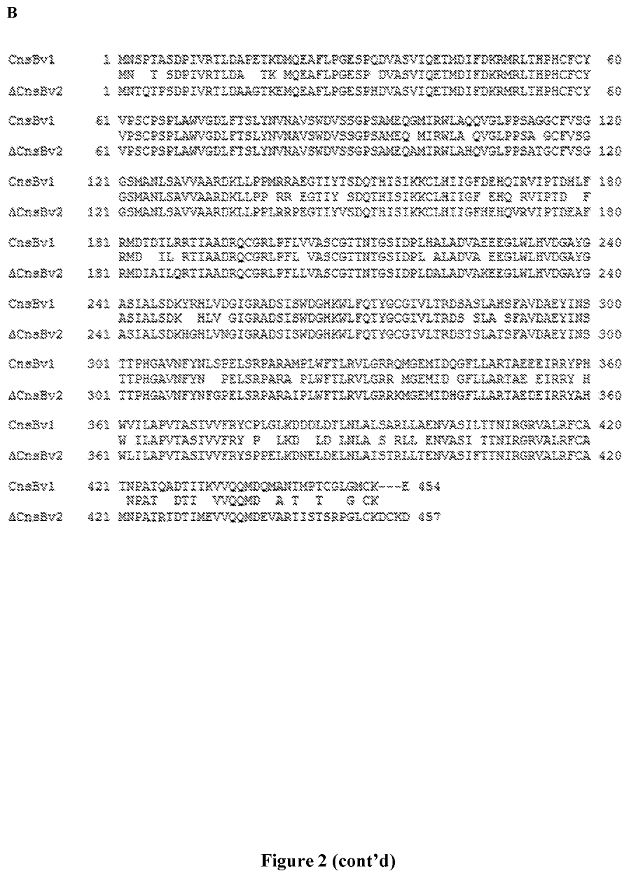

The inventors have also identified an active, truncated form of CnsBv2 (termed herein ‘ΔCnsBv2’), in which the 28 amino acid residues from the N-terminus of CnsBv2 are absent. The amino acid sequence of ΔCnsBv2 is set forth in SEQ ID NO: 5, and the encoding nucleotide sequence is set forth in SEQ ID NO: 6. CnsBv1 and CnsBv2 share 81% sequence identity across the full length of their amino acid sequences (FIG. 2A), while CnsBv1 and ΔCnsBv2 share 86% sequence identity (FIG. 2B).

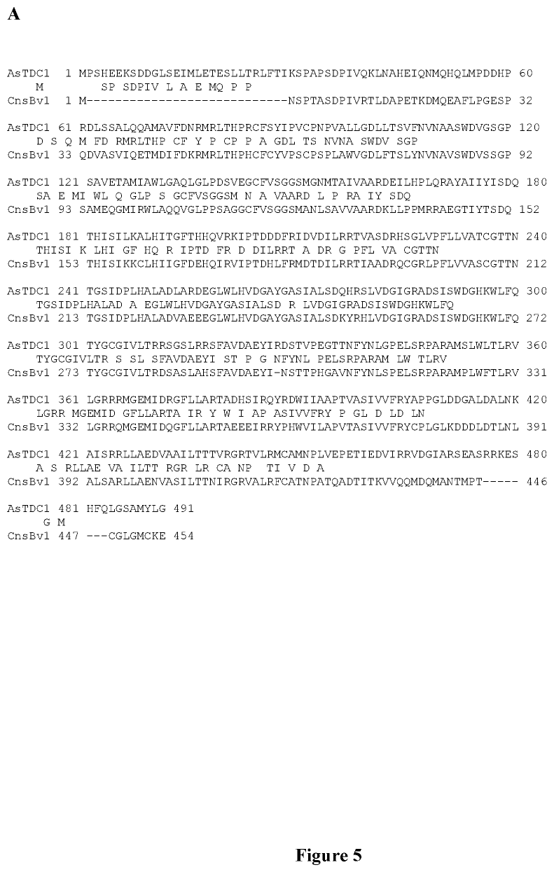

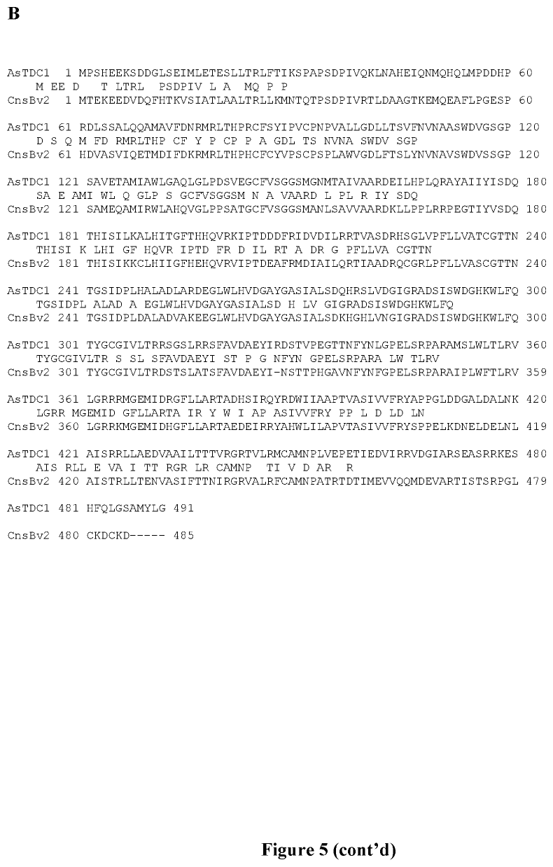

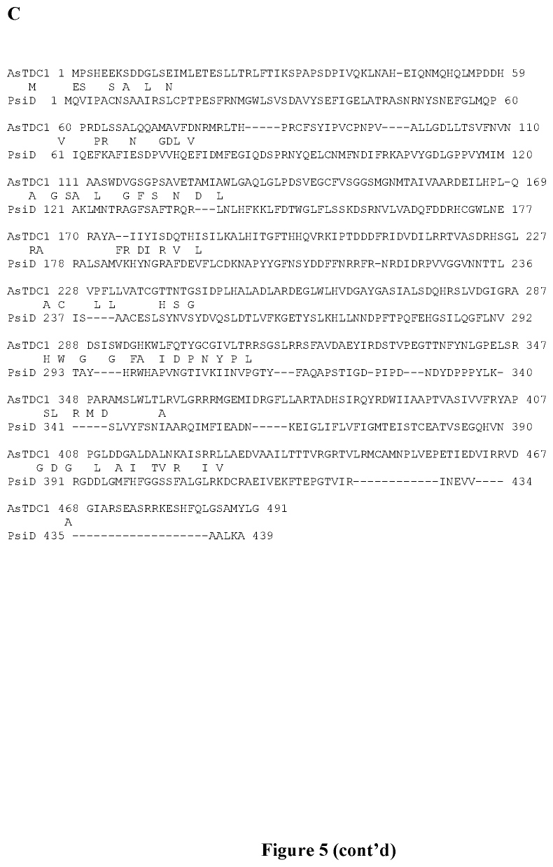

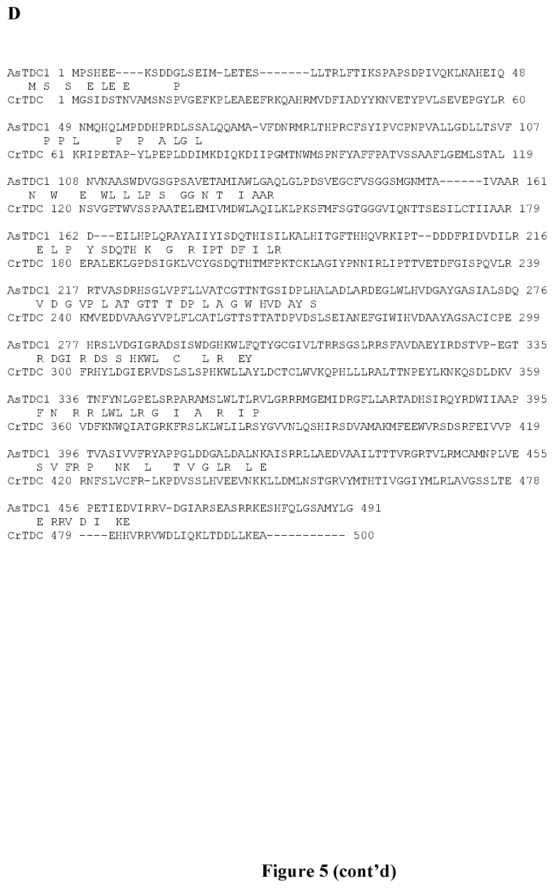

Further, the inventors have identified a PLP-dependent tryptophan decarboxylase (termed herein “AsTDC1”) from Aspergillus steynii (phylum Ascomycota, subphylum Pezizomycotina, class Eurotiomycetes). The amino acid sequence of the AsTDC1 polypeptide is set forth in SEQ ID NO: 7, and the encoding nucleotide sequence is set forth in SEQ ID NO: 8. asTDC1 shares 60.9% sequence identity with CnsBv1 and 62.1% sequence identity with CnsBv2 across the full length of their amino acid sequences (see FIG. 5).

Further, the inventors have identified a PLP-dependent tryptophan decarboxylase (termed herein “CrTDC”) from Catharanthus roseus (class Dicotyledons). The amino acid sequence of the CrTDC polypeptide is set forth in SEQ ID NO: 29, and the encoding nucleotide sequence is set forth in SEQ ID NO: 30.

Further, the inventors have identified a PLP-dependent tryptophan decarboxylase (termed herein “AcTDC”) from Aspergillus caelatus (phylum Ascomycota, subphylum Pezizomycotina, class Eurotiomycetes). The amino acid sequence of the AcTDC polypeptide is set forth in SEQ ID NO: 48.

The inventors have also identified various amino acid sequences that are conserved in the PLP-dependent tryptophan decarboxylase enzymes described herein (e.g., AsTDC1, AcTDC) that are enzymatically active and have confirmed that these conserved sequences are also found in other PLP-dependent tryptophan decarboxylases (e.g., non-Eurotiomycete fungi, bacterial and plants).

Accordingly, embodiments of the present disclosure relate to PLP-dependent tryptophan decarboxylases comprising one or all of the conserved sequences described herein, and to uses thereof.

Accordingly, embodiments of the present disclosure relate to fungal PLP-dependent tryptophan decarboxylases derived from members of the class Eurotiomycetes, and to uses thereof. Embodiments of the present disclosure also relate to PLP-dependent tryptophan decarboxylases that are at least about 80%, 81%, 82%, 83%, 84%, 85%, 86%, 87%, 88%, 89%, 90%, 91%, 92%, 93%, 94%, 95%, 96%, 97%, 98% or 99% identical to fungal PLP-dependent tryptophan decarboxylases derived from members of the class Eurotiomycetes, and to uses thereof. Aspects and embodiments of the present disclosure also relate to PLP-dependent tryptophan decarboxylases comprising one or more conservative amino acid substitutions with respect to fungal PLP-dependent tryptophan decarboxylases derived from members of the class Eurotiomycetes, and to uses thereof.

Fungi of the class Eurotiomycetes may belong, for example, to a subclass selected from Eurotiomycetidae, Mycocaliciomycetidae, Coryneliomycetidae, Sclerococcomycetidae, Cryptocaliciomycetidae, and Chaetothyriomycetidae. In exemplary embodiments, a fungal PLP-dependent tryptophan decarboxylase of the present disclosure is derived from a member of the subclass Eurotiomycetidae.

Embodiments of the present disclosure also relate to PLP-dependent tryptophan decarboxylases derived from members of the phylum Ascomycota and/or the phylum Basidiomycota, and to uses thereof. Embodiments of the present disclosure also relate to PLP-dependent tryptophan decarboxylases that are at least about 80%, 81%, 82%, 83%, 84%, 85%, 86%, 87%, 88%, 89%, 90%, 91%, 92%, 93%, 94%, 95%, 96%, 97%, 98% or 99% identical to PLP-dependent tryptophan decarboxylases derived from members of the phylum Ascomycota and/or the phylum Basidiomycota, and to uses thereof. Embodiments of the present disclosure also relate to PLP-dependent tryptophan decarboxylases comprising one or more conservative amino acid substitutions with respect to PLP-dependent tryptophan decarboxylases derived from members of the phylum Basidiomycota and/or the phylum Ascomycota, and to uses thereof.

Embodiments of the present disclosure also relate to PLP-dependent tryptophan decarboxylases derived from members of the class Dicotyledons, and to uses thereof. Embodiments of the present disclosure also relate to PLP-dependent tryptophan decarboxylases that are at least about 80%, 81%, 82%, 83%, 84%, 85%, 86%, 87%, 88%, 89%, 90%, 91%, 92%, 93%, 94%, 95%, 96%, 97%, 98% or 99% identical to PLP-dependent tryptophan decarboxylases derived from members of the class Dicotyledons, and to uses thereof. Embodiments of the present disclosure also relate to PLP-dependent tryptophan decarboxylases comprising one or more conservative amino acid substitutions with respect to PLP-dependent tryptophan decarboxylases derived from members of the class Dicotyledons, and to uses thereof.

Embodiments of the present disclosure also relate to PLP-dependent tryptophan decarboxylases derived from bacteria, and to uses thereof. Embodiments of the present disclosure also relate to PLP-dependent tryptophan decarboxylases that are at least about 80%, 81%, 82%, 83%, 84%, 85%, 86%, 87%, 88%, 89%, 90%, 91%, 92%, 93%, 94%, 95%, 96%, 97%, 98% or 99% identical to PLP-dependent tryptophan decarboxylases derived from bacteria, and to uses thereof. Embodiments of the present disclosure also relate to PLP-dependent tryptophan decarboxylases comprising one or more conservative amino acid substitutions with respect to PLP-dependent tryptophan decarboxylases derived from bacteria, and to uses thereof.

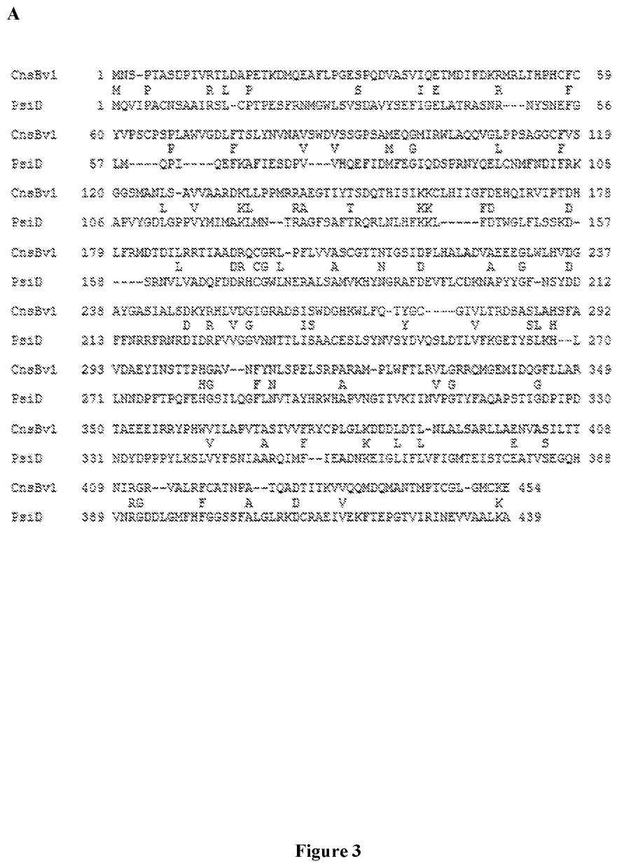

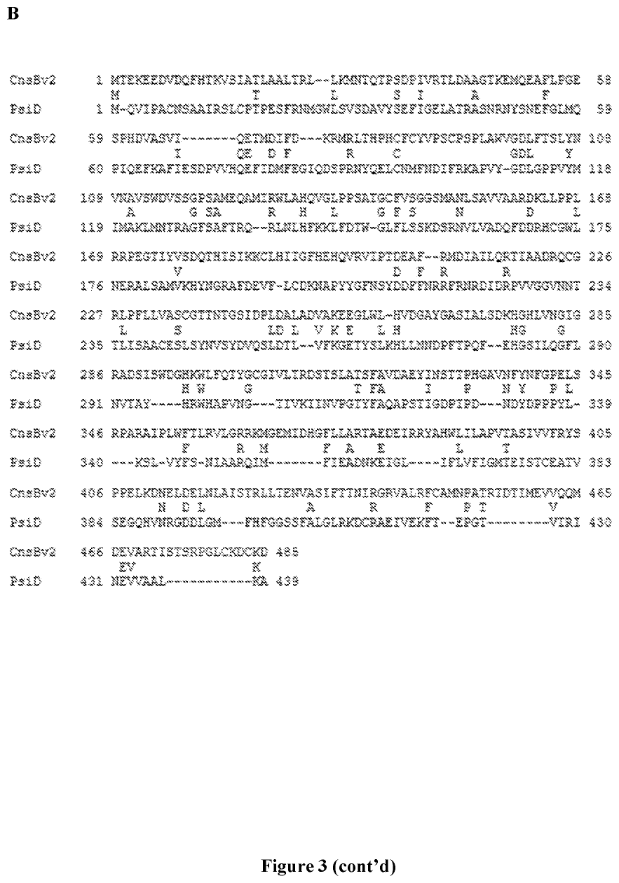

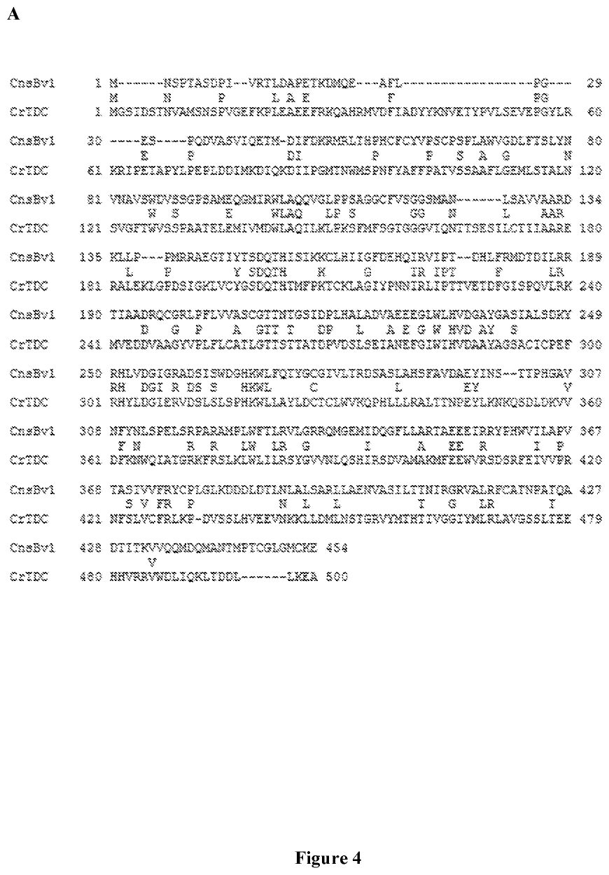

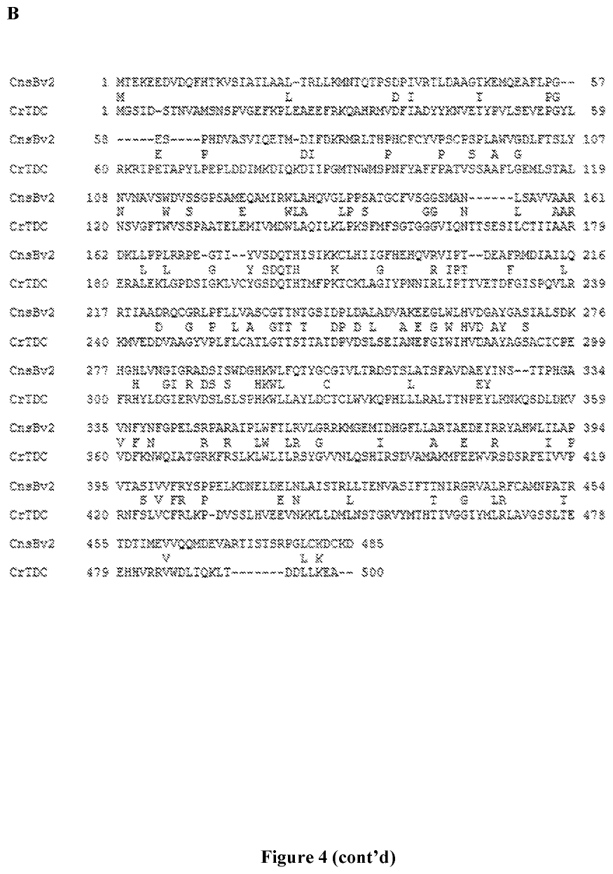

The present inventors have surprisingly found that modifying the yeast S. cerevisiae to express CnsBv1 or AsTDC1 significantly increases the expression of tryptamine when compared to a S. cerevisiae strain expressing PsiD from Psilocybe cubensis (PcPsiD; SEQ ID NO: 9). The PLP-dependent tryptophan decarboxylases of the present disclosure are only very distantly related to the non-PLP-dependent PcPsiD tryptophan decarboxylase. CnsBv1 and PcPsiD share only 16% sequence identity, CnsBv2 and PcPsiD share only 16.2% sequence identity, and ΔCnsBv2 and PcPsiD share only 15.3% sequence identity (see FIG. 3). Similarly, AsTDC1 and PcPsiD share only 14% sequence identity (see FIG. 5). The fungal PLP-dependent tryptophan decarboxylases of the present disclosure are also only distantly related to plant PLP-dependent tryptophan decarboxylases, such as CrTDC. CnsBv1 and CnsBv2 share only 24.3% and 23.5% sequence identity, respectively, with CrTDC (FIG. 4). AsTDC1 and CrTDC1 share only 25% sequence identity (FIG. 5).

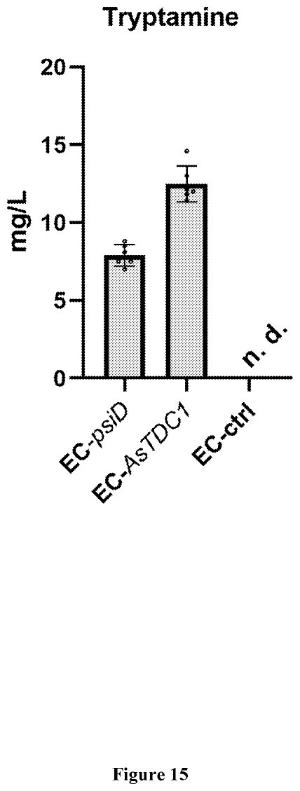

The present inventors have also surprisingly found that modifying the bacterial cell E. coli to express AsTDC1 significantly increases the expression of tryptamine when compared to a E. coli strain expressing PsiD from Psilocybe cubensis (FIG. 15). The PLP-dependent tryptophan decarboxylases of the present disclosure are only very distantly related to the non-PLP-dependent PcPsiD tryptophan decarboxylase.

The present disclosure provides a cell comprising an exogenous gene and capable of producing at least one tryptophan derivative, wherein the exogenous gene encodes a fungal pyridoxal phosphate (PLP)-dependent tryptophan decarboxylase derived from a member of the class Eurotiomycetes, or a PLP-dependent tryptophan decarboxylase at least about 80% identical to, or comprising one or more conservative amino acid substitutions with respect to, said fungal PLP-dependent tryptophan decarboxylase, and wherein the level of production of the at least one tryptophan derivative in the cell is increased relative to the level of production of the at least one tryptophan derivative in an equivalent cell that does not possess the exogenous gene and that comprises a non-PLP-dependent tryptophan decarboxylase, optionally PsiD. The cell may comprise one or more copies of a gene encoding said PLP-dependent tryptophan decarboxylase. Optionally, the equivalent cell comprises the PsiD tryptophan decarboxylase having the amino acid sequence of SEQ ID NO: 9. Optionally, the at least one tryptophan derivative the production of which is increased in a cell of the disclosure, is tryptamine or psilocybin.

The present disclosure also provides a cell comprising an exogenous gene and capable of producing at least one tryptophan derivative, wherein the exogenous gene encodes a PLP-dependent tryptophan decarboxylase having an amino acid sequence set forth in SEQ ID NO: 1, SEQ ID NO: 3 or SEQ ID NO: 5, or a sequence having at least about 70% sequence identity thereto, and wherein the level of production of the at least one tryptophan derivative in the cell is increased relative to the level of production of the at least one tryptophan derivative in an equivalent cell that does not possess the exogenous gene and that comprises a non-PLP-dependent tryptophan decarboxylase, optionally PsiD. The cell may comprise one or more copies of a gene encoding said PLP-dependent tryptophan decarboxylase. Optionally, the equivalent cell comprises the PsiD tryptophan decarboxylase having the amino acid sequence of SEQ ID NO: 9. Optionally, the at least one tryptophan derivative the production of which is increased in a cell of the disclosure, is tryptamine or psilocybin.

The present disclosure additionally provides a cell comprising an exogenous gene and capable of producing at least one tryptophan derivative, wherein the exogenous gene encodes a PLP-dependent tryptophan decarboxylase having an amino acid sequence set forth in SEQ ID NO: 7, or a sequence having at least about 70% sequence identity thereto, and wherein the level of production of the at least one tryptophan derivative in the cell is increased relative to the level of production of the at least one tryptophan derivative in an equivalent cell that does not possess the exogenous gene and that comprises a non-PLP-dependent tryptophan decarboxylase, optionally PsiD. The cell may comprise one or more copies of a gene encoding said PLP-dependent tryptophan decarboxylase. Optionally, the equivalent cell comprises the PsiD tryptophan decarboxylase having the amino acid sequence of SEQ ID NO: 9. Optionally, the at least one tryptophan derivative the production of which is increased in a cell of the disclosure, is tryptamine or psilocybin.

The present disclosure also provides a cell capable of producing at least one tryptophan derivative comprising at least one exogenous polynucleotide encoding a pyridoxal phosphate (PLP)-dependent tryptophan decarboxylase, wherein the level of production of the at least one tryptophan derivative in the cell is increased relative to the level of production of a second tryptophan derivative in an equivalent cell lacking the exogenous polynucleotide or comprising a second polynucleotide encoding a second tryptophan decarboxylase, and wherein the second polynucleotide is not the same as the exogenous polynucleotide.

The present disclosure also provides a cell capable of producing at least one tryptophan derivative comprising at least one exogenous polynucleotide encoding a pyridoxal phosphate (PLP)-dependent tryptophan decarboxylase, wherein the level of production of the at least one tryptophan derivative in the cell is increased relative to the level of production of a second tryptophan derivative in an equivalent cell lacking the exogenous polynucleotide or comprising a second polynucleotide encoding a second tryptophan decarboxylase, and wherein the second polynucleotide is not the same as the exogenous polynucleotide and wherein the PLP-dependent tryptophan decarboxylase comprises one or more or all of:
a) a first amino acid sequence of GX₁X₂X₃X₄X₅X₆X₇X₈X₉X₁₀X₁₁X₁₂X₁₃X₁₄X₁₅X₁₆X₁₇R wherein:
   X₁, X₂, X₃, X₄, X₈, X₉, X₁₀, X₁₁, X₁₂, X₁₃, X₁₄, X₁₅, and X₁₆ are any amino acid residue,
   X₅ and X₆ are an amino acid residue selected from the group consisting of glycine, alanine, serine and threonine,
   X₇ is an amino acid residue selected from the group consisting of serine and threonine, and
   X₁₇ is an amino acid residue selected from the group consisting of alanine and glycine;
b) a second amino acid sequence of X₁X₂X₃X₄X₅X₆HX₇X₈X₉X₁₀ KX₁₁X₁₂X₁₃X₁₄X₁₅X₁₆ wherein:
   X₁ is any amino acid residue selected from the group consisting of tyrosine, leucine, phenylalanine, valine, cysteine and tryptophan,
   X₂, X₄, X₅, X₆, X₇, X₈, X₉, X₁₀, X₁₁, X₁₂, X₁₃, X₁₄ and X₁₅ are any amino acid residue,
   X₃ is any amino acid residue selected from the group consisting of serine and threonine, and
   X₁₆ is an amino acid residue selected from the group consisting of glycine, alanine, serine and threonine;
c) a third amino acid sequence of DX₁X₂X₃X₄X₅X₆X₇X₈X₉X₁₀X₁₁X₁₂X₁₃X₁₄GX₁₅X₁₆X₁₇X₁₈X₁₉X₂₀X₂₁D wherein:
   X₁, X₂, X₃, X₄, X₅, X₆, X₈, X₉, X₁₀, X₁₁, X₁₂, X₁₃, X₁₄, X₁₅, X₁₇, X₁₈, X₁₉ and X₂₁ are any amino acid residue, and
   X₇ is an amino acid residue selected from the group consisting of proline and asparagine,
   X₁₆ is an amino acid residue selected from the group consisting of threonine, proline and valine, and
   X₂₀ is 1, 2, 3, 4, 5, or 6 amino acid residues and is any amino acid residue;
d) a fourth amino acid sequence of X₁X₂HX₃X₄X₅X₆X₇X₈X₉X₁₀ wherein:
   X₁ is an amino acid residue selected from the group consisting of tryptophan and phenylalanine,
   X₂, X₃, X₆, X₉ and X₁₀ are any amino acid residue,
   X₄ is an amino acid residue selected from the group consisting of aspartic acid and glutamic acid,
   X₅ is an amino acid residue selected from the group consisting of glycine, alanine and serine,
   X₇ is an amino acid residue selected from the group consisting of phenylalanine, tyrosine and tryptophan, and
   X₈ is an amino acid residue selected from the group consisting of glycine, alanine, serine and threonine; and
e) a fifth amino acid sequence of X₁X₂X₃X₄X₅X₆X₇HKX₈X₉X₁₀X₁₁X₁₂X₁₃X₁₄X₁₅X₁₆ wherein:
   X₁ is an amino acid residue selected from the group consisting of aspartic acid, glutamic acid, histidine and asparagine,
   X₂ is an amino acid residue selected from the group consisting of serine and threonine,
   X₃, X₄, X₅, X₇, X₁₀, X₁₁, X₁₂, X₁₃ and X₁₄ are any amino acid residue,
   X₆ is an amino acid residue selected from the group consisting of aspartic acid, glutamic acid, asparagine, serine and threonine,
   X₈ is an amino acid residue selected from the group consisting of tryptophan, phenylalanine, tyrosine, leucine, methionine and cysteine,
   X₉ is an amino acid residue selected from the group consisting of leucine, isoleucine, valine, methionine and phenylalanine,
   X₁₅ is an amino acid residue selected from the group consisting of cysteine, alanine and serine, and
   X₁₆ is an amino acid residue selected from the group consisting of glycine, serine, alanine and threonine.

The present disclosure also provides a cell capable of producing at least one tryptophan derivative comprising at least one exogenous polynucleotide encoding an amino acid sequence set forth in any one of SEQ ID NOs:1, 3, 5, 7, 29 and 48 or a sequence having at least about 70% sequence identity thereto, wherein the level of production of the at least one tryptophan derivative in the cell is increased relative to the level of production of a second tryptophan derivative in an equivalent cell lacking the exogenous polynucleotide or comprising a second polynucleotide encoding a second tryptophan decarboxylase, and wherein the second polynucleotide is not the same as the exogenous polynucleotide

As used herein the term “equivalent cell” refers to a cell of the same type, derivation, origin and constituency (i.e., the same genomic, proteinaceaous and carbohydrate etc. make up) as a cell of the disclosure, with the exception that the cell does not comprise an exogenous gene encoding a PLP-dependent tryptophan carboxylase of the disclosure, and does comprise a non-PLP-dependent, optionally PsiD, tryptophan decarboxylase. The “equivalent cell” is therefore used for comparative purposes, specifically for determination of production levels of one or more tryptophan derivatives in the presence of a PLP-dependent tryptophan carboxylase of the disclosure and in the presence of a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto, optionally a PsiD tryptophan decarboxylase. Typically, a cell of the disclosure and an “equivalent cell” are maintained, stored, and cultured under the same conditions.

In accordance with the present disclosure the cell may be a eukaryotic cell, a bacterial cell or a microalgal cell, typically a eukaryotic cell. In particular embodiments, the cell is not from species from which the original amino acid sequence or polynucleotide is derived (e.g., the cell is heterologous to the amino acid sequence or polynucleotide). In particular embodiments, the cell is not a P. expansum cell or an A. steynii cell. In some embodiments, the cell is not a A. caelatus cell.

In an exemplary embodiment, the cell is a yeast cell. The yeast cell may, for example, belong to the genus Saccharomyces, Yarrowia, Kluyveromyces, Schizosaccharomyces, Zygosaccharomyces, Candida, Cryptococcus (such as C. aerius), Pichia (also known as Komagataella), Debaromyces, Zygosaccharomyces (such as Z. bailii), Torulaspora (such as T. delbrueckii), Brettanomyces (such as B. bruxellensis), Penicillium, Rhizopus, Fusarium, Fusdium, Hansenula, Gibberella, Mucor, Mortierella, or Trichoderma. In a particular exemplary embodiment the yeast is a Saccharomyces species, such as S. cerevisiae, S. kluyveri, S. bayanus, S. exiguus, S. sevazzi, S. uvarum or S. boulardii. In another exemplary embodiment, the yeast is a Yarrowia species, such as Y. lipolytica. In another exemplary embodiment, the yeast is a Kluyveromyces species, such as K. dogzhanskii, K. lactis, K. marxianus var. marxianus, or K. thermotolerans. In another exemplary embodiment, the yeast is a Schizosaccharomyces species, such as S. pombe, S. japonicus or S. cryophilus. In another exemplary embodiment, the yeast is a Pichia (Komagataella) species, such as P. pastoris, P. stipidis, P. sorbitophila or K. phaffi.

In an exemplary embodiment, the cell is a filamentous fungi cell. The filamentous fungus may be, for example, an Aspergillus, such as A. nidulans, A. oryzae, A. niger or A. terreus. The filamentous fungus may be, for example, a Penicillium, such as P. chrysogenum, P. rubens, P. expansum or P. paxilli.

Alternatively, the eukaryotic cell may be a plant cell. By way of non-limiting example only, the plant may be selected from a species belonging to the genus Arabidopsis, such as A. thaliana, a species belonging to the genus Zea, such as Z. mays, a species belonging to the genus Medicago, such as M. truncatula, a species belonging to the genus Nicotiana, such as N. tabacum, or a species belonging to the genus Glycine, such as G. max.

Alternatively, the cell may be a bacterial cell. By way of non-limiting example only, the bacteria may be selected from a species belonging to the genus Bacillus, such as B. subtilis, a species belonging to the genus Escherichia, such as E. coli, a species belonging to the genus Lactobacillus, such as L. casei, a species belonging to the genus Lactococcus, such as L. lactis, a species belonging to the genus Corynebacterium, such as C. glutamicum, a species belonging to the genus Acetobacter, a species belonging to the genus Acinetobacter, a species belonging to the genus Pseudomonas, such as P. putida, or a species belonging to the genus Streptomyces, such as S. coelicolor.

The PLP-dependent tryptophan decarboxylase encoded by the exogenous gene may comprise the amino acid sequence set forth in SEQ ID NO: 1, or a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the tryptophan decarboxylase sequence set forth in SEQ ID NO: 1. Embodiments of the present disclosure provide a PLP-dependent tryptophan decarboxylase comprising the amino acid sequence set forth in SEQ ID NO: 1. Embodiments of the present disclosure provide a PLP-dependent tryptophan decarboxylase comprising an amino acid sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 1.

The PLP-dependent tryptophan decarboxylase encoded by the exogenous gene may comprise the amino acid sequence set forth in SEQ ID NO: 3, or a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the tryptophan decarboxylase sequence set forth in SEQ ID NO: 3. Embodiments of the present disclosure provide a PLP-dependent tryptophan decarboxylase comprising the amino acid sequence set forth in SEQ ID NO: 3. Embodiments of the present disclosure provide a PLP-dependent tryptophan decarboxylase comprising an amino acid sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 3.

The PLP-dependent tryptophan decarboxylase encoded by the exogenous gene may comprise the amino acid sequence set forth in SEQ ID NO: 5, or a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the tryptophan decarboxylase sequence set forth in SEQ ID NO: 5. Embodiments of the present disclosure provide a PLP-dependent tryptophan decarboxylase comprising the amino acid sequence set forth in SEQ ID NO: 5. Embodiments of the present disclosure provide a PLP-dependent tryptophan decarboxylase comprising an amino acid sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 5.

The PLP-dependent tryptophan decarboxylase encoded by the exogenous gene may comprise the amino acid sequence set forth in SEQ ID NO: 7, or a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the tryptophan decarboxylase sequence set forth in SEQ ID NO: 7. Embodiments of the present disclosure provide a PLP-dependent tryptophan decarboxylase comprising the amino acid sequence set forth in SEQ ID NO: 7. Embodiments of the present disclosure provide a PLP-dependent tryptophan decarboxylase comprising an amino acid sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 7.

The PLP-dependent tryptophan decarboxylase encoded by the exogenous gene may comprise the amino acid sequence set forth in SEQ ID NO: 29, or a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the tryptophan decarboxylase sequence set forth in SEQ ID NO: 29. Embodiments of the present disclosure provide a PLP-dependent tryptophan decarboxylase comprising the amino acid sequence set forth in SEQ ID NO: 29. Embodiments of the present disclosure provide a PLP-dependent tryptophan decarboxylase comprising an amino acid sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 29.

The PLP-dependent tryptophan decarboxylase encoded by the exogenous gene may comprise the amino acid sequence set forth in SEQ ID NO: 48, or a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the tryptophan decarboxylase sequence set forth in SEQ ID NO: 48. Embodiments of the present disclosure provide a PLP-dependent tryptophan decarboxylase comprising the amino acid sequence set forth in SEQ ID NO: 48. Embodiments of the present disclosure provide a PLP-dependent tryptophan decarboxylase comprising an amino acid sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 48.

The exogenous gene encoding the PLP-dependent tryptophan decarboxylase may comprise the nucleic acid sequence set forth in SEQ ID NO: 2, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 2, or nucleic acid sequence complementary thereto. Embodiments of the present disclosure provide an isolated nucleotide sequence, and a PLP-dependent tryptophan decarboxylase encoded by the nucleic acid sequence, set forth in SEQ ID NO: 2, or nucleic acid sequence complementary thereto. Embodiments of the present disclosure provide an isolated nucleotide sequence, and a PLP-dependent tryptophan decarboxylase encoded by a nucleic acid sequence, having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 2, or nucleic acid sequence complementary thereto.

The exogenous gene encoding the PLP-dependent tryptophan decarboxylase may comprise the nucleic acid sequence set forth in SEQ ID NO: 4, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 4, or nucleic acid sequence complementary thereto. Embodiments of the present disclosure provide an isolated nucleotide sequence, and a PLP-dependent tryptophan decarboxylase encoded by the nucleic acid sequence, set forth in SEQ ID NO: 4, or nucleic acid sequence complementary thereto. Embodiments of the present disclosure provide an isolated nucleotide sequence, and a PLP-dependent tryptophan decarboxylase encoded by a nucleic acid sequence, having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 4, or nucleic acid sequence complementary thereto.

The exogenous gene encoding the PLP-dependent tryptophan decarboxylase may comprise the nucleic acid sequence set forth in SEQ ID NO: 6, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 6, or nucleic acid sequence complementary thereto. Embodiments of the present disclosure provide an isolated nucleotide sequence, and a PLP-dependent tryptophan decarboxylase encoded by the nucleic acid sequence, set forth in SEQ ID NO: 6, or nucleic acid sequence complementary thereto. Embodiments of the present disclosure provide an isolated nucleotide sequence, and a PLP-dependent tryptophan decarboxylase encoded by a nucleic acid sequence, having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 6, or nucleic acid sequence complementary thereto.

The exogenous gene encoding the PLP-dependent tryptophan decarboxylase may comprise the nucleic acid sequence set forth in SEQ ID NO: 8, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 8, or nucleic acid sequence complementary thereto. Embodiments of the present disclosure provide an isolated nucleotide sequence, and a PLP-dependent tryptophan decarboxylase encoded by the nucleic acid sequence, set forth in SEQ ID NO: 8, or nucleic acid sequence complementary thereto. Embodiments of the present disclosure provide an isolated nucleotide sequence, and a PLP-dependent tryptophan decarboxylase encoded by a nucleic acid sequence, having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 8, or nucleic acid sequence complementary thereto.

The exogenous gene encoding the PLP-dependent tryptophan decarboxylase may comprise the nucleic acid sequence set forth in SEQ ID NO: 30, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 30, or nucleic acid sequence complementary thereto. Embodiments of the present disclosure provide an isolated nucleotide sequence, and a PLP-dependent tryptophan decarboxylase encoded by the nucleic acid sequence, set forth in SEQ ID NO: 30, or nucleic acid sequence complementary thereto. Embodiments of the present disclosure provide an isolated nucleotide sequence, and a PLP-dependent tryptophan decarboxylase encoded by a nucleic acid sequence, having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 30, or nucleic acid sequence complementary thereto.

The exogenous gene encoding the PLP-dependent tryptophan decarboxylase may comprise the nucleic acid sequence set forth in SEQ ID NO: 49, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 49, or nucleic acid sequence complementary thereto. Embodiments of the present disclosure provide an isolated nucleotide sequence, and a PLP-dependent tryptophan decarboxylase encoded by the nucleic acid sequence, set forth in SEQ ID NO: 49, or nucleic acid sequence complementary thereto. Embodiments of the present disclosure provide an isolated nucleotide sequence, and a PLP-dependent tryptophan decarboxylase encoded by a nucleic acid sequence, having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 49, or nucleic acid sequence complementary thereto.

The coding sequence of the PLP-dependent tryptophan decarboxylase gene may be codon optimized for expression in a host cell of choice. Methods for codon optimization of coding sequence are well known to those skilled in the art.

By way of example, the coding sequence of the PLP-dependent tryptophan decarboxylase gene may be codon optimized for expression in S. cerevisiae, and may comprise the nucleic acid sequence set forth in SEQ ID NO: 23, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 23, or nucleic acid sequence complementary thereto.

In another example, the coding sequence of a codon optimised PLP-dependent tryptophan decarboxylase gene may comprise the nucleic acid sequence set forth in SEQ ID NO: 31, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 31, or nucleic acid sequence complementary thereto.

Embodiments of the present disclosure provide an isolated nucleotide sequence, and a PLP-dependent tryptophan decarboxylase encoded by the nucleic acid sequence, set forth in SEQ ID NO: 2, or nucleic acid sequence complementary thereto 3. Embodiments of the present disclosure provide an isolated nucleotide sequence, and a PLP-dependent tryptophan decarboxylase encoded by a nucleic acid sequence, having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 23, or nucleic acid sequence complementary thereto.

Embodiments of the present disclosure provide an isolated nucleotide sequence, and a PLP-dependent tryptophan decarboxylase encoded by the nucleic acid sequence, set forth in SEQ ID NO: 31, or nucleic acid sequence complementary thereto. Embodiments of the present disclosure provide an isolated nucleotide sequence, and a PLP-dependent tryptophan decarboxylase encoded by a nucleic acid sequence, having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 31, or nucleic acid sequence complementary thereto.

In one example, the coding sequence of a codon optimised PLP-dependent tryptophan decarboxylase gene may comprise the nucleic acid sequence set forth in SEQ ID NO: 47, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 47, or nucleic acid sequence complementary thereto.

Embodiments of the present disclosure provide an isolated nucleotide sequence, and a PLP-dependent tryptophan decarboxylase encoded by the nucleic acid sequence, set forth in SEQ ID NO: 47, or nucleic acid sequence complementary thereto. Embodiments of the present disclosure provide an isolated nucleotide sequence, and a PLP-dependent tryptophan decarboxylase encoded by a nucleic acid sequence, having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 47, or nucleic acid sequence complementary thereto.

In one example, the coding sequence of a codon optimised PLP-dependent tryptophan decarboxylase gene may comprise the nucleic acid sequence set forth in SEQ ID NO: 50, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 50, or nucleic acid sequence complementary thereto.

Embodiments of the present disclosure provide an isolated nucleotide sequence, and a PLP-dependent tryptophan decarboxylase encoded by the nucleic acid sequence, set forth in SEQ ID NO: 50, or nucleic acid sequence complementary thereto. Embodiments of the present disclosure provide an isolated nucleotide sequence, and a PLP-dependent tryptophan decarboxylase encoded by a nucleic acid sequence, having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 50, or nucleic acid sequence complementary thereto.

In one example, the coding sequence of a codon optimised PLP-dependent tryptophan decarboxylase gene may comprise the nucleic acid sequence set forth in SEQ ID NO: 51, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 51, or nucleic acid sequence complementary thereto.

Embodiments of the present disclosure provide an isolated nucleotide sequence, and a PLP-dependent tryptophan decarboxylase encoded by the nucleic acid sequence, set forth in SEQ ID NO: 51, or nucleic acid sequence complementary thereto. Embodiments of the present disclosure provide an isolated nucleotide sequence, and a PLP-dependent tryptophan decarboxylase encoded by a nucleic acid sequence, having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 51, or nucleic acid sequence complementary thereto.

In particular embodiments of the present disclosure the exogenous gene encoding the PLP-dependent tryptophan decarboxylase is operably linked to a promoter and/or other regulatory elements. Typically, the promoter and/or other regulatory elements are not naturally associated with a gene encoding the PLP-dependent tryptophan decarboxylase of the disclosure (i.e., are heterologous). The promoter may be a constitutive promoter or an inducible promoter. Examples of constitutive promoters useful in yeast cells include, but are not limited to, PGK (phosphoglycerate kinase) promoters, ADH-1 (alcohol dehydrogenase) promoters, ENO (enolase) promoters, glyceraldehyde 3-phosphate dehydrogenase (GPD) promoters (also referred to as TDH3 promoters), constitutive cell wall (CCW) promoters, histone (HHF) promoters, hexose transporter (HXT) promoters, PEP carboxykinase (PCK) promoters, PYK-1 (pyruvate kinase) promoters, translation-elongation factor-1-alpha (TEF) promoters and CYC-1 (cytochrome c-oxidase promoter) promoters. In one embodiment, a yeast promoter is a S. cerevisiae promoter. Exemplary constitutive promoters include, but are not limited to, pTDH3, pCCW12, pPGK1, pTEF1, pHHF2, pHXT7, pTEF2, pHHF1, pADH2, pPCK1, pMLS1, pICLI and pPHO89.

In another embodiment, the constitutive promoter may not have been derived from yeast. Examples of such promoters include, but are not limited to, the cauliflower mosaic virus 35S promoter, the glucocorticoid response element, and the androgen response element. The constitutive promoter may be the naturally occurring molecule or a variant thereof comprising, for example, one, two or three nucleotide substitutions which do not abolish (and preferably enhance) promoter function.

Effective conditions for the culture of the cells of the present disclosure are known to those skilled in the art and include, but are not limited to, suitable media, bioreactor, temperature, pH and oxygen conditions that permit secondary metabolite production, and in particular production of tryptophan derivatives. A suitable medium refers to any medium in which a cell is cultured to produce tryptophan derivatives defined herein. Such medium typically comprises an aqueous medium having assimilable carbon, nitrogen and phosphate sources, and appropriate salts, minerals, metals and other nutrients, such as vitamins. Cells defined herein can be cultured in conventional fermentation bioreactors, shake flasks, test tubes, microtiter dishes, and petri plates. Suitable culturing conditions are within the expertise of one of ordinary skill in the art.

### Tryptophan Derivatives

Embodiments of the present disclosure provide cells and methods for the biosynthetic production of one or more tryptophan derivatives, in particular tryptamine and tryptamine-derived compounds such as tryptamine-derived alkaloids. The cnsB, asTDC1, AcTDC and CrTDC genes described herein encode PLP-dependent tryptophan decarboxylases, catalysing the decarboxylation of an aliphatic carboxylic acid converting L-tryptophan to tryptamine. Accordingly, particular embodiments of the disclosure provide cells and methods for the production of tryptamine.

Cells of the present disclosure, comprising an exogenous gene encoding a PLP-dependent tryptophan decarboxylase of the present disclosure may further comprise one or more additional genes (which may be endogenous to the host cell or exogenous) encoding products, typically enzymes, required for the biosynthetic production of one or more tryptamine-derived compounds. Such compounds include, but are not limited to, 4-hydroxytryptamine, norbaeocystin, baeocystin, psilocybin, psilocin, norpsilocin, N,N-dimethyltryptamine (DMT), 5-methoxy-N,N-dimethyltryptamine, 5-hydroxy-N,N-dimethyltryptamine (bufotenin), N,N,N-trimethyltryptamine, N-methyltryptamine, serotonin (5-hydroxytryptamine), N-acetylserotonin, melatonin, aeruginascin, communesins (such as communesin A, communesin B and communesin F), harmala alkaloids (such as deoxyvasicine, deoxyvasicinone harmine, harmaline, tetrahydroharmine, and vasicine), ajmalicine, ajmaline, yohimbine, quinine, vincristine, vinblastine and camptothecin. In exemplary embodiments, cells of the present disclosure are capable of producing one or more of 4-hydroxytryptamine, norbaeocystin, psilocybin, psilocin. The skilled person would understand that such compounds can also include halogenated, fluorinated, deuterated and/or substituted variants or fragments thereof.

A cell of the present disclosure, in addition to comprising an exogenous gene encoding a PLP-dependent tryptophan decarboxylase of the present disclosure may further comprise a gene encoding a tryptamine 4-monooxygenase, such as PsiH, typically for the production of 4-hydroxytryptamine. A cell of the disclosure may comprise one or more copies (e.g., one, two, three, four or more copies) of a tryptamine 4-monooxygenase gene, which may be endogenous to the cell or may be provided exogenously, for example in a vector of the disclosure. The tryptamine 4-monooxygenase may comprise the amino acid sequence set forth in SEQ ID NO: 11, or may comprise a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence set forth in SEQ ID NO: 11. The coding sequence of the tryptamine 4-monooxygenase gene may be codon optimized for expression in a host cell of choice, for example S. cerevisiae (as set forth in SEQ ID NO: 25). Methods for codon optimization of coding sequence are well known to those skilled in the art. The tryptamine 4-monooxygenase enzyme may be encoded by a nucleic acid sequence set forth in SEQ ID NO: 12 or 25, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 12 or 25. Optionally, the PsiH tryptamine 4-monooxygenase is derived from Psilocybe cubensis (SEQ ID NO: 11), however numerous other sources of PsiH may be employed as would be well known to those skilled in the art, including for example, Psilocybe cyanescens, Panaeolus cyanescens, Gymnopilus junonius and Gymnopilus dilepsis.

A cell of the present disclosure, in addition to comprising an exogenous gene encoding a PLP-dependent tryptophan decarboxylase of the present disclosure may further comprise a gene encoding a 4-hydroxytryptamine kinase, such as PsiK, typically for the production of norbaeocystin. A cell of the disclosure may comprise one or more copies (e.g., one, two, three, four or more copies) of a 4-hydroxytryptamine kinase gene, which may be endogenous to the cell or may be provided exogenously, for example in a vector of the disclosure. The 4-hydroxytryptamine kinase may comprise the amino acid sequence set forth in SEQ ID NO: 13, or may comprise a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence set forth in SEQ ID NO: 13. The coding sequence of the 4-hydroxytryptamine kinase gene may be codon optimized for expression in a host cell of choice, for example S. cerevisiae (as set forth in SEQ ID NO: 26). Methods for codon optimization of coding sequence are well known to those skilled in the art. The 4-hydroxytryptamine kinase enzyme may be encoded by a nucleic acid sequence set forth in SEQ ID NO: 14 or 26, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 14 or 26. Optionally, the PsiK 4-hydroxytryptamine kinase is derived from Psilocybe cubensis (SEQ ID NO: 11), however numerous other sources of PsiK may be employed as would be well known to those skilled in the art, including for example, Psilocybe cyanescens, Panaeolus cyanescens, Gymnopilus junonius and Gymnopilus dilepsis.

A cell of the present disclosure, in addition to comprising an exogenous gene encoding a PLP-dependent tryptophan decarboxylase of the present disclosure may further comprise a gene encoding a methyl transferase (psilocybin synthase), such as PsiM, typically for the production of psilocybin. A cell of the disclosure may comprise one or more copies (e.g., one, two, three, four or more copies) of a psilocybin synthase gene, which may be endogenous to the cell or may be provided exogenously, for example in a vector of the disclosure. The psilocybin synthase may comprise the amino acid sequence set forth in SEQ ID NO: 15, or may comprise a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence set forth in SEQ ID NO: 15. The coding sequence of the psilocybin synthase gene may be codon optimized for expression in a host cell of choice, for example S. cerevisiae (as set forth in SEQ ID NO: 27). Methods for codon optimization of coding sequence are well known to those skilled in the art. The psilocybin synthase enzyme may be encoded by a nucleic acid sequence set forth in SEQ ID NO: 16 or 27, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 16 or 27. Optionally, the PsiM psilocybin synthase is derived from Psilocybe cubensis (SEQ ID NO: 15), however numerous other sources of PsiM may be employed as would be well known to those skilled in the art, including for example, Psilocybe cyanescens, Panaeolus cyanescens, Gymnopilus junonius and Gymnopilus dilepsis.

A cell of the present disclosure, in addition to comprising an exogenous gene encoding a PLP-dependent tryptophan decarboxylase of the present disclosure may further comprise a gene encoding a methyl transferase, such as RmNMT. A cell of the disclosure may comprise one or more copies (e.g., one, two, three, four or more copies) of a gene encoding RmNMT, which may be endogenous to the cell or may be provided exogenously, for example in a vector of the disclosure. The RmNMT may comprise the amino acid sequence set forth in SEQ ID NO: 42, or may comprise a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence set forth in SEQ ID NO: 42. The coding sequence of RmNMT may be codon optimized for expression in a host cell of choice, for example S. cerevisiae (as set forth in SEQ ID NO: 44). Methods for codon optimization of coding sequence are well known to those skilled in the art. The RmNMT may be encoded by a nucleic acid sequence set forth in SEQ ID NO: 43 or 44, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 43 or 44.

For the production of psilocin, a cell of the present disclosure, in addition to comprising an exogenous gene encoding a PLP-dependent tryptophan decarboxylase of the present disclosure may further comprise a gene encoding a 4-hydroxytryptamine kinase such as PsiK as described hereinbefore. The PsiK may comprise the amino acid sequence set forth in SEQ ID NO: 13, or may comprise a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence set forth in SEQ ID NO: 13. The PsiK enzyme may be encoded by a nucleic acid sequence set forth in SEQ ID NO: 14 or 26, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 14 or 26. Optionally, the PsiK is derived from Psilocybe cubensis (SEQ ID NO: 11), however numerous other sources of PsiK may be employed as would be well known to those skilled in the art, including for example, Psilocybe cyanescens, Panaeolus cyanescens, Gymnopilus junonius and Gymnopilus dilepsis.

A cell of the present disclosure, in addition to comprising an exogenous gene encoding a PLP-dependent tryptophan decarboxylase of the present disclosure may further comprise a gene encoding a cytochrome P450 reductase, such as Cpr derived from Psilocybe cubensis. A cell of the disclosure may comprise one or more copies (e.g., one, two, three, four or more copies) of a cytochrome P450 reductase gene, which may be endogenous to the cell or may be provided exogenously, for example in a vector of the disclosure. The cytochrome P450 reductase may comprise the amino acid sequence set forth in SEQ ID NO: 17, or may comprise a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence set forth in SEQ ID NO: 17. The coding sequence of the cytochrome P450 reductase gene may be codon optimized for expression in a host cell of choice, for example S. cerevisiae (as set forth in SEQ ID NO: 28). Methods for codon optimization of coding sequence are well known to those skilled in the art. The cytochrome P450 reductase may be encoded by a nucleic acid sequence set forth in SEQ ID NO: 18 or 28, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 18 or 28.

A cell of the present disclosure, in addition to comprising an exogenous gene encoding a PLP-dependent tryptophan decarboxylase of the present disclosure may further comprise a gene encoding a cytochrome P450 reductase, such as OsCPR derived from Oryza sativa Japonica Group. A cell of the disclosure may comprise one or more copies (e.g., one, two, three, four or more copies) of OsCPR, which may be endogenous to the cell or may be provided exogenously, for example in a vector of the disclosure. The OsCPR may comprise the amino acid sequence set forth in SEQ ID NO: 39, or may comprise a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence set forth in SEQ ID NO: 39. The coding sequence of the OsCPR may be codon optimized for expression in a host cell of choice, for example S. cerevisiae (as set forth in SEQ ID NO: 41). Methods for codon optimization of coding sequence are well known to those skilled in the art. The OsCPR may be encoded by a nucleic acid sequence set forth in SEQ ID NO: 40 or 41, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 40 or 41.

In a particular exemplary embodiment, a cell of the disclosure comprises one or more copies (e.g., one, two, three, four or more copies) of an exogenous gene encoding a PLP-dependent tryptophan decarboxylase having an amino acid sequence set forth in SEQ ID NO: 1, SEQ ID NO: 3, SEQ ID NO: 5 or SEQ ID NO: 7, or a sequence having at least about 70% sequence identity thereto, one or more copies (e.g., one, two, three, four or more copies) of a gene encoding a tryptamine 4-monooxygenase (e.g., PsiH) having an amino acid sequence set forth in SEQ ID NO: 11 or a sequence having at least about 70% sequence identity thereto, one or more copies (e.g., one, two, three, four or more copies) of a gene encoding a 4-hydroxytryptamine kinase (e.g., PsiK) having an amino acid sequence set forth in SEQ ID NO: 13 or a sequence having at least about 70% sequence identity thereto, one or more copies (e.g., one, two, three, four or more copies) of a gene encoding a methyl transferase (psilocybin synthase) (e.g., PsiM) having an amino acid sequence set forth in SEQ ID NO: 15 or a sequence having at least about 70% sequence identity thereto, and optionally one or more copies (e.g., one, two, three, four or more copies) of a gene encoding a cytochrome 450 reductase (e.g., Cpr) having an amino acid sequence set forth in SEQ ID NO: 17 or a sequence having at least about 70% sequence identity thereto. Typically the cell is capable of producing psilocybin and/or psilocin.

In a particular embodiment, a cell of the disclosure comprises one or more copies (e.g., one, two, three, four or more copies) of an exogenous gene encoding a PLP-dependent tryptophan decarboxylase having an amino acid sequence set forth in any one of SEQ ID NOs: 1, 3, 5, 7, 29 and 48, or a sequence having at least about 70% sequence identity thereto, one or more copies (e.g., one, two, three, four or more copies) of a gene encoding a tryptamine 4-monooxygenase (e.g., PsiH) having an amino acid sequence set forth in SEQ ID NO: 11 or a sequence having at least about 70% sequence identity thereto, one or more copies (e.g., one, two, three, four or more copies) of a gene encoding a 4-hydroxytryptamine kinase (e.g., PsiK) having an amino acid sequence set forth in SEQ ID NO: 13 or a sequence having at least about 70% sequence identity thereto, one or more copies (e.g., one, two, three, four or more copies) of a gene encoding a methyl transferase (psilocybin synthase) (e.g., PsiM) having an amino acid sequence set forth in SEQ ID NO: 15 or a sequence having at least about 70% sequence identity thereto, and optionally one or more copies (e.g., one, two, three, four or more copies) of a gene encoding a cytochrome 450 reductase (e.g., Cpr) having an amino acid sequence set forth in SEQ ID NO: 17 or a sequence having at least about 70% sequence identity thereto.

In a particular embodiment, a cell of the disclosure comprises one or more copies (e.g., one, two, three, four or more copies) of an exogenous polynucleotide encoding a PLP-dependent tryptophan decarboxylase having an amino acid sequence set forth in any one of SEQ ID NOs: 1, 3, 5, 7, 29 and 48, or a sequence having at least about 70% sequence identity thereto, one or more copies (e.g., one, two, three, four or more copies) of a gene encoding a methyl transferase (e.g., RmNMT) having an amino acid sequence set forth in SEQ ID NO: 42 or a sequence having at least about 70% sequence identity thereto, one or more copies (e.g., one, two, three, four or more copies) of a gene encoding a cytochrome P450 reductase (e.g., OsCPR) having an amino acid sequence set forth in SEQ ID NO: 39 or a sequence having at least about 70% sequence identity thereto, one or more copies (e.g., one, two, three, four or more copies) of a gene encoding a methyl transferase (e.g., OsT5H) having an amino acid sequence set forth in SEQ ID NO: 36 or a sequence having at least about 70% sequence identity thereto.

In an exemplary embodiment, a cell of the disclosure comprises one copy of the exogenous gene encoding a PLP-dependent tryptophan decarboxylase having an amino acid sequence set forth in SEQ ID NO: 1, SEQ ID NO: 3, SEQ ID NO: 5 or SEQ ID NO: 7, or a sequence having at least about 70% sequence identity thereto. The cell may also comprise one or more copies of one or more additional tryptophan decarboxylase genes, including for example psiD and/or crTDC. The tryptophan decarboxylase genes may be provided to the cell on the same or different vectors.

In an embodiment, a cell of the disclosure comprises one copy of the exogenous polynucleotide encoding a PLP-dependent tryptophan decarboxylase having an amino acid sequence set forth in any one of SEQ ID NOs: 1, 3, 5, 7, 29 and 48, or a sequence having at least about 70% sequence identity thereto. The cell may also comprise one or more copies of one or more additional tryptophan decarboxylase genes, including for example psiD, AcTDC, AsTDC1 and/or crTDC. The tryptophan decarboxylase genes may be provided to the cell on the same or different vectors.

In an exemplary embodiment, a cell of the disclosure comprises two copies of the exogenous gene encoding a PLP-dependent tryptophan decarboxylase having an amino acid sequence set forth in SEQ ID NO: 1, SEQ ID NO: 3, SEQ ID NO: 5 or SEQ ID NO: 7, or a sequence having at least about 70% sequence identity thereto. The cell may also comprise one or more copies of one or more additional tryptophan decarboxylase genes, including for example psiD and/or crTDC. The tryptophan decarboxylase genes may be provided to the cell on the same or different vectors. In an exemplary embodiment, the cell further comprises two copies of a tryptamine 4-monooxygenase (e.g., PsiH) gene, two copies of a 4-hydroxytryptamine kinase (e.g., PsiK) gene, two or three copies of a methyl transferase (psilocybin synthase) (e.g., PsiM) gene, and two copies of a cytochrome 450 reductase (e.g., Cpr) gene. In an exemplary embodiment, a cell of the disclosure comprises three copies a methyl transferase (psilocybin synthase) (e.g., PsiM) gene.

In an embodiment, a cell of the disclosure comprises two copies of the exogenous gene encoding a PLP-dependent tryptophan decarboxylase having an amino acid sequence set forth in any one of SEQ ID NOs: 1, 3, 5, 7, 29 and 48, or a sequence having at least about 70% sequence identity thereto. The cell may also comprise one or more copies of one or more additional tryptophan decarboxylase genes, including for example psiD, AcTDC, AsTDC1 and/or crTDC. The tryptophan decarboxylase genes may be provided to the cell on the same or different vectors. In an exemplary embodiment, the cell further comprises two copies of a tryptamine 4-monooxygenase (e.g., PsiH) gene, two copies of a 4-hydroxytryptamine kinase (e.g., PsiK) gene, two or three copies of a methyl transferase (psilocybin synthase) (e.g., PsiM) gene, and two copies of a cytochrome 450 reductase (e.g., Cpr) gene. In an exemplary embodiment, a cell of the disclosure comprises three copies a methyl transferase (psilocybin synthase) (e.g., PsiM) gene.

The yeast adenosylhomocysteinase (Sah1) and adenosine kinase (Ado1) enzymes have been reported to recycle co-factors S-adenosylmethionine (SAM) and S-adenosylhomocysteine (SAH) and increase PsiM methyltransferase activity. Accordingly, cells of the disclosure may comprise one or more copies (e.g., two, three, four or more copies) of a gene encoding Sah1 and/or a gene encoding Ado1. The gene encoding Sah1 may be the S. cerevisiae SAH1 gene nucleic acid sequence set forth in SEQ ID NO: 20, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 20. The Sah1 enzyme may comprise the amino acid sequence set forth in SEQ ID NO: 19, or may comprise a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence set forth in SEQ ID NO: 19. The gene encoding Ado1 may be the S. cerevisiae ADO1 gene nucleic acid sequence set forth in SEQ ID NO: 22, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 22. The Ado1 enzyme may comprise the amino acid sequence set forth in SEQ ID NO: 21, or may comprise a sequence having at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence set forth in SEQ ID NO: 21.

In an exemplary embodiment, a cell of the disclosure comprises at least two copies of an SAH1 gene and at least two copies of an ADO1 gene. One or more copies of each gene may be endogenous to the cell. One or more copies of each gene may be exogenous to the cell. For example, in embodiment of the disclosure wherein the cell is S. cerevisiae, the cell contains SAH1 and ADO1 genes, and is also provided with an additional copy of the SAH1 and ADO1 genes.

In an exemplary embodiment, a cell of the disclosure comprises one copy of exogenous gene encoding a PLP-dependent tryptophan decarboxylase having an amino acid sequence set forth in SEQ ID NO: 1, SEQ ID NO: 3, SEQ ID NO: 5 or SEQ ID NO: 7, or a sequence having at least about 70% sequence identity thereto, two copies of a tryptamine 4-monooxygenase (e.g., PsiH) gene, two copies of a 4-hydroxytryptamine kinase (e.g., PsiK) gene, two or three copies of a methyl transferase (psilocybin synthase) (e.g., PsiM) gene, two copies of a cytochrome 450 reductase (e.g., Cpr) gene, one exogenously supplied copy of an SAH1 gene and one exogenously supplied copy of an ADO1 gene.

In an exemplary embodiment, a cell of the disclosure comprises two copies of an exogenous gene encoding a PLP-dependent tryptophan decarboxylase having an amino acid sequence set forth in SEQ ID NO: 1, SEQ ID NO: 3, SEQ ID NO: 5 or SEQ ID NO: 7, or a sequence having at least about 70% sequence identity thereto, two copies of a tryptamine 4-monooxygenase (e.g., PsiH) gene, two copies of a 4-hydroxytryptamine kinase (e.g., PsiK) gene, two or three copies of a methyl transferase (psilocybin synthase) (e.g., PsiM) gene, two copies of a cytochrome 450 reductase (e.g., Cpr) gene, one exogenously supplied copy of an SAH1 gene and one exogenously supplied copy of an ADO1 gene.

In an exemplary embodiment, a cell of the disclosure comprises one copy of an exogenous gene encoding a PLP-dependent tryptophan decarboxylase having an amino acid sequence set forth in SEQ ID NO: 1, SEQ ID NO: 3, SEQ ID NO: 5, SEQ ID NO: 7, or a sequence having at least about 70% sequence identity thereto, one copy of a gene encoding an additional tryptophan decarboxylase (e.g., PsiD or CrTDC), two copies of a tryptamine 4-monooxygenase (e.g., PsiH) gene, two copies of a 4-hydroxytryptamine kinase (e.g., PsiK) gene, two or three copies of a methyl transferase (psilocybin synthase) (e.g., PsiM) gene, two copies of a cytochrome 450 reductase (e.g., Cpr) gene, one exogenously supplied copy of an SAH1 gene and one exogenously supplied copy of an ADO1 gene.

In an embodiment, a cell of the disclosure comprises one copy of exogenous gene encoding a PLP-dependent tryptophan decarboxylase having an amino acid sequence set forth in any one of SEQ ID NOs: 1, 3, 5, 7, 29 and 48, or a sequence having at least about 70% sequence identity thereto, two copies of a tryptamine 4-monooxygenase (e.g., PsiH) gene, two copies of a 4-hydroxytryptamine kinase (e.g., PsiK) gene, two or three copies of a methyl transferase (psilocybin synthase) (e.g., PsiM) gene, two copies of a cytochrome 450 reductase (e.g., Cpr) gene, one exogenously supplied copy of an SAH1 gene and one exogenously supplied copy of an ADO1 gene.

In an embodiment, a cell of the disclosure comprises two copies of an exogenous gene encoding a PLP-dependent tryptophan decarboxylase having an amino acid sequence set forth in any one of SEQ ID NOs: 1, 3, 5, 7, 29 and 48, or a sequence having at least about 70% sequence identity thereto, two copies of a tryptamine 4-monooxygenase (e.g., PsiH) gene, two copies of a 4-hydroxytryptamine kinase (e.g., PsiK) gene, two or three copies of a methyl transferase (psilocybin synthase) (e.g., PsiM) gene, two copies of a cytochrome 450 reductase (e.g., Cpr) gene, one exogenously supplied copy of an SAH1 gene and one exogenously supplied copy of an ADO1 gene.

In an embodiment, a cell of the disclosure comprises one copy of an exogenous gene encoding a PLP-dependent tryptophan decarboxylase having an amino acid sequence set forth in any one of SEQ ID NOs: 1, 3, 5, 7, 29 and 48, or a sequence having at least about 70% sequence identity thereto, one copy of a gene encoding an additional tryptophan decarboxylase (e.g., PsiD or CrTDC), two copies of a tryptamine 4-monooxygenase (e.g., PsiH) gene, two copies of a 4-hydroxytryptamine kinase (e.g., PsiK) gene, two or three copies of a methyl transferase (psilocybin synthase) (e.g., PsiM) gene, two copies of a cytochrome 450 reductase (e.g., Cpr) gene, one exogenously supplied copy of an SAH1 gene and one exogenously supplied copy of an ADO1 gene.

The present disclosure provides methods for the biosynthetic production of one or more tryptophan derivatives, in particular tryptamine and tryptamine-derived compounds such as tryptamine-derived alkaloids as described herein. The methods comprise culturing cells of the disclosure under suitable conditions to facilitate the production of the one or more tryptophan derivatives. Reference to conditions suitable to facilitate the production of the one or more tryptophan derivatives should also be understood to encompass conditions suitable for the expression of one or more of the genes described herein required for the production of the one or more tryptophan derivatives. Such conditions will be well known to those skilled in the art.

As the cells and methods of the present disclosure relate to the biosynthesis of tryptophan derivatives, it may be desirable in certain embodiments to increase production of L-tryptophan in cells of the disclosure. For example, cells of the disclosure may overexpress one or more genes responsible for L-tryptophan production, such as TRP1, TRP2, TRP3, TRP4 or TRP5 in S. cerevisiae, or contain one or more mutations in such genes that facilitate increased production and hence availability of tryptophan. Alternatively, or in addition, cells of the disclosure may, for example, comprise one or more mutations in genes encoding transcriptional repressors of the aromatic amino acid precursor pathway, such as ARO1, ARO2, ARO3 and/or ARO4 in S. cerevisiae, so as to increase the production and hence availability of tryptophan.

Effective conditions for the culture of the cells of the present disclosure are known to those skilled in the art and include, but are not limited to, suitable media, bioreactor, temperature, pH and oxygen conditions that permit secondary metabolite production, and in particular production of tryptophan derivatives. A suitable medium refers to any medium in which a cell is cultured to produce tryptophan derivatives defined herein. Such medium typically comprises an aqueous medium having assimilable carbon, nitrogen and phosphate sources, and appropriate salts, minerals, metals and other nutrients, such as vitamins. Cells defined herein can be cultured in conventional fermentation bioreactors, shake flasks, test tubes, microtiter dishes, and petri plates. Suitable culturing conditions are within the expertise of one of ordinary skill in the art.

Also provided herein are methods for the extraction of the one or more tryptophan derivatives produced in accordance with a method of the present disclosure. The extraction may comprise lysing the cells to release the compound(s) enabling the compound(s) to be purified or isolated by methods well known to those skilled in the art. Alternatively, the compound(s) may be secreted by the cells into the culture medium allowing the compound(s) to be isolated or purified therefrom using methods well known to those skilled in the art.

### Cell Free Production

The present disclosure also provides methods for cell free production of one or more tryptophan derivatives, in particular tryptamine and tryptamine-derived compounds such as tryptamine-derived alkaloids as described herein. The methods comprise contacting tryptophan with at least one pyridoxal phosphate (PLP)-dependent tryptophan decarboxylase described herein under suitable conditions to facilitate the production of the one or more tryptophan derivatives. Reference to conditions suitable to facilitate the production of the one or more tryptophan derivatives should also be understood to encompass conditions suitable for the decarboxylation, hydroxylation, phosphorylation, N-methylation and/or phosphorylation required for the production of the one or more tryptophan derivatives. Such conditions will be well known to those skilled in the art.

In some embodiments, the PLP-dependent tryptophan decarboxylase and/or tryptophan may be provided as a cell lysate or a purified and/or isolated protein. Methods for isolating proteins and/or producing recombinant proteins are known in the art and described, for example, in J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988).

Effective conditions for the production of the tryptophan derivatives are known to those skilled in the art and include, but are not limited to, suitable media, bioreactor, temperature, pH and oxygen conditions that permit secondary metabolite production, and in particular production of tryptophan derivatives. A suitable substrate refers to any substrate in which a PLP-dependent tryptophan decarboxylase is known to produce tryptophan derivatives defined herein. Suitable conditions are within the expertise of one of ordinary skill in the art.

Also provided herein are methods for the extraction of the one or more tryptophan derivatives produced in accordance with a method of the present disclosure. The extraction may comprise lysing the cells to release the compound(s) enabling the compound(s) to be purified or isolated by methods well known to those skilled in the art. Alternatively, the compound(s) may be secreted by the cells into the culture medium allowing the compound(s) to be isolated or purified therefrom using methods well known to those skilled in the art.

### Compositions and Uses

Also provided herein are compositions comprising cells of the present disclosure or one or more tryptophan derivatives extracted from cells of the present disclosure. The compositions can also comprise additional ingredients such as diluents, stabilizers, excipients, and adjuvants. Compositions comprising cells of the present disclosure, for example yeast cells, may be administered to subjects as probiotics.

In particular examples, provided are pharmaceutical compositions comprising one or more tryptophan derivatives extracted from a cell of the present disclosure and a pharmaceutically acceptable carrier. The carriers, diluents and adjuvants can include buffers such as phosphate, citrate, or other organic acids; antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

Compositions of the present disclosure may be employed in the treatment or prevention of diseases or disorders in which one or more tryptophan derivatives as described herein may be of therapeutic benefit. Typically, the disease or disorder is a neurodevelopmental, neurological, neuromuscular, movement, psychiatric, or psychological disease, disorder or syndrome. Such diseases, disorders and syndromes include, by way of non-limiting example only, depression, such as major depressive disorder or treatment-resistant depression, anxiety disorders, obsessive-compulsive disorder, personality disorders, substance addiction or dependence such as alcohol or tobacco addiction or dependence, post-traumatic stress disorder, migraine and chronic headache. Accordingly, provided herein are methods for treating or preventing a disease or disorder, as described hereinbefore, comprising administering to the subject a composition of the present disclosure. The subject may suffer from, be suspected of suffering from, or be at risk of suffering from a disease or disorder including depression or a depressive disorder, an anxiety disorder, obsessive-compulsive disorder, a personality disorder, substance addiction or dependence, post-traumatic stress disorder, migraine and/or chronic headache.

### Nucleotide Sequences, Nucleic Acid Constructs and Vectors

Provided herein are isolated nucleotide sequences comprising a gene encoding a fungal PLP-dependent tryptophan decarboxylase derived from the class Eurotiomycetes. Optionally, the gene is operably linked to a heterologous promoter capable of directing expression of the gene in a cell that does not naturally express the gene.

Also provided herein are isolated nucleotide sequences comprising a gene encoding a PLP-dependent tryptophan decarboxylase derived from the class Dicotyledons.

Provided herein are isolated nucleotide sequences comprising a gene encoding a tryptophan decarboxylase having an amino acid sequence set forth in SEQ ID NO: 1, SEQ ID NO: 3 or SEQ ID NO: 5, or a sequence having at least about 70% sequence identity thereto. Optionally the gene is operably linked to a heterologous promoter capable of directing expression of the gene in a cell that does not naturally express the gene.

Provided herein are isolated nucleotide sequences comprising a gene encoding a tryptophan decarboxylase having an amino acid sequence set forth in SEQ ID NO: 7, or a sequence having at least about 70% sequence identity thereto. Optionally the gene is operably linked to a heterologous promoter capable of directing expression of the gene in a cell that does not naturally express the gene.

Also provided herein are isolated nucleotide sequences comprising a gene encoding a tryptophan decarboxylase having an amino acid sequence set forth in any one of SEQ ID NOs: 7, 29 and 48, or a sequence having at least about 70% sequence identity thereto.

The present disclosure also provides nucleic acid constructs and vectors containing heterologous polynucleotide sequences. In particular, provided are nucleic acid constructs and vectors comprising at least one copy (e.g., one, two, three, four or more copies) of an exogenous gene encoding a PLP-dependent tryptophan decarboxylase of the present disclosure. Such nucleic acid constructs and vectors may be used, for example, to introduce the exogenous gene into a host cell, by any means known to those skilled in the art.

Thus, provided herein are nucleic acid constructs and vectors comprising a tryptophan decarboxylase gene comprising the nucleic acid sequence set forth in SEQ ID NO: 2, SEQ ID NO: 4 or SEQ ID NO: 6, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 2, SEQ ID NO: 4 or SEQ ID NO: 6, or a tryptophan decarboxylase gene comprising the nucleic acid sequence set forth in SEQ ID NO: 23 or 51, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 23 or 51.

Also provided herein are nucleic acid constructs and vectors comprising a tryptophan decarboxylase gene comprising the nucleic acid sequence set forth in SEQ ID NO: 8, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 8, or a tryptophan decarboxylase gene comprising the nucleic acid sequence set forth in SEQ ID NO: 31, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 31.

Also provided herein are nucleic acid constructs and vectors comprising a tryptophan decarboxylase gene comprising the nucleic acid sequence set forth in SEQ ID NO: 30, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 30, or a tryptophan decarboxylase gene comprising the nucleic acid sequence set forth in SEQ ID NO: 47, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 47.

Also provided herein are nucleic acid constructs and vectors comprising a tryptophan decarboxylase gene comprising the nucleic acid sequence set forth in SEQ ID NO: 49, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 49, or a tryptophan decarboxylase gene comprising the nucleic acid sequence set forth in SEQ ID NO: 51, or a sequence having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence set forth in SEQ ID NO: 50.

The tryptophan decarboxylase gene(s) present in the nucleic acid construct or vector may be operably linked to a heterologous promoter, such as a promoter described hereinabove, and/or other regulatory elements that are operable in the cell into which the vector is to be introduced. Nucleic acid constructs and vectors of the present disclosure may also comprise at least one copy (e.g., one, two, three, four or more copies) of one or more additional genes involved, directly or indirectly, in the biosynthesis of one or more tryptophan derivatives as described hereinbefore.

In particular embodiments, there is provided a vector comprising a gene encoding a PLP-dependent tryptophan decarboxylase of the present disclosure optionally operably linked to a heterologous promoter capable of directing expression of the gene in a cell that does not naturally express said gene, and wherein the tryptophan decarboxylase encoded by said gene is capable of a higher level of production of at least one tryptophan derivative in a cell relative to the level of production of the at least one tryptophan derivative in an equivalent cell that does not possess said gene and that comprises a PsiD tryptophan decarboxylase. Optionally, the equivalent cell comprises the PsiD tryptophan decarboxylase having the amino acid sequence of SEQ ID NO: 9. Optionally, the at least one tryptophan derivative the production of which is increased in a cell of the disclosure, is tryptamine or psilocybin. Optionally, the at least one tryptophan derivative, the production of which is increased in a cell of the disclosure, is N,N-Dimethyltryptamine, serotonin or bufotenin. In one embodiment, the at least one tryptophan derivative, the production of which is increased in a cell of the disclosure, is 5-methoxy-N,N-dimethyltryptamine, 5-methoxy-N,N-dimethyltryptamine, N-acetyl-serotonin and melatonin. In one embodiment, the compounds described herein can also include halogenated, fluorinated, deuterated and/or substituted variants or fragments thereof.

Also provided herein are nucleic acid constructs and vectors comprising one or more copies of one or more of the additional genes described above, encoding an additional tryptophan decarboxylase (e.g., PsiD, AcTDC, AsTDC1 or CrTDC), a tryptamine 4-monooxygenase (e.g., PsiH), a 4-hydroxytryptamine kinase (e.g., PsiK), a methyl transferase (psilocybin synthase) (e.g., PsiM or RmNMT), a cytochrome 450 reductase (e.g., Cpr, PcCpr or OsCPR), an tryptamine 5-hydroxylase (e.g., OsT5H) an adenosylhomocysteinase (e.g., Sah1) and/or an adenosine kinase (e.g., Ado1).

In nucleic acid constructs and vectors of the present disclosure the genes described are typically operably linked to a promoter, optionally a heterologous promoter, suitable to direct expression of the genes in a host cell, optionally a yeast cell, and/or other regulatory elements. The promoter may be a constitutive promoter or an inducible promoter. Examples of constitutive promoters useful in yeast cells include, but are not limited to, PGK (phosphoglycerate kinase) promoters, ADH-1 (alcohol dehydrogenase) promoters, ENO (enolase) promoters, glyceraldehyde 3-phosphate dehydrogenase (GPD) promoters (also referred to as TDH3 promoters), constitutive cell wall (CCW) promoters, histone (HHF) promoters, hexose transporter (HXT) promoters, PEP carboxykinase (PCK) promoters, PYK-1 (pyruvate kinase) promoters, translation-elongation factor-1-alpha (TEF) promoters and CYC-1 (cytochrome c-oxidase promoter) promoters. In one embodiment, a yeast promoter is a S. cerevisiae promoter. Exemplary constitutive promoters include, but are not limited to, pTDH3, pCCW12, pPGK1, pTEF1, pHHF2, pHXT7, pTEF2, pHHF1, pADH2, pPCK1, pMLS1, pICLI and pPHO89.

In another embodiment, the constitutive promoter may not have been derived from yeast. Examples of such promoters include, but are not limited to, the cauliflower mosaic virus 35S promoter, the glucocorticoid response element, and the androgen response element. The constitutive promoter may be the naturally occurring molecule or a variant thereof comprising, for example, one, two or three nucleotide substitutions which do not abolish (and preferably enhance) promoter function.

An exemplary nucleic acid construct or vector described herein comprises psiH, psiK and psiM, each operably linked to a promoter. Another exemplary nucleic acid construct or vector described herein comprises psiH, psiK, psiM and Pccpr, each operably linked to a promoter.

Another exemplary nucleic acid construct or vector described herein comprises cnsBv1, psiH, psiK, psiM and Pccpr, each operably linked to a promoter. Another exemplary nucleic acid construct or vector described herein comprises psiD, psiH, psiK, psiM and Pccpr, each operably linked to a promoter.

Another exemplary nucleic acid construct or vector described herein comprises SAH1 and ADO1, each operably linked to a promoter. Another exemplary nucleic acid construct or vector described herein comprises SAH1, ADO1 and psiM, each operably linked to a promoter.

Another exemplary nucleic acid construct or vector described herein comprises RmNMT, OsCPR and OsT5H, each operably linked to a promoter.

Nucleic acid constructs and vectors of the present disclosure are typically suitable for transforming or transducing a host cell and facilitating expression of the one or more genes, either directly, or via integration of the genes into the host cell genome. The nucleic acid constructs and vectors described above may be employed in any combination to produce cells according to the disclosure, capable of production of one or more tryptophan derivatives.

Vectors, such as plasmids, suitable for use in eukaryotic and prokaryotic host cells are widely described and well-known in the art. Those skilled in the art would appreciate that vectors may also contain additional sequences and elements useful for the replication of the vector in prokaryotic and/or eukaryotic cells, selection of the vector and the expression of a heterologous sequence in a variety of host cells. Plasmid vectors typically include additional nucleic acid sequences that provide for easy selection, amplification, and transformation of the expression cassette in cells, e.g., pYES-derived vectors, pUC-derived vectors, pSK-derived vectors, pGEM-derived vectors, pSP-derived vectors, or pBS-derived vectors. Suitable yeast expression vectors include the pPIC series of vectors, yeast integrating plasmids (YIp), yeast replicating plasmids (YRp), yeast centromere plasmids (YCp), and yeast episomal plasmids (YEp). Additional nucleic acid sequences include origins of replication to provide for autonomous replication of the vector, selectable marker genes, for example encoding antibiotic resistance, unique multiple cloning sites providing for multiple sites to insert nucleic acid sequences or genes encoded in the nucleic acid construct, and sequences that enhance transformation or transduction of cells.

In some examples, the vector is an expression vector, which can direct gene expression in a cell. Expression vectors are capable of transforming a host cell and of effecting expression of one or more specified polynucleotide molecule(s). Expression vectors useful for the present disclosure contain regulatory sequences such as transcription control sequences, translation control sequences, origins of replication, and other regulatory sequences that are compatible with the host cell and that control the expression of exogenous genes as described herein. In particular, vectors useful for the present disclosure include transcription control sequences. Transcription control sequences are sequences which control the initiation, elongation, and termination of transcription. Particularly important transcription control sequences are those which control transcription initiation, such as promoter and enhancer sequences. Suitable transcription control sequences include any transcription control sequence that can function in a host cell, and the choice of the regulatory sequences used may depend on the target host cell. A variety of such transcription control sequences are known to those skilled in the art.

Nucleic acid constructs and vectors can be constructed using known techniques, including, without limitation, the standard techniques of restriction endonuclease digestion, ligation, transformation, plasmid purification, in vitro or chemical synthesis of DNA, and DNA sequencing. The nucleic acid constructs and vectors may be introduced into a host cell using any method known in the art. Accordingly, the present disclosure also provides host cells comprising a vector comprising an exogenous gene described herein.

To facilitate identification of transformants when a nucleic acid construct or vector as described above is introduced into a cell, the vector desirably comprises a selectable or screenable marker gene in addition to the exogenous gene. Marker genes impart a distinct phenotype to cells expressing the marker gene and thus allows such transformed cells to be distinguished from cells that do not have the marker. A selectable marker gene confers a trait for which one can “select” based on resistance to a selective agent (e.g., an antibiotic or other treatment damaging to untransformed cells). A screenable marker gene (or reporter gene) confers a trait that one can identify through observation or testing, i.e., by “screening” (e.g., β-glucuronidase, luciferase, green fluorescent protein (GFP) or other enzyme activity not present in untransformed cells). The marker gene and the nucleotide sequence of interest do not have to be linked. The actual choice of a marker is not crucial as long as it is functional (i.e., selective) in combination with the cells of choice. Examples of selectable markers are markers that confer antibiotic resistance such as hygromycin, nourseothricin, ampicillin, erythromycin, chloramphenicol or tetracycline resistance.

In one example, marker-free integration methods are used for genomic integration. For example, the marker-free integration method can comprise CRISPR/Cas systems. Methods for marker-free integration using CRISPR/Cas systems are known in the art (such as those described in Chi et al, 2019 and Jessop-Fabre et al, 2016: Biotechnol J).

Any method can be used to introduce a nucleic acid construct or vector comprising the exogenous gene(s) into a cell and numerous such methods are well known to those skilled in the art. Yeast cells are typically transformed by chemical methods. For example, the cells may be treated with lithium acetate to achieve transformation efficiencies of approximately 10⁴ colony-forming units (transformed cells)/μg of DNA. Other common procedures for transforming yeast include utilising the production of yeast spheroplasts, the biolistic method where DNA coated metal microprojectiles are shot into the cells, and the glass bead methods which relies on the agitation of the yeast cells with glass beads and the DNA to be delivered to the cell. Of course, any suitable means of introducing nucleic acids into cells such as yeast cells, can be used.

Recombinant DNA technologies can be used to improve expression of a transformed nucleic acid molecule comprising the exogenous gene(s), for example by manipulating the number of copies of the nucleic acid molecule within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules defined herein include, but are not limited to, integration of the polynucleotide molecule into one or more host cell chromosomes, addition of stability sequences to mRNAs, substitutions or modifications of transcription control signals (e.g., promoters, operators, enhancers), substitutions or modifications of translational control signals (e.g., ribosome binding sites, Shine-Dalgarno sequences), modification of polynucleotide molecules to correspond to the codon usage of the host cell, and the deletion of sequences that destabilize transcripts.

A nucleic acid construct or vector comprising the exogenous gene(s) may be contained within a cell in any form. For example, the nucleic acid construct can be integrated into the genome of the cell (e.g., by homologous recombination or random integration) or maintained in an episomal state that can stably be passed on to daughter cells. Such extra-chromosomal genetic elements (such as plasmids) can additionally contain selection markers that ensure the presence of such genetic elements in daughter cells. Moreover, the cells can be stably or transiently transformed. In addition, the cells can contain a single copy, or multiple copies of the nucleic acid molecule.

Unless otherwise indicated, the recombinant DNA and cell culture techniques described and utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988).

In order that the disclosure may be readily understood and put into practical effect, particular preferred embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure is further described in the following numbered paragraphs:
1. A cell comprising an exogenous gene and capable of producing at least one tryptophan derivative, wherein the exogenous gene encodes a fungal pyridoxal phosphate (PLP)-dependent tryptophan decarboxylase derived from the class Eurotiomycetes, or a PLP-dependent tryptophan decarboxylase at least about 80% identical to, or comprising one or more conservative amino acid substitutions with respect to, said fungal PLP-dependent tryptophan decarboxylase, and wherein the level of production of the at least one tryptophan derivative in the cell is increased relative to the level of production of the at least one tryptophan derivative in an equivalent cell that does not possess the exogenous gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.
2. A cell comprising an exogenous gene and capable of producing at least one tryptophan derivative, wherein the exogenous gene encodes a tryptophan decarboxylase having an amino acid sequence set forth in SEQ ID NO: 1, SEQ ID NO: 3 or SEQ ID NO: 5 or a sequence having at least about 70% sequence identity thereto, and wherein the level of production of the at least one tryptophan derivative in the cell is increased relative to the level of production of the at least one tryptophan derivative in an equivalent cell that does not possess the exogenous gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.
3. The cell of paragraph 2, wherein the tryptophan decarboxylase encoded by the exogenous gene comprises the amino acid sequence set forth in SEQ ID NO: 1.
4. A cell comprising an exogenous gene and capable of producing at least one tryptophan derivative, wherein the exogenous gene encodes a tryptophan decarboxylase having an amino acid sequence set forth in SEQ ID NO: 7 or a sequence having at least about 70% sequence identity thereto, and wherein the level of production of the at least one tryptophan derivative in the cell is increased relative to the level of production of the at least one tryptophan derivative in an equivalent cell that does not possess the exogenous gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.
5. The cell of any one of paragraphs 1 to 4, wherein the level of production of the at least one tryptophan derivative in the cell is increased relative to the level of production of the at least one tryptophan derivative in an equivalent cell that does not possess the exogenous gene and that comprises a non-PLP-dependent tryptophan decarboxylase.
6. The cell of any one of paragraphs 1 to 5, wherein the non-PLP-dependent tryptophan decarboxylase is a PsiD tryptophan decarboxylase, optionally comprising the amino acid sequence set forth in SEQ ID NO: 9.
7. The cell of any one of paragraphs 1 to 6, wherein the cell comprises two or more copies of the exogenous gene.
8. The cell of any one of paragraphs 1 to 7, wherein the exogenous gene is operably linked to a heterologous promoter.
9. The cell of any one of paragraphs 1 to 8, further comprising a nucleic acid sequence encoding an additional tryptophan decarboxylase.
10. The cell of paragraph 9, wherein the additional tryptophan decarboxylase is PsiD, optionally comprising the amino acid sequence set forth in SEQ ID NO: 9 or a sequence having at least about 70% sequence identity thereto.
11. The cell of any one of paragraphs 1 to 10, wherein the cell further comprises a nucleic acid sequence encoding a tryptamine 4-monooxygenase comprising the amino acid sequence set forth in SEQ ID NO: 11 or a sequence having at least about 70% sequence identity thereto.
12. The cell of paragraph 11, wherein the tryptamine 4-monooxygenase is PsiH.
13. The cell of paragraph 12, comprising two or more copies of a gene encoding PsiH.
14. The cell of any one of paragraphs 1 to 13, wherein the cell further comprises a nucleic acid sequence encoding a 4-hydroxytryptamine kinase comprising the amino acid sequence set forth in SEQ ID NO: 13 or a sequence having at least about 70% sequence identity thereto.
15. The cell of paragraph 14, wherein the 4-hydroxytryptamine kinase is PsiK.
16. The cell of paragraph 15, comprising two or more copies of a gene encoding PsiK.
17. The cell of any one of paragraphs 1 to 16, wherein the cell further comprises a nucleic acid sequence encoding a methyl transferase comprising the amino acid sequence set forth in SEQ ID NO: 15 or a sequence having at least about 70% sequence identity thereto.
18. The cell of paragraph 17, wherein the methyl transferase is PsiM.
19. The cell of paragraph 18, comprising two or more copies of a gene encoding PsiM.
20. The cell of any one of paragraphs 1 to 19, wherein the cell further comprises a nucleic acid sequence encoding a cytochrome P450 reductase comprising the amino acid sequence set forth in SEQ ID NO: 17 or a sequence having at least about 70% sequence identity thereto.
21. The cell of paragraph 20, wherein the cytochrome P450 reductase is PcCpr.
22. The cell of paragraph 21, comprising two or more copies of a gene encoding PcCpr.
23. The cell of any one of paragraphs 1 to 22, wherein the cell further comprises one or more, optionally at least two, copies of a gene encoding an adenosylhomocysteinase comprising the amino acid sequence set forth in SEQ ID NO: 19 or a sequence having at least about 70% sequence identity thereto.
24. The cell of any one of paragraphs 1 to 23, wherein the cell further comprises one or more, optionally at least two, copies of a gene encoding an adenosine kinase comprising the amino acid sequence set forth in SEQ ID NO: 21 or a sequence having at least about 70% sequence identity thereto.
25. The cell of any one of paragraphs 1 to 24, wherein the cell is a yeast cell.
26. The cell of paragraph 25, wherein the yeast cell is a Saccharomyces sp. cell.
27. The cell of paragraph 26, wherein the Saccharomyces sp. is S. cerevisiae.
28. The cell of any one of paragraphs 1 to 27, wherein the at least one tryptophan derivative is tryptamine or a tryptamine-derived alkaloid.
29. The cell of paragraph 28, wherein the tryptophan derivative is tryptamine.
30. The cell of paragraph 28, wherein the tryptamine-derived alkaloid is psilocybin.
31. A cell capable of producing at least one tryptophan derivative, comprising:
(a) one or more copies of a gene encoding a fungal PLP-dependent tryptophan decarboxylase derived from the class Eurotiomycetes, or a PLP-dependent tryptophan decarboxylase at least about 80% identical to, or comprising one or more conservative amino acid substitutions with respect to, said fungal PLP-dependent tryptophan decarboxylase;
(b) one or more copies of a gene encoding a PsiH tryptamine 4-monooxygenase;
(c) one or more copies of a gene encoding a PsiK hydroxytryptamine kinase; and
(d) one or more copies of a gene encoding a PsiM methyl transferase.
32. A cell capable of producing at least one tryptophan derivative, comprising:
(a) one or more copies of a gene encoding a tryptophan decarboxylase having an amino acid sequence set forth in SEQ ID NO: 1, SEQ ID NO: 3 or SEQ ID NO: 5, or a sequence having at least about 70% sequence identity thereto;
(b) one or more copies of a gene encoding a PsiH tryptamine 4-monooxygenase;
(c) one or more copies of a gene encoding a PsiK hydroxytryptamine kinase;
(d) one or more copies of a gene encoding a PsiM methyl transferase.
33. A cell capable of producing at least one tryptophan derivative, comprising:
(a) one or more copies of a gene encoding a tryptophan decarboxylase having an amino acid sequence set forth in SEQ ID NO: 7, or a sequence having at least about 70% sequence identity thereto;
(b) one or more copies of a gene encoding a PsiH tryptamine 4-monooxygenase;
(c) one or more copies of a gene encoding a PsiK hydroxytryptamine kinase;
(d) one or more copies of a gene encoding a PsiM methyl transferase.
34. The cell of any one of paragraphs 31 to 33, wherein the cell is capable of a higher level of production of at least one tryptophan derivative relative to the level of production of the at least one tryptophan derivative in an equivalent cell that does not possess (a), and that comprises a tryptophan decarboxylase encoded by a gene other than (a), optionally a gene encoding a PsiD tryptophan decarboxylase.
35. The cell of any one of paragraphs 31 to 34, wherein the tryptophan derivative is tryptamine or psilocybin.
36. The cell of any one of paragraphs 31 to 35, wherein the cell further comprises one or more copies of a gene encoding a cytochrome P450 reductase, optionally a PcCpr cytochrome P450 reductase.
37. The cell of any one of paragraphs 31 to 36, wherein the cell further comprises one or more copies of a gene encoding an adenosylhomocysteinase, optionally an Sah1 adenosylhomocysteinase.
38. The cell of any one of paragraphs 31 to 37, wherein the cell further comprises one or more copies of a gene encoding an adenosine kinase, optionally an Ado1 adenosine kinase.
39. The cell of any one of paragraphs 31 to 38, wherein the cell further comprises one or more copies of a gene encoding an additional tryptophan decarboxylase, optionally a PsiD and/or CrTDC tryptophan decarboxylase.
40. A vector comprising a gene encoding a fungal PLP-dependent tryptophan decarboxylase derived from the class Eurotiomycetes, or a PLP-dependent tryptophan decarboxylase at least about 80% identical to, or comprising one or more conservative amino acid substitutions with respect to, said fungal PLP-dependent tryptophan decarboxylase, optionally operably linked to a heterologous promoter capable of directing expression of the gene in a cell that does not naturally express said gene, and wherein the tryptophan decarboxylase encoded by said gene is capable of a higher level of production of at least one tryptophan derivative in a cell relative to the level of production of the at least one tryptophan derivative in an equivalent cell that does not possess said gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.
41. A vector comprising a gene encoding a tryptophan decarboxylase having an amino acid sequence set forth in SEQ ID NO: 1, SEQ ID NO: 3 or SEQ ID NO: 5, or a sequence having at least about 70% sequence identity thereto, optionally operably linked to a heterologous promoter capable of directing expression of the gene in a cell that does not naturally express said gene, and wherein the tryptophan decarboxylase encoded by said gene is capable of a higher level of production of at least one tryptophan derivative in a cell relative to the level of production of the at least one tryptophan derivative in an equivalent cell that does not possess said gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.
42. A vector comprising a gene encoding a tryptophan decarboxylase having an amino acid sequence set forth in SEQ ID NO: 7, or a sequence having at least about 70% sequence identity thereto, optionally operably linked to a heterologous promoter capable of directing expression of the gene in a cell that does not naturally express said gene, and wherein the tryptophan decarboxylase encoded by said gene is capable of a higher level of production of at least one tryptophan derivative in a cell relative to the level of production of the at least one tryptophan derivative in an equivalent cell that does not possess said gene and that comprises a tryptophan decarboxylase other than a PLP-dependent tryptophan decarboxylase derived from the class Eurotiomycetes or a tryptophan decarboxylase at least about 80% identical thereto, or comprising one or more conservative amino acid substitutions with respect thereto.
43. A host cell comprising a vector of any one paragraphs 40 to 42.
44. A method for producing at least one tryptophan derivative, comprising culturing the cell of any one of paragraphs 1 to 39 or 43 under conditions suitable for the production of the at least one tryptophan derivative.
45. The method of paragraph 44, further comprising extracting the at least one tryptophan derivative from the cell.
46. The method of paragraph 45, wherein the extracted tryptophan derivative is tryptamine or a tryptamine-derived alkaloid.
47. The method of paragraph 46, wherein the tryptophan derivative is tryptamine.
48. The method of paragraph 46, wherein the tryptamine-derived alkaloid is psilocybin.
49. A tryptophan derivative produced by the method of any one of paragraphs 44 to 48.
50. A composition comprising the cell of any one of paragraphs 1 to 39 or 43, or the extracted tryptophan derivative of paragraph 49.
51. A method for treating or preventing a disease or disorder in a subject, comprising administering to the subject a composition of paragraph 50.
52. The method of paragraph 51, wherein the disease or disorder is a neurodevelopmental, neurological, neuromuscular, movement, psychiatric, or psychological disease, disorder or syndrome.

### EXAMPLES

### Example 1. Plasmid Assembly and Strain Construction

The coding sequences of the tryptophan decarboxylase genes cnsBv1 (SEQ ID NO: 2) and cnsBv2 (SEQ ID NO: 4) from Pencillium expansum (Lin et al., 2015, Angew Chem Int Ed Engl, 54: 3004-7), the tryptophan decarboxylase gene represented by SEQ ID NO: 8 from Aspergillus steynii (herein termed “asTDC1”), psiD, psiH, psiK, psiM from Psilocybe cubensis FSU12409 (Fricke et al., 2017, Angew Chem Int Ed Engl, 56: 12352-55; GenBank: KY984101.1, GenBank: MF000993.1, GenBank: KY984099.1, GenBank: KY984100.1, respectively) and Pccpr (Milne et al, Metab Eng, 60: 25-36.) were synthesized by Twist Biosciences as synthetic gene fragments. Wild-type cnsBv1, cnsBv2, AcnsBv2, asTDC1, psiD, psiH, psiK, psiM and Pccpr coding sequences are provided in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16 and SEQ ID NO: 18, respectively. The amino acid sequences of the CnsBv1, CnsBv2, ΔCnsBv2, AsTDC1, PsiD, PsiH, PsiK, PsiM and PcCpr polypeptides encoded by these coding sequences are provided in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15 and SEQ ID NO: 17, respectively.

The coding sequence of the S-adenosylhomocysteine hydrolyse SAH1 gene from S. cerevisiae (GenBank: NM_001178934.3) was amplified by PCR from S. cerevisiae genomic DNA with primers 5′-GGTCTCTAGGATGTCTGCTCCAGCTCAAAACT-3′ SAHIF; SEQ ID NO: 32) and 5′-GGTCTCAAACTTCAATATCTGTAGTGGTCGGCC-3′ (SAH1R; SEQ ID NO: 33). The coding sequence of the adenosine kinase ADO1 gene from S. cerevisiae (GenBank NP_010961.3) was amplified by PCR from S. cerevisiae genomic DNA with primers 5′-GGTCTCTGCAATGACCGCACCATTGGTAG-3′ (ADO1F; SEQ ID NO: 34) and 5′-GGTCTCATGTGCTATTTAGAGTAAGATATTTTTTCGGAAGG-3′ (ADO1R; SEQ ID NO: 35). The wild-type SAH1 andADO1 coding sequences are provided in SEQ ID NO: 20 and SEQ ID NO: 22, respectively. The amino acid sequences of Sah1 and Ado1 are provided in SEQ ID NO: 19 and SEQ ID NO: 21, respectively.

Alignments of the CnsB and PsiD polypeptide sequences are shown in FIG. 3. Based on pairwise alignment using the Needleman-Wunsch algorithm CnsBv1 and PsiD share only 16.0% sequence identity, CnsBv2 and PsiD share only 16.2% sequence identity, and ΔCnsBv2 and PsiD share only 15.3% sequence identity. Shown in FIG. 4 are alignments of the CnsB amino acid sequences (of SEQ ID NOs:1, 3 and 5) with that of CrTDC (SEQ ID NO: 29), the tryptophan decarboxylase from Catharanthus roseus. Sequence identities are 24.3% between CnsBv1 and CrTDC, 23.5% between CnsBv2 and CrTDC, and 23.5% between ΔCnsBv2 and CrTDC. PsiD and CrTDC share 15.9% sequence identity.

The cnsBv1, asTDC1, psiD, psiH, psiK, psiMIand Pccpr coding sequences were codon optimized for expression in Saccharomyces cerevisiae and featured 5′- and 3′-end restriction sites for Golden Gate cloning. The codon optimized coding sequences of cnsBv1 and asTDC1 are provided in SEQ ID NOs:23 and 31, respectively. The codon optimized coding sequences of psiD psiH, psiK, psiM and Pccpr are provided in SEQ ID Nos:24-28, respectively.

Plasmids pNMT-cnsBv1, pNMT-asTDC1 and pNMT-psiD were constructed from individual parts with a BsaI Golden Gate assembly (Engler et al., 2008, PLoS One, 3: e3647). The plasmids are identical with the exception of the tryptophan decarboxylase gene, and contain the following components: pTDH3 promoter and tENO1 terminator flanking the respective decarboxylase gene (the gene operably linked to the pTDH3 promoter), ampicillin resistance gene, ColE1 origin of replication, URA3 auxotrophic marker and homology arms for expression cassette integration into the URA3 locus of the S. cerevisiae genome. E. coli 10-beta cells were used for plasmid cloning and propagation (cultured at 37° C. and 180 rpm).

Plasmids pNMT-BHKMC and pNMT-DHKMC were constructed from individual parts with a BsaI Golden Gate assembly. The plasmids are identical except for the tryptophan decarboxylase gene and contain the following components: pTDH3 promoter and tENO1 terminator flanking the respective decarboxylase gene (pNMT-BHKMC contains cnsBv1 operably linked to the pTDH3 promoter; pNMT-DHKMC contains psiD operably linked to the pTDH3 promoter); pCCW12 promoter and tSSAl terminator flanking psiH (operably linked to the pCCW12 promoter); pPGK1 promoter and tADHI terminator flanking Pccpr (operably linked to the pPGK1 promoter); pHHF2 promoter and tPGK1 terminator flanking psiK (operably linked to the pHHF2 promoter); pTEF1 promoter and tENO2 terminator flanking psiM (operably linked to the pTEF1 promoter); ampicillin resistance gene; ColE1 origin of replication; URA3 auxotrophic marker; and homology arms for expression cassette integration into the URA3 locus of the S. cerevisiae genome.

Plasmid pNMT-HKMC was constructed from individual parts with aBsaI Golden Gate assembly and contains the following components: pCCW12 promoter and tSSA1 terminator flanking psiH (operably linked to the pCCW12 promoter); pPGK1 promoter and tADH1 terminator flanking Pccpr (operably linked to the pPGK1 promoter); pHHF2 promoter and tPGK1 terminator flanking psiK (operably linked to the pHHF2 promoter); pTEF1 promoter and tENO2 terminator flanking psiM (operably linked to the pTEF1 promoter); ampicillin resistance gene; ColE1 origin of replication; LEU2 auxotrophic marker; and homology arms for expression cassette integration into the LEU2 locus of the S. cerevisiae genome.

Plasmid pNMT-MASB was constructed from individual parts with a BsaI Golden Gate assembly and contains the following components: pTDH3 promoter and tENO1 terminator flanking cnsBv1 (operably linked to the pTDH3 promoter); pTEF1 promoter and tENO2 terminator flanking psiM (operably linked to the pTEF1 promoter); pCCW12 promoter and tSSAl terminator flanking ADO1 (operably linked to the pCCW12 promoter); pTDH3 promoter and tENO1 terminator flanking SAH1 (operably linked to the pTDH3 promoter); ampicillin resistance gene; ColE1 origin of replication; HIS3 auxotrophic marker; and homology arms for expression cassette integration into the HIS3 locus of the S. cerevisiae genome.

Plasmid pNMT-MAS was constructed from individual parts with a BsaI Golden Gate assembly and contains the following components: pTEF1 promoter and tENO2 terminator flanking psiM (operably linked to the pTEF1 promoter); pCCW12 promoter and tSSAl terminator flanking ADO1 (operably linked to the pCCW12 promoter); pTDH3 promoter and tENO1 terminator flanking SAH1 (operably linked to the pTDH3 promoter); ampicillin resistance gene; ColE1 origin of replication; HIS3 auxotrophic marker; and homology arms for expression cassette integration into the HIS3 locus of the S. cerevisiae genome.

Plasmid pNMT-M was constructed from individual parts with a BsaI Golden Gate assembly and contains the following components: pTEF1 promoter and tENO2 terminator flanking psiM (operably linked to the pTEF1 promoter), ampicillin resistance gene, ColE1 origin of replication, HIS3 auxotrophic marker and homology arms for expression cassette integration into the HIS3 locus of the S. cerevisiae genome.

S. cerevisiae strain BY4741 (cultured at 30° C. and 250 rpm) was used as the parent strain for all transformations. All transformations of S. cerevisiae were performed using the LiAc/SS carrier DNA/PEG method (Gietz and Schiestl, 2007, Nature Protocols, 2: 1-4).

Transformation of S. cerevisiae with pNMT-cnsBv1, pNMT-asTDC1 and pNMT-psiD yielded strains SC-cnsBv1, SC-asTDC1 and SC-psiD, respectively. Each strain contains a single copy of the relevant tryptophan decarboxylase gene.

Transformation of S. cerevisiae with both pNMT-DHKMC and pNMT-HKMC yielded strain SC-D, whereas transformation with both pNMT-BHKMC and pNMT-HKMC yielded strain SC-B. Strain SC-D contains a single copy of psiD and two copies of each of psiH, psiK, psiM and Pccpr. Strain SC-B contains a single copy of cnsBv1 and two copies of each of psiH, psiK, psiMand Pccpr.

Transformation ofS. cerevisiae with both pNMT-DHKMC, pNMT-HKMC and pNMT-M yielded strain SC-DM, whereas transformation with both pNMT-BHKMC, pNMT-HKMC and pNMT-M yielded strain SC-BM. Strain SC-DM contains a single copy of psiD, two copies of each of psiH, psiK and Pccpr, and three copies of psiM. Strain SC-BM contains a single copy of cnsB, two copies of each of psiH, psiK and Pccpr, and three copies of psiM.

Transformation ofS. cerevisiae with both pNMT-DHKMC, pNMT-HKMC and pNMT-MAS yielded strain SC-DMAS, whereas transformation with both pNMT-BHKMC, pNMT-HKMC and pNMT-MAS yielded strain SC-BMAS. Strain SC-DMAS contains a single copy of psiD, two copies of each of psiH, psiK and Pccpr, three copies of psiM and a second copy of each of the endogenous S. cerevisiae genes SAHI and ADO1. Strain SC-BMAS contains a single copy of cnsB, two copies of each of psiH, psiK and Pccpr, three copies of psiM and a second copy of each of the endogenous S. cerevisiae genes SAHI and ADO1.

Transformation ofS. cerevisiae with both pNMT-DHKMC, pNMT-HKMC and pNMT-MASB yielded strain SC-DMASB, whereas transformation with both pNMT-BHKMC, pNMT-HKMC and pNMT-MASB yielded strain SC-BMASB. Strain SC-DMASB contains a single copy of psiD, a single copy of cnsB, two copies of each of psiH, psiK and Pccpr, three copies of psiM and a second copy of each of the endogenous S. cerevisiae genes SAHI and ADO1. Strain SC-BMASB contains two copies of cnsB, two copies of each of psiH, psiK and Pccpr, three copies of psiM and a second copy of each of the endogenous S. cerevisiae genes SAHI and ADO1.

### Example 2. Tryptamine Biosynthesis

The ability of S. cerevisiae strains SC-cnsBv1 and SC-psiD to produce tryptamine was determined. 2 mL cultures of dropout medium (6.7 g/L yeast nitrogen base without amino acids, 1.4 g/L yeast synthetic dropout medium without trp/leu/his/ura, 20 g/L glucose, 76 mg/L tryptophan, 500 mg/L leucine, 125 mg/L histidine) were inoculated with single colonies of S. cerevisiae. After overnight culturing, 2 mL of 2×YPD (40 g/L glucose, 40 g/L peptone, 2 g/L yeast extract) were added to each culture.

Samples of 400 μL of culture broth were taken on day 5 and day 6 and extractions of cells and medium was performed separately after separation by centrifugation. The cell pellet was extracted from by the addition of 400 μL of methanol, followed by thorough vortexing, centrifugation, transferral of the supernatant to a new microcentrifuge tube, drying down and resuspension in 200 μL of methanol (cell extraction). The culture medium was treated with 500 μL of n-butanol, the organic layer was transferred to a new microcentrifuge tube, dried down and resuspended in 200 μL of methanol (cell growth medium extraction).

For analysis, 4 μL of the cell extraction or 3 μL of the cell growth medium extraction was injected into an Agilent 1260 liquid chromatography system with a Kinetex C18 column (2.1×100 mm, 2.6 μm), coupled to an Agilent 6130 Quadrupole mass spectrometer with an ESI source. The mobile phase was a 10 min linear gradient of 5-95% acetonitrile-water containing 0.1% formic acid. Tryptamine peaks in the extraction LC-MS chromatograms were identified by comparison with the chromatogram of an authentic tryptamine standard. To determine tryptamine titers, known quantities of tryptamine were injected into the LC-MS and the corresponding peak areas were used to craft a standard curve. The standard curve was then used to calculate tryptamine titers in extractions from observed tryptamine chromatogram peak areas.

As shown in Table 2 and FIG. 5, tryptamine production was significantly higher at both timepoints (day 5 and day 6), as determined in both cells and growth medium, using the SC-cnsBv1 strain in comparison to the SC-psiD strain.

In a separate experiment, the ability of S. cerevisiae strains SC-cnsBv1, SC-asTDC1 and SC-psiD to produce tryptamine was determined. 1 mL cultures of YPD (20 g/L glucose, 20 g/L peptone, 10 g/L yeast extract) were inoculated with single colonies of S. cerevisiae. Samples of 100 μL of culture broth were taken on day 3, day 5 and day 6. Extractions were performed by the addition of 200 μL of methanol, followed by thorough vortexing. The extracts were thoroughly centrifuged to remove cell debris.

For analysis, 1 μL of the extraction was injected into an Agilent 1260 liquid chromatography system with a Kinetex C18 column (2.1×100 mm, 2.6 μm), coupled to an Agilent 6130 Quadrupole mass spectrometer with an ESI source. The mobile phase was a 10 min linear gradient of 5-95% acetonitrile-water containing 0.1% formic acid. Tryptamine peaks in the extraction LC-MS chromatograms were identified by comparison with the chromatogram of an authentic tryptamine standard. To determine tryptamine titers, known quantities of tryptamine were injected into the LC-MS and the corresponding peak areas were used to craft a standard curve. The standard curve was then used to calculate tryptamine titers in extractions from observed tryptamine chromatogram peak areas.

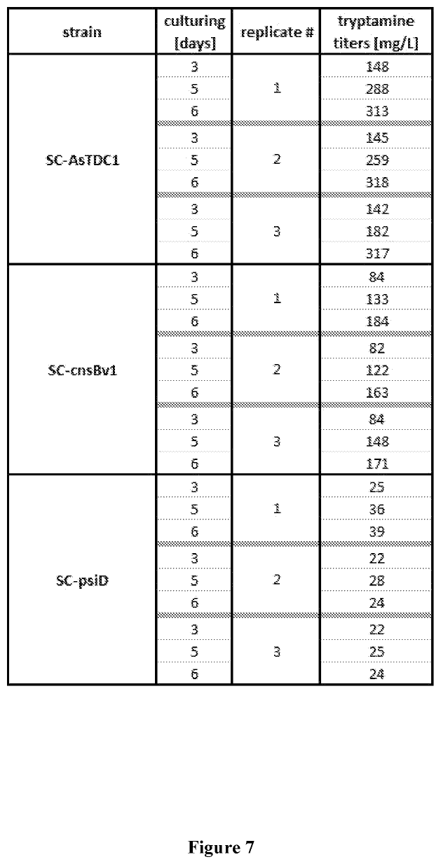

As shown in FIG. 7, tryptamine production in the presence of asTDC1 was significantly higher at all three timepoints (days 3, 5 and 6), than in the presence of either cnsBv1 or psiD.

### Example 3. Psilocybin and Psilocin Biosynthesis

The ability of S. cerevisiae strains SC-D, SC-B, SC-DM, SC-BM, SC-DMAS, SC-BMAS, SC-BMASB and SC-DMASB to produce psilocybin and psilocin was determined. 4 mL cultures of dropout medium (adjusted to pH 4 and with 20 g/L KH₂PO₄ and 1 μM thiamine) were inoculated with single colonies of S. cerevisiae. Samples of 400 μL of culture broth were taken on day 2 and day 3 and extractions of cells and medium was performed separately after separation by centrifugation. The cell pellet was extracted from by addition of 400 μL of methanol, followed by thorough vortexing, centrifugation, transferal of the supernatant to a new microcentrifuge tube, drying down and resuspension in 200 μL of methanol. The culture medium was washed twice with hexane, dried down and resuspended in 200 μL of methanol.

For analysis, 1 μL of the cell extraction or 1 μL of the media extraction was injected into a Thermo Scientific Vanquish ultra-high-performance liquid chromatography system with an Agilent C18 column (2.1×100 mm, 2.7 μm), coupled to an Orbitrap Exploris 120 mass spectrometer. The mobile phase was a 10 min linear gradient of 5-95% acetonitrile-water containing 0.1% formic acid.

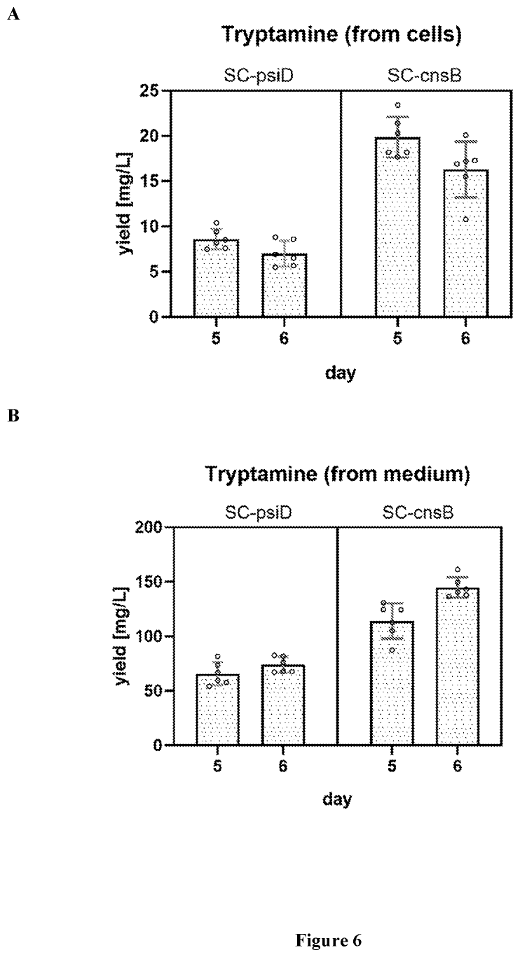

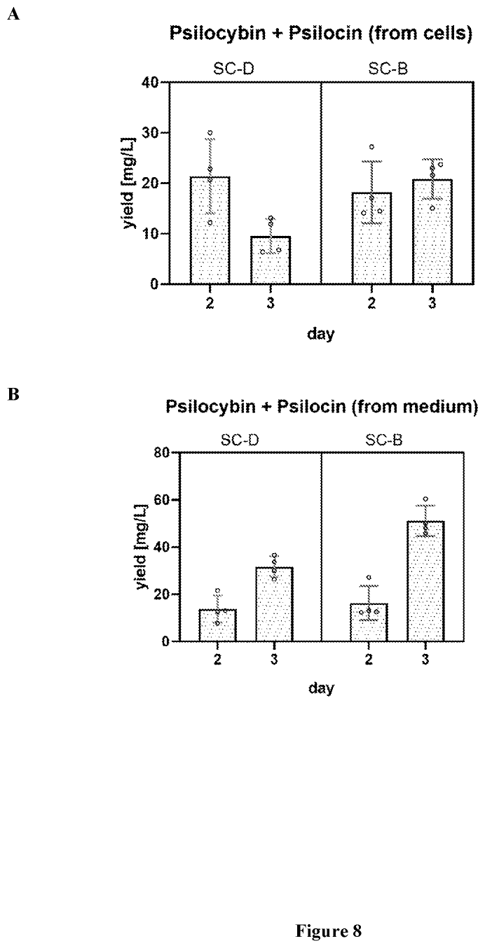

As shown in Table 3 and FIG. 8, psilocybin and psilocin production were slightly higher as determined in growth medium, using SC-B strain in comparison to SC-D strain. The difference in psilocybin and psilocin production did not correspond to the significantly higher tryptamine production shown in Table 2 and FIG. 6, indicating a bottleneck in the psilocybin pathway downstream of the tryptophan decarboxylase.

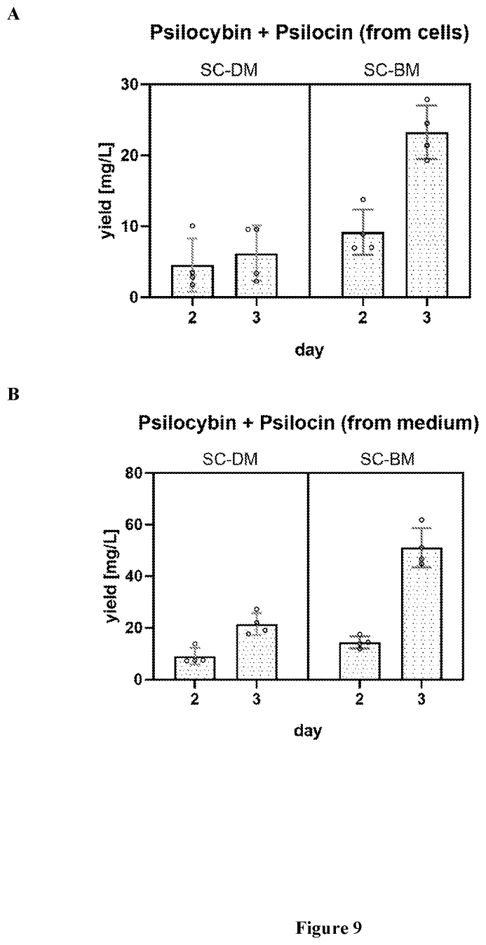

As shown in Table 4 and FIG. 9, the addition of a third copy of psiM in SC-BM and SC-DM resulted in a larger difference in psilocybin and psilocin production as determined in both cells and growth medium using SC-BM strain in comparison to SC-DM strain.

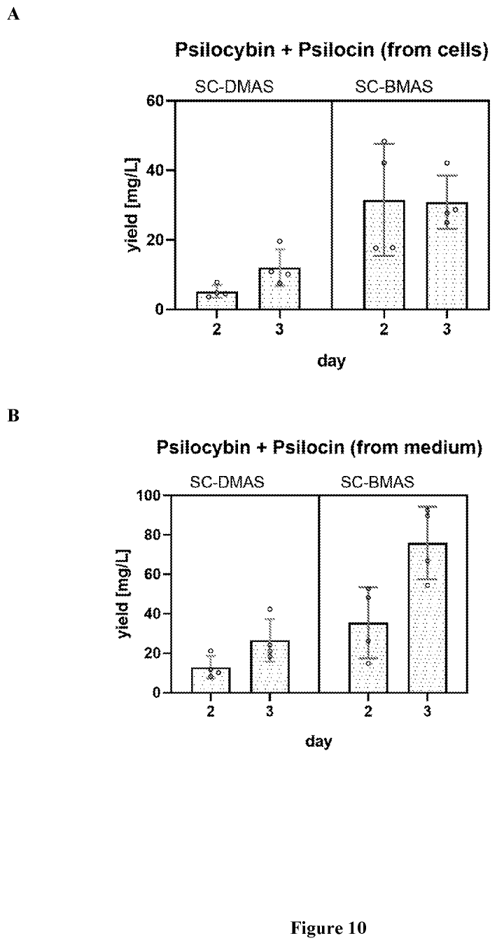

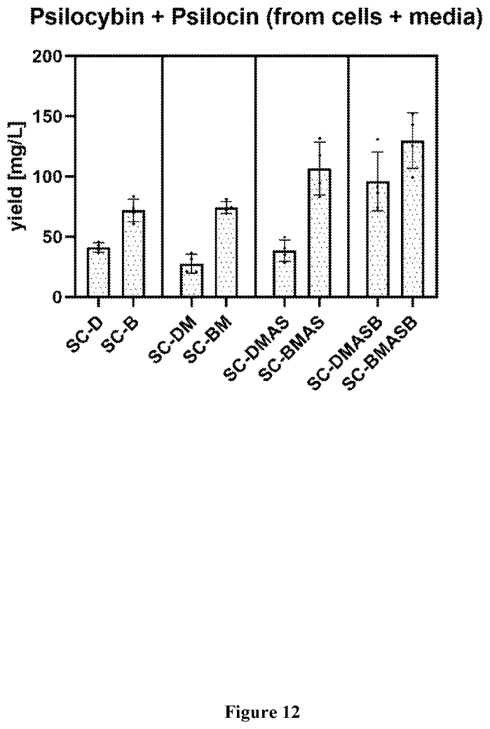

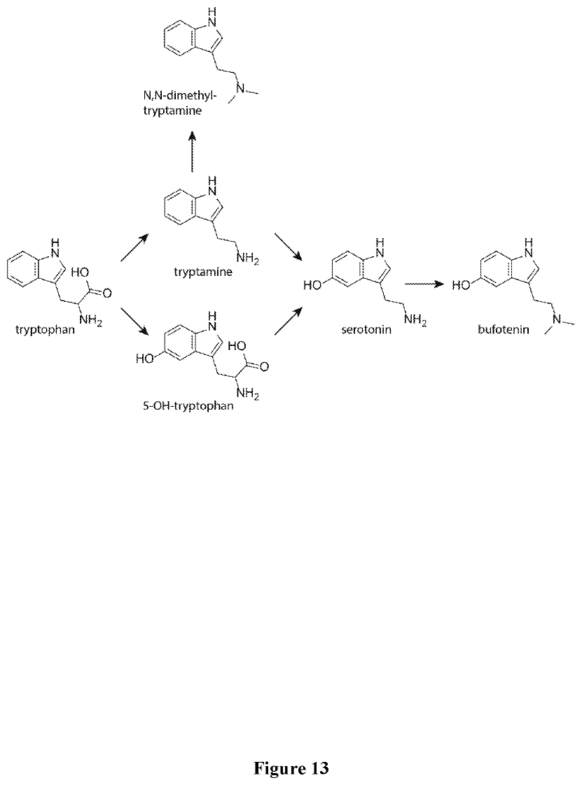

As shown in Table 5 and FIG. 10, the addition of a second copy each of native S. cerevisiae genes SAH1 and ADO1 in SC-BMAS and SC-DMAS resulted in an even larger difference in psilocybin and psilocin production using SC-BM strain in comparison to SC-DM strain, as well as a significant increase in overall production using SC-DMAS strain in comparison to SC-DM strain and using SC-BMAS strain in comparison to SC-BM strain, as shown in FIG. 12. This indicated a bottleneck in tryptamine conversion to psilocybin present in strains SC-B and SC-BM had been alleviated in strain SC-BMAS. Therefore, the significant difference in psilocybin and psilocin production using SC-BMAS strain in comparison to SC-DMAS strain was a result of the higher tryptamine production rate of cnsBv1 in comparison to psiD.

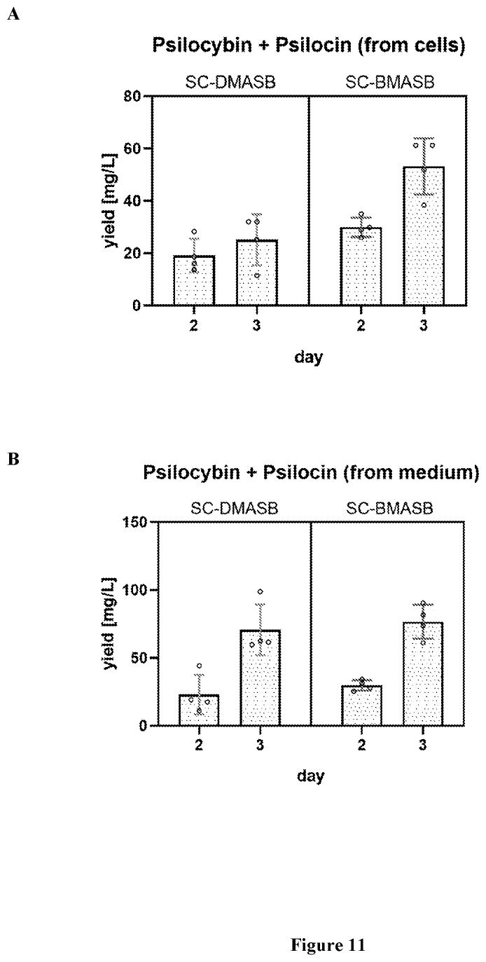

As shown in Table 6, FIG. 11 and FIG. 12 the addition of a copy of cnsBv1 in SC-BMASB and SC-DMASB resulted in major increase in production using SC-DMAS strain in comparison to SC-DMASB strain and a minor increase using SC-BMAS strain in comparison to SC-BMASB strain, indicating that single copy of cnsBv1 produced tryptamine in sufficient amounts to almost saturate downstream pathway enzymes psiH, psiK and psiM, whereas psiD supplied only a fraction of the tryptamine that downstream enzymes were capable of converting.

### Example 4. N-methyltransferase

### Plasmid Assembly and Strain Construction

The coding sequences of osTSH, osCPR and rmNMT (Chen X, Li J, Yu L, et al. Bioproduction platform using a novel cane toad (Rhinella marina) N-methyltransferase for psychedelic-inspired drug discovery. Research Square; 2023. DOI: 10.21203/rs.3.rs-2667175/v1.) were synthesized by Twist Biosciences as synthetic gene fragments.

Wild-type osTSH, osCPR and rmNMT coding sequences are provided in SEQ ID NOs: 37, 40 and 43 respectively. The amino acid sequences of the OsT5H, OsCPR and RmNMT polypeptides encoded by these coding sequences are provided in SEQ ID NOs: 36, 39 and 42, respectively.

The osTSH, osCPR and rmNMT coding sequences were codon optimized for expression in Saccharomyces cerevisiae and featured 5′- and 3′-end restriction sites for Golden Gate cloning. The codon optimized coding sequences of osTSH, osCPR and rmNMT are provided in SEQ ID NOs: 38, 41 and 44, respectively.

Plasmid pNMT-rmNMTi was constructed from individual parts with a BsaI Golden Gate assembly and contains the following components: pTDH3 promoter and tTDH1 terminator flanking asTDC1 (operably linked to the pTDH3 promoter); pPGK1 promoter and tSSA1 terminator flanking osCPR (operably linked to the pPGK1 promoter); pCCW12 promoter and tPGK1 terminator flanking osTSH (operably linked to the pCCW12 promoter); pTEF2 promoter and tENO1 terminator flanking rmNMT (operably linked to the pTEF2 promoter); ampicillin resistance gene; ColE1 origin of replication; URA3 auxotrophic marker; and homology arms for expression cassette integration into the URA3 locus of the S. cerevisiae genome.

Plasmid pNMT-RmNMT2 was constructed from individual parts with a BsaI Golden Gate assembly and contains the following components: pTDH3 promoter and tTDH1 terminator flanking asTDC1 (operably linked to the pTDH3 promoter); pTEF2 promoter and tENO1 terminator flanking rmNMT (operably linked to the pTEF2 promoter); ampicillin resistance gene; ColE1 origin of replication; URA3 auxotrophic marker; and homology arms for expression cassette integration into the URA3 locus of the S. cerevisiae genome.

Plasmid pNMT-rmNMT3 was constructed from individual parts with a BsaI Golden Gate assembly and contains the following components: pTDH3 promoter and tTDH1 terminator flanking cnsBv1 (operably linked to the pTDH3 promoter); pPGK1 promoter and tSSA1 terminator flanking osCPR (operably linked to the pPGK1 promoter); pCCW12 promoter and tPGK1 terminator flanking osTSH (operably linked to the pCCW12 promoter); pTEF2 promoter and tENO1 terminator flanking rmNMT (operably linked to the pTEF2 promoter); ampicillin resistance gene; ColE1 origin of replication; URA3 auxotrophic marker; and homology arms for expression cassette integration into the URA3 locus of the S. cerevisiae genome.

Plasmid pNMT-RmNMT4 was constructed from individual parts with a BsaI Golden Gate assembly and contains the following components: pTDH3 promoter and tTDH1 terminator flanking cnsBv1 (operably linked to the pTDH3 promoter); pTEF2 promoter and tENO1 terminator flanking rmNMT (operably linked to the pTEF2 promoter); ampicillin resistance gene; ColE1 origin of replication; URA3 auxotrophic marker; and homology arms for expression cassette integration into the URA3 locus of the S. cerevisiae genome.

Plasmid pNMT-RmNMT5 was constructed from individual parts with a BsaI Golden Gate assembly and contains the following components: pPGK1 promoter and tSSA1 terminator flanking osCPR (operably linked to the pPGK1 promoter); pCCW12 promoter and tPGK1 terminator flanking osTSH (operably linked to the pCCW12 promoter); pTEF2 promoter and tENO1 terminator flanking rmNMT (operably linked to the pTEF2 promoter); ampicillin resistance gene; ColE1 origin of replication; URA3 auxotrophic marker; and homology arms for expression cassette integration into the URA3 locus of the S. cerevisiae genome.

Transformation of S. cerevisiae with pNMT-rmNMT1-5 yielded strains SC-rmNMT1-5, respectively. S. cerevisiae strain BY4741 (cultured at 30° C. and 250 rpm) was used as the parent strain for all transformations. All transformations of S. cerevisiae were performed using the LiAc/SS carrier DNA/PEG method (Gietz and Schiestl, 2007, Nature Protocols, 2: 1-4).

### Biosynthesis

The ability of S. cerevisiae strains SC-rmNMT1 1-5 to produce tryptamine, 5-OH-tryptophan, serotonin, N,N-dimethyltryptamine and bufotenine was determined. 1 mL cultures of YPD (20 g/L glucose, 20 g/L peptone, 10 g/L yeast extract) were inoculated with single colonies of S. cerevisiae. Samples of 100 μL of culture broth were taken on day 6. Extractions were performed by the addition of 200 μL of methanol, followed by thorough vortexing. The extracts were thoroughly centrifuged to remove cell debris.

For analysis, 1 μL of the extraction was injected into a Vanquish UPLC system with a Kinetex C18 column (2.1×100 mm, 2.6 μm), coupled to an Orbitrap Exploris 120 MS (OE120) mass spectrometer with an ESI source. The mobile phase was a 10 min linear gradient of 5-95% acetonitrile-water containing 0.1% formic acid. Tryptamine, 5-OH-tryptophan, serotonin and N,N-dimethyltryptamine peaks in the extracted LC-MS chromatogmms were identified by comparison with the extracted chromatograms of tryptamine, 5-OH-tryptophan, serotonin and N,N-dimethyltryptamine authentic standards.

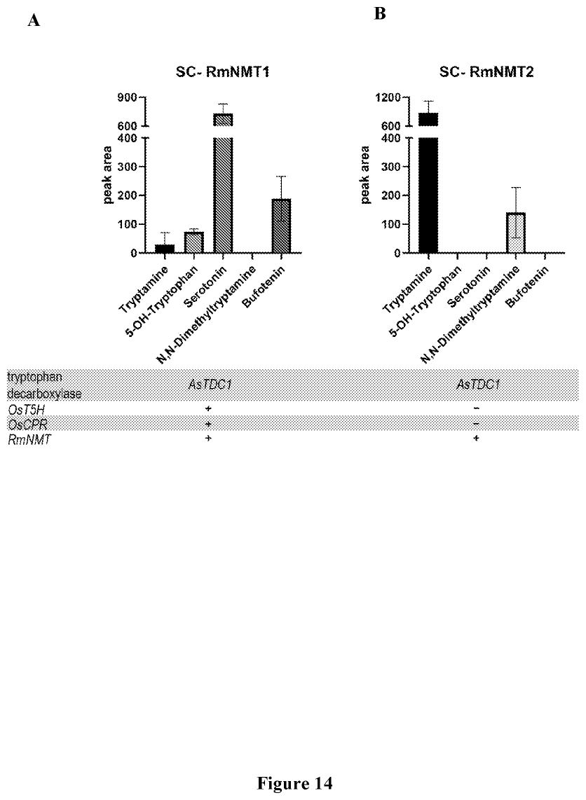

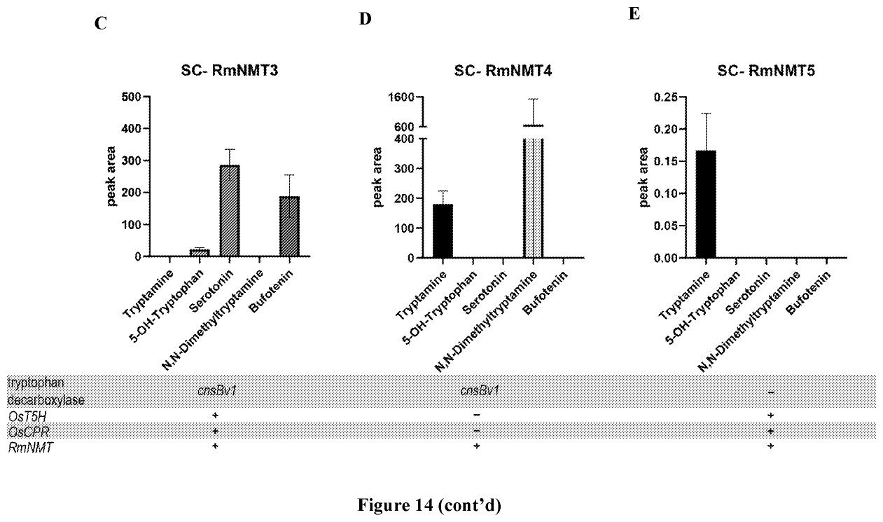

As shown in FIG. 14, PLP-dependent tryptophan carboxylases like cnsBv1 and asTDC1 can be used to supply tryptamine for the production of serotonin, N,N-dimethyltryptamine and bufotenin in the context of the appropriate biosynthetic pathway.

### Example 5. Escherichia coli with asTDC1

### Plasmid Assembly and Strain Construction

The coding sequences of psiD and asTDC1 were synthesized by Twist Biosciences as synthetic gene fragments.

The psiD and asTDC1 coding sequences were codon optimized for expression in E. coli and inserted into the NdeI-XhoI restriction site of pET28a. The codon optimized coding sequences of psiD and asTDC1 are provided in SEQ ID NOs: 45 and 46, respectively.

Transformation of E. coli BL21 (DE3) with pET28a-psiD, pET28a-asTDC1 and pET28a yielded strains EC-psiD, EC-asTDC1 and EC-ctrl respectively.

The ability of E. coli to produce tryptamine, psilocybin and psilocin is determined substantially as described in Examples 2 and 4.

### Tryptamine Biosynthesis

The ability of E. coli strains EC-psiD, EC-asTDC1 and EC-ctrl to produce tryptamine was determined. 1 mL cultures of Lysogeny broth (LB) (10 g/L tryptone, 10 g/L NaCl, 5 g/L yeast extract) were inoculated with single colonies of E. coli, incubated at 37° C. for 6 hours and 1 mM isopropyl β-d-1-thiogalactopyranoside (IPTG) was added. Samples of 100 μL of culture broth were taken on day 3. Extractions were performed by the addition of 200 μL of methanol, followed by thorough vortexing. The extracts were thoroughly centrifuged to remove cell debris.

LC-MS-based analysis and quantification of tryptamine yields was performed as described in Examples 2 and 4.

As shown in FIG. 15, representative PLP-dependent tryptophan carboxylase asTDC1 is active in E. coli and produces about 50% more tryptamine than PsiD.

### Example 6. S. cerevisiae with crTDC, AcTDC and cnsBv2

### Plasmid Assembly and Strain Construction

The coding sequences of crTDC, acTDC and cnsBv2 were synthesized by Twist Biosciences as synthetic gene fragments.

Wild-type crTDC and acTDC coding sequences are provided in SEQ ID NO: 30 and 50, respectively. The amino acid sequences of the CrTDC and AcTDC polypeptides encoded by these coding sequences are provided in SEQ ID NO: 29 and 48, respectively.

The crTDC, acTDC and cnsBv2 coding sequences were codon optimized for expression in Saccharomyces cerevisiae and featured 5′- and 3′-end restriction sites for Golden Gate cloning. The codon optimized coding sequences of crTDC, acTDC and cnsBv2 are provided in SEQ ID NOs: 47, 50 and 51, respectively.

Plasmids pNMT-crTDC, pNMT-acTDC and pNMT-cnsBv2 were constructed from individual parts with a BsaI Golden Gate assembly (Engler et al., 2008, PLoS One, 3: e3647). The plasmids are identical with the exception of the tryptophan decarboxylase gene and contain the following components: pTDH3 promoter and tENO1 terminator flanking the respective decarboxylase gene (the gene operably linked to the pTDH3 promoter), ampicillin resistance gene, ColE1 origin of replication, URA3 auxotrophic marker and homology arms for expression cassette integration into the URA3 locus of the S. cerevisiae genome.

Transformation of S. cerevisiae withpNMT-crTDC, pNMT-acTDC and pNMT-cnsBv2 yielded strains SC-crTDC, SC-acTDC and SC-cnsBv2, respectively. Each strain contains a single copy of the relevant tryptophan decarboxylase gene.

### Tryptamine Biosynthesis

The ability of S. cerevisiae strains SC-crTDC, SC-acTDC, SC-cnsBv1, SC-cnsBv2, SC-asTDC1 and SC-psiD to produce tryptamine was determined. 1 mL cultures of YPD (20 g/L glucose, 20 g/L peptone, 10 g/L yeast extract) were inoculated with single colonies of S. cerevisiae. Samples of 100 μL of culture broth were taken on day 3 of culturing. Extractions were performed by the addition of 200 μL of methanol, followed by thorough vortexing. The extracts were thoroughly centrifuged to remove cell debris.

LC-MS-based analysis and quantification of tryptamine yields was performed as described in Examples 2 and 4.

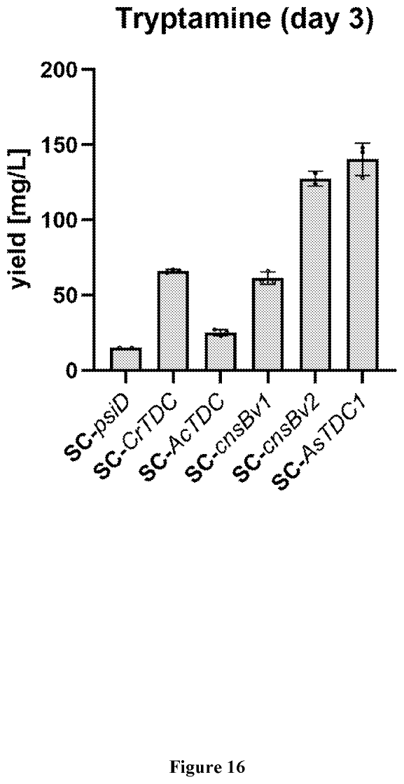

As shown in FIG. 16, different PLP-dependent tryptophan carboxylases exhibit varying tryptamine production rates, with CnsBv2 and asTDC1 outperforming commonly used TDCs PsiD and CrTDC.

### Example 7. Yarrowia with TDCs

Plasmid assembly is performed using the coding sequences of the enzymes substantially as described in Examples 1 and 4-6.

Strain construction and transformation of Yarrowia is performed substantially as described in Holkenbrink et al. Biotechnol J. 2018; 13(9):e1700543.

The ability of Yarrowia to produce tryptamine, psilocybin and psilocin is determined substantially as described in Examples 2 and 4.

### Example 8. Aspergillus

Plasmid assembly is performed using the coding sequences of the enzymes substantially as described in Examples 1 and 4-6.

Strain construction and transformation of Aspergillus is performed substantially as described in Roux and Chooi. Methods Mol Biol. 2022:2489:75-92.

The ability of Aspergillus to produce tryptamine is determined substantially as described in Example 2.

The ability ofAspergillus to produce psilocybin and psilocin is determined substantially as described in Examples 2 and 4-6.

### Example 9. Corynebacterium glutamicum

Plasmid assembly is performed using the coding sequences of the enzymes substantially as described in Examples 1 and 4-6.

Strain construction and transformation of C. glutamicum is performed substantially as described in Kim et al., 2023, CSBJ; Dunican & Shivnan, 1989, BioTechnology; Ruan et al. 2015, Biotechnology Letters; and Li et al., 2016, J. Microbiol. Methods.

The ability of C. glutamicum to produce tryptamine, psilocybin and psilocin is determined substantially as described in Examples 2 and 4.

### Example 10. Cell Free Production of Tryptophan Derivatives

Plasmid assembly is performed using the coding sequences of the enzymes is substantially as described in Examples 1 and 4-6.

Cell free production of tryptophan derivatives using enzymes as described herein is performed substantially as described in Fricke et al., 2020, Biosynthesis; and Blei et al., 2018, Biosynthesis.

TABLE 2
tryptamine [mg/L]

| | day 5 - cells | | | | | | | |
|---|---|---|---|---|---|---|---|---|
| | biological replicate # | | | | | | | fold |
| | 1 | 2 | 3 | 4 | 5 | 6 | average | change |
| SC-psiD | 7.5 | 7.6 | 8.2 | 8.5 | 9.4 | 10.4 | 8.6 | 2.3 |
| SC -cnsB | 18.2 | 20.3 | 18.2 | 23.4 | 21.4 | 17.7 | 19.9 | |
| | day 6 - cells | | | | | | | |
| | biological replicate # | | | | | | | fold |
| | 1 | 2 | 3 | 4 | 5 | 6 | average | change |
| SC-psiD | 5.7 | 6.5 | 5.5 | 6.9 | 8.8 | 8.6 | 7.0 | 2.3 |
| SC-cnsB | 10.8 | 16.9 | 15.5 | 17.3 | 17.2 | 20.1 | 16.3 | |
| | day 5 - medium | | | | | | | |
| | biological replicate # | | | | | | | fold |
| | 1 | 2 | 3 | 4 | 5 | 6 | average | change |
| SC-psiD | 66.9 | 54.4 | 57.9 | 59.7 | 73.8 | 81.7 | 65.7 | 1.7 |
| SC-cnsB | 124.4 | 124.7 | 105.1 | 130.7 | 87.4 | 112.6 | 114.2 | |
| | day 6 - medium | | | | | | | |
| | biological replicate # | | | | | | | fold |
| | 1 | 2 | 3 | 4 | 5 | 6 | average | change |
| SC-psiD | 67.4 | 76.3 | 67.7 | 68.2 | 82.7 | 81.9 | 74.0 | 2.0 |
| SC-cnsB | 136.5 | 149.5 | 143.4 | 161.6 | 138.0 | 140.8 | 144.9 | |

TABLE 3
psilocybin + psilocin [mg/L]

day 2 - cells

| | |
|---|---|
| biological replicate # | fold |

| | 1 | 2 | 3 | 4 | average | change |
|---|---|---|---|---|---|---|
| SC-D | 30.0 | 20.7 | 12.2 | 22.8 | 21.4 | 0.8 |
| SC-B | 14.5 | 14.1 | 17.1 | 27.2 | 18.2 | |

day 3 - cells

| | |
|---|---|
| biological replicate # | fold |

| | 1 | 2 | 3 | 4 | average | change |
|---|---|---|---|---|---|---|
| SC-D | 6.4 | 11.9 | 13.1 | 6.8 | 9.5 | 2.2 |
| SC-B | 15.1 | 21.6 | 23.7 | 23.0 | 20.9 | |

day 2 - medium

| | |
|---|---|
| biological replicate # | fold |

| | 1 | 2 | 3 | 4 | average | change |
|---|---|---|---|---|---|---|
| SC-D | 21.6 | 13.1 | 7.8 | 12.7 | 13.8 | 1.2 |
| SC-B | 12.6 | 12.4 | 13.0 | 27.2 | 16.3 | |

day 3 - medium

| | |
|---|---|
| biological replicate # | fold |

| | 1 | 2 | 3 | 4 | average | change |
|---|---|---|---|---|---|---|
| SC-D | 36.6 | 33.8 | 26.4 | 30.1 | 31.7 | 1.6 |
| SC-B | 45.8 | 48.0 | 50.1 | 60.4 | 51.1 | |

TABLE 4
psilocybin + psilocin [mg/L]

day 2 - cells

| | |
|---|---|
| biological replicate # | fold |

| | 1 | 2 | 3 | 4 | average | change |
|---|---|---|---|---|---|---|
| SC-DM | 10.1 | 3.5 | 1.8 | 2.9 | 4.5 | 2.0 |
| SC-BM | 13.8 | 7.0 | 7.1 | 8.9 | 9.2 | |

| | day 3 - cells | | | | | |
|---|---|---|---|---|---|---|
| | biological replicate # | | | | | fold |
| | 1 | 2 | 3 | 4 | average | change |
| SC-DM | 9.6 | 9.6 | 3.4 | 2.3 | 6.2 | 3.7 |
| SC-BM | 19.3 | 21.4 | 27.9 | 24.5 | 23.3 | |
| | day 2 - medium | | | | | |
| | biological replicate # | | | | | fold |
| | 1 | 2 | 3 | 4 | average | change |
| SC-DM | 13.8 | 7.5 | 7.3 | 7.5 | 9.0 | 1.6 |
| SC-BM | 17.6 | 14.5 | 13.8 | 11.9 | 14.4 | |
| | day 3 - medium | | | | | |
| | biological replicate # | | | | | fold |
| | 1 | 2 | 3 | 4 | average | change |
| SC-DM | 27.2 | 22.1 | 17.7 | 19.1 | 21.5 | 2.4 |
| SC-BM | 61.8 | 51.1 | 46.6 | 44.8 | 51.1 | |

TABLE 5
psilocybin + psilocin [mg/L]

| | day 2 - cells | | | | | |
|---|---|---|---|---|---|---|
| | biological replicate # | | | | | fold |
| | 1 | 2 | 3 | 4 | average | change |
| SC-DMAS | 7.8 | 4.5 | 3.6 | 4.8 | 5.2 | 6.1 |
| SC-BMAS | 42.2 | 17.8 | 48.4 | 17.7 | 31.5 | |
| | day 3 - cells | | | | | |
| | biological replicate # | | | | | fold |
| | 1 | 2 | 3 | 4 | average | change |
| SC-DMAS | 7.5 | 19.6 | 10.2 | 10.9 | 12.0 | 2.6 |
| SC-BMAS | 42.1 | 27.7 | 25.0 | 28.7 | 30.9 | |
| | day 2 - medium | | | | | |
| | biological replicate # | | | | | fold |
| | 1 | 2 | 3 | 4 | average | change |
| SC-DMAS | 21.2 | 10.2 | 8.2 | 11.8 | 12.9 | 2.8 |
| SC-BMAS | 48.2 | 25.2 | 52.7 | 14.8 | 35.4 | |
| | day 3 - medium | | | | | |
| | biological replicate # | | | | | fold |
| | 1 | 2 | 3 | 4 | average | change |
| SC-DMAS | 42.3 | 21.1 | 18.4 | 24.2 | 26.5 | 2.9 |
| SC-BMAS | 89.7 | 66.8 | 92.6 | 54.4 | 75.9 | |

TABLE 6
psilocybin + psilocin [mg/L]

| | day 2 - cells | | | | | |
|---|---|---|---|---|---|---|
| | biological replicate # | | | | | fold |
| | 1 | 2 | 3 | 4 | average | change |
| SC-DMASB | 28.3 | 16.0 | 13.8 | 18.6 | 19.2 | 1.6 |
| SC-BMASB | 29.8 | 35.0 | 26.0 | 29.0 | 30.0 | |
| | day 3 - cells | | | | | |
| | biological replicate # | | | | | fold |
| | 1 | 2 | 3 | 4 | average | change |
| SC-DMASB | 32.0 | 25.2 | 11.4 | 32.0 | 25.1 | 2.1 |
| SC-BMASB | 61.2 | 61.2 | 52.1 | 38.4 | 53.2 | |
| | day 2 - medium | | | | | |
| | biological replicate # | | | | | fold |
| | 1 | 2 | 3 | 4 | average | change |
| SC-DMASB | 44.2 | 10.9 | 19.0 | 17.6 | 22.9 | 1.3 |
| SC-BMASB | 34.4 | 31.3 | 28.2 | 25.4 | 29.8 | |
| | day 3 - medium | | | | | |
| | biological replicate # | | | | | fold |
| | 1 | 2 | 3 | 4 | average | change |
| SC-DMASB | 98.8 | 61.6 | 62.6 | 59.8 | 70.7 | 1.1 |
| SC-BMASB | 90.4 | 81.7 | 73.7 | 61.0 | 76.7 | |

TABLE 7
Sequences of genes and encoded polypeptides

| SEQ | | |
|---|---|---|
| ID NO: | Description | Sequence |
| 1 | CnsBv1 polypeptide | MNSPTASDPIVRTLDAPETKDMQEAFLPGESPQDVASVIQETMDIFD |
| | (GenBank: | KRMRLTHPHCFCYVPSCPSPLAWVGDLFTSLYNVNAVSWDVSSGPSA |
| | A0A0A2IDH4.1) | MEQGMIRWLAQQVGLPPSAGGCFVSGGSMANLSAVVAARDKLLPPMR |
| | | RAEGTIYTSDQTHISIKKCLHIIGFDEHQIRVIPTDHLFRMDTDILR |
| | | RTIAADRQCGRLPFLVVASCGTTNTGSIDPLHALADVAEEEGLWLHV |
| | | DGAYGASIALSDKYRHLVDGIGRADSISWDGHKWLFQTYGCGIVLTR |
| | | DSASLAHSFAVDAEYINSTTPHGAVNFYNLSPELSRPARAMPLWFTL |
| | | RVLGRRQMGEMIDQGFLLARTAEEEIRRYPHWVILAPVTASIVVFRY |
| | | CPLGLKDDDLDTLNLALSARLLAENVASILTTNIRGRVALRFCATNP |
| | | ATQADTITKVVQQMDQMANTMPTCGLGMCKE |
| 2 | cnsBv1 coding | ATGAATTCCCCAACTGCCTCTGATCCTATTGTCCGAACACTGGATGC |
| | sequence encoding | CCCTGAAACGAAAGACATGCAAGAGGCATTCCTCCCGGGAGAGAGCC |
| | CnsBv1 of SEQ ID | CACAAGACGTCGCATCAGTTATCCAGGAAACCATGGATATTTTCGAC |
| | NO: 1 (GenBank: | AAACGCATGCGTCTCACGCACCCACACTGCTTTTGCTATGTACCGTC |
| | JQFZ01000090) | ATGTCCATCTCCGCTAGCCTGGGTGGGGGACCTGTTCACCTCACTCT |
| | | ACAATGTAAACGCGGTTAGTTGGGATGTGAGTTCTGGTCCAAGTGCA |
| | | ATGGAACAAGGAATGATCAGGTGGCTGGCCCAGCAGGTTGGTCTTCC |
| | | TCCTTCTGCTGGTGGATGCTTTGTCTCCGGTGGCTCTATGGCAAACC |
| | | TATCCGCCGTCGTGGCAGCTCGAGATAAGTTGCTTCCCCCTATGCGA |
| | | CGGGCTGAGGGAACCATATACACATCGGACCAAACGCATATATCGAT |
| | | AAAGAAATGTCTCCATATAATCGGCTTCGACGAACACCAAATCCGCG |
| | | TTATCCCCACAGACCACCTATTCCGCATGGACACTGATATCCTGCGA |
| | | CGAACGATTGCAGCCGACCGCCAATGTGGTCGCCTACCTTTTCTCGT |
| | | CGTGGCCAGCTGTGGAACCACAAATACGGGCAGCATCGATCCCCTCC |
| | | ATGCTCTAGCCGACGTGGCTGAAGAAGAAGGACTGTGGCTGCACGTG |
| | | GATGGTGCGTACGGTGCGTCAATTGCCCTCTCCGACAAGTACCGCCA |
| | | TCTCGTGGATGGAATTGGTCGCGCAGACAGTATATCATGGGACGGAC |
| | | ATAAATGGCTTTTCCAAACATATGGTTGTGGAATTGTCCTCACTCGT |
| | | GATAGTGCGTCCCTCGCCCACAGCTTTGCCGTGGATGCAGAGTATAT |
| | | CAACTCGACGACACCGCACGGGGCCGTGAATTTCTACAACCTTAGTC |
| | | CTGAACTCAGCCGCCCAGCGAGAGCCATGCCGCTTTGGTTTACGCTG |
| | | AGGGTGCTGGGACGTCGGCAGATGGGAGAGATGATTGATCAAGGGTT |
| | | CCTTCTCGCCCGCACCGCCGAAGAGGAGATTCGACGATATCCGCATT |
| | | GGGTGATCCTGGCACCAGTAACAGCGAGTATTGTCGTCTTCCGATAC |
| | | TGTCCATTGGGGTTGAAGGATGATGATCTGGATACACTGAACCTGGC |
| | | CCTCTCAGCTCGACTTTTGGCAGAGAATGTTGCGTCCATCCTCACAA |
| | | CAAACATCCGTGGTCGGGTAGCACTACGTTTCTGTGCAACAAACCCT |
| | | GCCACCCAAGCAGATACAATCACGAAGGTGGTTCAACAAATGGACCA |
| | | AATGGCCAACACAATGCCCACTTGCGGACTTGGAATGTGCAAGGAAT |
| | | AG |
| 3 | CnsBv2 polypeptide | MTEKEEDVDQFHTKVSIATLAALTRLLKMNTQTPSDPIVRTLDAAGT |
| | (GenBank: | KEMQEAFLPGESPHDVASVIQETMDIFDKRMRLTHPHCFCYVPSCPS |
| | AMQ76109.1) | PLAWVGDLFTSLYNVNAVSWDVSSGPSAMEQAMIRWLAHQVGLPPSA |
| | | TGCFVSGGSMANLSAVVAARDKLLPPLRRPEGTIYVSDQTHISIKKC |
| | | LHIIGFHEHQVRVIPTDEAFRMDIAILQRTIAADRQCGRLPFLLVAS |
| | | CGTTNTGSIDPLDALADVAKEEGLWLHVDGAYGASIALSDKHGHLVN |
| | | GIGRADSISWDGHKWLFQTYGCGIVLTRDSTSLATSFAVDAEYINST |
| | | TPHGAVNFYNFGPELSRPARAIPLWFTLRVLGRRKMGEMIDHGFLLA |
| | | RTAEDEIRRYAHWLILAPVTASIVVFRYSPPELKDNELDELNLAIST |
| | | RLLTENVASIFTTNIRGRVALRFCAMNPATRTDTIMEVVQQMDEVAR |
| | | TISTSRPGLCKDCKD |
| 4 | cnsBv2 coding | ATGACTGAAAAAGAGGAAGATGTTGATCAATTCCATACAAAAGTTTC |
| | sequence (5′-3′) | AATCGCAACTCTAGCCGCGCTGACGCGACTACTAAAAATGAACACTC |
| | encoding CnsBv2 of | AAACTCCCTCCGATCCCATTGTCCGAACACTGGATGCTGCTGGAACA |
| | SEQ ID NO: 3 | AAAGAAATGCAAGAGGCATTCCTCCCAGGAGAGAGCCCACACGACGT |
| | (GenBank: | CGCGTCAGTTATCCAGGAAACTATGGATATTTTTGACAAACGTATGC |
| | KU932036.1) | GTCTCACGCACCCACACTGCTTTTGCTATGTACCGTCATGTCCATCG |
| | | CCGCTCGCCTGGGTGGGAGATCTGTTCACCTCGCTCTATAATGTGAA |
| | | CGCGGTCAGTTGGGACGTGAGTTCCGGTCCTAGTGCAATGGAACAGG |
| | | CCATGATCAGGTGGCTAGCCCATCAGGTTGGTCTTCCTCCTTCTGCT |
| | | ACGGGATGCTTCGTCTCCGGTGGCTCTATGGCAAACCTATCCGCCGT |
| | | TGTGGCAGCTCGAGATAAATTACTCCCCCCTTTACGGCGGCCTGAGG |
| | | GCACCATATACGTATCAGACCAGACGCATATATCGATAAAGAAATGT |
| | | CTCCACATAATCGGCTTCCACGAACACCAAGTCCGCGTTATTCCCAC |
| | | AGACGAGGCATTCCGCATGGACATAGCCATTCTACAGCGAACGATTG |
| | | CAGCCGACCGCCAATGTGGCCGGCTCCCTTTTCTCCTCGTGGCTAGC |
| | | TGTGGGACTACTAATACGGGAAGCATTGATCCCCTCGATGCTTTAGC |
| | | TGATGTGGCCAAAGAAGAGGGACTGTGGCTGCACGTGGATGGTGCCT |
| | | ACGGTGCATCAATTGCCCTCTCCGACAAGCACGGCCATCTCGTCAAT |
| | | GGAATCGGTCGCGCAGATAGTATATCATGGGATGGGCACAAATGGCT |
| | | TTTCCAGACATATGGCTGTGGAATTGTCCTCACTCGTGATAGCACAT |
| | | CCCTCGCCACCAGTTTTGCCGTGGATGCGGAATATATCAACTCAACG |
| | | ACACCGCACGGGGCCGTGAATTTTTATAACTTTGGCCCCGAGCTCAG |
| | | CCGCCCAGCGCGAGCTATACCGCTTTGGTTTACGCTGAGAGTGCTAG |
| | | GTCGTCGGAAGATGGGAGAGATGATTGACCACGGCTTCCTTCTCGCC |
| | | CGCACTGCCGAAGACGAAATTCGACGATATGCGCATTGGTTGATCCT |
| | | GGCGCCAGTAACAGCGAGTATAGTCGTCTTTCGATACTCTCCACCGG |
| | | AGTTAAAGGACAATGAGCTGGATGAACTGAACTTAGCCATCTCAACT |
| | | CGACTTTTGACGGAAAATGTTGCTTCCATCTTCACGACAAACATTCG |
| | | TGGCCGGGTGGCCCTCCGTTTCTGTGCGATGAATCCTGCCACCCGAA |
| | | CAGATACGATCATGGAGGTAGTGCAACAAATGGACGAAGTGGCCAGG |
| | | ACGATTTCTACTTCCAGACCTGGATTGTGCAAGGACTGCAAGGACTA |
| | | G |
| 5 | ΔCnsBv2 polypeptide | MNTQTPSDPIVRTLDAAGTKEMQEAFLPGESPHDVASVIQETMDIFD |
| | (28 aa truncation of | KRMRLTHPHCFCYVPSCPSPLAWVGDLFTSLYNVNAVSWDVSSGPSA |
| | SEQ ID NO: 3) | MEQAMIRWLAHQVGLPPSATGCFVSGGSMANLSAVVAARDKLLPPLR |
| | | RPEGTIYVSDQTHISIKKCLHIIGFHEHQVRVIPTDEAFRMDIAILQ |
| | | RTIAADRQCGRLPFLLVASCGTTNTGSIDPLDALADVAKEEGLWLHV |
| | | DGAYGASIALSDKHGHLVNGIGRADSISWDGHKWLFQTYGCGIVLTR |
| | | DSTSLATSFAVDAEYINSTTPHGAVNFYNFGPELSRPARAIPLWFTL |
| | | RVLGRRKMGEMIDHGFLLARTAEDEIRRYAHWLILAPVTASIVVFRY |
| | | SPPELKDNELDELNLAISTRLLTENVASIFTTNIRGRVALRFCAMNP |
| | | ATRTDTIMEVVQQMDEVARTISTSRPGLCKDCKD |
| 6 | ΔcnsBv2 coding | ATGAACACTCAAACTCCCTCCGATCCCATTGTCCGAACACTGGATGC |
| | sequence encoding | TGCTGGAACAAAAGAAATGCAAGAGGCATTCCTCCCAGGAGAGAGCC |
| | ΔCnsBv2 of SEQ ID | CACACGACGTCGCGTCAGTTATCCAGGAAACTATGGATATTTTTGAC |
| | NO: 5 | AAACGTATGCGTCTCACGCACCCACACTGCTTTTGCTATGTACCGTC |
| | | ATGTCCATCGCCGCTCGCCTGGGTGGGAGATCTGTTCACCTCGCTCT |
| | | ATAATGTGAACGCGGTCAGTTGGGACGTGAGTTCCGGTCCTAGTGCA |
| | | ATGGAACAGGCCATGATCAGGTGGCTAGCCCATCAGGTTGGTCTTCC |
| | | TCCTTCTGCTACGGGATGCTTCGTCTCCGGTGGCTCTATGGCAAACC |
| | | TATCCGCCGTTGTGGCAGCTCGAGATAAATTACTCCCCCCTTTACGG |
| | | CGGCCTGAGGGCACCATATACGTATCAGACCAGACGCATATATCGAT |
| | | AAAGAAATGTCTCCACATAATCGGCTTCCACGAACACCAAGTCCGCG |
| | | TTATTCCCACAGACGAGGCATTCCGCATGGACATAGCCATTCTACAG |
| | | CGAACGATTGCAGCCGACCGCCAATGTGGCCGGCTCCCTTTTCTCCT |
| | | CGTGGCTAGCTGTGGGACTACTAATACGGGAAGCATTGATCCCCTCG |
| | | ATGCTTTAGCTGATGTGGCCAAAGAAGAGGGACTGTGGCTGCACGTG |
| | | GATGGTGCCTACGGTGCATCAATTGCCCTCTCCGACAAGCACGGCCA |
| | | TCTCGTCAATGGAATCGGTCGCGCAGATAGTATATCATGGGATGGGC |
| | | ACAAATGGCTTTTCCAGACATATGGCTGTGGAATTGTCCTCACTCGT |
| | | GATAGCACATCCCTCGCCACCAGTTTTGCCGTGGATGCGGAATATAT |
| | | CAACTCAACGACACCGCACGGGGCCGTGAATTTTTATAACTTTGGCC |
| | | CCGAGCTCAGCCGCCCAGCGCGAGCTATACCGCTTTGGTTTACGCTG |
| | | AGAGTGCTAGGTCGTCGGAAGATGGGAGAGATGATTGACCACGGCTT |
| | | CCTTCTCGCCCGCACTGCCGAAGACGAAATTCGACGATATGCGCATT |
| | | GGTTGATCCTGGCGCCAGTAACAGCGAGTATAGTCGTCTTTCGATAC |
| | | TCTCCACCGGAGTTAAAGGACAATGAGCTGGATGAACTGAACTTAGC |
| | | CATCTCAACTCGACTTTTGACGGAAAATGTTGCTTCCATCTTCACGA |
| | | CAAACATTCGTGGCCGGGTGGCCCTCCGTTTCTGTGCGATGAATCCT |
| | | GCCACCCGAACAGATACGATCATGGAGGTAGTGCAACAAATGGACGA |
| | | AGTGGCCAGGACGATTTCTACTTCCAGACCTGGATTGTGCAAGGACT |
| | | GCAAGGACTAG |
| 7 | AsTDC1 polypeptide | MPSHEEKSDDGLSEIMLETESLLTRLFTIKSPAPSDPIVQKLNAHEI |
| | (GenBank: | QNMQHQLMPDDHPRDLSSALQQAMAVFDNRMRLTHPRCFSYIPVCPN |
| | XP_024705640.1) | PVALLGDLLTSVFNVNAASWDVGSGPSAVETAMIAWLGAQLGLPDSV |
| | | EGCFVSGGSMGNMTAIVAARDEILHPLQRAYAIIYISDQTHISILKA |
| | | LHITGFTHHQVRKIPTDDDFRIDVDILRRTVASDRHSGLVPFLLVAT |
| | | CGTTNTGSIDPLHALADLARDEGLWLHVDGAYGASIALSDQHRSLVD |
| | | GIGRADSISWDGHKWLFQTYGCGIVLTRRSGSLRRSFAVDAEYIRDS |
| | | TVPEGTTNFYNLGPELSRPARAMSLWLTLRVLGRRRMGEMIDRGFLL |
| | | ARTADHSIRQYRDWIIAAPTVASIVVFRYAPPGLDDGALDALNKAIS |
| | | RRLLAEDVAAILTTTVRGRTVLRMCAMNPLVEPETIEDVIRRVDGIA |
| | | RSEASRRKESHFQLGSAMYLG |
| 8 | asTDC1 coding | ATGCCTTCCCATGAAGAAAAGTCCGATGACGGGCTGTCTGAGATCAT |
| | sequence (5′-3′) | GCTGGAAACAGAATCTCTCTTGACTCGACTTTTCACGATCAAATCTC |
| | encoding AsTDC1 of | CAGCACCATCCGATCCCATTGTTCAAAAGCTCAATGCCCATGAGATA |
| | SEQ ID NO: 7 | CAAAACATGCAGCATCAACTGATGCCTGATGACCATCCTCGAGATCT |
| | (GenBank: | TTCTTCCGCACTTCAACAAGCGATGGCAGTCTTCGATAACCGCATGC |
| | XM_024851485.1) | GCCTCACCCACCCCCGTTGCTTCTCCTACATCCCCGTCTGTCCCAAC |
| | | CCCGTTGCCCTGTTGGGTGACCTCCTCACTTCTGTCTTCAATGTAAA |
| | | CGCTGCCAGTTGGGACGTGGGCTCTGGTCCCAGCGCGGTCGAAACAG |
| | | CCATGATAGCCTGGCTGGGCGCCCAGCTTGGACTGCCCGACTCAGTC |
| | | GAAGGATGCTTTGTCTCCGGCGGCTCGATGGGCAACATGACAGCCAT |
| | | CGTTGCGGCGCGAGACGAGATCCTTCATCCCCTGCAGCGCGCATACG |
| | | CCATCATCTACATCTCCGACCAGACACACATCTCCATCCTCAAGGCC |
| | | CTCCACATCACCGGCTTCACGCACCACCAGGTCCGCAAGATCCCCAC |
| | | CGACGATGACTTCCGCATCGATGTCGACATCCTGCGACGCACCGTCG |
| | | CTTCCGACCGCCACTCCGGCCTCGTTCCTTTCCTGCTTGTCGCCACC |
| | | TGCGGCACCACCAACACCGGCAGCATCGACCCTCTGCATGCGCTAGC |
| | | GGATCTCGCGCGCGACGAAGGCCTCTGGCTCCATGTCGACGGCGCAT |
| | | ACGGTGCCTCCATCGCGCTCTCCGATCAGCACCGCTCCCTCGTTGAC |
| | | GGGATCGGTCGCGCAGACAGCATCTCCTGGGACGGCCACAAATGGCT |
| | | CTTCCAGACCTACGGCTGCGGGATCGTACTCACGCGTCGCTCTGGCT |
| | | CGCTTCGCCGCAGCTTCGCCGTGGACGCCGAGTACATCCGCGACTCA |
| | | ACGGTACCCGAGGGGACCACGAACTTCTACAACCTCGGCCCCGAGCT |
| | | CAGTCGGCCTGCGCGCGCCATGTCTCTCTGGCTCACTTTGCGAGTCC |
| | | TCGGTCGTCGTCGCATGGGCGAGATGATCGACCGGGGTTTCCTCCTT |
| | | GCGCGCACCGCAGACCACAGCATCCGTCAGTACAGGGACTGGATCAT |
| | | CGCGGCGCCGACGGTCGCCAGCATTGTAGTTTTTCGGTACGCGCCGC |
| | | CGGGCCTGGACGACGGGGCGCTGGACGCGTTGAACAAGGCCATTTCA |
| | | CGGCGTCTCCTGGCCGAGGATGTTGCTGCTATTCTCACGACTACGGT |
| | | TCGCGGACGCACTGTGCTTCGGATGTGTGCTATGAATCCTCTTGTGG |
| | | AGCCGGAGACGATCGAGGATGTGATACGTCGAGTGGATGGGATCGCG |
| | | CGGTCGGAGGCGTCCAGACGCAAGGAGAGCCATTTTCAATTGGGATC |
| | | TGCCATGTATCTTGGCTGA |
| 9 | PsiD polypeptide | MQVIPACNSAAIRSLCPTPESFRNMGWLSVSDAVYSEFIGELATRAS |
| | (GenBank: | NRNYSNEFGLMQPIQEFKAFIESDPVVHQEFIDMFEGIQDSPRNYQE |
| | ASU62239.1) | LCNMFNDIFRKAPVYGDLGPPVYMIMAKLMNTRAGFSAFTRQRLNLH |
| | | FKKLFDTWGLFLSSKDSRNVLVADQFDDRHCGWLNERALSAMVKHYN |
| | | GRAFDEVFLCDKNAPYYGFNSYDDFFNRRFRNRDIDRPVVGGVNNTT |
| | | LISAACESLSYNVSYDVQSLDTLVFKGETYSLKHLLNNDPFTPQFEH |
| | | GSILQGFLNVTAYHRWHAPVNGTIVKIINVPGTYFAQAPSTIGDPIP |
| | | DNDYDPPPYLKSLVYFSNIAARQIMFIEADNKEIGLIFLVFIGMTEI |
| | | STCEATVSEGQHVNRGDDLGMFHFGGSSFALGLRKDCRAEIVEKFTE |
| | | PGTVIRINEVVAALKA |
| 10 | psiD coding | ATGCAGGTGATACCCGCGTGCAACTCGGCAGCAATAAGATCACTATG |
| | sequence (5′-3′) | TCCTACTCCCGAGTCTTTTAGAAACATGGGATGGCTCTCTGTCAGCG |
| | (GenBank: | ATGCGGTCTACAGCGAGTTCATAGGAGAGTTGGCTACCCGCGCTTCC |
| | KY984101.1) | AATCGAAATTACTCCAACGAGTTCGGCCTCATGCAACCTATCCAGGA |
| | | ATTCAAGGCTTTCATTGAAAGCGACCCGGTGGTGCACCAAGAATTTA |
| | | TTGACATGTTCGAGGGCATTCAGGACTCTCCAAGGAATTATCAGGAA |
| | | CTATGTAATATGTTCAACGATATCTTTCGCAAAGCTCCCGTCTACGG |
| | | AGACCTTGGCCCTCCCGTTTATATGATTATGGCCAAATTAATGAACA |
| | | CCCGAGCGGGCTTCTCTGCATTCACGAGACAAAGGTTGAACCTTCAC |
| | | TTCAAAAAACTTTTCGATACCTGGGGATTGTTCCTGTCTTCGAAAGA |
| | | TTCTCGAAATGTTCTTGTGGCCGACCAGTTCGACGACAGACATTGCG |
| | | GCTGGTTGAACGAGCGGGCCTTGTCTGCTATGGTTAAACATTACAAT |
| | | GGACGCGCATTTGATGAAGTCTTCCTCTGCGATAAAAATGCCCCATA |
| | | CTACGGCTTCAACTCTTACGACGACTTCTTTAATCGCAGATTTCGAA |
| | | ACCGAGATATCGACCGACCTGTAGTCGGTGGAGTTAACAACACCACC |
| | | CTCATTTCTGCTGCTTGCGAATCACTTTCCTACAACGTCTCTTATGA |
| | | CGTCCAGTCTCTCGACACTTTAGTTTTCAAAGGAGAGACTTATTCGC |
| | | TTAAGCATTTGCTGAATAATGACCCTTTCACCCCACAATTCGAGCAT |
| | | GGGAGTATTCTACAAGGATTCTTGAACGTCACCGCTTACCACCGATG |
| | | GCACGCACCCGTCAATGGGACAATCGTCAAAATCATCAACGTTCCAG |
| | | GTACCTACTTTGCGCAAGCCCCGAGCACGATTGGCGACCCTATCCCG |
| | | GATAACGATTACGACCCACCTCCTTACCTTAAGTCTCTTGTCTACTT |
| | | CTCTAATATTGCCGCAAGGCAAATTATGTTTATTGAAGCCGACAACA |
| | | AGGAAATTGGCCTCATTTTCCTTGTGTTCATCGGCATGACCGAAATC |
| | | TCGACATGTGAAGCCACGGTGTCCGAAGGTCAACACGTCAATCGTGG |
| | | CGATGACTTGGGAATGTTCCATTTCGGTGGTTCTTCGTTCGCGCTTG |
| | | GTCTGAGGAAGGATTGCAGGGCAGAGATCGTTGAAAAGTTCACCGAA |
| | | CCCGGAACAGTGATCAGAATCAACGAAGTCGTCGCTGCTCTAAAGGC |
| | | TTAG |
| 11 | PsiH polypeptide | MIAVLFSFVIAGCIYYIVSRRVRRSRLPPGPPGIPIPFIGNMFDMPE |
| | (GenBank: | ESPWLTFLQWGRDYNTDILYVDAGGTEMVILNTLETITDLLEKRGSI |
| | ASU62246.1) | YSGRLESTMVNELMGWEFDLGFITYGDRWREERRMFAKEFSEKGIKQ |
| | | FRHAQVKAAHQLVQQLTKTPDRWAQHIRHQIAAMSLDIGYGIDLAED |
| | | DPWLEATHLANEGLAIASVPGKFWVDSFPSLKYLPAWFPGAVFKRKA |
| | | KVWREAADHMVDMPYETMRKLAPQGLTRPSYASARLQAMDLNGDLEH |
| | | QEHVIKNTAAEVNVGGGDTTVSAMSAFILAMVKYPEVQRKVQAELDA |
| | | LTNNGQIPDYDEEDDSLPYLTACIKELFRWNQIAPLAIPHKLMKDDV |
| | | YRGYLIPKNTLVFANTWAVLNDPEVYPDPSVFRPERYLGPDGKPDNT |
| | | VRDPRKAAFGYGRRNCPGIHLAQSTVWIAGATLLSAFNIERPVDQNG |
| | | KPIDIPADFTTGFFRHPVPFQCRFVPRTEQVSQSVSGP |
| 12 | psiH coding | ATGATCGCTGTACTATTCTCCTTCGTCATTGCAGGATGCATATACTA |
| | sequence (5′-3′) | CATCGTTTCTCGTAGAGTGAGGCGGTCGCGCTTGCCACCAGGGCCGC |
| | (GenBank: | CTGGCATTCCTATTCCCTTCATTGGGAACATGTTTGATATGCCTGAA |
| | MF000993.1) | GAATCTCCATGGTTAACATTTCTACAATGGGGACGGGATTACAGTCT |
| | | GTCTTGCCGCGTTGACTTCTAATATATGAACAGCTAATATATTGTCA |
| | | GACACCGATATTCTCTACGTGGATGCTGGAGGGACAGAAATGGTTAT |
| | | TCTTAACACGTTGGAGACCATTACCGATCTATTAGAAAAGCGAGGGT |
| | | CCATTTATTCTGGCCGGTGAGCTGATGTTGAGTTTTTTGCAATTGAA |
| | | TTTGTGGTCACACGTTTCCAGACTTGAGAGTACAATGGTCAACGAAC |
| | | TTATGGGGTGGGAGTTTGACTTAGGGTTCATCACATACGGCGACAGG |
| | | TGGCGCGAAGAAAGGCGCATGTTCGCCAAGGAGTTCAGTGAGAAGGG |
| | | CATCAAGCAATTTCGCCATGCTCAAGTGAAAGCTGCCCATCAGCTTG |
| | | TCCAACAGCTTACCAAAACGCCAGACCGCTGGGCACAACATATTCGC |
| | | CAGTAAGTACTACTTGAGGAAAATAGCGTACGCTTCGCTGACCGGTC |
| | | CGTACATCAAAGTCAGATAGCGGCAATGTCACTGGATATTGGTTATG |
| | | GAATTGATCTTGCAGAAGACGACCCTTGGCTGGAAGCGACCCATTTG |
| | | GCTAATGAAGGCCTCGCCATAGCATCAGTGCCGGGCAAATTTTGGGT |
| | | CGATTCGTTCCCTTCTCGTGAGCATCCTTCTTCTATGTAGGAAGGGA |
| | | AGGAGTCTAACAAGTGTTAGTAAAATACCTTCCTGCTTGGTTCCCAG |
| | | GTGCTGTCTTCAAGCGCAAAGCGAAGGTCTGGCGAGAAGCCGCCGAC |
| | | CATATGGTTGACATGCCTTATGAAACTATGAGGAAATTAGCAGTTAG |
| | | TCAAATGCGTTCTCCCCGTATTTTTTCAATACTCTAACTTCAGCTCA |
| | | CAGCCTCAAGGATTGACTCGTCCGTCGTATGCTTCAGCTCGTCTGCA |
| | | AGCCATGGATCTCAACGGTGACCTTGAGCATCAAGAACACGTAATCA |
| | | AGAACACAGCCGCAGAGGTTAATGTCGGTAAGTCAAAAGCGTCCGTC |
| | | GGCAATTCAAAATTCAGGCGCTAAAGTGGGTCTTCTCACCAAGGTGG |
| | | AGGCGATACTGTAAGGATTTCTCAATCGTTAGAGTATAAGTGTTCTA |
| | | ATGCAGTACATACTCCACCAACCAGACTGTCTCTGCTATGTCTGCGT |
| | | TCATCTTGGCCATGGTGAAGTACCCTGAGGTCCAGCGAAAGGTTCAA |
| | | GCGGAGCTTGATGCTCTGACCAATAACGGCCAAATTCCTGACTATGA |
| | | CGAAGAAGATGACTCCTTGCCATACCTCACCGCATGTATCAAGGAGC |
| | | TTTTCCGGTGGAATCAAATCGCACCCCTCGCTATACCGCACAAATTA |
| | | ATGAAGGACGACGTGTACCGCGGGTATCTGATTCCCAAGAACACTCT |
| | | AGTCTTCGCAAACACCTGGTGAGGCTGTCCATTCATTCCTAGTACAT |
| | | CCGTTGCCCCACTAATAGCATCTTGATAACAGGGCAGTATTAAACGA |
| | | TCCAGAAGTCTATCCAGATCCCTCTGTGTTCCGCCCAGAAAGATATC |
| | | TTGGTCCTGACGGGAAGCCTGATAACACTGTACGCGACCCACGTAAA |
| | | GCGGCATTTGGCTATGGACGACGAAATTGGTAAGTGCGCTTTCAGAA |
| | | CCCCCCCTTCCGTTGACTAGTGCCATGCGCGCATACAATATCGCTAT |
| | | TGATCTGATATAACTTCCCTGCGGCATTTATTTTGGCATTCCTTTAG |
| | | TCCCGGAATTCATCTAGCGCAGTCGACGGTTTGGATTGCAGGGGCAA |
| | | CCCTCTTATCAGCGTTCAATATCGAGCGACCTGTCGATCAGAATGGG |
| | | AAGCCCATTGACATACCGGCTGATTTTACTACAGGATTCTTCAGGTA |
| | | GCTAATTTCCGTCTTTGTGTGCATAATACCCCTAACGACGCACGTTT |
| | | ACCTTTTTGTAAAGACACCCAGTGCCTTTCCAGTGCAGGTTTGTTCC |
| | | TCGAACAGAGCAAGTCTCACAGTCGGTATCCGGACCCTGA |
| 13 | PsiK polypeptide | MAFDLKTEDGLITYLTKHLSLDVDTSGVKRLSGGFVNVTWRIKLNAP |
| | (GenBank: | YQGHTSIILKHAQPHMSTDEDFKIGVERSVYEYQAIKLMMANREVLG |
| | ASU62237.1) | GVDGIVSVPEGLNYDLENNALIMQDVGKMKTLLDYVTAKPPLATDIA |
| | | RLVGTEIGGFVARLHNIGRERRDDPEFKFFSGNIVGRTTSDQLYQTI |
| | | IPNAAKYGVDDPLLPTVVKDLVDDVMHSEETLVMADLWSGNILLQLE |
| | | EGNPSKLQKIYILDWELCKYGPASLDLGYFLGDCYLISRFQDEQVGT |
| | | TMRQAYLQSYARTSKHSINYAKVTAGIAAHIVMWTDFMQWGSEEERI |
| | | NFVKKGVAAFHDARGNNDNGEITSTLLKESSTA |
| 14 | psiK coding | ATGGCGTTCGATCTCAAGACTGAAGACGGCCTCATCACATATCTCAC |
| | sequence (5′-3′) | TAAACATCTTTCTTTGGACGTCGACACGAGCGGAGTGAAGCGCCTTA |
| | (GenBank: | GCGGAGGCTTTGTCAATGTAACCTGGCGCATTAAGCTCAATGCTCCT |
| | KY984099.1) | TATCAAGGTCATACGAGCATCATCCTGAAGCATGCTCAGCCGCACAT |
| | | GTCTACGGATGAGGATTTTAAGATAGGTGTAGAACGTTCGGTTTACG |
| | | AATACCAGGCTATCAAGCTCATGATGGCCAATCGGGAGGTTCTGGGA |
| | | GGCGTGGATGGCATAGTTTCTGTGCCAGAAGGCCTGAACTACGACTT |
| | | AGAGAATAATGCATTGATCATGCAAGATGTCGGGAAGATGAAGACCC |
| | | TTTTAGATTATGTCACCGCCAAACCGCCACTTGCGACGGATATAGCC |
| | | CGCCTTGTTGGGACAGAAATTGGGGGGTTCGTTGCCAGACTCCATAA |
| | | CATAGGCCGCGAGAGGCGAGACGATCCTGAGTTCAAATTCTTCTCTG |
| | | GAAATATTGTCGGAAGGACGACTTCAGACCAGCTGTATCAAACCATC |
| | | ATACCCAACGCAGCGAAATATGGCGTCGATGACCCCTTGCTGCCTAC |
| | | TGTGGTTAAGGACCTTGTGGACGATGTCATGCACAGCGAAGAGACCC |
| | | TTGTCATGGCGGACCTGTGGAGTGGAAATATTCTTCTCCAGTTGGAG |
| | | GAGGGAAACCCATCGAAGCTGCAGAAGATATATATCCTGGATTGGGA |
| | | ACTTTGCAAGTACGGCCCAGCGTCGTTGGACCTGGGCTATTTCTTGG |
| | | GTGACTGCTATTTGATATCCCGCTTTCAAGACGAGCAGGTCGGTACG |
| | | ACGATGCGGCAAGCCTACTTGCAAAGCTATGCGCGTACGAGCAAGCA |
| | | TTCGATCAACTACGCCAAAGTCACTGCAGGTATTGCTGCTCATATTG |
| | | TGATGTGGACCGACTTTATGCAGTGGGGGAGCGAGGAAGAAAGGATA |
| | | AATTTTGTGAAAAAGGGGGTAGCTGCCTTTCACGACGCCAGGGGCAA |
| | | CAACGACAATGGGGAAATTACGTCTACCTTACTGAAGGAATCATCCA |
| | | CTGCGTAA |
| 15 | PsiM polypeptide | MHIRNPYRTPIDYQALSEAFPPLKPFVSVNADGTSSVDLTIPEAQRA |
| | (GenBank: | FTAALLHRDFGLTMTIPEDRLCPTVPNRLNYVLWIEDIFNYTNKTLG |
| | ASU62238.1) | LSDDRPIKGVDIGTGASAIYPMLACARFKAWSMVGTEVERKCIDTAR |
| | | LNVVANNLQDRLSILETSIDGPILVPIFEATEEYEYEFTMCNPPFYD |
| | | GAADMQTSDAAKGFGFGVGAPHSGTVIEMSTEGGESAFVAQMVRESL |
| | | KLRTRCRWYTSNLGKLKSLKEIVGLLKELEISNYAINEYVQGSTRRY |
| | | AVAWSFTDIQLPEELSRPSNPELSSLF |
| 16 | psiM coding | ATGCATATCAGAAATCCTTACCGTACACCAATTGACTATCAAGCACT |
| | sequence (5′-3′) | TTCAGAGGCCTTCCCTCCCCTCAAGCCATTTGTGTCTGTCAATGCAG |
| | (GenBank: | ATGGTACCAGTTCTGTTGACCTCACTATCCCAGAAGCCCAGAGGGCG |
| | KY984100.1) | TTCACGGCCGCTCTTCTTCATCGTGACTTCGGGCTCACCATGACCAT |
| | | ACCAGAAGACCGTCTGTGCCCAACAGTCCCCAATAGGTTGAACTACG |
| | | TTCTGTGGATTGAAGATATTTTCAACTACACGAACAAAACCCTCGGC |
| | | CTGTCGGATGACCGTCCTATTAAAGGCGTTGATATTGGTACAGGAGC |
| | | CTCCGCAATTTATCCTATGCTTGCCTGTGCTCGGTTCAAGGCATGGT |
| | | CTATGGTTGGAACAGAGGTCGAGAGGAAGTGCATTGACACGGCCCGC |
| | | CTCAATGTCGTCGCGAACAATCTCCAAGACCGTCTCTCGATATTAGA |
| | | GACATCCATTGATGGTCCTATTCTCGTCCCCATTTTCGAGGCGACTG |
| | | AAGAATACGAATACGAGTTTACTATGTGTAACCCTCCATTCTACGAC |
| | | GGTGCTGCCGATATGCAGACTTCGGATGCTGCCAAAGGATTTGGATT |
| | | TGGCGTGGGCGCTCCCCATTCTGGAACAGTCATCGAAATGTCGACTG |
| | | AGGGAGGTGAATCGGCTTTCGTCGCTCAGATGGTCCGTGAGAGCTTG |
| | | AAGCTTCGAACACGATGCAGATGGTACACGAGTAACTTGGGAAAGCT |
| | | GAAATCCTTGAAAGAAATAGTGGGGCTGCTGAAAGAACTTGAGATAA |
| | | GCAACTATGCCATTAACGAATACGTTCAGGGGTCCACACGTCGTTAT |
| | | GCCGTTGCGTGGTCTTTCACTGATATTCAACTGCCTGAGGAGCTTTC |
| | | TCGTCCCTCTAACCCCGAGCTCAGCTCTCTTTTCTAG |
| 17 | PcCpr polypeptide | MASSSSDVFVLGLGVVLAALYIFRDQLFAASKPKVAPVSTTKPANGS |
| | | ANPRDFIAKMKQGKKRIVIFYGSQTGTAEEYAIRLAKEAKQKFGLAS |
| | | LVCDPEEYDFEKLDQLPEDSIAFFVVATYGEGEPTDNAVQLLQNLQD |
| | | ESFEFSSGERKLSGLKYVVFGLGNKTYEHYNLIGRTVDAQLAKMGAI |
| | | RIGERGEGDDDKSMEEDYLEWKDGMWEAFATAMGVEEGQGGDSADFV |
| | | VSELESHPPEKVYQGEFSARALTKTKGIHDAKNPFAAPIAVARELFQ |
| | | SVVDRNCVHVEFNIEGSGITYQHGDHVGLWPLNPDVEVERLLCVLGL |
| | | AEKRDAVISIESLDPALAKVPFPVPTTYGAVLRHYIDISAVAGRQIL |
| | | GTLSKFAPTPEAEAFLRNLNTNKEEYHNVVANGCLKLGEILQIATGN |
| | | DITVPPTTANTTKWPIPFDIIVSAIPRLQPRYYSISSSPKIHPNTIH |
| | | ATVVVLKYENVPTEPIPRKWVYGVGSNFLLNLKYAVNKEPVPYITQN |
| | | GEQRVGVPEYLIAGPRGSYKTESFYKAPIHVRRSTFRLPTNPKSPVI |
| | | MIGPGTGVAPFRGFVQERVALARRSIEKNGPDSLADWGRISLFYGCR |
| | | RSDEDFLYKDEWPQYEAELKGKFKLHCAFSRQNYKPDGSKIYVQDLI |
| | | WEDREHIADAILNGKGYVYICGEAKSMSKQVEEVLAKILGEAKGGSG |
| | | PVEGVAEVKLLKERSRLMLDVWS |
| 18 | Pccpr coding | ATGGCTTCTTCTTCTTCTGACGTTTTCGTTTTGGGTTTGGGTGTTGT |
| | sequence (5′-3′) | TTTGGCTGCTTTGTACATCTTCAGAGACCAATTGTTCGCTGCTTCTA |
| | From Milne et al, | AGCCAAAGGTTGCTCCAGTTTCTACTACTAAGCCAGCTAACGGTTCT |
| | Metab Eng, 60, | GCTAACCCAAGAGACTTCATCGCTAAGATGAAGCAAGGTAAGAAGAG |
| | 25-36. | AATCGTTATCTTCTACGGTTCTCAAACTGGTACTGCTGAAGAATACG |
| | | CTATCAGATTGGCTAAGGAAGCTAAGCAAAAGTTCGGTTTGGCTTCT |
| | | TTGGTTTGTGACCCAGAAGAATACGACTTCGAAAAGTTGGACCAATT |
| | | GCCAGAAGACTCTATCGCTTTCTTCGTTGTTGCTACTTACGGTGAAG |
| | | GTGAACCAACTGACAACGCTGTTCAATTGTTGCAAAACTTGCAAGAC |
| | | GAATCTTTCGAATTCTCTTCTGGTGAAAGAAAGTTGTCTGGTTTGAA |
| | | GTACGTTGTTTTCGGTTTGGGTAACAAGACTTACGAACACTACAACT |
| | | TGATCGGTAGAACTGTTGACGCTCAATTGGCTAAGATGGGTGCTATC |
| | | AGAATCGGTGAAAGAGGTGAAGGTGACGACGACAAGTCTATGGAAGA |
| | | AGACTACTTGGAATGGAAGGACGGTATGTGGGAAGCTTTCGCTACTG |
| | | CTATGGGTGTTGAAGAAGGTCAAGGTGGTGACTCTGCTGACTTCGTT |
| | | GTTTCTGAATTGGAATCTCACCCACCAGAAAAGGTTTACCAAGGTGA |
| | | ATTCTCTGCTAGAGCTTTGACTAAGACTAAGGGTATCCACGACGCTA |
| | | AGAACCCATTCGCTGCTCCAATCGCTGTTGCTAGAGAATTGTTCCAA |
| | | TCTGTTGTTGACAGAAACTGTGTTCACGTTGAATTCAACATCGAAGG |
| | | TTCTGGTATCACTTACCAACACGGTGACCACGTTGGTTTGTGGCCAT |
| | | TGAACCCAGACGTTGAAGTTGAAAGATTGTTGTGTGTTTTGGGTTTG |
| | | GCTGAAAAGAGAGACGCTGTTATCTCTATCGAATCTTTGGACCCAGC |
| | | TTTGGCTAAGGTTCCATTCCCAGTTCCAACTACTTACGGTGCTGTTT |
| | | TGAGACACTACATCGACATCTCTGCTGTTGCTGGTAGACAAATCTTG |
| | | GGTACTTTGTCTAAGTTCGCTCCAACTCCAGAAGCTGAAGCTTTCTT |
| | | GAGAAACTTGAACACTAACAAGGAAGAATACCACAACGTTGTTGCTA |
| | | ACGGTTGTTTGAAGTTGGGTGAAATCTTGCAAATCGCTACTGGTAAC |
| | | GACATCACTGTTCCACCAACTACTGCTAACACTACTAAGTGGCCAAT |
| | | CCCATTCGACATCATCGTTTCTGCTATCCCAAGATTGCAACCAAGAT |
| | | ACTACTCTATCTCTTCTTCTCCAAAGATCCACCCAAACACTATCCAC |
| | | GCTACTGTTGTTGTTTTGAAGTACGAAAACGTTCCAACTGAACCAAT |
| | | CCCAAGAAAGTGGGTTTACGGTGTTGGTTCTAACTTCTTGTTGAACT |
| | | TGAAGTACGCTGTTAACAAGGAACCAGTTCCATACATCACTCAAAAC |
| | | GGTGAACAAAGAGTTGGTGTTCCAGAATACTTGATCGCTGGTCCAAG |
| | | AGGTTCTTACAAGACTGAATCTTTCTACAAGGCTCCAATCCACGTTA |
| | | GAAGATCTACTTTCAGATTGCCAACTAACCCAAAGTCTCCAGTTATC |
| | | ATGATCGGTCCAGGTACTGGTGTTGCTCCATTCAGAGGTTTCGTTCA |
| | | AGAAAGAGTTGCTTTGGCTAGAAGATCTATCGAAAAGAACGGTCCAG |
| | | ACTCTTTGGCTGACTGGGGTAGAATCTCTTTGTTCTACGGTTGTAGA |
| | | AGATCTGACGAAGACTTCTTGTACAAGGACGAATGGCCACAATACGA |
| | | AGCTGAATTGAAGGGTAAGTTCAAGTTGCACTGTGCTTTCTCTAGAC |
| | | AAAACTACAAGCCAGACGGTTCTAAGATCTACGTTCAAGACTTGATC |
| | | TGGGAAGACAGAGAACACATCGCTGACGCTATCTTGAACGGTAAGGG |
| | | TTACGTTTACATCTGTGGTGAAGCTAAGTCTATGTCTAAGCAAGTTG |
| | | AAGAAGTTTTGGCTAAGATCTTGGGTGAAGCTAAGGGTGGTTCTGGT |
| | | CCAGTTGAAGGTGTTGCTGAAGTTAAGTTGTTGAAGGAAAGATCTAG |
| | | ATTGATGTTGGACGTTTGGTCTTAA |
| 19 | Sah1 polypeptide | MSAPAQNYKIADISLAAFGRKEIELAEHEMPGLMAIRKAYGDVQPLK |
| | (GenBank: | GARIAGCLHMTIQTAVLIETLVALGAEVTWSSCNIYSTQDHAAAAIA |
| | NP_010961.3) | ASGVPVFAWKGETEEEYLWCIEQQLFAFKDNKKLNLILDDGGDLTTL |
| | | VHEKHPEMLEDCFGLSEETTTGVHHLYRMVKEGKLKVPAINVNDSVT |
| | | KSKFDNLYGCRESLVDGIKRATDVMLAGKVAVVAGYGDVGKGCAAAL |
| | | RGMGARVLVTEIDPINALQAAMEGYQVVTMEDASHIGQVFVTTTGCR |
| | | DIINGEHFINMPEDAIVCNIGHFDIEIDVAWLKANAKECINIKPQVD |
| | | RYLLSSGRHVILLANGRLVNLGCATGHSSFVMSCSFSNQVLAQIALF |
| | | KSNDKSFREKHIEFQKTGPFEVGVHVLPKILDEAVAKFHLGNLGVRL |
| | | TKLSKVQSEYLGIPEEGPFKADHYRY |
| 20 | SAH1 coding | ATGTCTGCTCCAGCTCAAAACTACAAAATCGCTGATATCTCTTTGGC |
| | sequence (5′-3′) | TGCCTTCGGTAGAAAGGAAATCGAATTGGCTGAACATGAAATGCCAG |
| | (GenBank: | GTTTGATGGCCATCAGAAAGGCTTACGGTGACGTCCAACCTTTGAAA |
| | NM_001178934.3) | GGCGCCCGTATTGCTGGTTGTTTGCACATGACCATTCAAACTGCTGT |
| | | TTTAATTGAAACTTTAGTTGCTTTGGGTGCCGAAGTTACCTGGTCCT |
| | | CTTGTAACATCTATTCGACTCAAGATCATGCCGCCGCTGCTATTGCC |
| | | GCTTCCGGTGTTCCAGTTTTTGCCTGGAAGGGTGAAACTGAAGAAGA |
| | | GTATTTGTGGTGTATTGAACAACAATTGTTTGCCTTCAAGGACAACA |
| | | AGAAATTGAACTTGATCTTAGATGATGGTGGTGATTTAACCACTTTA |
| | | GTTCATGAAAAGCACCCTGAAATGCTGGAAGACTGCTTTGGTCTTTC |
| | | CGAAGAAACTACCACCGGTGTTCACCACTTATACAGAATGGTCAAAG |
| | | AAGGCAAGTTAAAGGTTCCTGCCATTAACGTTAACGACTCCGTCACT |
| | | AAGTCCAAGTTTGACAACTTGTACGGCTGTAGAGAATCCTTAGTCGA |
| | | CGGTATTAAGAGAGCCACTGATGTCATGTTGGCTGGTAAGGTTGCCG |
| | | TTGTTGCTGGTTACGGTGATGTCGGTAAGGGTTGTGCTGCTGCCTTA |
| | | AGAGGAATGGGTGCTCGTGTCTTGGTTACCGAAATTGACCCAATCAA |
| | | CGCTTTACAAGCTGCCATGGAAGGCTACCAAGTTGTTACCATGGAAG |
| | | ATGCATCCCACATTGGTCAAGTTTTCGTTACCACCACTGGTTGTAGA |
| | | GATATTATCAACGGTGAACATTTCATCAACATGCCAGAAGATGCCAT |
| | | TGTTTGTAACATTGGCCATTTCGATATCGAAATTGATGTCGCCTGGT |
| | | TAAAGGCTAACGCTAAAGAATGTATTAACATCAAACCACAAGTCGAC |
| | | CGTTACTTGTTGTCTTCTGGTAGACACGTCATCTTGTTGGCTAACGG |
| | | TAGATTAGTTAACTTGGGTTGTGCTACTGGTCACTCATCTTTCGTTA |
| | | TGTCTTGTTCCTTCTCTAACCAAGTCTTAGCTCAAATTGCTTTGTTC |
| | | AAGTCTAACGATAAGTCTTTCAGAGAAAAGCACATTGAATTCCAAAA |
| | | GACAGGCCCATTCGAAGTTGGTGTCCACGTTTTGCCAAAGATCTTGG |
| | | ATGAAGCTGTCGCTAAGTTCCACTTGGGCAACTTGGGTGTTAGATTG |
| | | ACTAAATTGAGTAAAGTCCAATCTGAATACTTGGGTATTCCAGAAGA |
| | | AGGTCCATTCAAGGCCGACCACTACAGATATTGA |
| 21 | Ado1 polypeptide | MTAPLVVLGNPLLDFQADVTAEYLAKYSLKENDAILVDAKSGDAKMA |
| | (GenBank: | IFDELLQMPETKLVAGGAAQNTARGAAYVLGAGQVVYFGSVGKDKFS |
| | NP_012639.1) | ERLLNENEKAGVKSMYQVQNDIGTGKCAALITGHNRSLVTDLGAANF |
| | | FTPDHLDKHWDLVEAAKLFYIGGFHLTVSPDAIVKLGQHAKENSKPF |
| | | VLNFSAPFIPHVFKDALARVLPYATVIIANESEAEAFCDAFQLDCAN |
| | | TDLEAIAQRIVKDSPVEKTVIFTHGVEPTVVVSSKGTSTYPVKPLDS |
| | | SKIVDTNGAGDAFAGGFMAGLTKGEDLETSIDMGQWLAALSIQEVGP |
| | | SYPSEKISYSK |
| 22 | ADO1 coding | ATGACCGCACCATTGGTAGTATTGGGTAACCCACTTTTAGATTTCCA |
| | sequence (5′-3′) | AGCCGACGTCACGGCTGAATACCTGGCCAAGTATTCTCTAAAGGAAA |
| | (GenBank: | ACGACGCAATTTTGGTCGATGCCAAATCAGGCGATGCTAAGATGGCT |
| | NM_001181763.1) | ATTTTTGACGAGCTCTTACAGATGCCAGAAACAAAGCTTGTTGCAGG |
| | | TGGTGCTGCTCAAAACACTGCTAGAGGGGCAGCATACGTTTTGGGCG |
| | | CCGGCCAGGTGGTGTACTTCGGTTCCGTCGGTAAGGACAAGTTCAGC |
| | | GAGAGATTGCTTAACGAAAACGAAAAAGCTGGTGTCAAGTCTATGTA |
| | | CCAAGTTCAAAATGATATTGGTACCGGTAAGTGTGCCGCATTAATCA |
| | | CTGGCCATAACCGGTCCTTGGTCACTGACTTGGGTGCTGCCAATTTC |
| | | TTTACTCCAGACCACTTGGACAAGCATTGGGACTTGGTCGAAGCAGC |
| | | TAAGCTCTTCTACATCGGTGGTTTCCACTTGACCGTGTCTCCAGACG |
| | | CTATCGTTAAGTTGGGCCAACATGCTAAAGAGAACAGCAAACCTTTC |
| | | GTGTTGAACTTTAGTGCTCCTTTCATTCCTCATGTCTTCAAAGACGC |
| | | ATTGGCCAGAGTTTTGCCTTATGCTACCGTCATCATCGCTAACGAAT |
| | | CGGAGGCCGAAGCCTTTTGCGACGCCTTCCAATTAGACTGTGCCAAC |
| | | ACTGATTTGGAAGCTATTGCTCAAAGAATTGTCAAGGACTCTCCAGT |
| | | TGAAAAGACTGTCATCTTCACCCACGGTGTCGAACCAACAGTGGTCG |
| | | TGTCCTCCAAGGGTACCAGCACATATCCAGTCAAACCTTTGGACTCT |
| | | TCTAAGATCGTCGACACCAACGGTGCTGGTGACGCCTTCGCTGGTGG |
| | | CTTTATGGCTGGGTTGACTAAAGGTGAAGATTTGGAAACCTCTATTG |
| | | ACATGGGTCAATGGCTAGCTGCTTTGTCTATTCAAGAAGTTGGTCCC |
| | | TCTTACCCTTCCGAAAAAATATCTTACTCTAAATAG |
| 23 | cnsBv1 coding | ATGAACTCACCCACCGCATCCGACCCCATAGTGCGTACATTAGACGC |
| | sequence (5′-3′)- | GCCGGAAACCAAGGACATGCAGGAAGCATTCCTTCCTGGAGAGTCAC |
| | codon optimized, | CGCAGGACGTCGCGTCAGTCATCCAGGAAACGATGGACATCTTCGAC |
| | encoding CnsBv1 of | AAGAGGATGCGTCTAACGCACCCTCACTGCTTCTGCTACGTCCCTAG |
| | SEQ ID NO: 1 | TTGCCCTTCCCCGTTAGCGTGGGTAGGAGATCTGTTCACGTCACTTT |
| | | ACAACGTGAACGCAGTAAGCTGGGACGTGTCCAGTGGGCCGTCCGCC |
| | | ATGGAGCAGGGAATGATTAGGTGGCTTGCACAGCAGGTCGGACTACC |
| | | ACCCTCAGCGGGCGGATGCTTCGTGAGTGGCGGCAGCATGGCAAACC |
| | | TGTCCGCGGTTGTCGCAGCACGTGACAAGCTGTTACCCCCGATGAGA |
| | | AGGGCCGAGGGAACCATATACACGTCCGACCAGACACACATAAGTAT |
| | | AAAGAAGTGCCTACACATCATCGGATTCGACGAGCACCAGATCAGGG |
| | | TCATACCTACAGACCACCTTTTCCGTATGGACACGGACATACTGAGA |
| | | AGGACAATCGCAGCAGACAGGCAGTGCGGAAGGTTACCCTTCTTAGT |
| | | AGTGGCGAGTTGCGGGACGACGAACACCGGATCAATAGACCCTTTAC |
| | | ACGCCCTTGCGGACGTGGCAGAGGAAGAGGGATTATGGTTACACGTA |
| | | GACGGGGCATACGGCGCCAGCATCGCACTGTCGGACAAGTACAGGCA |
| | | CTTAGTAGACGGCATCGGAAGGGCGGACTCAATATCATGGGACGGAC |
| | | ACAAGTGGCTATTCCAGACGTACGGATGCGGCATAGTGCTAACACGT |
| | | GACTCAGCAAGCCTGGCACACTCATTCGCGGTGGACGCAGAGTACAT |
| | | CAACTCGACGACACCTCACGGGGCCGTCAACTTCTACAACTTATCCC |
| | | CTGAGCTATCCAGGCCCGCGCGTGCAATGCCGCTGTGGTTCACCTTA |
| | | CGTGTATTAGGGAGAAGGCAGATGGGCGAGATGATAGACCAGGGATT |
| | | CTTACTGGCCCGTACAGCCGAAGAGGAGATCAGGCGTTACCCCCACT |
| | | GGGTAATATTAGCCCCTGTCACAGCCTCGATAGTCGTCTTTCGTTAC |
| | | TGCCCTCTAGGACTTAAGGACGACGACTTAGACACACTTAACTTAGC |
| | | CTTATCCGCAAGACTTCTTGCGGAGAACGTAGCATCCATACTAACCA |
| | | CCAACATAAGGGGACGTGTCGCATTAAGGTTCTGCGCCACAAACCCT |
| | | GCAACACAGGCCGACACCATAACAAAGGTAGTCCAGCAGATGGACCA |
| | | GATGGCAAACACAATGCCTACATGCGGATTAGGCATGTGCAAGGAGT |
| | | GA |
| 24 | psiD coding | ATGCAAGTAATACCCGCTTGTAATTCAGCAGCTATTAGATCTTTATG |
| | sequence | TCCAACGCCTGAGTCTTTTAGGAATATGGGATGGTTGTCAGTATCTG |
| | (5′-3′)-codon | ACGCAGTATACTCCGAATTTATAGGTGAATTGGCGACTAGGGCTTCA |
| | optimized | AACAGAAACTATTCCAACGAATTTGGTTTAATGCAACCTATTCAGGA |
| | | ATTCAAAGCTTTTATCGAGTCTGATCCAGTTGTTCACCAGGAGTTTA |
| | | TTGACATGTTCGAAGGCATTCAGGACTCACCAAGAAATTACCAGGAG |
| | | CTTTGCAACATGTTTAACGACATATTTCGTAAGGCACCTGTTTATGG |
| | | TGACTTAGGTCCCCCTGTATACATGATTATGGCCAAATTAATGAATA |
| | | CGCGTGCCGGATTTTCTGCCTTTACCCGTCAGCGTTTGAATTTACAC |
| | | TTTAAGAAATTGTTTGACACCTGGGGATTATTCTTGAGTTCGAAGGA |
| | | TTCAAGAAATGTTCTAGTCGCTGATCAGTTCGATGATAGACACTGTG |
| | | GCTGGTTGAATGAGAGAGCTTTATCCGCAATGGTCAAACACTATAAT |
| | | GGCAGAGCATTTGACGAAGTATTCTTATGTGATAAGAACGCGCCCTA |
| | | TTACGGTTTCAATTCGTATGACGATTTCTTTAATAGAAGGTTTAGAA |
| | | ATAGGGACATTGACCGTCCTGTAGTCGGCGGGGTGAATAACACCACC |
| | | TTGATCAGTGCGGCTTGCGAATCATTGTCATACAACGTTTCCTACGA |
| | | CGTCCAATCTCTAGACACTTTAGTATTTAAGGGTGAGACTTATTCAT |
| | | TGAAGCATTTGTTGAACAACGATCCGTTCACCCCTCAATTTGAACAT |
| | | GGATCAATTTTGCAAGGATTCTTGAATGTGACAGCTTACCACAGGTG |
| | | GCACGCACCCGTCAATGGAACTATCGTTAAGATAATAAATGTTCCAG |
| | | GTACTTATTTTGCACAGGCTCCTTCTACAATAGGGGACCCGATCCCA |
| | | GACAACGACTATGATCCCCCACCTTATTTGAAATCTTTAGTTTACTT |
| | | CTCCAATATTGCGGCTAGACAAATTATGTTCATTGAGGCAGATAACA |
| | | AGGAGATCGGCTTAATTTTCCTGGTGTTCATTGGAATGACAGAGATT |
| | | TCCACGTGTGAGGCTACCGTTTCAGAAGGGCAACATGTTAACAGAGG |
| | | TGATGACTTAGGAATGTTTCATTTTGGTGGTTCTAGTTTTGCGTTGG |
| | | GTTTGCGTAAGGATTGCAGAGCAGAAATAGTGGAAAAGTTTACAGAA |
| | | CCAGGTACGGTGATAAGAATAAATGAGGTTGTCGCCGCATTAAAGGC |
| | | GTGA |
| 25 | psiH coding | ATGATTGCTGTTTTGTTTTCATTCGTAATTGCGGGCTGCATCTACTA |
| | sequence | TATCGTTTCGAGGAGAGTCCGTAGATCAAGGTTGCCGCCCGGCCCAC |
| | (5′-3′)-codon | CTGGTATCCCTATCCCCTTCATTGGCAACATGTTTGACATGCCAGAA |
| | optimized | GAATCTCCATGGCTTACGTTTCTTCAGTGGGGTAGAGACTACAATAC |
| | | AGACATTTTGTATGTAGACGCAGGTGGTACAGAGATGGTTATTTTAA |
| | | ATACACTTGAAACAATTACCGACTTGTTGGAGAAGCGTGGTTCTATA |
| | | TATTCGGGCAGATTAGAAAGTACTATGGTCAATGAGTTGATGGGTTG |
| | | GGAATTTGATCTAGGATTTATTACTTACGGTGACAGGTGGCGTGAGG |
| | | AGCGTAGGATGTTTGCAAAAGAATTTAGCGAGAAAGGCATCAAACAA |
| | | TTTAGGCACGCGCAAGTGAAGGCCGCTCATCAGCTTGTGCAACAGCT |
| | | AACCAAAACACCGGATAGATGGGCCCAACACATAAGACACCAAATCG |
| | | CTGCTATGTCTTTAGATATTGGGTACGGCATTGATCTAGCAGAAGAT |
| | | GATCCCTGGCTAGAGGCTACACACCTTGCTAATGAAGGATTAGCAAT |
| | | AGCGTCTGTTCCTGGTAAATTCTGGGTTGATTCATTCCCCTCTTTAA |
| | | AGTACTTGCCCGCTTGGTTCCCAGGGGCCGTCTTTAAGAGAAAAGCT |
| | | AAGGTGTGGCGTGAGGCCGCTGACCACATGGTGGACATGCCATACGA |
| | | GACTATGCGTAAGTTAGCCCCTCAAGGACTGACTAGGCCATCTTACG |
| | | CCAGTGCTAGGTTACAGGCGATGGACTTGAATGGTGACCTAGAGCAC |
| | | CAAGAGCACGTGATCAAGAACACTGCTGCTGAGGTAAATGTGGGTGG |
| | | TGGTGACACGACCGTTTCCGCTATGAGCGCATTCATTCTGGCAATGG |
| | | TGAAGTACCCCGAAGTCCAAAGGAAAGTGCAGGCGGAGCTTGATGCT |
| | | TTGACTAATAACGGGCAAATCCCGGATTACGACGAAGAGGACGACTC |
| | | ACTTCCCTATTTAACGGCATGTATTAAAGAGTTGTTTAGATGGAACC |
| | | AGATTGCTCCTCTTGCAATACCCCATAAATTGATGAAAGACGATGTA |
| | | TATAGAGGGTATCTAATCCCAAAGAACACATTAGTCTTTGCAAACAC |
| | | GTGGGCAGTCTTAAACGACCCAGAGGTTTATCCAGACCCTTCGGTAT |
| | | TTAGACCCGAGCGTTATCTTGGACCAGATGGTAAGCCAGACAATACC |
| | | GTCCGTGACCCTAGAAAGGCTGCTTTCGGTTATGGGAGAAGAAATTG |
| | | CCCTGGGATTCACTTAGCTCAATCGACAGTATGGATCGCTGGGGCAA |
| | | CATTACTGTCTGCTTTTAATATCGAGAGGCCTGTGGACCAAAACGGT |
| | | AAGCCCATTGACATTCCTGCGGACTTTACAACAGGTTTCTTCAGGCA |
| | | CCCTGTCCCGTTCCAGTGCAGGTTTGTTCCTAGAACAGAGCAGGTGT |
| | | CTCAAAGCGTGTCCGGCCCATAG |
| 26 | psiK coding | ATGGCCTTCGACCTAAAGACAGAAGATGGATTGATCACATACCTGAC |
| | sequence | TAAGCACCTGTCTCTAGACGTGGATACCAGTGGAGTCAAAAGATTAA |
| | (5′-3′)-codon | GCGGTGGATTCGTTAATGTTACATGGAGAATTAAATTAAACGCCCCT |
| | optimized | TACCAAGGACATACCTCGATTATATTGAAGCACGCCCAGCCACATAT |
| | | GAGTACAGACGAGGACTTTAAGATAGGCGTCGAGCGTAGTGTGTACG |
| | | AATACCAAGCTATAAAGCTGATGATGGCCAACAGAGAGGTATTAGGC |
| | | GGTGTCGACGGGATAGTTAGTGTTCCAGAAGGTTTGAACTACGACTT |
| | | GGAGAATAACGCTTTAATTATGCAGGATGTTGGAAAGATGAAAACTT |
| | | TGTTGGATTATGTAACAGCTAAACCACCTTTAGCAACTGACATCGCC |
| | | AGGTTGGTTGGTACGGAGATCGGTGGATTTGTTGCTCGTCTACATAA |
| | | CATCGGTCGTGAAAGACGTGACGATCCAGAATTCAAATTCTTTTCAG |
| | | GAAACATAGTGGGACGTACTACAAGTGACCAACTGTATCAGACCATT |
| | | ATACCAAACGCAGCAAAGTACGGTGTCGACGATCCCTTGTTGCCCAC |
| | | CGTTGTTAAAGACCTTGTTGACGATGTCATGCATTCTGAGGAGACAC |
| | | TGGTCATGGCTGATCTTTGGTCAGGCAACATTTTGCTACAGCTAGAG |
| | | GAAGGTAATCCTTCGAAGTTGCAGAAGATATACATATTGGATTGGGA |
| | | GTTATGTAAGTACGGCCCAGCTTCTTTAGATCTAGGATATTTTCTAG |
| | | GAGACTGTTATCTTATATCGAGATTCCAAGATGAACAGGTCGGCACC |
| | | ACTATGAGACAAGCCTACTTGCAGTCGTATGCAAGGACATCAAAGCA |
| | | TTCTATTAATTATGCCAAAGTTACTGCTGGTATAGCGGCCCACATCG |
| | | TAATGTGGACCGATTTCATGCAATGGGGCTCCGAAGAGGAGAGGATA |
| | | AATTTCGTGAAGAAGGGTGTAGCCGCATTCCACGACGCAAGGGGAAA |
| | | CAACGATAATGGCGAAATCACGAGTACATTATTGAAGGAGTCTAGCA |
| | | CTGCTTAG |
| 27 | psiM coding | ATGCATATTAGAAATCCATACAGGACCCCTATCGACTATCAGGCCTT |
| | sequence | GTCAGAGGCCTTTCCACCACTAAAGCCGTTCGTGTCGGTAAACGCCG |
| | (5′-3′)-codon | ATGGCACTTCCTCGGTGGATTTGACTATACCTGAAGCTCAAAGGGCA |
| | optimized | TTCACTGCTGCGCTTCTTCACAGGGATTTTGGTTTAACGATGACCAT |
| | | ACCTGAGGACAGGCTATGTCCTACTGTGCCGAATAGATTAAATTACG |
| | | TGCTATGGATTGAAGATATATTCAACTACACAAATAAAACGCTAGGA |
| | | CTAAGCGACGATAGACCCATAAAGGGAGTTGACATCGGAACCGGGGC |
| | | ATCCGCAATTTATCCCATGTTAGCTTGCGCTAGGTTTAAAGCATGGA |
| | | GCATGGTCGGTACAGAAGTAGAAAGGAAATGTATTGACACAGCAAGA |
| | | CTTAATGTTGTGGCCAATAACCTTCAGGACCGTTTATCCATTCTAGA |
| | | AACTAGCATAGACGGTCCTATATTGGTTCCCATTTTCGAAGCCACTG |
| | | AAGAGTACGAGTATGAGTTCACGATGTGTAACCCACCGTTCTATGAT |
| | | GGCGCTGCCGACATGCAGACATCTGATGCAGCAAAGGGATTTGGCTT |
| | | CGGAGTCGGTGCACCCCATTCGGGTACTGTCATAGAGATGTCTACAG |
| | | AGGGCGGCGAATCCGCATTTGTTGCTCAGATGGTTCGTGAGTCATTA |
| | | AAATTAAGGACAAGATGTAGGTGGTACACCTCTAACTTGGGCAAGTT |
| | | GAAGTCTTTGAAGGAGATAGTGGGTTTGCTAAAAGAGTTAGAGATCT |
| | | CTAATTATGCTATCAATGAATACGTTCAGGGTAGTACTAGAAGATAC |
| | | GCTGTGGCCTGGTCTTTCACTGACATCCAGTTGCCCGAGGAATTATC |
| | | AAGGCCCTCGAATCCAGAGCTTAGTTCGCTATTTTGA |
| 28 | Pccpr coding | ATGGCATCATCATCATCTGACGTGTTCGTCTTAGGCTTAGGCGTTGT |
| | sequence (5′-3′)- | ATTAGCTGCGCTTTACATATTTAGAGATCAGTTGTTCGCAGCATCAA |
| | codon optimized | AACCAAAGGTTGCACCGGTTTCAACTACAAAACCAGCGAACGGGAGT |
| | | GCTAACCCAAGGGATTTTATTGCAAAGATGAAACAAGGAAAGAAGAG |
| | | AATCGTTATCTTTTATGGTAGTCAAACAGGAACGGCTGAGGAGTACG |
| | | CCATTAGATTAGCAAAGGAAGCAAAACAGAAGTTTGGTCTGGCCAGT |
| | | CTGGTCTGCGATCCTGAGGAATATGACTTCGAAAAGTTGGATCAACT |
| | | ACCAGAGGACAGTATAGCCTTCTTCGTTGTTGCGACTTATGGTGAGG |
| | | GTGAGCCTACGGACAATGCTGTGCAATTATTGCAAAATCTGCAAGAT |
| | | GAGAGTTTCGAATTCTCTTCAGGAGAGAGAAAGCTTTCTGGTCTTAA |
| | | GTACGTAGTGTTTGGCCTTGGCAACAAAACGTATGAGCATTACAACT |
| | | TAATTGGTAGAACTGTAGACGCACAGTTGGCTAAAATGGGAGCCATT |
| | | CGTATAGGCGAGAGAGGCGAAGGCGATGACGACAAATCTATGGAAGA |
| | | AGATTACCTAGAATGGAAGGATGGTATGTGGGAAGCATTCGCTACGG |
| | | CTATGGGTGTAGAGGAAGGCCAGGGCGGTGATTCTGCCGATTTTGTG |
| | | GTATCTGAACTGGAGTCGCACCCACCTGAGAAGGTATATCAAGGGGA |
| | | ATTCAGCGCACGTGCTTTGACAAAGACCAAAGGTATTCATGACGCTA |
| | | AGAATCCTTTCGCTGCACCTATAGCTGTGGCAAGAGAACTTTTCCAA |
| | | TCCGTGGTGGACAGAAATTGTGTTCACGTAGAGTTTAACATTGAAGG |
| | | CTCAGGTATAACGTATCAGCATGGTGACCATGTTGGTCTGTGGCCGT |
| | | TAAACCCGGACGTGGAAGTCGAGAGATTGCTTTGCGTTTTGGGTTTG |
| | | GCTGAGAAGAGAGATGCCGTAATAAGTATCGAGAGCCTGGACCCAGC |
| | | GCTTGCCAAGGTTCCATTTCCAGTTCCAACGACTTATGGTGCAGTTC |
| | | TAAGACACTATATAGATATTTCAGCAGTAGCAGGTAGACAAATTTTG |
| | | GGCACTTTATCAAAGTTTGCGCCAACCCCAGAGGCCGAAGCATTCCT |
| | | ACGTAATTTAAACACAAATAAGGAAGAGTATCACAATGTAGTCGCGA |
| | | ACGGCTGCTTAAAGTTGGGCGAGATCTTGCAGATTGCCACAGGTAAC |
| | | GATATTACTGTACCACCAACGACTGCGAATACTACCAAGTGGCCTAT |
| | | TCCGTTCGACATTATCGTGTCGGCTATACCACGTTTACAACCAAGAT |
| | | ACTATTCAATCTCTTCATCCCCTAAGATTCATCCCAACACTATACAC |
| | | GCTACTGTGGTTGTCCTAAAATATGAGAATGTACCAACTGAGCCTAT |
| | | ACCCAGAAAGTGGGTGTACGGTGTCGGCTCAAATTTCTTGTTAAATC |
| | | TGAAATATGCAGTGAACAAAGAACCGGTTCCATACATAACGCAGAAC |
| | | GGGGAACAAAGAGTTGGAGTCCCAGAATATCTTATAGCAGGACCTAG |
| | | AGGTAGCTATAAAACCGAATCCTTTTACAAGGCTCCTATTCACGTTA |
| | | GAAGGAGTACATTTAGATTACCTACCAATCCGAAGTCGCCAGTTATC |
| | | ATGATAGGTCCAGGGACAGGTGTGGCTCCGTTTAGAGGTTTCGTGCA |
| | | GGAGAGAGTGGCGTTGGCCCGTAGAAGTATCGAGAAGAATGGTCCAG |
| | | ATTCATTGGCAGATTGGGGTAGAATCTCCCTGTTCTATGGCTGTAGA |
| | | AGGTCTGATGAAGATTTTCTATATAAAGATGAATGGCCTCAATACGA |
| | | AGCCGAATTAAAAGGGAAGTTTAAATTGCACTGCGCTTTCTCGAGGC |
| | | AGAACTATAAACCTGATGGGAGCAAGATATACGTGCAAGATTTAATC |
| | | TGGGAAGATAGAGAGCACATCGCCGACGCAATTCTAAACGGTAAGGG |
| | | ATATGTATACATCTGTGGTGAAGCGAAATCGATGTCCAAGCAGGTTG |
| | | AGGAAGTTTTGGCCAAAATCCTGGGAGAGGCAAAAGGCGGTTCAGGT |
| | | CCTGTTGAAGGAGTTGCTGAGGTTAAATTGCTAAAAGAGAGATCGAG |
| | | ATTAATGTTAGATGTGTGGTCTTGA |
| 29 | CrTDC polypeptide | MGSIDSTNVAMSNSPVGEFKPLEAEEFRKQAHRMVDFIADYYKNVET |
| | (GenBank: | YPVLSEVEPGYLRKRIPETAPYLPEPLDDIMKDIQKDIIPGMTNWMS |
| | AYA72254.1) | PNFYAFFPATVSSAAFLGEMLSTALNSVGFTWVSSPAATELEMIVMD |
| | | WLAQILKLPKSFMFSGTGGGVIQNTTSESILCTIIAARERALEKLGP |
| | | DSIGKLVCYGSDQTHTMFPKTCKLAGIYPNNIRLIPTTVETDFGISP |
| | | QVLRKMVEDDVAAGYVPLFLCATLGTTSTTATDPVDSLSEIANEFGI |
| | | WIHVDAAYAGSACICPEFRHYLDGIERVDSLSLSPHKWLLAYLDCTC |
| | | LWVKQPHLLLRALTTNPEYLKNKQSDLDKVVDFKNWQIATGRKFRSL |
| | | KLWLILRSYGVVNLQSHIRSDVAMAKMFEEWVRSDSRFEIVVPRNFS |
| | | LVCFRLKPDVSSLHVEEVNKKLLDMLNSTGRVYMTHTIVGGIYMLRL |
| | | AVGSSLTEEHHVRRVWDLIQKLTDDLLKEA |
| 30 | crTDC coding | ATGGGCAGCATTGATTCAACAAATGTAGCCATGTCCAATTCTCCAGT |
| | sequence (5′-3′) | TGGAGAATTTAAGCCACTTGAAGCTGAGGAATTCCGAAAACAAGCCC |
| | (GenBank: | ATCGTATGGTAGATTTCATAGCCGATTATTACAAAAATGTGGAAACA |
| | MG748691.1) | TATCCGGTCCTTAGCGAAGTCGAACCTGGATATCTCCGAAAACGTAT |
| | | CCCCGAAACCGCTCCTTACCTCCCCGAACCACTTGACGACATCATGA |
| | | AAGATATTCAGAAGGATATTATCCCAGGAATGACAAATTGGATGAGC |
| | | CCTAATTTTTATGCATTTTTTCCTGCCACTGTTAGTTCAGCTGCCTT |
| | | TTTAGGAGAAATGTTGTCTACTGCCCTAAATTCAGTAGGCTTTACTT |
| | | GGGTTTCTTCACCAGCCGCCACCGAATTAGAAATGATTGTTATGGAT |
| | | TGGTTGGCTCAGATCCTTAAACTCCCCAAATCTTTCATGTTTTCAGG |
| | | TACCGGTGGCGGCGTCATCCAAAACACCACTAGCGAGTCCATTCTTT |
| | | GTACAATCATTGCCGCCCGGGAAAGGGCCCTGGAGAAGCTCGGTCCC |
| | | GATAGTATTGGAAAACTTGTCTGTTACGGATCCGATCAAACCCATAC |
| | | CATGTTCCCCAAAACTTGCAAATTGGCGGGAATTTATCCGAATAATA |
| | | TTAGGTTAATACCTACGACCGTCGAAACGGATTTCGGCATCTCACCT |
| | | CAAGTTCTACGAAAAATGGTCGAGGATGACGTGGCGGCCGGATATGT |
| | | ACCGCTGTTCTTATGCGCTACCCTGGGTACCACCTCGACCACGGCTA |
| | | CCGATCCTGTGGACTCACTTTCTGAAATCGCTAACGAGTTTGGTATT |
| | | TGGATCCACGTGGATGCTGCTTATGCGGGAAGCGCCTGTATATGTCC |
| | | CGAGTTTAGACATTACTTGGATGGAATCGAACGAGTTGACTCACTGA |
| | | GTCTGAGTCCACACAAATGGCTACTCGCTTACTTAGATTGCACTTGC |
| | | TTGTGGGTCAAGCAACCACATTTGTTACTAAGGGCACTCACTACGAA |
| | | TCCTGAGTATTTAAAAAATAAACAGAGTGATTTAGACAAAGTTGTGG |
| | | ACTTCAAAAATTGGCAAATCGCAACGGGACGAAAATTTCGGTCGCTG |
| | | AAACTTTGGCTCATTTTACGTAGCTATGGAGTTGTTAATTTACAGAG |
| | | TCATATTCGTTCTGACGTCGCAATGGCGAAAATGTTCGAAGAATGGG |
| | | TTAGATCAGACTCCAGATTCGAAATTGTGGTACCGAGAAACTTTTCT |
| | | CTTGTTTGTTTTAGATTAAAACCTGACGTTTCGAGTTTACATGTAGA |
| | | AGAAGTGAATAAGAAACTTTTGGACATGCTTAACTCGACGGGACGAG |
| | | TTTATATGACTCATACTATTGTGGGAGGCATATACATGCTAAGACTG |
| | | GCTGTTGGCTCATCGCTAACTGAAGAACATCATGTACGCCGTGTTTG |
| | | GGATTTGATTCAAAAATTAACCGATGATTTGCTCAAAGAAGCTTGA |
| 31 | asTDC1 coding | ATGCCGTCACACGAAGAAAAGTCCGACGATGGTCTGTCCGAGATAAT |
| | sequence (5′-3′)- | GTTAGAGACAGAGTCACTGTTAACGAGGTTATTCACAATAAAGTCCC |
| | codon optimized, | CCGCCCCGTCCGACCCTATAGTGCAGAAGCTTAACGCCCACGAGATC |
| | encoding AsTDC1 of | CAGAACATGCAGCACCAGCTAATGCCTGACGACCACCCTCGTGACCT |
| | SEQ ID NO: 7 | ATCATCCGCCTTACAGCAGGCCATGGCAGTATTCGACAACAGGATGC |
| | | GTCTTACGCACCCCAGGTGCTTCTCATACATACCCGTATGCCCTAAC |
| | | CCTGTGGCGTTATTAGGCGACCTTCTTACAAGTGTCTTTAACGTAAA |
| | | CGCGGCATCCTGGGACGTAGGCTCCGGGCCTTCCGCAGTAGAGACAG |
| | | CCATGATAGCATGGTTAGGCGCACAGCTGGGCCTACCTGACAGTGTG |
| | | GAAGGCTGCTTCGTATCAGGTGGGAGTATGGGAAACATGACGGCAAT |
| | | AGTAGCAGCCAGGGACGAGATACTTCACCCTCTTCAGCGTGCATACG |
| | | CGATAATATACATCAGTGACCAGACACACATCTCCATATTAAAGGCC |
| | | CTACACATCACAGGATTCACACACCACCAGGTACGTAAGATCCCGAC |
| | | AGACGACGACTTCAGGATCGACGTGGACATACTAAGGCGTACAGTAG |
| | | CAAGTGACCGTCACTCCGGACTTGTGCCTTTCTTATTAGTAGCCACA |
| | | TGCGGAACCACAAACACAGGGAGTATAGACCCCTTACACGCCCTGGC |
| | | CGACCTAGCCCGTGACGAGGGGCTATGGCTGCACGTGGACGGAGCGT |
| | | ACGGCGCAAGTATAGCACTTTCGGACCAGCACAGGTCACTAGTGGAC |
| | | GGAATAGGCAGGGCCGACTCGATAAGTTGGGACGGGCACAAGTGGCT |
| | | TTTCCAGACATACGGGTGCGGAATCGTGCTGACAAGGAGAAGTGGAA |
| | | GTCTGAGAAGGTCCTTCGCAGTCGACGCAGAGTACATAAGGGACTCC |
| | | ACCGTACCCGAGGGCACGACGAACTTCTACAACCTTGGACCCGAGTT |
| | | AAGTAGGCCGGCAAGGGCAATGAGCCTTTGGTTAACACTAAGGGTCT |
| | | TAGGACGTAGGCGTATGGGAGAGATGATCGACAGGGGATTCCTACTG |
| | | GCAAGGACCGCAGACCACTCGATACGTCAGTACAGGGACTGGATAAT |
| | | AGCAGCACCTACCGTCGCGTCAATAGTCGTATTCAGGTACGCGCCCC |
| | | CGGGATTAGACGACGGCGCACTAGACGCACTAAACAAGGCAATATCA |
| | | AGGCGTTTACTTGCAGAGGACGTCGCCGCCATCTTAACCACCACCGT |
| | | CAGGGGCAGGACGGTATTAAGGATGTGCGCCATGAACCCGCTAGTAG |
| | | AGCCTGAGACAATCGAGGACGTAATCAGAAGGGTAGACGGCATAGCC |
| | | CGTAGCGAGGCAAGCCGTAGGAAGGAGAGTCACTTCCAGTTAGGATC |
| | | AGCAATGTACTTAGGGTAG |
| 32 | SAH1F primer | GGTCTCTAGGATGTCTGCTCCAGCTCAAAACT |
| | (5′-3′) | |
| 33 | SAH1R primer | GGTCTCAAACTTCAATATCTGTAGTGGTCGGCC |
| | (5′-3′) | |
| 34 | ADO1F primer | GGTCTCTGCAATGACCGCACCATTGGTAG |
| | (5′-3′) | |
| 35 | ADO1R primer | GGTCTCATGTGCTATTTAGAGTAAGATATTTTTTCGGAAGG |
| | (5′-3′) | |
| 36 | OsT5H polypeptide | MELTMASTMSLALLVLSAAYVLVALRRSRSSSSKPRRLPPSPPGWPV |
| | (GenBank: | IGHLHLMSGMPHHALAELARTMRAPLFRMRLGSVPAVVISKPDLARA |
| | NP_001410381) | ALTTNDAALASRPHLLSGQFLSFGCSDVTFAPAGPYHRMARRVVVSE |
| | | LLSARRVATYGAVRVKELRRLLAHLTKNTSPAKPVDLSECFLNLAND |
| | | VLCRVAFGRRFPHGEGDKLGAVLAEAQDLFAGFTIGDFFPELEPVAS |
| | | TVTGLRRRLKKCLADLREACDVIVDEHISGNRQRIPGDRDEDFVDVL |
| | | LRVQKSPDLEVPLTDDNLKALVLDMFVAGTDTTFATLEWVMTELVRH |
| | | PRILKKAQEEVRRVVGDSGRVEESHLGELHYMRAIIKETFRLHPAVP |
| | | LLVPRESVAPCTLGGYDIPARTRVFINTFAMGRDPEIWDNPLEYSPE |
| | | RFESAGGGGEIDLKDPDYKLLPFGGGRRGCPGYTFALATVQVSLASL |
| | | LYHFEWALPAGVRAEDVNLDETFGLATRKKEPLFVAVRKSDAYEFKG |
| | | EELSEV |
| 37 | osT5H coding | ATGGAGCTCACCATGGCGTCGACGATGTCGCTCGCGCTGCTCGTGCT |
| | sequence (5′-3′) | CTCCGCGGCGTACGTGTTGGTCGCGTTGAGGAGGAGCCGGTCGTCGT |
| | encoding OsT5H of | CGTCAAAGCCACGGCGGCTGCCGCCGTCGCCGCCGGGGTGGCCGGTG |
| | SEQ ID NO: 36 | ATCGGGCACCTCCACCTCATGTCCGGCATGCCGCACCACGCGCTGGC |
| | (GenBank: | CGAGCTGGCGCGCACCATGCGCGCGCCGCTGTTCCGGATGCGGCTGG |
| | NM_001423452) | GGAGCGTGCCGGCGGTGGTGATCTCCAAGCCGGACCTCGCCCGCGCC |
| | | GCGCTCACCACCAACGACGCCGCGCTGGCGTCGCGGCCGCACCTGCT |
| | | CTCCGGCCAGTTCCTGTCGTTCGGCTGCTCCGACGTGACGTTCGCGC |
| | | CGGCGGGGCCGTACCACCGGATGGCGCGCCGCGTGGTGGTGTCGGAG |
| | | CTCCTGTCGGCGCGTCGCGTCGCCACGTACGGCGCCGTCAGGGTCAA |
| | | GGAGCTCCGCCGCCTGCTCGCGCACCTCACCAAGAACACCTCGCCGG |
| | | CGAAGCCCGTCGACCTCAGCGAGTGCTTCCTCAACCTCGCCAACGAC |
| | | GTGCTCTGCCGCGTCGCGTTCGGCCGCCGGTTCCCGCACGGCGAGGG |
| | | CGACAAGCTCGGCGCGGTGCTCGCCGAGGCGCAGGACCTCTTCGCCG |
| | | GGTTCACCATCGGCGACTTCTTCCCCGAGCTCGAGCCCGTCGCCAGC |
| | | ACCGTCACCGGACTCCGCCGCCGCCTCAAGAAGTGCCTCGCCGACCT |
| | | CCGCGAGGCCTGCGACGTGATCGTGGACGAACACATCAGCGGCAACC |
| | | GCCAGCGCATCCCCGGCGACCGCGACGAGGACTTCGTCGACGTCCTC |
| | | CTCCGCGTCCAGAAATCCCCCGACCTCGAGGTCCCCCTAACCGACGA |
| | | CAATCTCAAGGCCCTCGTCCTGGACATGTTCGTCGCCGGCACGGACA |
| | | CCACGTTCGCGACGCTGGAGTGGGTGATGACGGAGCTAGTCCGCCAC |
| | | CCACGGATCCTCAAGAAGGCGCAGGAGGAGGTCCGGCGAGTCGTCGG |
| | | CGACAGCGGCCGCGTCGAGGAGTCCCACCTCGGCGAGCTCCACTACA |
| | | TGCGCGCCATCATCAAGGAGACGTTCCGGCTGCACCCGGCGGTGCCG |
| | | TTGCTAGTGCCGCGCGAGTCCGTCGCGCCGTGCACGCTGGGCGGCTA |
| | | CGACATCCCGGCGAGGACGCGGGTGTTCATCAACACGTTCGCCATGG |
| | | GGCGCGACCCGGAGATCTGGGACAACCCGCTGGAGTACTCGCCGGAG |
| | | AGGTTCGAGAGCGCCGGCGGCGGCGGCGAGATCGACCTCAAGGACCC |
| | | GGACTACAAGCTGCTGCCGTTCGGCGGCGGGCGGCGAGGGTGCCCCG |
| | | GCTACACGTTCGCGCTCGCCACCGTGCAGGTGTCGCTCGCCAGCTTG |
| | | CTCTACCACTTCGAGTGGGCGCTGCCCGCCGGCGTGCGCGCCGAGGA |
| | | CGTCAACCTCGACGAGACGTTCGGCCTCGCCACGAGGAAGAAGGAGC |
| | | CGCTCTTCGTCGCCGTCAGGAAGAGCGACGCGTACGAGTTTAAGGGA |
| | | GAGGAGCTTAGTGAGGTTTAA |
| 38 | osT5H, coding | ATGGAATTGACAATGGCAAGCACTATGTCACTGGCATTATTAGTACT |
| | sequence (5′-3′), | ATCGGCTGCTTACGTGCTGGTTGCCCTTCGTAGGTCCAGGTCATCTT |
| | codon optimized, | CATCTAAGCCCAGAAGGCTGCCCCCTTCCCCACCCGGATGGCCTGTT |
| | encoding OsT5H of | ATTGGTCACCTTCACTTGATGTCAGGTATGCCTCATCATGCTTTAGC |
| | SEQ ID NO: 36 | GGAGTTAGCTAGGACTATGAGGGCCCCTCTTTTCAGAATGAGACTAG |
| | | GTAGCGTACCTGCTGTGGTTATATCAAAACCTGACTTAGCTCGTGCC |
| | | GCTTTAACAACAAATGACGCTGCCTTAGCTTCTAGGCCACATCTTCT |
| | | TTCTGGACAGTTCTTGTCCTTCGGGTGCTCAGACGTCACTTTCGCCC |
| | | CAGCTGGGCCATATCACAGGATGGCGAGAAGGGTGGTGGTCAGTGAA |
| | | TTGCTTTCTGCCAGGAGAGTAGCGACTTACGGCGCTGTGAGGGTAAA |
| | | AGAGCTTAGAAGGTTGTTGGCTCACTTAACTAAGAATACGTCCCCTG |
| | | CTAAACCAGTCGACTTATCTGAATGCTTTCTAAACTTAGCTAACGAT |
| | | GTACTATGTAGGGTCGCCTTTGGCAGACGTTTCCCCCACGGAGAGGG |
| | | TGACAAATTGGGTGCGGTATTGGCAGAGGCCCAGGACTTGTTCGCAG |
| | | GTTTCACTATAGGAGACTTCTTCCCGGAGTTAGAGCCCGTCGCAAGT |
| | | ACGGTAACTGGGTTAAGAAGAAGATTAAAGAAATGTCTAGCTGACCT |
| | | AAGAGAGGCTTGTGACGTAATAGTCGACGAGCATATATCTGGGAACA |
| | | GACAGAGGATACCAGGTGACAGAGATGAGGACTTCGTTGACGTCCTA |
| | | TTAAGAGTTCAAAAGAGTCCCGACTTGGAAGTTCCCTTAACTGATGA |
| | | CAACTTAAAGGCCTTAGTACTTGACATGTTTGTCGCAGGTACAGACA |
| | | CTACGTTTGCTACATTGGAGTGGGTCATGACTGAATTAGTGAGGCAT |
| | | CCGCGTATATTGAAGAAGGCCCAGGAAGAAGTCAGAAGAGTGGTAGG |
| | | TGATAGTGGAAGGGTGGAAGAGAGCCATCTGGGCGAATTGCATTATA |
| | | TGAGGGCGATTATAAAGGAAACGTTCAGGCTACACCCTGCCGTGCCT |
| | | TTATTGGTGCCCAGGGAGAGTGTAGCACCATGCACGTTGGGCGGATA |
| | | TGACATACCTGCTAGAACTAGAGTGTTTATAAACACCTTTGCGATGG |
| | | GACGTGATCCCGAGATATGGGACAATCCCTTAGAATACAGCCCCGAG |
| | | AGATTCGAGTCTGCGGGTGGTGGCGGCGAGATAGACCTAAAAGACCC |
| | | TGACTACAAGCTGTTACCTTTCGGCGGCGGTAGGAGAGGATGCCCTG |
| | | GTTACACATTTGCATTAGCTACCGTGCAGGTATCACTGGCGAGTTTG |
| | | TTGTACCACTTCGAATGGGCGTTACCAGCCGGAGTCCGTGCCGAGGA |
| | | CGTCAACCTTGACGAGACTTTCGGCTTAGCTACAAGGAAGAAGGAGC |
| | | CCTTATTCGTCGCTGTCAGAAAGAGCGACGCTTATGAATTTAAAGGG |
| | | GAAGAATTGTCCGAGGTATAA |
| 39 | OsCPR polypeptide | MALALEAARSWAASVLPPELAAAAGGDPLAALAATAAALVAGVVILA |
| | (GenBank: | VWFRSGGGAPPKAAAPPPRPPPVKIEADADADDGRKRVTVFFGTQTG |
| | XP_015636692) | TAEGFAKAMAEEARARYEKAVFKVVDLDDYAAEDEEYEEKLRKETIV |
| | | LLFLATYGDGEPTDNAARFYKWFTEGKEKEVWLKDLKYAVFGLGNRQ |
| | | YEHFNKVAKVVDELLEEQGGKRLVPVGLGDDDQCIEDDFTAWKEQVW |
| | | PELDQLLRDEDDTTGASTPYTAAIPEYRIVFIDKSDVSFQDKSWSLA |
| | | NGSGVIDIHHPVRSNVAVRKELHKPASDRSCIHLEFDISGTGLVYET |
| | | GDHVGVYSENAIETVEQAEKLLDLSPDTFFSVHADAEDGSPRKGGGS |
| | | LAPPFPSPCTLRTALLRYADLLNSPKKAALVALAAHASDLAEAERLR |
| | | FLASPAGKDEYSQWVVASQRSLLEVMAAFPSAKPPLGVFFAAVAPRL |
| | | QPRYYSISSSPKMAPSRIHVTCALVYGPTPTGRIHQGVCSTWMKNAI |
| | | PSEYSEECSWAPIYVRQSNFKLPADPTTPIIMIGPGTGLAPFRGFLQ |
| | | ERLALKQSGVELGNSVLFFGCRNRNMDYIYEDELQNFIQEGALSELI |
| | | VAFSREGPAKEYVQHKMTEKATEIWNIVSQGGYIYVCGDAKGMARDV |
| | | HRALHTIVQEQGSLDSSKTESYVKSLQMDGRYLRDVW |
| 40 | osCPR coding | ATGGCGCTGGCGCTGGAGGCGGCGAGGTCGTGGGCGGCGTCCGTGCT |
| | sequence (5′-3′) | GCCGCCGGAGCTCGCCGCCGCGGCGGGGGGCGACCCGCTCGCCGCCC |
| | encoding OsCPR of | TCGCCGCCACGGCCGCGGCGCTCGTCGCCGGGGTGGTCATCCTCGCC |
| | SEQ ID NO: 39 | GTGTGGTTCCGATCCGGTGGAGGGGCGCCGCCCAAGGCCGCCGCGCC |
| | (GenBank: | GCCGCCGCGCCCGCCGCCGGTCAAGATCGAGGCCGACGCCGACGCCG |
| | XM_015781206) | ACGACGGCCGGAAGCGCGTCACCGTCTTCTTCGGCACGCAGACCGGC |
| | | ACCGCCGAGGGCTTCGCTAAGGCGATGGCAGAGGAGGCGAGGGCGCG |
| | | GTACGAGAAGGCGGTGTTCAAAGTTGTGGATCTGGATGACTATGCAG |
| | | CTGAGGATGAGGAGTACGAGGAGAAGCTCAGGAAGGAAACTATCGTG |
| | | TTGCTCTTCCTAGCAACATATGGGGATGGGGAGCCTACCGACAATGC |
| | | TGCAAGATTCTACAAATGGTTCACTGAGGGGAAGGAAAAGGAAGTCT |
| | | GGCTTAAGGATCTTAAGTATGCTGTTTTTGGCCTGGGAAATAGGCAG |
| | | TATGAGCACTTTAATAAGGTTGCAAAGGTGGTGGACGAACTGCTAGA |
| | | GGAGCAGGGTGGCAAGCGTCTTGTCCCAGTTGGCCTTGGAGATGATG |
| | | ATCAGTGCATTGAGGATGACTTCACAGCATGGAAAGAACAAGTTTGG |
| | | CCAGAATTGGACCAACTGCTCCGTGATGAAGATGATACAACTGGTGC |
| | | ATCCACTCCTTACACTGCTGCTATACCTGAATACAGAATTGTATTTA |
| | | TTGATAAATCAGATGTATCGTTCCAAGACAAATCGTGGTCACTTGCC |
| | | AATGGCAGTGGTGTTATTGATATTCACCATCCTGTCAGGTCTAATGT |
| | | TGCTGTTCGAAAGGAACTCCATAAGCCAGCTTCAGATCGCTCTTGCA |
| | | TTCATCTGGAGTTCGATATCTCAGGCACTGGTCTTGTGTATGAAACT |
| | | GGTGACCATGTTGGTGTTTATTCAGAAAATGCTATCGAGACAGTGGA |
| | | GCAGGCTGAAAAGCTGCTAGATCTTTCCCCAGATACATTTTTCTCCG |
| | | TCCATGCAGACGCAGAAGATGGATCACCTCGTAAAGGGGGTGGTTCT |
| | | TTGGCCCCACCTTTCCCTTCTCCTTGCACCTTACGGACTGCACTTCT |
| | | AAGATATGCTGATCTACTTAATTCTCCAAAGAAGGCTGCTTTGGTTG |
| | | CTTTAGCTGCTCATGCGTCTGACCTAGCTGAAGCTGAAAGATTGAGA |
| | | TTTTTGGCTTCCCCTGCTGGAAAGGATGAATATTCTCAATGGGTAGT |
| | | AGCTAGCCAAAGAAGCCTTCTTGAAGTTATGGCTGCATTCCCTTCAG |
| | | CTAAGCCCCCACTGGGAGTATTCTTTGCAGCTGTTGCACCTCGCCTA |
| | | CAACCGCGTTACTACTCCATATCATCATCTCCAAAGATGGCTCCTTC |
| | | CAGAATTCATGTAACATGTGCGTTAGTTTATGGACCTACACCAACGG |
| | | GAAGGATTCACCAAGGAGTTTGTTCGACATGGATGAAGAACGCAATC |
| | | CCATCAGAATATAGTGAAGAGTGTAGCTGGGCACCAATTTATGTGAG |
| | | GCAGTCAAATTTTAAGTTGCCTGCAGATCCGACCACTCCAATTATCA |
| | | TGATTGGTCCTGGTACAGGGTTAGCACCATTTAGAGGCTTCTTGCAG |
| | | GAAAGATTAGCTCTAAAACAATCTGGAGTTGAACTGGGCAATTCAGT |
| | | TCTCTTCTTTGGATGCAGGAACCGTAATATGGACTACATATATGAAG |
| | | ACGAGTTGCAAAACTTCATTCAGGAGGGTGCACTTTCTGAGCTGATT |
| | | GTTGCCTTCTCTCGTGAAGGACCAGCCAAAGAATACGTGCAGCATAA |
| | | GATGACAGAGAAGGCCACAGAAATTTGGAACATCGTCTCACAAGGTG |
| | | GTTACATATATGTGTGCGGTGATGCCAAGGGTATGGCAAGAGACGTA |
| | | CACAGAGCTCTTCATACAATTGTTCAAGAGCAGGGATCTTTGGATAG |
| | | CTCCAAAACCGAGAGCTATGTAAAGAGCCTACAGATGGACGGCAGGT |
| | | ACCTCCGTGACGTATGGTGA |
| 41 | osCPR, coding | ATGGCGCTTGCATTAGAAGCAGCAAGAAGTTGGGCAGCCTCTGTTTT |
| | sequence (5′-3′), | ACCGCCAGAGTTGGCTGCCGCCGCAGGCGGCGATCCTTTGGCCGCAT |
| | codon optimized, | TGGCAGCCACAGCTGCTGCTTTGGTAGCAGGCGTGGTCATCTTAGCA |
| | encoding OsCPR of | GTTTGGTTTAGAAGCGGTGGTGGTGCCCCGCCTAAGGCTGCAGCTCC |
| | SEQ ID NO: 39 | CCCACCCAGACCCCCACCAGTAAAGATAGAAGCAGATGCGGACGCAG |
| | | ACGACGGACGTAAAAGGGTGACAGTGTTCTTTGGCACCCAGACGGGA |
| | | ACGGCAGAGGGTTTCGCAAAAGCTATGGCTGAAGAGGCCAGGGCGAG |
| | | GTATGAGAAGGCTGTCTTTAAAGTCGTTGATCTTGACGACTATGCGG |
| | | CCGAGGACGAGGAGTACGAGGAAAAGTTAAGAAAGGAAACTATCGTA |
| | | TTACTGTTCTTGGCGACATACGGCGACGGTGAGCCTACTGACAATGC |
| | | GGCCAGGTTTTACAAGTGGTTTACAGAGGGAAAGGAGAAAGAGGTAT |
| | | GGCTTAAGGACCTGAAGTACGCAGTCTTTGGATTGGGTAATAGGCAG |
| | | TACGAACACTTCAACAAGGTTGCGAAAGTTGTGGACGAACTACTTGA |
| | | AGAACAGGGTGGCAAGAGACTGGTTCCAGTAGGTCTGGGTGACGACG |
| | | ACCAGTGCATTGAGGATGACTTTACCGCCTGGAAGGAGCAAGTTTGG |
| | | CCCGAGCTTGATCAGCTGTTGCGTGACGAGGACGACACTACTGGCGC |
| | | CTCAACTCCATACACTGCCGCGATACCGGAGTATCGTATAGTATTCA |
| | | TTGACAAATCCGATGTGTCTTTTCAAGACAAGTCGTGGAGCTTAGCT |
| | | AATGGCTCCGGCGTCATAGACATTCACCATCCAGTTAGATCTAACGT |
| | | TGCCGTACGTAAAGAGTTACATAAGCCGGCATCCGACAGGTCTTGCA |
| | | TCCATTTAGAGTTTGATATTAGTGGAACCGGGCTTGTTTATGAGACA |
| | | GGTGACCACGTCGGTGTCTACTCCGAGAACGCAATAGAGACTGTGGA |
| | | ACAGGCCGAGAAGCTGCTAGACTTATCCCCTGACACCTTCTTTTCTG |
| | | TCCATGCCGACGCAGAGGATGGCTCACCAAGGAAAGGTGGCGGCTCT |
| | | CTAGCTCCACCCTTTCCTTCCCCATGCACGTTGCGTACTGCTTTATT |
| | | GAGGTACGCTGATTTGTTAAACAGTCCTAAGAAAGCTGCTTTGGTTG |
| | | CTCTAGCGGCCCACGCTTCAGACCTAGCTGAGGCGGAGAGATTGCGT |
| | | TTCTTAGCTTCCCCAGCAGGGAAGGACGAATATTCACAATGGGTTGT |
| | | GGCTTCTCAGCGTTCTCTGCTTGAAGTGATGGCAGCTTTTCCAAGTG |
| | | CCAAACCACCCTTGGGAGTATTCTTCGCAGCCGTGGCGCCAAGGTTA |
| | | CAGCCAAGATATTATTCCATATCTTCTTCACCGAAGATGGCGCCTTC |
| | | CAGGATACACGTCACCTGCGCATTAGTATATGGACCAACACCGACAG |
| | | GAAGAATACACCAGGGTGTATGCTCTACCTGGATGAAGAATGCTATA |
| | | CCTTCAGAGTATTCTGAGGAGTGTTCTTGGGCCCCTATATACGTACG |
| | | TCAGAGTAACTTCAAGCTTCCGGCCGATCCTACCACGCCAATTATAA |
| | | TGATTGGACCAGGAACAGGACTGGCGCCATTTCGTGGATTTCTGCAG |
| | | GAGAGACTGGCTTTGAAGCAATCTGGAGTAGAGCTGGGAAACTCAGT |
| | | TCTTTTCTTCGGTTGCCGTAATAGGAATATGGACTATATATACGAAG |
| | | ATGAGTTACAGAACTTTATTCAGGAAGGAGCGCTTTCTGAATTGATC |
| | | GTCGCCTTCTCTAGAGAGGGTCCGGCAAAGGAGTATGTCCAGCATAA |
| | | GATGACGGAGAAAGCCACGGAGATATGGAACATCGTCAGCCAGGGCG |
| | | GTTACATTTATGTGTGTGGCGACGCCAAGGGTATGGCCCGTGACGTG |
| | | CACAGGGCCCTTCATACCATCGTCCAAGAGCAGGGCAGTTTGGACTC |
| | | ATCAAAGACGGAGTCTTACGTTAAAAGCCTTCAGATGGACGGGCGTT |
| | | ACTTGAGGGACGTTTGGTAG |
| 42 | RmNMT polypeptide | MENSYSAQMYIDEFDPVHYYQTYYSSGKGGIAREWTDFALQNLHETF |
| | (GenBank: | GPGGVKGDILIDFGAGPTIYQLLSACEVFNSIITSDFLEQNREQLEK |
| | WKR38372) | WLRKDPDALDWSHFTKYVCELEGNRDNWEKKEETLRRKVTKVLKCDA |
| | | LAEKPFDDVPMPEADCLISCLCLENPCQDQEAYINILKKLKELLKPG |
| | | GHIIIQSILNCSYYHIGNSCFSHLSLSKDDVEKSFKEAGYEIVKLKV |
| | | LPRSVMSEMEISDSNGYYFIHARKPQKE |
| 43 | rmNMT coding | ATGGAGAACTCATACTCTGCACAGATGTACATTGATGAATTTGATCC |
| | sequence (5′-3′) | TGTACACTACTACCAGACATACTATTCTTCGGGGAAAGGAGGTATCG |
| | encoding RmNMT of | CTAGAGAATGGACAGACTTTGCCTTACAGAATCTGCATGAAACATTT |
| | SEQ ID NO: 42 | GGTCCAGGAGGTGTGAAAGGAGACATACTAATTGACTTTGGAGCTGG |
| | (GenBank: | ACCCACAATTTATCAACTGCTCTCTGCTTGTGAAGTGTTCAACAGCA |
| | OQ557631) | TTATAACCTCTGATTTTCTTGAGCAAAACCGAGAGCAATTGGAAAAA |
| | | TGGCTGAGAAAGGACCCAGATGCCTTAGACTGGTCTCATTTTACCAA |
| | | ATATGTGTGTGAACTGGAAGGTAACAGAGATAACTGGGAAAAAAAGG |
| | | AAGAAACGCTGAGGAGAAAAGTAACCAAAGTGTTGAAGTGTGACGCT |
| | | CTTGCGGAGAAGCCATTTGACGACGTGCCTATGCCAGAAGCCGACTG |
| | | TCTGATTAGCTGCCTCTGCTTAGAAAATCCTTGTCAAGACCAAGAAG |
| | | CTTATATCAATATATTGAAAAAGCTTAAGGAGCTACTTAAACCCGGT |
| | | GGTCACATTATAATTCAGAGTATTCTTAATTGTTCATACTACCATAT |
| | | TGGTAACAGCTGTTTTTCCCATCTGTCCTTATCTAAGGATGACGTGG |
| | | AAAAATCATTTAAAGAGGCCGGGTATGAAATTGTTAAATTAAAGGTG |
| | | CTGCCACGATCTGTAATGTCAGAAATGGAGATCAGTGATTCTAATGG |
| | | CTATTACTTTATCCATGCTCGTAAACCACAGAAAGAGTAA |
| 44 | rmNMT, coding | ATGGAGAACTCTTACTCCGCTCAGATGTACATAGATGAATTTGATCC |
| | sequence (5′-3′), | TGTTCACTACTACCAGACTTACTATTCCTCGGGCAAAGGCGGCATAG |
| | codon optimized, | CTAGAGAATGGACCGACTTTGCACTACAGAATCTACATGAAACATTT |
| | encoding RmNMT of | GGACCGGGTGGGGTTAAAGGCGACATATTGATTGACTTTGGCGCTGG |
| | SEQ ID NO: 42 | ACCTACAATCTATCAACTACTGTCAGCATGTGAAGTGTTCAACAGCA |
| | | TTATAACATCCGATTTTCTGGAGCAAAACAGAGAGCAACTAGAAAAG |
| | | TGGTTAAGAAAGGACCCCGATGCCCTTGACTGGTCCCATTTTACTAA |
| | | ATATGTATGTGAACTTGAAGGTAACAGAGATAACTGGGAAAAGAAGG |
| | | AAGAAACCCTGCGTAGAAAAGTCACAAAGGTTCTAAAGTGTGACGCG |
| | | TTGGCCGAGAAACCTTTTGACGACGTACCGATGCCAGAAGCCGACTG |
| | | TCTTATTAGTTGCTTATGCTTAGAAAATCCATGTCAAGACCAAGAGG |
| | | CTTATATCAACATCTTGAAGAAGTTGAAAGAGTTGCTAAAGCCGGGC |
| | | GGTCACATCATAATTCAAAGCATCTTAAATTGTAGCTACTACCATAT |
| | | CGGAAATTCGTGCTTTTCTCATCTATCGTTGTCTAAGGATGACGTGG |
| | | AAAAGTCGTTTAAAGAGGCCGGGTATGAAATTGTAAAACTAAAGGTC |
| | | CTACCAAGGTCTGTAATGTCAGAAATGGAGATTTCCGATTCTAATGG |
| | | ATATTACTTTATCCATGCCAGAAAACCACAGAAAGAGTGA |
| 45 | psiD, coding | ATGCAGGTTATACCGGCATGTAACTCTGCGGCGATACGTAGCTTATG |
| | sequence (5′-3′), | CCCAACACCGGAATCATTTCGTAACATGGGATGGCTTAGCGTGTCTG |
| | codon optimized for | ACGCGGTCTATAGTGAATTCATCGGTGAGCTTGCGACTCGTGCTAGC |
| | E. coli, encoding | AACCGGAACTATTCTAATGAATTCGGTTTAATGCAGCCGATCCAGGA |
| | PsiD of SEQ ID NO: | ATTTAAGGCTTTTATTGAAAGTGATCCGGTTGTTCACCAGGAATTCA |
| | 9 | TTGACATGTTTGAAGGCATCCAGGACAGTCCGCGTAACTATCAGGAA |
| | | CTGTGCAATATGTTCAACGACATTTTTAGAAAAGCCCCAGTCTATGG |
| | | CGATCTGGGGCCCCCCGTATATATGATTATGGCCAAATTGATGAATA |
| | | CTCGTGCTGGTTTTAGTGCTTTTACTCGTCAGCGCCTCAACCTGCAT |
| | | TTTAAAAAGCTGTTCGATACGTGGGGGCTGTTTTTATCTAGTAAAGA |
| | | TAGCCGCAATGTTTTAGTAGCCGATCAGTTCGACGATAGACACTGTG |
| | | GTTGGCTTAACGAACGTGCGTTAAGCGCAATGGTGAAACACTATAAC |
| | | GGGAGAGCCTTCGATGAGGTCTTTCTGTGTGATAAAAATGCGCCATA |
| | | TTATGGTTTTAATTCTTATGATGATTTCTTTAACCGCCGCTTTCGTA |
| | | ATCGCGATATTGATCGCCCGGTGGTTGGTGGTGTTAATAATACGACG |
| | | TTAATATCCGCCGCGTGTGAATCTTTGTCTTATAATGTATCATATGA |
| | | TGTGCAATCGCTCGATACCCTTGTCTTTAAAGGAGAAACATATTCTC |
| | | TGAAACATCTGCTTAACAATGATCCTTTTACCCCACAATTCGAACAT |
| | | GGATCCATCTTGCAGGGTTTCTTAAACGTCACGGCGTACCATAGATG |
| | | GCACGCTCCAGTTAATGGGACCATTGTTAAGATCATTAACGTGCCAG |
| | | GCACTTATTTCGCGCAGGCACCGTCCACGATCGGTGACCCGATACCA |
| | | GACAATGATTACGATCCGCCCCCCTATTTGAAGTCATTAGTGTACTT |
| | | CTCTAATATCGCGGCACGTCAGATCATGTTTATAGAAGCCGACAACA |
| | | AAGAGATCGGGTTAATCTTTTTGGTATTCATTGGTATGACTGAAATT |
| | | TCTACTTGTGAGGCAACCGTATCAGAAGGGCAACATGTCAACCGTGG |
| | | CGACGACCTTGGGATGTTCCATTTCGGCGGCTCTTCTTTTGCTCTCG |
| | | GCTTGCGAAAAGACTGCAGAGCCGAAATAGTAGAAAAATTCACTGAG |
| | | CCAGGTACTGTCATTCGGATTAATGAAGTGGTCGCTGCTCTTAAGGC |
| | | ATGA |
| 46 | asTDC1, coding | ATGCCGTCTCATGAGGAAAAATCAGATGATGGCCTGTCGGAAATCAT |
| | sequence (5′-3′), | GCTGGAAACCGAGTCCTTATTAACGCGGCTCTTCACAATTAAAAGTC |
| | codon optimized for | CAGCCCCGAGCGATCCGATTGTACAAAAGCTGAACGCACATGAAATT |
| | E. coli, encoding | CAGAACATGCAGCACCAGCTGATGCCGGATGATCATCCACGGGATTT |
| | AsTDC1 of SEQ ID | ATCTAGTGCTTTGCAGCAGGCGATGGCAGTGTTCGATAACCGCATGA |
| | NO: 7 | GATTAACTCATCCGCGTTGTTTTAGTTATATACCTGTTTGCCCTAAT |
| | | CCGGTGGCCTTACTTGGGGATCTTCTTACATCCGTCTTCAACGTGAA |
| | | TGCAGCGTCTTGGGACGTCGGTAGTGGGCCTAGCGCTGTTGAGACCG |
| | | CGATGATAGCTTGGCTGGGTGCACAACTGGGACTTCCAGATTCTGTT |
| | | GAAGGTTGTTTCGTGTCGGGTGGATCAATGGGCAACATGACCGCTAT |
| | | TGTTGCGGCCCGCGATGAAATTCTGCATCCGTTACAGCGTGCTTACG |
| | | CCATTATTTATATTTCGGATCAGACTCATATTAGTATCCTGAAAGCG |
| | | CTGCATATTACCGGTTTCACCCATCACCAAGTACGGAAAATACCGAC |
| | | TGATGACGATTTTCGTATCGATGTTGATATTCTGCGGCGTACCGTGG |
| | | CGAGCGATCGCCACAGTGGACTGGTGCCTTTTCTCCTGGTTGCCACA |
| | | TGTGGGACTACAAATACAGGATCAATCGATCCTCTTCATGCATTGGC |
| | | TGACTTGGCACGAGATGAAGGTCTGTGGCTCCATGTTGACGGAGCGT |
| | | ATGGCGCGTCGATCGCCTTGTCTGATCAGCATCGCTCATTAGTGGAT |
| | | GGAATTGGTCGCGCTGATAGTATTAGTTGGGATGGACATAAATGGTT |
| | | ATTCCAAACATATGGATGCGGAATCGTTTTAACTCGGCGTAGCGGCT |
| | | CTTTACGCCGCTCCTTCGCCGTTGATGCAGAGTATATCCGGGACAGT |
| | | ACAGTTCCCGAAGGGACCACGAACTTTTACAACCTTGGTCCGGAGTT |
| | | AAGCCGCCCAGCTCGTGCGATGAGCCTTTGGTTGACTCTGCGTGTGC |
| | | TTGGTCGTCGCCGTATGGGTGAGATGATTGATCGCGGTTTTTTGTTG |
| | | GCTCGAACGGCCGACCACTCGATCCGTCAGTATCGTGATTGGATAAT |
| | | CGCCGCTCCTACAGTCGCCTCCATTGTCGTGTTCCGGTATGCTCCTC |
| | | CAGGGCTGGACGACGGAGCCCTCGACGCACTCAATAAGGCAATTAGT |
| | | CGTCGTCTGCTGGCAGAAGACGTTGCAGCGATTCTTACTACTACCGT |
| | | ACGTGGCCGTACGGTTCTCCGTATGTGTGCGATGAATCCACTGGTGG |
| | | AACCGGAGACTATCGAGGATGTGATTAGAAGAGTGGATGGTATTGCA |
| | | CGTAGTGAGGCGAGCCGCCGTAAGGAGTCTCATTTCCAGTTAGGGTC |
| | | TGCTATGTATCTGGGCTAA |
| 47 | crTDC coding | ATGGGAAGCATAGACTCAACAAACGTAGCAATGTCGAACTCCCCCGT |
| | sequence (5′-3′), | CGGAGAGTTCAAGCCGCTTGAGGCAGAGGAGTTCAGGAAGCAGGCCC |
| | codon optimized, | ACAGGATGGTAGACTTCATCGCCGACTACTACAAGAACGTGGAGACA |
| | encoding CrTDC of | TACCCCGTACTTAGCGAGGTGGAGCCCGGCTACTTAAGGAAGAGGAT |
| | SEQ ID NO: 29 | ACCCGAAACGGCACCTTACCTGCCCGAGCCCCTAGACGACATCATGA |
| | | AGGACATACAGAAGGACATAATCCCCGGAATGACCAACTGGATGAGT |
| | | CCCAACTTCTACGCATTCTTCCCTGCAACAGTCTCTTCCGCAGCGTT |
| | | CTTAGGGGAGATGCTAAGTACAGCGTTAAACTCCGTCGGATTCACAT |
| | | GGGTAAGTTCACCCGCCGCGACCGAGTTAGAGATGATCGTCATGGAC |
| | | TGGCTAGCGCAGATATTAAAGCTTCCGAAGTCCTTCATGTTCTCAGG |
| | | GACAGGCGGCGGCGTAATACAGAACACGACATCAGAGTCGATCCTTT |
| | | GCACGATCATAGCAGCAAGGGAGCGTGCACTAGAGAAGTTAGGACCT |
| | | GACAGTATCGGCAAGTTAGTATGCTACGGCTCCGACCAGACCCACAC |
| | | CATGTTCCCTAAGACATGCAAGTTAGCCGGGATATACCCTAACAACA |
| | | TACGTCTAATACCTACGACGGTCGAGACAGACTTCGGCATAAGTCCG |
| | | CAGGTGCTGAGGAAGATGGTTGAGGACGACGTCGCCGCAGGATACGT |
| | | ACCTCTATTCTTATGCGCCACCTTAGGGACGACATCAACCACCGCGA |
| | | CGGACCCCGTGGACTCACTTTCAGAGATCGCAAACGAGTTCGGAATC |
| | | TGGATACACGTAGACGCCGCGTACGCAGGAAGTGCCTGCATATGCCC |
| | | GGAGTTCAGGCACTACCTGGACGGAATAGAGCGTGTGGACTCATTAT |
| | | CGTTATCACCTCACAAGTGGTTACTTGCCTACTTAGACTGCACGTGC |
| | | CTATGGGTAAAGCAGCCGCACTTACTACTAAGGGCCCTGACAACCAA |
| | | CCCGGAGTACTTAAAGAACAAGCAGAGTGACCTAGACAAGGTAGTAG |
| | | ACTTCAAGAACTGGCAGATAGCCACAGGAAGGAAGTTCAGGAGCTTA |
| | | AAGCTATGGCTGATCCTGAGGTCGTACGGGGTCGTGAACTTACAGTC |
| | | ACACATACGTAGTGACGTGGCAATGGCAAAGATGTTCGAGGAGTGGG |
| | | TGCGTTCGGACTCCCGTTTCGAGATCGTGGTCCCTAGGAACTTCTCG |
| | | CTTGTATGCTTCCGTTTAAAGCCCGATGTCTCATCCCTTCACGTGGA |
| | | AGAGGTAAACAAGAAGTTACTTGACATGTTAAACTCCACGGGCCGTG |
| | | TCTACATGACACACACGATAGTCGGTGGCATCTACATGCTTCGTCTT |
| | | GCCGTCGGGAGCTCATTAACGGAAGAGCACCACGTACGTCGTGTATG |
| | | GGACTTAATACAGAAGCTAACCGACGACCTGTTGAAAGAGGCCTGA |
| 48 | Tryptophan | MGSKGPEGKPDDGLSEIMLEAESFLTKLLTINPPAPSDPIVRKLNTH |
| | decarboxylase from | EIQHMQHEVMPDDHPRDLSSVLQEAMAIFDNRMRLTHPHCFSYIPAC |
| | Aspergillus | PTSLAYLGDLLTSICNVNAASWDVSSGPSAVEKAMIAWLGAQLGLPD |
| | caelatus | SVGGCFVSGGSMANMAAIIAARDEVLHPRQRGDAVIYISDQTHISIL |
| | (GenBank: | KALHITGFMDYQVHKIPTDDEFRIDVDILRRAIITDRQSGRVPFLLV |
| | XP_031931155) | ANCGSTNTGSIDPLHALADLARDEGIWLHVDGAYGASIALSDRHHDL |
| | (herein “AcTSCD1”) | IDGIGRADSVSWDGHKWLFQTYGCGIVLTRDVHSLRRSFAVDAEYIS |
| | | STLAPRDNTNFYNLGPELSRPARAMSLWLTLRTLGRQRMGEMIDQGF |
| | | LLARTADGKIRQYKNWIIPAPTVASIVVFRYAPPGIDEEGLDTLNKA |
| | | ISQRLLSENIAAILTTKIRGRTTLRMCAMNPLVKPETIEDLIRQVDI |
| | | LAQSEASERHKSYPKMAKMLHAHCQERPGVDVFQHSH |
| 49 | acTDC coding | ATGGGTTCCAAGGGGCCAGAAGGAAAACCAGATGACGGACTGTCCGA |
| | sequence (5′-3′) | GATCATGTTAGAGGCGGAGTCTTTCTTGACGAAACTGCTCACGATTA |
| | encoding AcTDC of | ATCCTCCGGCACCGTCCGACCCTATTGTTCGAAAGCTTAATACCCAT |
| | SEQ ID NO: 48 | GAGATACAACACATGCAACATGAGGTTATGCCCGATGACCATCCTCG |
| | (GenBank: | AGATCTTTCATCCGTCCTTCAAGAAGCTATGGCAATCTTTGATAACC |
| | XM_032069937) | GGATGCGCCTCACCCACCCACATTGCTTTTCCTATATACCAGCTTGC |
| | | CCAACTTCCCTTGCCTATCTAGGTGATCTCCTCACTTCTATTTGCAA |
| | | CGTAAATGCTGCTAGTTGGGATGTGAGCTCTGGTCCAAGTGCGGTTG |
| | | AAAAAGCCATGATAGCCTGGCTGGGTGCCCAGCTGGGACTGCCAGAC |
| | | TCAGTTGGAGGTTGCTTTGTCTCAGGTGGCTCTATGGCAAACATGGC |
| | | GGCTATAATTGCAGCCCGAGATGAGGTTCTTCATCCTAGGCAGCGAG |
| | | GAGATGCGGTCATCTACATTTCTGACCAGACACATATCTCTATTCTT |
| | | AAGGCGCTACATATAACCGGCTTCATGGATTATCAAGTCCACAAGAT |
| | | ACCCACCGATGATGAATTTCGGATAGATGTGGACATCTTACGACGCG |
| | | CTATCATTACAGACCGCCAATCCGGACGTGTACCCTTTCTTCTTGTC |
| | | GCCAATTGTGGGAGCACCAACACAGGGAGTATCGACCCTCTCCATGC |
| | | ACTCGCTGATCTTGCACGAGATGAAGGCATATGGCTACATGTGGATG |
| | | GTGCATATGGTGCTTCCATCGCGCTCTCTGACAGGCATCACGATCTC |
| | | ATTGATGGGATCGGTCGCGCAGATAGTGTTTCTTGGGATGGGCATAA |
| | | GTGGCTCTTCCAGACATATGGCTGTGGAATCGTGCTCACGCGCGACG |
| | | TTCATTCTCTTAGGCGCAGCTTCGCCGTTGATGCCGAGTATATCAGC |
| | | AGTACATTGGCACCACGGGACAACACCAATTTCTACAACCTTGGTCC |
| | | AGAGCTCAGTCGGCCAGCGCGTGCCATGTCTCTTTGGCTCACCCTCA |
| | | GAACCCTAGGTCGTCAGCGTATGGGTGAAATGATCGACCAAGGTTTC |
| | | CTTCTTGCCCGTACTGCAGATGGTAAGATCCGTCAGTACAAGAACTG |
| | | GATTATTCCTGCTCCAACAGTTGCTAGCATTGTGGTTTTTCGATACG |
| | | CACCGCCTGGGATTGACGAAGAAGGGCTAGACACATTAAACAAGGCT |
| | | ATTTCACAGCGACTCTTGTCGGAGAATATTGCTGCTATACTCACCAC |
| | | CAAAATTCGCGGACGGACCACACTTCGAATGTGTGCTATGAACCCCC |
| | | TTGTGAAGCCAGAAACTATCGAGGATCTTATACGCCAGGTTGATATC |
| | | TTGGCCCAATCTGAGGCTTCTGAGCGACACAAGTCTTATCCAAAGAT |
| | | GGCTAAAATGCTTCATGCGCATTGTCAAGAACGCCCTGGTGTTGATG |
| | | TTTTTCAACATAGTCATTGA |
| 50 | acTDC coding | ATGGGCTCAAAAGGTCCTGAAGGTAAGCCTGACGATGGACTTTCAGA |
| | sequence (5′-3′), | GATCATGCTAGAGGCAGAGAGTTTCCTTACGAAGCTGTTAACCATAA |
| | codon optimized, | ACCCACCCGCACCCAGCGACCCCATCGTAAGGAAGTTAAACACACAC |
| | encoding AcTDC of | GAGATACAGCACATGCAGCACGAGGTAATGCCGGACGACCACCCGCG |
| | SEQ ID NO: 48 | TGACTTATCGTCCGTCTTACAAGAGGCAATGGCCATATTCGACAACC |
| | | GTATGAGGTTAACGCACCCTCACTGCTTCTCATACATACCTGCCTGC |
| | | CCTACAAGTCTTGCGTACCTAGGCGACTTACTGACCTCAATATGCAA |
| | | CGTGAACGCCGCGTCATGGGATGTCTCGTCCGGACCTTCGGCAGTAG |
| | | AGAAGGCAATGATAGCGTGGCTAGGAGCACAGTTAGGACTACCCGAC |
| | | TCAGTCGGTGGATGCTTTGTCTCCGGTGGAAGTATGGCAAACATGGC |
| | | CGCCATCATAGCGGCAAGGGACGAGGTCTTACACCCTAGGCAGAGGG |
| | | GCGACGCAGTAATATACATCAGCGACCAGACACACATCAGCATCTTA |
| | | AAGGCACTGCACATCACCGGATTCATGGACTACCAGGTACACAAGAT |
| | | CCCCACCGACGACGAGTTCCGTATAGACGTAGACATCTTACGTCGTG |
| | | CAATCATAACAGACAGGCAGAGTGGACGTGTACCGTTCTTATTAGTC |
| | | GCGAACTGCGGGTCAACAAACACCGGATCAATCGACCCGCTTCACGC |
| | | ACTTGCAGACCTAGCAAGGGACGAGGGGATCTGGTTACACGTCGACG |
| | | GCGCCTACGGCGCCAGCATCGCCTTATCGGACAGGCACCACGACCTG |
| | | ATAGACGGAATAGGCCGTGCCGACTCAGTCAGTTGGGACGGCCACAA |
| | | GTGGTTATTCCAGACATACGGCTGCGGCATCGTCCTAACGCGTGACG |
| | | TGCACAGCTTAAGAAGGAGTTTCGCGGTCGACGCCGAGTACATCAGC |
| | | TCCACACTGGCACCTAGGGACAACACCAACTTCTACAACTTAGGGCC |
| | | CGAGCTAAGCCGTCCTGCACGTGCCATGAGCTTATGGTTAACCCTGA |
| | | GGACCTTAGGGAGACAGCGTATGGGTGAGATGATAGACCAGGGATTC |
| | | CTGTTAGCAAGGACAGCAGACGGGAAGATCCGTCAGTACAAGAACTG |
| | | GATCATCCCCGCCCCTACAGTCGCAAGTATCGTGGTATTCCGTTACG |
| | | CGCCGCCTGGAATCGACGAGGAAGGACTAGACACGCTTAACAAGGCA |
| | | ATCTCGCAGCGTCTGTTATCAGAGAACATCGCAGCCATACTAACAAC |
| | | AAAGATACGTGGCAGGACGACATTAAGGATGTGCGCCATGAACCCTC |
| | | TTGTGAAGCCTGAGACAATCGAGGACCTAATCAGGCAGGTAGACATC |
| | | TTAGCACAGAGCGAGGCAAGTGAGCGTCACAAGAGCTACCCTAAGAT |
| | | GGCAAAGATGTTACACGCCCACTGCCAGGAGAGGCCTGGAGTCGACG |
| | | TATTCCAGCACAGCCACTGA |
| 51 | cnsBv2 coding | ATGACTGAGAAAGAAGAGGACGTCGACCAATTCCACACGAAGGTGTC |
| | sequence (5′-3′), | GATCGCCACCCTAGCCGCCCTTACAAGGCTATTAAAGATGAACACAC |
| | codon optimized, | AGACGCCTTCAGACCCCATCGTCAGGACACTTGACGCCGCAGGAACC |
| | encoding CnsBv2 of | AAGGAGATGCAAGAGGCATTCCTGCCTGGCGAGAGCCCTCACGACGT |
| | SEQ ID NO: 3 | CGCAAGCGTCATACAGGAGACTATGGACATATTCGACAAGAGGATGA |
| | | GGCTTACGCACCCTCACTGCTTCTGCTACGTCCCGTCCTGCCCTTCG |
| | | CCGTTAGCCTGGGTGGGAGATCTTTTCACCTCATTATACAACGTCAA |
| | | CGCAGTGTCCTGGGATGTCTCATCCGGCCCTAGCGCCATGGAGCAGG |
| | | CAATGATCAGGTGGTTAGCACACCAGGTCGGACTACCCCCGTCCGCA |
| | | ACCGGCTGCTTCGTAAGCGGTGGCTCCATGGCGAACTTATCCGCCGT |
| | | GGTTGCCGCCAGGGACAAGTTATTACCCCCTCTACGTCGTCCTGAGG |
| | | GGACAATATACGTGTCCGACCAGACCCACATCTCAATAAAGAAGTGC |
| | | CTTCACATCATCGGGTTCCACGAGCACCAGGTGAGGGTCATCCCTAC |
| | | GGACGAGGCGTTCCGTATGGACATCGCCATCCTTCAGAGGACGATCG |
| | | CAGCAGACCGTCAGTGCGGACGTTTACCTTTCTTACTAGTCGCAAGC |
| | | TGCGGCACGACCAACACGGGGAGCATCGACCCTCTAGACGCCCTGGC |
| | | CGACGTAGCCAAGGAAGAGGGACTTTGGTTACACGTGGACGGAGCCT |
| | | ACGGAGCCTCGATAGCGCTTAGTGACAAGCACGGCCACTTAGTAAAC |
| | | GGAATCGGAAGGGCAGACAGTATAAGCTGGGACGGACACAAGTGGTT |
| | | ATTCCAGACATACGGGTGCGGCATCGTCCTGACCAGGGACTCAACCT |
| | | CGCTAGCAACATCATTCGCCGTGGACGCCGAGTACATAAACTCCACA |
| | | ACACCTCACGGAGCAGTGAACTTCTACAACTTCGGGCCCGAGTTATC |
| | | AAGGCCCGCCCGTGCAATCCCTCTTTGGTTCACGTTAAGGGTATTAG |
| | | GACGTAGGAAGATGGGAGAGATGATAGACCACGGATTCTTACTTGCA |
| | | AGGACAGCAGAGGACGAGATAAGGCGTTACGCACACTGGCTGATACT |
| | | TGCCCCGGTGACCGCAAGTATAGTGGTATTCCGTTACTCGCCGCCCG |
| | | AGCTAAAGGACAACGAGCTAGACGAGCTAAACCTAGCCATATCAACA |
| | | CGTTTACTTACCGAGAACGTGGCAAGTATATTCACAACCAACATCCG |
| | | TGGGAGAGTCGCCTTAAGGTTCTGCGCAATGAACCCTGCGACGAGGA |
| | | CAGACACAATCATGGAAGTAGTACAGCAGATGGACGAGGTGGCGCGT |
| | | ACCATCTCGACGAGTAGGCCGGGGCTTTGCAAGGACTGCAAGGACTG |
| | | A |

1. A cell capable of producing at least one tryptophan derivative comprising at least one exogenous polynucleotide encoding a pyridoxal phosphate (PLP)-dependent tryptophan decarboxylase,
wherein the level of production of the at least one tryptophan derivative in the cell is increased relative to the level of production of a second tryptophan derivative in an equivalent cell lacking the exogenous polynucleotide or comprising a second polynucleotide encoding a second tryptophan decarboxylase, and wherein the second polynucleotide is not the same as the exogenous polynucleotide.

2. A cell capable of producing at least one tryptophan derivative comprising at least one exogenous polynucleotide encoding a pyridoxal phosphate (PLP)-dependent tryptophan decarboxylase, wherein the level of production of the at least one tryptophan derivative in the cell is increased relative to the level of production of a second tryptophan derivative in an equivalent cell lacking the exogenous polynucleotide or comprising a second polynucleotide encoding a second tryptophan decarboxylase, and wherein the second polynucleotide is not the same as the exogenous polynucleotide and wherein the PLP-dependent tryptophan decarboxylase comprises one or more or all of:
a) a first amino acid sequence of GX₁X₂X₃X₄X₅X₆X₇X₈X₉X₁₀X₁₁X₁₂X₁₃X₁₄X₁₅X₁₆X₁₇R wherein:
X₁, X₂, X₃, X₄, X₈, X₉, X₁₀, X₁₁, X₁₂, X₁₃, X₁₄, X₁₅, and X₁₆ are any amino acid residue,
X₅ and X₆ are an amino acid residue selected from the group consisting of glycine, alanine, serine and threonine,
X₇ is an amino acid residue selected from the group consisting of serine and threonine, and
X₁₇ is an amino acid residue selected from the group consisting of alanine and glycine;
b) a second amino acid sequence of X₁X₂X₃X₄X₅X₆HX₇X₈X₉X₁₀KX₁₁X₁₂X₁₃X₁₄X₁₅X₁₆ wherein:
X₁ is any amino acid residue selected from the group consisting of tyrosine, leucine, phenylalanine, valine, cysteine and tryptophan,
X₂, X₄, X₅, X₆, X₇, X₈, X₉, X₁₀, X₁₁, X₁₂, X₁₃, X₁₄ and X₁₅ are any amino acid residue,
X₃ is any amino acid residue selected from the group consisting of serine and threonine, and
X₁₆ is an amino acid residue selected from the group consisting of glycine, alanine, serine and threonine;
c) a third amino acid sequence of DX₁X₂X₃X₄X₅X₆X₇X₈X₉X₁₀X₁₁X₁₂X₁₃X₁₄GX₁₅X₁₆X₁₇X₁₈X₁₉X₂₀X₂₁D wherein:
X₁, X₂, X₃, X₄, X₅, X₆, X₈, X₉, X₁₀, X₁₁, X₁₂, X₁₃, X₁₄, X₁₅, X₁₇, X₁₈, X₁₉ and X₂₁ are any amino acid residue, and
X₇ is an amino acid residue selected from the group consisting of proline and asparagine,
X₁₆ is an amino acid residue selected from the group consisting of threonine, proline and valine, and
X₂₀ is 1, 2, 3, 4, 5, or 6 amino acid residues and is any amino acid residue;
d) a fourth amino acid sequence of X₁X₂HX₃X₄X₅X₆X₇X₈X₉X₁₀ wherein:
X₁ is an amino acid residue selected from the group consisting of tryptophan and phenylalanine,
X₂, X₃, X₆, X₉ and X₁₀ are any amino acid residue,
X₄ is an amino acid residue selected from the group consisting of aspartic acid and glutamic acid,
X₅ is an amino acid residue selected from the group consisting of glycine, alanine and serine,
X₇ is an amino acid residue selected from the group consisting of phenylalanine, tyrosine and tryptophan, and
X₈ is an amino acid residue selected from the group consisting of glycine, alanine, serine and threonine; and
e) a fifth amino acid sequence of X₁X₂X₃X₄X₅X₆X₇HKX₈X₉X₁₀X₁₁X₁₂X₁₃X₁₄X₁₅X₁₆ wherein:
X₁ is an amino acid residue selected from the group consisting of aspartic acid, glutamic acid, histidine and asparagine,
X₂ is an amino acid residue selected from the group consisting of serine and threonine,
X₃, X₄, X₅, X₇, X₁₀, X₁₁, X₁₂, X₁₃ and X₁₄ are any amino acid residue,
X₆ is an amino acid residue selected from the group consisting of aspartic acid, glutamic acid, asparagine, serine and threonine,
X₈ is an amino acid residue selected from the group consisting of tryptophan, phenylalanine, tyrosine, leucine, methionine and cysteine,
X₉ is an amino acid residue selected from the group consisting of leucine, isoleucine, valine, methionine and phenylalanine,
X₁₅ is an amino acid residue selected from the group consisting of cysteine, alanine and serine, and
X₁₆ is an amino acid residue selected from the group consisting of glycine, serine, alanine and threonine.

3. The cell of claim 2, wherein:
a) the first amino acid sequence of GX₁X₂X₃X₄X₅X₆X₇X₈X₉X₁₀X₁₁X₁₂X₁₃X₁₄X₁₅X₁₆X₁₇R wherein:
X₁, X₂, X₃, X₈, X₉, X₁₂, X₁₃, X₁₄, X₁₅, and X₁₆ are any amino acid residue,
X₄ is an amino acid residue selected from the group consisting of serine, threonine and asparagine,
X₅ and X₆ are an amino acid residue selected from the group consisting of glycine, alanine, serine and threonine,
X₇ is an amino acid residue selected from the group consisting of serine and threonine,
X₁₀ is an amino acid residue selected from the group consisting of asparagine, isoleucine and valine,
X₁₁ is an amino acid residue selected from the group consisting of leucine, isoleucine, methionine and valine, and
X₁₇ is an amino acid residue selected from the group consisting of alanine and glycine;
b) the second amino acid sequence of X₁X₂X₃X₄X₅X₆HX₇X₈X₉X₁₀KX₁₁X₁₂X₁₃X₁₄X₁₅X₁₆ wherein:
X₁ is any amino acid residue selected from the group consisting of tyrosine, leucine, phenylalanine, valine, cysteine and tryptophan,
X₂, X₄, X₅, X₆, X₇, X₉, X₁₀, X₁₁, X₁₂, X₁₃, X₁₄ and X₁₅ are any amino acid residue,
X₃ is any amino acid residue selected from the group consisting of serine and threonine,
X₈ is any amino acid residue selected from the group consisting of serine and methionine, and
X₁₆ is an amino acid residue selected from the group consisting of glycine, alanine, serine and threonine;
c) the third amino acid sequence of DX₁X₂X₃X₄X₅X₆X₇X₈X₉X₁₀X₁₁X₁₂X₁₃X₁₄GX₁₅X₁₆X₁₇X₁₈X₁₉X₂₀X₂₁D wherein:
X₁, X₂, X₃, X₅, X₆, X₉, X₁₀, X₁₁, X₁₅, X₁₇ and X₁₈ are any amino acid residue,
X₄ is an amino acid residue selected from the group consisting of glycine, alanine, serine, aspartic acid and asparagine,
X₇ is an amino acid residue selected from the group consisting of proline and asparagine
X₈ is an amino acid residue selected from the group consisting of phenylalanine, isoleucine, leucine, threonine, tyrosine and tryptophan,
X₁₂ is an amino acid residue selected from the group consisting of alanine and glycine,
X₁₃ is an amino acid residue selected from the group consisting of serine, threonine and asparagine,
X₁₄ is an amino acid residue selected from the group consisting of cysteine, alanine, serine, threonine, leucine, glycine and valine, and
X₁₆ is an amino acid residue selected from the group consisting of threonine, proline and valine,
X₁₉ is an amino acid residue selected from the group consisting of glycine, alanine, serine and threonine,
X₂₀ is 1, 2, 3, 4, 5 or 6 amino acid residues and is any amino acid residue, and
X₂₁ is an amino acid residue selected from the group consisting of isoleucine, leucine, valine and threonine;
d) the fourth amino acid sequence of X₁X₂HX₃X₄X₅X₆X₇X₈X₉X₁₀ wherein:
X₁ is an amino acid residue selected from the group consisting of tryptophan and phenylalanine,
X₂, X₃, X₆ and X₁₀ are any amino acid residue,
X₄ is an amino acid residue selected from the group consisting of aspartic acid and glutamic acid,
X₅ is an amino acid residue selected from the group consisting of glycine, alanine and serine,
X₇ is an amino acid residue selected from the group consisting of phenylalanine, tyrosine and tryptophan,
X₈ is an amino acid residue selected from the group consisting of glycine, alanine, serine and threonine, and
X₉ is an amino acid residue selected from the group consisting of alanine, glycine, leucine, serine and threonine; and
e) the fifth amino acid sequence of X₁X₂X₃X₄X₅X₆X₇HKX₈X₉X₁₀X₁X₁₂X₁₃X₁₄X₁₅X₁₆ wherein:
X₁ is an amino acid residue selected from the group consisting of aspartic acid, glutamic acid, histidine and asparagine,
X₂ is an amino acid residue selected from the group consisting of serine and threonine,
X₃, X₄, X₅, X₇, X₁₀, X₁₂ and X₁₄ are any amino acid residue,
X₆ is an amino acid residue selected from the group consisting of aspartic acid, glutamic acid, asparagine, serine and threonine,
X₈ is an amino acid residue selected from the group consisting of tryptophan, phenylalanine, tyrosine and cysteine,
X₉ is an amino acid residue selected from the group consisting of leucine, isoleucine, valine, methionine and phenylalanine,
X₁₁ is an amino acid residue selected from the group consisting of glutamine, valine, threonine and alanine,
X₁₃ is an amino acid residue selected from the group consisting of tyrosine, phenylalanine, leucine, isoleucine and valine,
X₁₅ is an amino acid residue selected from the group consisting of cysteine, alanine and serine, and
X₁₆ is an amino acid residue selected from the group consisting of glycine, serine, alanine and threonine.

4. The cell of claim 3, wherein:
a) the first amino acid sequence of GX₁X₂X₃X₄X₅X₆X₇X₈X₉X₁₀ X₁₁X₁₂X₁₃X₁₄X₁₅X₁₆X₁₇R wherein:
X₁, X₂, X₃, X₈, X₁₂, X₁₃, X₁₄, X₁₅, and X₁₆ are any amino acid residue,
X₄ is an amino acid residue selected from the group consisting of serine, threonine and asparagine,
X₅ and X₆ are an amino acid residue selected from the group consisting of glycine and threonine,
X₇ is an amino acid residue selected from the group consisting of serine and threonine,
X₉ is an amino acid residue selected from the group consisting of alanine, glycine, cysteine and serine,
X₁₀ is an amino acid residue selected from the group consisting of asparagine, isoleucine and valine,
X₁₁ is an amino acid residue selected from the group consisting of leucine, methionine and valine, and
X₁₇ is an amino acid residue selected from the group consisting of alanine and glycine;
b) the second amino acid sequence of X₁X₂X₃X₄X₅X₆HX₇X₈X₉X₁₀KX₁₁X₁₂X₁₃X₁₄X₁₅X₁₆ wherein:
X₁ is any amino acid residue selected from the group consisting of tyrosine, leucine, phenylalanine, valine, cysteine and tryptophan,
X₂, X₄, X₇, X₉, X₁₀, X₁₁, X₁₂, X₁₃, X₁₄ and X₁₅ are any amino acid residue,
X₃ is any amino acid residue selected from the group consisting of serine and threonine,
X₅ is any amino acid residue selected from the group consisting of glutamine and glutamic acid,
X₆ is any amino acid residue selected from the group consisting of glycine, alanine, serine and threonine,
X₈ is the amino acid residue serine, and
X₁₆ is the amino acid residue glycine;
c) the third amino acid sequence of DX₁X₂X₃X₄X₅X₆X₇X₈X₉X₁₀X₁₁X₁₂X₁₃X₁₄GX₁₅X₁₆X₁₇X₁₈X₁₉X₂₀X₂₁D wherein:
X₁, X₂, X₃, X₅, X₆, X₉, X₁₁, X₁₅, X₁₈ and X₂₀ are any amino acid residue,
X₄ is an amino acid residue selected from the group consisting of glycine, alanine, serine, aspartic acid and asparagine,
X₇ is the amino acid residue proline,
X₈ is an amino acid residue selected from the group consisting of phenylalanine, threonine and tyrosine,
X₁₀ is an amino acid residue selected from the group consisting of isoleucine, leucine, valine, alanine and glycine,
X₁₂ is an amino acid residue selected from the group consisting of alanine and glycine,
X₁₃ is an amino acid residue selected from the group consisting of serine, threonine and asparagine,
X₁₄ is an amino acid residue selected from the group consisting of cysteine, serine, threonine, leucine, glycine and valine,
X₁₆ is an amino acid residue selected from the group consisting of threonine, proline and valine,
X₁₇ is an amino acid residue selected from the group consisting of asparagine, lysine, arginine, aspartic acid, serine and valine,
X₁₉ is an amino acid residue selected from the group consisting of glycine, alanine, serine and threonine, and
X₂₁ is an amino acid residue selected from the group consisting of isoleucine, leucine, valine and threonine;
d) the fourth amino acid sequence of X₁X₂HX₃X₄X₅X₆X₇X₈X₉X₁₀ wherein:
X₁ is an amino acid residue selected from the group consisting of tryptophan and phenylalanine,
X₂ and X₃ are any amino acid residue,
X₄ is the amino acid residue aspartic acid,
X₅ is an amino acid residue selected from the group consisting of glycine, alanine and serine,
X₆ is an amino acid residue selected from the group consisting of alanine, valine and serine,
X₇ is an amino acid residue selected from the group consisting of phenylalanine, tyrosine and tryptophan,
X₈ is an amino acid residue selected from the group consisting of glycine, alanine, serine and threonine,
X₉ is an amino acid residue selected from the group consisting of alanine, glycine, leucine, serine and threonine, and
X₁₀ is an amino acid residue selected from the group consisting of serine, alanine, phenylalanine and threonine; and
e) the fifth amino acid sequence of X₁X₂X₃X₄X₅X₆X₇HKX₈X₉X₁₀X₁₁X₁₂X₁₃X₁₄X₁₅X₁₆ wherein:
X₁ is an amino acid residue selected from the group consisting of aspartic acid, glutamic acid, histidine and asparagine,
X₂ is an amino acid residue selected from the group consisting of serine and threonine,
X₃, X₄ and X₁₄ are any amino acid residue,
X₅ is the amino acid residue tryptophan,
X₆ is an amino acid residue aspartic acid,
X₇ is an amino acid residue selected from the group consisting of alanine and glycine,
X₈ is the amino acid residue tryptophan,
X₉ is the amino acid residue leucine,
X₁₀ is an amino acid residue selected from the group consisting of phenylalanine, leucine, glycine, asparagine and methionine,
X₁₁ is the amino acid residue glutamine,
X₁₂ is an amino acid residue selected from the group consisting of threonine, serine, leucine, isoleucine, valine and alanine,
X₁₃ is an amino acid residue selected from the group consisting of tyrosine, phenylalanine, leucine, isoleucine and valine,
X₁₅ is an amino acid residue selected from the group consisting of cysteine and serine, and
X₁₆ is an amino acid residue selected from the group consisting of glycine and serine.

5. The cell of any one of claims 2 to 4, wherein the exogenous polynucleotide comprises in order from 5′ to 3′ the first amino acid sequence, the second amino acid sequence, the third amino acid sequence, the fourth amino acid sequence and the fifth amino acid sequence.

6. The cell of claim 5, wherein there are about 2 to about 50 amino acids, or about 10 to about 35 amino acids, or about 12 to about 25 amino acids between the first and the second amino acid sequence.

7. The cell of claim 5 or 6, wherein there are about 2 to about 50 amino acids, or about 20 to about 40 amino acids, or about 25 to about 35 amino acids between the second and the third amino acid sequence.

8. The cell of any one of claims 5 to 7, wherein there are about 5 to about 50, or about 10 to about 25, or about 12 to about 22 amino acids between the third and the fourth amino acid sequence.

9. The cell of any one of claims 5 to 8, wherein there are about 5 to about 50 amino acids, or about 10 to about 30, or about 15 to about 20 amino acids between the fourth and the fifth amino acid sequence.

10. A cell capable of producing at least one tryptophan derivative comprising at least one exogenous polynucleotide encoding an amino acid sequence set forth in any one of SEQ ID NOs:1, 3, 5, 7, 29 and 48 or a sequence having at least about 70% sequence identity thereto,
wherein the level of production of the at least one tryptophan derivative in the cell is increased relative to the level of production of a second tryptophan derivative in an equivalent cell lacking the exogenous polynucleotide or comprising a second polynucleotide encoding a second tryptophan decarboxylase, and wherein the second polynucleotide is not the same as the exogenous polynucleotide.

11. The cell of claim 10, wherein the exogenous polynucleotide encodes an amino acid sequence set forth in SEQ ID NO: 1.

12. The cell of claim 10, wherein the exogenous polynucleotide encodes an amino acid sequence set forth in SEQ ID NO: 7.

13. The cell of claim 10, wherein the exogenous polynucleotide encodes an amino acid sequence set forth in SEQ ID NO: 9.

14. The cell of any one of claims 1 to 13, wherein the PLP-independent tryptophan decarboxylase is a PsiD tryptophan decarboxylase, and optionally comprises an amino acid sequence set forth in SEQ ID NO: 9.

15. The cell of any one of claims 1 to 14, wherein the cell comprises two or more copies of the exogenous polynucleotide.

16. The cell of any one of claims 1 to 15, wherein the exogenous polynucleotide is operably linked to a heterologous promoter.

17. The cell of any one of claims 1 to 16, further comprising a polynucleotide encoding an additional tryptophan decarboxylase.

18. The cell of claim 17, wherein the additional tryptophan decarboxylase is PsiD, optionally comprising the amino acid sequence set forth in SEQ ID NO: 9 or a sequence having at least about 70% sequence identity thereto.

19. The cell of any one of claims 1 to 18, wherein the cell further comprises a polynucleotide encoding a tryptamine 4-monooxygenase.

20. The cell of claim 19, wherein the tryptamine 4-monooxygenase is PsiH, optionally comprising the amino acid sequence set forth in SEQ ID NO: 11 or a sequence having at least about 70% sequence identity thereto.

21. The cell of claim 20, wherein the cell comprises two or more copies of the polynucleotide encoding PsiH.

22. The cell of any one of claims 1 to 21, wherein the cell further comprises a polynucleotide encoding a 4-hydroxytryptamine kinase.

23. The cell of claim 22, wherein the 4-hydroxytryptamine kinase is PsiK, optionally comprising the amino acid sequence set forth in SEQ ID NO: 13 or a sequence having at least about 70% sequence identity thereto.

24. The cell of claim 23, wherein the cell comprises two or more copies of the polynucleotide encoding PsiK.

25. The cell of any one of claims 1 to 24, wherein the cell further comprises a polynucleotide encoding a methyl transferase.

26. The cell of claim 25, wherein the methyl transferase is:
PsiM, optionally comprising the amino acid sequence set forth in SEQ ID NO: 15 or a sequence having at least about 70% sequence identity thereto; or
RmNMT optionally comprising the amino acid sequence set forth in SEQ ID NO: 42 or a sequence having at least about 70% sequence identity thereto.

27. The cell of claim 26, wherein the cell comprises at least two polynucleotides encoding PsiM and/or RmNMT.

28. The cell of any one of claims 1 to 27, wherein the cell further comprises a polynucleotide encoding a cytochrome P450 reductase.

29. The cell of claim 28, wherein the cytochrome P450 reductase is:
PcCpr, optionally comprising the amino acid sequence set forth in SEQ ID NO: 17 or a sequence having at least about 70% sequence identity thereto; or
OsCPR, optionally comprising the amino acid sequence set forth in SEQ ID NO: 39 or a sequence having at least about 70% sequence identity thereto.

30. The cell of claim 29, wherein the cell comprises at least two polynucleotides encoding PcCpr and/or OsCPR.

31. The cell of any one of claims 1 to 30, wherein the cell further comprises at least one copy of a polynucleotide encoding an adenosylhomocysteinase, optionally comprising the amino acid sequence set forth in SEQ ID NO: 19 or a sequence having at least about 70% sequence identity thereto.

32. The cell of any one of claims 1 to 31, wherein the cell further comprises at least one exogenous polynucleotide encoding an adenosine kinase, optionally comprising the amino acid sequence set forth in SEQ ID NO: 21 or a sequence having at least about 70% sequence identity thereto.

33. The cell of any one of claims 1 to 31, wherein the cell further comprises at least one exogenous polynucleotide encoding a tryptamine 5-hydroxylase, optionally comprising the amino acid sequence set forth in SEQ ID NO: 36 or a sequence having at least about 70% sequence identity thereto.

34. The cell of any one of claims 1 to 33, wherein the cell is a yeast cell.

35. The cell of claim 34, wherein the yeast cell is a Saccharomyces sp.

36. The cell of claim 35, wherein the Saccharomyces sp. is S. cerevisiae.

37. The cell of claim 36, wherein the yeast cell is a Yarrowia sp.

38. The cell of any one of claims 1 to 33, wherein the cell is abacterial cell.

39. The cell of claim 38, wherein the bacterial cell is an Escherichia sp. or a Corynebacterium sp.

40. The cell of claim 39, wherein the Escherichia sp. is E. coli.

41. The cell of claim 40, wherein the Corynebacterium sp. is C. glutamicum.

42. The cell of any one of claims 1 to 33, wherein the cell is a fungal cell.

43. The cell of claim 42, wherein the fungal cell is a Aspergillus sp.

44. The cell of any one of claims 1 to 43, wherein the at least one tryptophan derivative is tryptamine or a tryptamine-derived alkaloid.

45. The cell of any one of claims 1 to 44, wherein the tryptophan derivative is tryptamine.

46. The cell of claim 45, wherein the tryptamine-derived alkaloid is psilocybin.

47. A cell capable of producing at least one tryptophan derivative, comprising:
a) at least one exogenous polynucleotide encoding a PLP-dependent tryptophan decarboxylase as defined in any one of claims 1 to 10;
b) at least one exogenous polynucleotide encoding a PsiH tryptamine 4-monooxygenase;
c) at least one exogenous polynucleotide encoding a PsiK hydroxytryptamine kinase; and
d) at least one exogenous polynucleotide encoding a PsiM methyl transferase.

48. A cell capable of producing at least one tryptophan derivative, comprising:
a) at least one exogenous polynucleotide encoding a PLP-dependent tryptophan decarboxylase having an amino acid sequence set forth in any one of SEQ ID NOs: 1, 3, 5, 7, 29 and 48, or a sequence having at least about 70% sequence identity thereto;
b) at least one exogenous polynucleotide encoding a PsiH tryptamine 4-monooxygenase;
c) at least one exogenous polynucleotide encoding a PsiK hydroxytryptamine kinase; and
d) at least one exogenous polynucleotide encoding a PsiM methyl transferase.

49. The cell of claim 48 or 49, wherein the cell is capable of an increased level of production of the at least one tryptophan derivative relative to the level of production of at least one tryptophan derivative in an equivalent cell comprising a second polynucleotide encoding a second tryptophan decarboxylase, wherein the second polynucleotide is not the same as (a), and optionally wherein the second polynucleotide encodes a PsiD tryptophan decarboxylase.

50. The cell of any one of claims 48 to 49, wherein the tryptophan derivative is tryptamine or psilocybin.

51. The cell of any one of claims 47 to 50, wherein the cell further comprises at least one exogenous polynucleotide encoding a cytochrome P450 reductase, and optionally wherein the cytochrome P450 reductase a PcCpr cytochrome P450 reductase.

52. The cell of any one of claims 47 to 51, wherein the cell further comprises at least one exogenous polynucleotide encoding an adenosylhomocysteinase, and optionally wherein the adenosylhomocysteinase is an Sah1 adenosylhomocysteinase.

53. The cell of any one of claims 47 to 52, wherein the cell further comprises at least one polynucleotide encoding an adenosine kinase, and optionally an Ado1 adenosine kinase.

54. The cell of any one of claims 47 to 53, wherein the cell further comprises at least one polynucleotide encoding an additional tryptophan decarboxylase, and optionally wherein the additional tryptophan decarboxylase is a PsiD and/or a CrTDC tryptophan decarboxylase.

55. A vector comprising a polynucleotide encoding a PLP-dependent tryptophan decarboxylase as defined in any one of claims 1 to 10, optionally wherein the polynucleotide is operably linked to a heterologous promoter capable of directing expression of the polynucleotide.

56. A vector comprising a polynucleotide encoding a PLP-dependent tryptophan decarboxylase having an amino acid sequence set forth in any one of SEQ ID NOs: 1, 3, 5, 7, 29 and 48, optionally wherein the polynucleotide is operably linked to a heterologous promoter capable of directing expression of the polynucleotide in a cell that does not naturally express said polynucleotide.

57. A host cell comprising a vector of claim 55 or 56.

58. A method for producing at least one tryptophan derivative, the method comprising culturing the cell of any one of claims 1 to 54 and 57 under conditions suitable for the production of the at least one tryptophan derivative.

59. The method of claim 58, further comprising extracting the at least one tryptophan derivative from the cell.

60. The method of claim 59, wherein the extracted tryptophan derivative is tryptamine or a tryptamine-derived alkaloid.

61. The method of claim 60, wherein the tryptophan derivative is tryptamine.

62. The method of claim 60, wherein the tryptamine-derived alkaloid is psilocybin.

63. A method for producing at least one tryptophan derivative, the method comprising contacting tryptophan with at least one pyridoxal phosphate (PLP)-dependent tryptophan decarboxylase as defined in any one of claims 1 to 10, under conditions suitable for the production of the at least one tryptophan derivative.

64. A method for producing at least one tryptophan derivative, the method comprising contacting tryptophan with at least one pyridoxal phosphate (PLP)-dependent tryptophan decarboxylase comprising an amino acid sequence set forth in any one of SEQ ID NOs:1, 3, 5, 7, 29 and 48 or a sequence having at least about 70% sequence identity thereto, under conditions suitable for the production of the at least one tryptophan derivative.

65. The method of claim 63 or 64, wherein the method is performed in a bioreactor.

66. A tryptophan derivative produced by the method of any one of claims 58 to 65.

67. A composition comprising the cell of any one of claims 1 to 54 and 57, or the extracted tryptophan derivative of claim 67.

68. A method for treating or preventing a disease or disorder in a subject, comprising administering to the subject a composition of claim 67.

69. The method of claim 68, wherein the disease or disorder is a neurodevelopmental, neurological, neuromuscular, movement, psychiatric, or psychological disease, disorder or syndrome.


SEQUENCE LISTING (51 sequences)

SEQ ID NO: 1 - protein, 454 aa, synthetic construct
MNSPTASDPIVRTLDAPETKDMQEAFLPGESPQDVASVIQETMDIFDKRMRLTHPHCFCYVPSCPSPLAWVGDLFTSLYNVNAVSWDVSSGPSAMEQGMIRWLAQQVGLPPSAGGCFVSGGSMANLSAVVAARDKLLPPMRRAEGTIYTSDQTHISIKKCLHIIGFDEHQIRVIPTDHLFRMDTDILRRTIAADRQCGRLPFLVVASCGTTNTGSIDPLHALADVAEEEGLWLHVDGAYGASIALSDKYRHLVDGIGRADSISWDGHKWLFQTYGCGIVLTRDSASLAHSFAVDAEYINSTTPHGAVNFYNLSPELSRPARAMPLWFTLRVLGRRQMGEMIDQGFLLARTAEEEIRRYPHWVILAPVTASIVVFRYCPLGLKDDDLDTLNLALSARLLAENVASILTTNIRGRVALRFCATNPATQADTITKVVQQMDQMANTMPTCGLGMCKE

SEQ ID NO: 2 - DNA, 1365 bp, synthetic construct
  source 1..1365: mol_type = other DNA
atgaattccccaactgcctctgatcctattgtccgaacactggatgcccctgaaacgaaagacatgcaagaggcattcctcccgggagagagcccacaagacgtcgcatcagttatccaggaaaccatggatattttcgacaaacgcatgcgtctcacgcacccacactgcttttgctatgtaccgtcatgtccatctccgctagcctgggtgggggacctgttcacctcactctacaatgtaaacgcggttagttgggatgtgagttctggtccaagtgcaatggaacaaggaatgatcaggtggctggcccagcaggttggtcttcctccttctgctggtggatgctttgtctccggtggctctatggcaaacctatccgccgtcgtggcagctcgagataagttgcttccccctatgcgacgggctgagggaaccatatacacatcggaccaaacgcatatatcgataaagaaatgtctccatataatcggcttcgacgaacaccaaatccgcgttatccccacagaccacctattccgcatggacactgatatcctgcgacgaacgattgcagccgaccgccaatgtggtcgcctaccttttctcgtcgtggccagctgtggaaccacaaatacgggcagcatcgatcccctccatgctctagccgacgtggctgaagaagaaggactgtggctgcacgtggatggtgcgtacggtgcgtcaattgccctctccgacaagtaccgccatctcgtggatggaattggtcgcgcagacagtatatcatgggacggacataaatggcttttccaaacatatggttgtggaattgtcctcactcgtgatagtgcgtccctcgcccacagctttgccgtggatgcagagtatatcaactcgacgacaccgcacggggccgtgaatttctacaaccttagtcctgaactcagccgcccagcgagagccatgccgctttggtttacgctgagggtgctgggacgtcggcagatgggagagatgattgatcaagggttccttctcgcccgcaccgccgaagaggagattcgacgatatccgcattgggtgatcctggcaccagtaacagcgagtattgtcgtcttccgatactgtccattggggttgaaggatgatgatctggatacactgaacctggccctctcagctcgacttttggcagagaatgttgcgtccatcctcacaacaaacatccgtggtcgggtagcactacgtttctgtgcaacaaaccctgccacccaagcagatacaatcacgaaggtggttcaacaaatggaccaaatggccaacacaatgcccacttgcggacttggaatgtgcaaggaatag

SEQ ID NO: 3 - protein, 485 aa, synthetic construct
MTEKEEDVDQFHTKVSIATLAALTRLLKMNTQTPSDPIVRTLDAAGTKEMQEAFLPGESPHDVASVIQETMDIFDKRMRLTHPHCFCYVPSCPSPLAWVGDLFTSLYNVNAVSWDVSSGPSAMEQAMIRWLAHQVGLPPSATGCFVSGGSMANLSAVVAARDKLLPPLRRPEGTIYVSDQTHISIKKCLHIIGFHEHQVRVIPTDEAFRMDIAILQRTIAADRQCGRLPFLLVASCGTTNTGSIDPLDALADVAKEEGLWLHVDGAYGASIALSDKHGHLVNGIGRADSISWDGHKWLFQTYGCGIVLTRDSTSLATSFAVDAEYINSTTPHGAVNFYNFGPELSRPARAIPLWFTLRVLGRRKMGEMIDHGFLLARTAEDEIRRYAHWLILAPVTASIVVFRYSPPELKDNELDELNLAISTRLLTENVASIFTTNIRGRVALRFCAMNPATRTDTIMEVVQQMDEVARTISTSRPGLCKDCKD

SEQ ID NO: 4 - DNA, 1458 bp, synthetic construct
  source 1..1458: mol_type = other DNA
atgactgaaaaagaggaagatgttgatcaattccatacaaaagtttcaatcgcaactctagccgcgctgacgcgactactaaaaatgaacactcaaactccctccgatcccattgtccgaacactggatgctgctggaacaaaagaaatgcaagaggcattcctcccaggagagagcccacacgacgtcgcgtcagttatccaggaaactatggatatttttgacaaacgtatgcgtctcacgcacccacactgcttttgctatgtaccgtcatgtccatcgccgctcgcctgggtgggagatctgttcacctcgctctataatgtgaacgcggtcagttgggacgtgagttccggtcctagtgcaatggaacaggccatgatcaggtggctagcccatcaggttggtcttcctccttctgctacgggatgcttcgtctccggtggctctatggcaaacctatccgccgttgtggcagctcgagataaattactcccccctttacggcggcctgagggcaccatatacgtatcagaccagacgcatatatcgataaagaaatgtctccacataatcggcttccacgaacaccaagtccgcgttattcccacagacgaggcattccgcatggacatagccattctacagcgaacgattgcagccgaccgccaatgtggccggctcccttttctcctcgtggctagctgtgggactactaatacgggaagcattgatcccctcgatgctttagctgatgtggccaaagaagagggactgtggctgcacgtggatggtgcctacggtgcatcaattgccctctccgacaagcacggccatctcgtcaatggaatcggtcgcgcagatagtatatcatgggatgggcacaaatggcttttccagacatatggctgtggaattgtcctcactcgtgatagcacatccctcgccaccagttttgccgtggatgcggaatatatcaactcaacgacaccgcacggggccgtgaatttttataactttggccccgagctcagccgcccagcgcgagctataccgctttggtttacgctgagagtgctaggtcgtcggaagatgggagagatgattgaccacggcttccttctcgcccgcactgccgaagacgaaattcgacgatatgcgcattggttgatcctggcgccagtaacagcgagtatagtcgtctttcgatactctccaccggagttaaaggacaatgagctggatgaactgaacttagccatctcaactcgacttttgacggaaaatgttgcttccatcttcacgacaaacattcgtggccgggtggccctccgtttctgtgcgatgaatcctgccacccgaacagatacgatcatggaggtagtgcaacaaatggacgaagtggccaggacgatttctacttccagacctggattgtgcaaggactgcaaggactag

SEQ ID NO: 5 - protein, 457 aa, synthetic construct
MNTQTPSDPIVRTLDAAGTKEMQEAFLPGESPHDVASVIQETMDIFDKRMRLTHPHCFCYVPSCPSPLAWVGDLFTSLYNVNAVSWDVSSGPSAMEQAMIRWLAHQVGLPPSATGCFVSGGSMANLSAVVAARDKLLPPLRRPEGTIYVSDQTHISIKKCLHIIGFHEHQVRVIPTDEAFRMDIAILQRTIAADRQCGRLPFLLVASCGTTNTGSIDPLDALADVAKEEGLWLHVDGAYGASIALSDKHGHLVNGIGRADSISWDGHKWLFQTYGCGIVLTRDSTSLATSFAVDAEYINSTTPHGAVNFYNFGPELSRPARAIPLWFTLRVLGRRKMGEMIDHGFLLARTAEDEIRRYAHWLILAPVTASIVVFRYSPPELKDNELDELNLAISTRLLTENVASIFTTNIRGRVALRFCAMNPATRTDTIMEVVQQMDEVARTISTSRPGLCKDCKD

SEQ ID NO: 6 - DNA, 1374 bp, synthetic construct
  source 1..1374: mol_type = other DNA
atgaacactcaaactccctccgatcccattgtccgaacactggatgctgctggaacaaaagaaatgcaagaggcattcctcccaggagagagcccacacgacgtcgcgtcagttatccaggaaactatggatatttttgacaaacgtatgcgtctcacgcacccacactgcttttgctatgtaccgtcatgtccatcgccgctcgcctgggtgggagatctgttcacctcgctctataatgtgaacgcggtcagttgggacgtgagttccggtcctagtgcaatggaacaggccatgatcaggtggctagcccatcaggttggtcttcctccttctgctacgggatgcttcgtctccggtggctctatggcaaacctatccgccgttgtggcagctcgagataaattactcccccctttacggcggcctgagggcaccatatacgtatcagaccagacgcatatatcgataaagaaatgtctccacataatcggcttccacgaacaccaagtccgcgttattcccacagacgaggcattccgcatggacatagccattctacagcgaacgattgcagccgaccgccaatgtggccggctcccttttctcctcgtggctagctgtgggactactaatacgggaagcattgatcccctcgatgctttagctgatgtggccaaagaagagggactgtggctgcacgtggatggtgcctacggtgcatcaattgccctctccgacaagcacggccatctcgtcaatggaatcggtcgcgcagatagtatatcatgggatgggcacaaatggcttttccagacatatggctgtggaattgtcctcactcgtgatagcacatccctcgccaccagttttgccgtggatgcggaatatatcaactcaacgacaccgcacggggccgtgaatttttataactttggccccgagctcagccgcccagcgcgagctataccgctttggtttacgctgagagtgctaggtcgtcggaagatgggagagatgattgaccacggcttccttctcgcccgcactgccgaagacgaaattcgacgatatgcgcattggttgatcctggcgccagtaacagcgagtatagtcgtctttcgatactctccaccggagttaaaggacaatgagctggatgaactgaacttagccatctcaactcgacttttgacggaaaatgttgcttccatcttcacgacaaacattcgtggccgggtggccctccgtttctgtgcgatgaatcctgccacccgaacagatacgatcatggaggtagtgcaacaaatggacgaagtggccaggacgatttctacttccagacctggattgtgcaaggactgcaaggactag

SEQ ID NO: 7 - protein, 491 aa, synthetic construct
MPSHEEKSDDGLSEIMLETESLLTRLFTIKSPAPSDPIVQKLNAHEIQNMQHQLMPDDHPRDLSSALQQAMAVFDNRMRLTHPRCFSYIPVCPNPVALLGDLLTSVFNVNAASWDVGSGPSAVETAMIAWLGAQLGLPDSVEGCFVSGGSMGNMTAIVAARDEILHPLQRAYAIIYISDQTHISILKALHITGFTHHQVRKIPTDDDFRIDVDILRRTVASDRHSGLVPFLLVATCGTTNTGSIDPLHALADLARDEGLWLHVDGAYGASIALSDQHRSLVDGIGRADSISWDGHKWLFQTYGCGIVLTRRSGSLRRSFAVDAEYIRDSTVPEGTTNFYNLGPELSRPARAMSLWLTLRVLGRRRMGEMIDRGFLLARTADHSIRQYRDWIIAAPTVASIVVFRYAPPGLDDGALDALNKAISRRLLAEDVAAILTTTVRGRTVLRMCAMNPLVEPETIEDVIRRVDGIARSEASRRKESHFQLGSAMYLG

SEQ ID NO: 8 - RNA, 1476 nt, synthetic construct
  source 1..1476: mol_type = other RNA
atgccttcccatgaagaaaagtccgatgacgggctgtctgagatcatgctggaaacagaatctctcttgactcgacttttcacgatcaaatctccagcaccatccgatcccattgttcaaaagctcaatgcccatgagatacaaaacatgcagcatcaactgatgcctgatgaccatcctcgagatctttcttccgcacttcaacaagcgatggcagtcttcgataaccgcatgcgcctcacccacccccgttgcttctcctacatccccgtctgtcccaaccccgttgccctgttgggtgacctcctcacttctgtcttcaatgtaaacgctgccagttgggacgtgggctctggtcccagcgcggtcgaaacagccatgatagcctggctgggcgcccagcttggactgcccgactcagtcgaaggatgctttgtctccggcggctcgatgggcaacatgacagccatcgttgcggcgcgagacgagatccttcatcccctgcagcgcgcatacgccatcatctacatctccgaccagacacacatctccatcctcaaggccctccacatcaccggcttcacgcaccaccaggtccgcaagatccccaccgacgatgacttccgcatcgatgtcgacatcctgcgacgcaccgtcgcttccgaccgccactccggcctcgttcctttcctgcttgtcgccacctgcggcaccaccaacaccggcagcatcgaccctctgcatgcgctagcggatctcgcgcgcgacgaaggcctctggctccatgtcgacggcgcatacggtgcctccatcgcgctctccgatcagcaccgctccctcgttgacgggatcggtcgcgcagacagcatctcctgggacggccacaaatggctcttccagacctacggctgcgggatcgtactcacgcgtcgctctggctcgcttcgccgcagcttcgccgtggacgccgagtacatccgcgactcaacggtacccgaggggaccacgaacttctacaacctcggccccgagctcagtcggcctgcgcgcgccatgtctctctggctcactttgcgagtcctcggtcgtcgtcgcatgggcgagatgatcgaccggggtttcctccttgcgcgcaccgcagaccacagcatccgtcagtacagggactggatcatcgcggcgccgacggtcgccagcattgtagtttttcggtacgcgccgccgggcctggacgacggggcgctggacgcgttgaacaaggccatttcacggcgtctcctggccgaggatgttgctgctattctcacgactacggttcgcggacgcactgtgcttcggatgtgtgctatgaatcctcttgtggagccggagacgatcgaggatgtgatacgtcgagtggatgggatcgcgcggtcggaggcgtccagacgcaaggagagccattttcaattgggatctgccatgtatcttggctga

SEQ ID NO: 9 - protein, 439 aa, synthetic construct
MQVIPACNSAAIRSLCPTPESFRNMGWLSVSDAVYSEFIGELATRASNRNYSNEFGLMQPIQEFKAFIESDPVVHQEFIDMFEGIQDSPRNYQELCNMFNDIFRKAPVYGDLGPPVYMIMAKLMNTRAGFSAFTRQRLNLHFKKLFDTWGLFLSSKDSRNVLVADQFDDRHCGWLNERALSAMVKHYNGRAFDEVFLCDKNAPYYGFNSYDDFFNRRFRNRDIDRPVVGGVNNTTLISAACESLSYNVSYDVQSLDTLVFKGETYSLKHLLNNDPFTPQFEHGSILQGFLNVTAYHRWHAPVNGTIVKIINVPGTYFAQAPSTIGDPIPDNDYDPPPYLKSLVYFSNIAARQIMFIEADNKEIGLIFLVFIGMTEISTCEATVSEGQHVNRGDDLGMFHFGGSSFALGLRKDCRAEIVEKFTEPGTVIRINEVVAALKA

SEQ ID NO: 10 - RNA, 1320 nt, synthetic construct
  source 1..1320: mol_type = other RNA
atgcaggtgatacccgcgtgcaactcggcagcaataagatcactatgtcctactcccgagtcttttagaaacatgggatggctctctgtcagcgatgcggtctacagcgagttcataggagagttggctacccgcgcttccaatcgaaattactccaacgagttcggcctcatgcaacctatccaggaattcaaggctttcattgaaagcgacccggtggtgcaccaagaatttattgacatgttcgagggcattcaggactctccaaggaattatcaggaactatgtaatatgttcaacgatatctttcgcaaagctcccgtctacggagaccttggccctcccgtttatatgattatggccaaattaatgaacacccgagcgggcttctctgcattcacgagacaaaggttgaaccttcacttcaaaaaacttttcgatacctggggattgttcctgtcttcgaaagattctcgaaatgttcttgtggccgaccagttcgacgacagacattgcggctggttgaacgagcgggccttgtctgctatggttaaacattacaatggacgcgcatttgatgaagtcttcctctgcgataaaaatgccccatactacggcttcaactcttacgacgacttctttaatcgcagatttcgaaaccgagatatcgaccgacctgtagtcggtggagttaacaacaccaccctcatttctgctgcttgcgaatcactttcctacaacgtctcttatgacgtccagtctctcgacactttagttttcaaaggagagacttattcgcttaagcatttgctgaataatgaccctttcaccccacaattcgagcatgggagtattctacaaggattcttgaacgtcaccgcttaccaccgatggcacgcacccgtcaatgggacaatcgtcaaaatcatcaacgttccaggtacctactttgcgcaagccccgagcacgattggcgaccctatcccggataacgattacgacccacctccttaccttaagtctcttgtctacttctctaatattgccgcaaggcaaattatgtttattgaagccgacaacaaggaaattggcctcattttccttgtgttcatcggcatgaccgaaatctcgacatgtgaagccacggtgtccgaaggtcaacacgtcaatcgtggcgatgacttgggaatgttccatttcggtggttcttcgttcgcgcttggtctgaggaaggattgcagggcagagatcgttgaaaagttcaccgaacccggaacagtgatcagaatcaacgaagtcgtcgctgctctaaaggcttag

SEQ ID NO: 11 - protein, 508 aa, synthetic construct
MIAVLFSFVIAGCIYYIVSRRVRRSRLPPGPPGIPIPFIGNMFDMPEESPWLTFLQWGRDYNTDILYVDAGGTEMVILNTLETITDLLEKRGSIYSGRLESTMVNELMGWEFDLGFITYGDRWREERRMFAKEFSEKGIKQFRHAQVKAAHQLVQQLTKTPDRWAQHIRHQIAAMSLDIGYGIDLAEDDPWLEATHLANEGLAIASVPGKFWVDSFPSLKYLPAWFPGAVFKRKAKVWREAADHMVDMPYETMRKLAPQGLTRPSYASARLQAMDLNGDLEHQEHVIKNTAAEVNVGGGDTTVSAMSAFILAMVKYPEVQRKVQAELDALTNNGQIPDYDEEDDSLPYLTACIKELFRWNQIAPLAIPHKLMKDDVYRGYLIPKNTLVFANTWAVLNDPEVYPDPSVFRPERYLGPDGKPDNTVRDPRKAAFGYGRRNCPGIHLAQSTVWIAGATLLSAFNIERPVDQNGKPIDIPADFTTGFFRHPVPFQCRFVPRTEQVSQSVSGP

SEQ ID NO: 12 - DNA, 2100 bp, synthetic construct
  source 1..2100: mol_type = other DNA
atgatcgctgtactattctccttcgtcattgcaggatgcatatactacatcgtttctcgtagagtgaggcggtcgcgcttgccaccagggccgcctggcattcctattcccttcattgggaacatgtttgatatgcctgaagaatctccatggttaacatttctacaatggggacgggattacagtctgtcttgccgcgttgacttctaatatatgaacagctaatatattgtcagacaccgatattctctacgtggatgctggagggacagaaatggttattcttaacacgttggagaccattaccgatctattagaaaagcgagggtccatttattctggccggtgagctgatgttgagttttttgcaattgaatttgtggtcacacgtttccagacttgagagtacaatggtcaacgaacttatggggtgggagtttgacttagggttcatcacatacggcgacaggtggcgcgaagaaaggcgcatgttcgccaaggagttcagtgagaagggcatcaagcaatttcgccatgctcaagtgaaagctgcccatcagcttgtccaacagcttaccaaaacgccagaccgctgggcacaacatattcgccagtaagtactacttgaggaaaatagcgtacgcttcgctgaccggtccgtacatcaaagtcagatagcggcaatgtcactggatattggttatggaattgatcttgcagaagacgacccttggctggaagcgacccatttggctaatgaaggcctcgccatagcatcagtgccgggcaaattttgggtcgattcgttcccttctcgtgagcatccttcttctatgtaggaagggaaggagtctaacaagtgttagtaaaataccttcctgcttggttcccaggtgctgtcttcaagcgcaaagcgaaggtctggcgagaagccgccgaccatatggttgacatgccttatgaaactatgaggaaattagcagttagtcaaatgcgttctccccgtattttttcaatactctaacttcagctcacagcctcaaggattgactcgtccgtcgtatgcttcagctcgtctgcaagccatggatctcaacggtgaccttgagcatcaagaacacgtaatcaagaacacagccgcagaggttaatgtcggtaagtcaaaagcgtccgtcggcaattcaaaattcaggcgctaaagtgggtcttctcaccaaggtggaggcgatactgtaaggatttctcaatcgttagagtataagtgttctaatgcagtacatactccaccaaccagactgtctctgctatgtctgcgttcatcttggccatggtgaagtaccctgaggtccagcgaaaggttcaagcggagcttgatgctctgaccaataacggccaaattcctgactatgacgaagaagatgactccttgccatacctcaccgcatgtatcaaggagcttttccggtggaatcaaatcgcacccctcgctataccgcacaaattaatgaaggacgacgtgtaccgcgggtatctgattcccaagaacactctagtcttcgcaaacacctggtgaggctgtccattcattcctagtacatccgttgccccactaatagcatcttgataacagggcagtattaaacgatccagaagtctatccagatccctctgtgttccgcccagaaagatatcttggtcctgacgggaagcctgataacactgtacgcgacccacgtaaagcggcatttggctatggacgacgaaattggtaagtgcgctttcagaacccccccttccgttgactagtgccatgcgcgcatacaatatcgctattgatctgatataacttccctgcggcatttattttggcattcctttagtcccggaattcatctagcgcagtcgacggtttggattgcaggggcaaccctcttatcagcgttcaatatcgagcgacctgtcgatcagaatgggaagcccattgacataccggctgattttactacaggattcttcaggtagctaatttccgtctttgtgtgcataatacccctaacgacgcacgtttacctttttgtaaagacacccagtgcctttcca

SEQ ID NO: 13 - protein, 362 aa, synthetic construct
MAFDLKTEDGLITYLTKHLSLDVDTSGVKRLSGGFVNVTWRIKLNAPYQGHTSIILKHAQPHMSTDEDFKIGVERSVYEYQAIKLMMANREVLGGVDGIVSVPEGLNYDLENNALIMQDVGKMKTLLDYVTAKPPLATDIARLVGTEIGGFVARLHNIGRERRDDPEFKFFSGNIVGRTTSDQLYQTIIPNAAKYGVDDPLLPTVVKDLVDDVMHSEETLVMADLWSGNILLQLEEGNPSKLQKIYILDWELCKYGPASLDLGYFLGDCYLISRFQDEQVGTTMRQAYLQSYARTSKHSINYAKVTAGIAAHIVMWTDFMQWGSEEERINFVKKGVAAFHDARGNNDNGEITSTLLKESSTA

SEQ ID NO: 14 - RNA, 1050 nt, synthetic construct
  source 1..1050: mol_type = other RNA
atggcgttcgatctcaagactgaagacggcctcatcacatatctcactaaacatctttctttggacgtcgacacgagcggagtgaagcgccttagcggaggctttgtcaatgtaacctggcgcattaagctcaatgctccttatcaaggtcatacgagcatcatcctgaagcatgctcagccgcacatgtctacggatgaggattttaagataggtgtagaacgttcggtttacgaataccaggctatcaagctcatgatggccaatcgggaggttctgggaggcgtggatggcatagtttctgtgccagaaggcctgaactacgacttagagaataatgcattgatcatgcaagatgtcgggaagatgaagacccttttagattatgtcaccgccaaaccgccacttgcgacggatatagcccgccttgttgggacagaaattggggggttcgttgccagactccataacataggccgcgagaggcgagacgatcctgagttcaaattcttctctggaaatattgtcggaaggacgacttcagaccagctgtatcaaaccatcatacccaacgcagcgaaatatggcgtcgatgaccccttgctgcctactgtggttaaggaccttgtggacgatgtcatgcacagcgaagagacccttgtcatggcggacctgtggagtggaaatattcttctccagttggaggagggaaacccatcgaagctgcagaagatatatatcctggattgggaactttgcaagtacggcccagcgtcgttggacctgggctatttcttgggtgactgctatttgatatcccgctttcaagacgagcaggtcggtacgacgatgcggcaagcctacttgcaaagctatgcgcgtacgagcaagcattcgatcaactacgccaaagtcactgcaggtattgctgctcatattgtgatgtggaccgactttatgcagtgggggagcgaggaagaaaggataaattttgtgaaaaagggggtagctgcctttcacgacgccaggggcaacaacgacaatggggaa

SEQ ID NO: 15 - protein, 309 aa, synthetic construct
MHIRNPYRTPIDYQALSEAFPPLKPFVSVNADGTSSVDLTIPEAQRAFTAALLHRDFGLTMTIPEDRLCPTVPNRLNYVLWIEDIFNYTNKTLGLSDDRPIKGVDIGTGASAIYPMLACARFKAWSMVGTEVERKCIDTARLNVVANNLQDRLSILETSIDGPILVPIFEATEEYEYEFTMCNPPFYDGAADMQTSDAAKGFGFGVGAPHSGTVIEMSTEGGESAFVAQMVRESLKLRTRCRWYTSNLGKLKSLKEIVGLLKELEISNYAINEYVQGSTRRYAVAWSFTDIQLPEELSRPSNPELSSLF

SEQ ID NO: 16 - RNA, 930 nt, synthetic construct
  source 1..930: mol_type = other RNA
atgcatatcagaaatccttaccgtacaccaattgactatcaagcactttcagaggccttccctcccctcaagccatttgtgtctgtcaatgcagatggtaccagttctgttgacctcactatcccagaagcccagagggcgttcacggccgctcttcttcatcgtgacttcgggctcaccatgaccataccagaagaccgtctgtgcccaacagtccccaataggttgaactacgttctgtggattgaagatattttcaactacacgaacaaaaccctcggcctgtcggatgaccgtcctattaaaggcgttgatattggtacaggagcctccgcaatttatcctatgcttgcctgtgctcggttcaaggcatggtctatggttggaacagaggtcgagaggaagtgcattgacacggcccgcctcaatgtcgtcgcgaacaatctccaagaccgtctctcgatattagagacatccattgatggtcctattctcgtccccattttcgaggcgactgaagaatacgaatacgagtttactatgtgtaaccctccattctacgacggtgctgccgatatgcagacttcggatgctgccaaaggatttggatttggcgtgggcgctccccattctggaacagtcatcgaaatgtcgactgagggaggtgaatcggctttcgtcgctcagatggtccgtgagagcttgaagcttcgaacacgatgcagatggtacacgagtaacttgggaaagctgaaatccttgaaagaaatagtggggctgctgaaagaacttgagataagcaactatgccattaacgaatacgttcaggggtccacacgtcgttatgccgttgcgtggtctttcactgatattcaactgcctgaggagctttctcgtccctctaaccccgagctcagctctcttttctag

SEQ ID NO: 17 - protein, 728 aa, synthetic construct
MASSSSDVFVLGLGVVLAALYIFRDQLFAASKPKVAPVSTTKPANGSANPRDFIAKMKQGKKRIVIFYGSQTGTAEEYAIRLAKEAKQKFGLASLVCDPEEYDFEKLDQLPEDSIAFFVVATYGEGEPTDNAVQLLQNLQDESFEFSSGERKLSGLKYVVFGLGNKTYEHYNLIGRTVDAQLAKMGAIRIGERGEGDDDKSMEEDYLEWKDGMWEAFATAMGVEEGQGGDSADFVVSELESHPPEKVYQGEFSARALTKTKGIHDAKNPFAAPIAVARELFQSVVDRNCVHVEFNIEGSGITYQHGDHVGLWPLNPDVEVERLLCVLGLAEKRDAVISIESLDPALAKVPFPVPTTYGAVLRHYIDISAVAGRQILGTLSKFAPTPEAEAFLRNLNTNKEEYHNVVANGCLKLGEILQIATGNDITVPPTTANTTKWPIPFDIIVSAIPRLQPRYYSISSSPKIHPNTIHATVVVLKYENVPTEPIPRKWVYGVGSNFLLNLKYAVNKEPVPYITQNGEQRVGVPEYLIAGPRGSYKTESFYKAPIHVRRSTFRLPTNPKSPVIMIGPGTGVAPFRGFVQERVALARRSIEKNGPDSLADWGRISLFYGCRRSDEDFLYKDEWPQYEAELKGKFKLHCAFSRQNYKPDGSKIYVQDLIWEDREHIADAILNGKGYVYICGEAKSMSKQVEEVLAKILGEAKGGSGPVEGVAEVKLLKERSRLMLDVWS

SEQ ID NO: 18 - DNA, 2187 bp, synthetic construct
  source 1..2187: mol_type = other DNA
atggcttcttcttcttctgacgttttcgttttgggtttgggtgttgttttggctgctttgtacatcttcagagaccaattgttcgctgcttctaagccaaaggttgctccagtttctactactaagccagctaacggttctgctaacccaagagacttcatcgctaagatgaagcaaggtaagaagagaatcgttatcttctacggttctcaaactggtactgctgaagaatacgctatcagattggctaaggaagctaagcaaaagttcggtttggcttctttggtttgtgacccagaagaatacgacttcgaaaagttggaccaattgccagaagactctatcgctttcttcgttgttgctacttacggtgaaggtgaaccaactgacaacgctgttcaattgttgcaaaacttgcaagacgaatctttcgaattctcttctggtgaaagaaagttgtctggtttgaagtacgttgttttcggtttgggtaacaagacttacgaacactacaacttgatcggtagaactgttgacgctcaattggctaagatgggtgctatcagaatcggtgaaagaggtgaaggtgacgacgacaagtctatggaagaagactacttggaatggaaggacggtatgtgggaagctttcgctactgctatgggtgttgaagaaggtcaaggtggtgactctgctgacttcgttgtttctgaattggaatctcacccaccagaaaaggtttaccaaggtgaattctctgctagagctttgactaagactaagggtatccacgacgctaagaacccattcgctgctccaatcgctgttgctagagaattgttccaatctgttgttgacagaaactgtgttcacgttgaattcaacatcgaaggttctggtatcacttaccaacacggtgaccacgttggtttgtggccattgaacccagacgttgaagttgaaagattgttgtgtgttttgggtttggctgaaaagagagacgctgttatctctatcgaatctttggacccagctttggctaaggttccattcccagttccaactacttacggtgctgttttgagacactacatcgacatctctgctgttgctggtagacaaatcttgggtactttgtctaagttcgctccaactccagaagctgaagctttcttgagaaacttgaacactaacaaggaagaataccacaacgttgttgctaacggttgtttgaagttgggtgaaatcttgcaaatcgctactggtaacgacatcactgttccaccaactactgctaacactactaagtggccaatcccattcgacatcatcgtttctgctatcccaagattgcaaccaagatactactctatctcttcttctccaaagatccacccaaacactatccacgctactgttgttgttttgaagtacgaaaacgttccaactgaaccaatcccaagaaagtgggtttacggtgttggttctaacttcttgttgaacttgaagtacgctgttaacaaggaaccagttccatacatcactcaaaacggtgaacaaagagttggtgttccagaatacttgatcgctggtccaagaggttcttacaagactgaatctttctacaaggctccaatccacgttagaagatctactttcagattgccaactaacccaaagtctccagttatcatgatcggtccaggtactggtgttgctccattcagaggtttcgttcaagaaagagttgctttggctagaagatctatcgaaaagaacggtccagactctttggctgactggggtagaatctctttgttctacggttgtagaagatctgacgaagacttcttgtacaaggacgaatggccacaatacgaagctgaattgaagggtaagttcaagttgcactgtgctttctctagacaaaactacaagccagacggttctaagatctacgttcaagacttgatctgggaagacagagaacacatcgctgacgctatcttgaacggtaagggttacgtttacatctgtggtgaagctaagtctatgtctaagcaagttgaagaagttttggctaagatcttgggtgaagctaagggtggttctggtccagttgaaggtgttgctgaagttaagttgttgaaggaaagatctagattgatgttggacgtttggtcttaa

SEQ ID NO: 19 - protein, 449 aa, synthetic construct
MSAPAQNYKIADISLAAFGRKEIELAEHEMPGLMAIRKAYGDVQPLKGARIAGCLHMTIQTAVLIETLVALGAEVTWSSCNIYSTQDHAAAAIAASGVPVFAWKGETEEEYLWCIEQQLFAFKDNKKLNLILDDGGDLTTLVHEKHPEMLEDCFGLSEETTTGVHHLYRMVKEGKLKVPAINVNDSVTKSKFDNLYGCRESLVDGIKRATDVMLAGKVAVVAGYGDVGKGCAAALRGMGARVLVTEIDPINALQAAMEGYQVVTMEDASHIGQVFVTTTGCRDIINGEHFINMPEDAIVCNIGHFDIEIDVAWLKANAKECINIKPQVDRYLLSSGRHVILLANGRLVNLGCATGHSSFVMSCSFSNQVLAQIALFKSNDKSFREKHIEFQKTGPFEVGVHVLPKILDEAVAKFHLGNLGVRLTKLSKVQSEYLGIPEEGPFKADHYRY

SEQ ID NO: 20 - RNA, 1350 nt, synthetic construct
  source 1..1350: mol_type = other RNA
atgtctgctccagctcaaaactacaaaatcgctgatatctctttggctgccttcggtagaaaggaaatcgaattggctgaacatgaaatgccaggtttgatggccatcagaaaggcttacggtgacgtccaacctttgaaaggcgcccgtattgctggttgtttgcacatgaccattcaaactgctgttttaattgaaactttagttgctttgggtgccgaagttacctggtcctcttgtaacatctattcgactcaagatcatgccgccgctgctattgccgcttccggtgttccagtttttgcctggaagggtgaaactgaagaagagtatttgtggtgtattgaacaacaattgtttgccttcaaggacaacaagaaattgaacttgatcttagatgatggtggtgatttaaccactttagttcatgaaaagcaccctgaaatgctggaagactgctttggtctttccgaagaaactaccaccggtgttcaccacttatacagaatggtcaaagaaggcaagttaaaggttcctgccattaacgttaacgactccgtcactaagtccaagtttgacaacttgtacggctgtagagaatccttagtcgacggtattaagagagccactgatgtcatgttggctggtaaggttgccgttgttgctggttacggtgatgtcggtaagggttgtgctgctgccttaagaggaatgggtgctcgtgtcttggttaccgaaattgacccaatcaacgctttacaagctgccatggaaggctaccaagttgttaccatggaagatgcatcccacattggtcaagttttcgttaccaccactggttgtagagatattatcaacggtgaacatttcatcaacatgccagaagatgccattgtttgtaacattggccatttcgatatcgaaattgatgtcgcctggttaaaggctaacgctaaagaatgtattaacatcaaaccacaagtcgaccgttacttgttgtcttctggtagacacgtcatcttgttggctaacggtagattagttaacttgggttgtgctactggtcactcatctttcgttatgtcttgttccttctctaaccaagtcttagctcaaattgctttgttcaagtctaacgataagtctttcagagaaaagcacattgaattccaaaagacaggcccattcgaagttggtgtccacgttttgccaaagatcttggatgaagctgtcgctaagttccacttgggcaacttgggtgttagattgactaaattgagtaaagtccaatctgaatacttgggtattccagaagaaggtccattcaaggccgaccactacagatattga

SEQ ID NO: 21 - protein, 340 aa, synthetic construct
MTAPLVVLGNPLLDFQADVTAEYLAKYSLKENDAILVDAKSGDAKMAIFDELLQMPETKLVAGGAAQNTARGAAYVLGAGQVVYFGSVGKDKFSERLLNENEKAGVKSMYQVQNDIGTGKCAALITGHNRSLVTDLGAANFFTPDHLDKHWDLVEAAKLFYIGGFHLTVSPDAIVKLGQHAKENSKPFVLNFSAPFIPHVFKDALARVLPYATVIIANESEAEAFCDAFQLDCANTDLEAIAQRIVKDSPVEKTVIFTHGVEPTVVVSSKGTSTYPVKPLDSSKIVDTNGAGDAFAGGFMAGLTKGEDLETSIDMGQWLAALSIQEVGPSYPSEKISYSK

SEQ ID NO: 22 - RNA, 1023 nt, synthetic construct
  source 1..1023: mol_type = other RNA
atgaccgcaccattggtagtattgggtaacccacttttagatttccaagccgacgtcacggctgaatacctggccaagtattctctaaaggaaaacgacgcaattttggtcgatgccaaatcaggcgatgctaagatggctatttttgacgagctcttacagatgccagaaacaaagcttgttgcaggtggtgctgctcaaaacactgctagaggggcagcatacgttttgggcgccggccaggtggtgtacttcggttccgtcggtaaggacaagttcagcgagagattgcttaacgaaaacgaaaaagctggtgtcaagtctatgtaccaagttcaaaatgatattggtaccggtaagtgtgccgcattaatcactggccataaccggtccttggtcactgacttgggtgctgccaatttctttactccagaccacttggacaagcattgggacttggtcgaagcagctaagctcttctacatcggtggtttccacttgaccgtgtctccagacgctatcgttaagttgggccaacatgctaaagagaacagcaaacctttcgtgttgaactttagtgctcctttcattcctcatgtcttcaaagacgcattggccagagttttgccttatgctaccgtcatcatcgctaacgaatcggaggccgaagccttttgcgacgccttccaattagactgtgccaacactgatttggaagctattgctcaaagaattgtcaaggactctccagttgaaaagactgtcatcttcacccacggtgtcgaaccaacagtggtcgtgtcctccaagggtaccagcacatatccagtcaaacctttggactcttctaagatcgtcgacaccaacggtgctggtgacgccttcgctggtggctttatggctgggttgactaaaggtgaagatttggaaacctctattgacatgggtcaatggctagctgctttgtctattcaagaagttggtccctcttacccttccgaaaaaatatcttactctaaatag

SEQ ID NO: 23 - DNA, 1365 bp, synthetic construct
  source 1..1365: mol_type = other DNA
atgaactcacccaccgcatccgaccccatagtgcgtacattagacgcgccggaaaccaaggacatgcaggaagcattccttcctggagagtcaccgcaggacgtcgcgtcagtcatccaggaaacgatggacatcttcgacaagaggatgcgtctaacgcaccctcactgcttctgctacgtccctagttgcccttccccgttagcgtgggtaggagatctgttcacgtcactttacaacgtgaacgcagtaagctgggacgtgtccagtgggccgtccgccatggagcagggaatgattaggtggcttgcacagcaggtcggactaccaccctcagcgggcggatgcttcgtgagtggcggcagcatggcaaacctgtccgcggttgtcgcagcacgtgacaagctgttacccccgatgagaagggccgagggaaccatatacacgtccgaccagacacacataagtataaagaagtgcctacacatcatcggattcgacgagcaccagatcagggtcatacctacagaccaccttttccgtatggacacggacatactgagaaggacaatcgcagcagacaggcagtgcggaaggttacccttcttagtagtggcgagttgcgggacgacgaacaccggatcaatagaccctttacacgcccttgcggacgtggcagaggaagagggattatggttacacgtagacggggcatacggcgccagcatcgcactgtcggacaagtacaggcacttagtagacggcatcggaagggcggactcaatatcatgggacggacacaagtggctattccagacgtacggatgcggcatagtgctaacacgtgactcagcaagcctggcacactcattcgcggtggacgcagagtacatcaactcgacgacacctcacggggccgtcaacttctacaacttatcccctgagctatccaggcccgcgcgtgcaatgccgctgtggttcaccttacgtgtattagggagaaggcagatgggcgagatgatagaccagggattcttactggcccgtacagccgaagaggagatcaggcgttacccccactgggtaatattagcccctgtcacagcctcgatagtcgtctttcgttactgccctctaggacttaaggacgacgacttagacacacttaacttagccttatccgcaagacttcttgcggagaacgtagcatccatactaaccaccaacataaggggacgtgtcgcattaaggttctgcgccacaaaccctgcaacacaggccgacaccataacaaaggtagtccagcagatggaccagatggcaaacacaatgcctacatgcggattaggcatgtgcaaggagtga

SEQ ID NO: 24 - DNA, 1320 bp, synthetic construct
  source 1..1320: mol_type = other DNA
atgcaagtaatacccgcttgtaattcagcagctattagatctttatgtccaacgcctgagtcttttaggaatatgggatggttgtcagtatctgacgcagtatactccgaatttataggtgaattggcgactagggcttcaaacagaaactattccaacgaatttggtttaatgcaacctattcaggaattcaaagcttttatcgagtctgatccagttgttcaccaggagtttattgacatgttcgaaggcattcaggactcaccaagaaattaccaggagctttgcaacatgtttaacgacatatttcgtaaggcacctgtttatggtgacttaggtccccctgtatacatgattatggccaaattaatgaatacgcgtgccggattttctgcctttacccgtcagcgtttgaatttacactttaagaaattgtttgacacctggggattattcttgagttcgaaggattcaagaaatgttctagtcgctgatcagttcgatgatagacactgtggctggttgaatgagagagctttatccgcaatggtcaaacactataatggcagagcatttgacgaagtattcttatgtgataagaacgcgccctattacggtttcaattcgtatgacgatttctttaatagaaggtttagaaatagggacattgaccgtcctgtagtcggcggggtgaataacaccaccttgatcagtgcggcttgcgaatcattgtcatacaacgtttcctacgacgtccaatctctagacactttagtatttaagggtgagacttattcattgaagcatttgttgaacaacgatccgttcacccctcaatttgaacatggatcaattttgcaaggattcttgaatgtgacagcttaccacaggtggcacgcacccgtcaatggaactatcgttaagataataaatgttccaggtacttattttgcacaggctccttctacaataggggacccgatcccagacaacgactatgatcccccaccttatttgaaatctttagtttacttctccaatattgcggctagacaaattatgttcattgaggcagataacaaggagatcggcttaattttcctggtgttcattggaatgacagagatttccacgtgtgaggctaccgtttcagaagggcaacatgttaacagaggtgatgacttaggaatgtttcattttggtggttctagttttgcgttgggtttgcgtaaggattgcagagcagaaatagtggaaaagtttacagaaccaggtacggtgataagaataaatgaggttgtcgccgcattaaaggcgtga

SEQ ID NO: 25 - DNA, 1527 bp, synthetic construct
  source 1..1527: mol_type = other DNA
atgattgctgttttgttttcattcgtaattgcgggctgcatctactatatcgtttcgaggagagtccgtagatcaaggttgccgcccggcccacctggtatccctatccccttcattggcaacatgtttgacatgccagaagaatctccatggcttacgtttcttcagtggggtagagactacaatacagacattttgtatgtagacgcaggtggtacagagatggttattttaaatacacttgaaacaattaccgacttgttggagaagcgtggttctatatattcgggcagattagaaagtactatggtcaatgagttgatgggttgggaatttgatctaggatttattacttacggtgacaggtggcgtgaggagcgtaggatgtttgcaaaagaatttagcgagaaaggcatcaaacaatttaggcacgcgcaagtgaaggccgctcatcagcttgtgcaacagctaaccaaaacaccggatagatgggcccaacacataagacaccaaatcgctgctatgtctttagatattgggtacggcattgatctagcagaagatgatccctggctagaggctacacaccttgctaatgaaggattagcaatagcgtctgttcctggtaaattctgggttgattcattcccctctttaaagtacttgcccgcttggttcccaggggccgtctttaagagaaaagctaaggtgtggcgtgaggccgctgaccacatggtggacatgccatacgagactatgcgtaagttagcccctcaaggactgactaggccatcttacgccagtgctaggttacaggcgatggacttgaatggtgacctagagcaccaagagcacgtgatcaagaacactgctgctgaggtaaatgtgggtggtggtgacacgaccgtttccgctatgagcgcattcattctggcaatggtgaagtaccccgaagtccaaaggaaagtgcaggcggagcttgatgctttgactaataacgggcaaatcccggattacgacgaagaggacgactcacttccctatttaacggcatgtattaaagagttgtttagatggaaccagattgctcctcttgcaataccccataaattgatgaaagacgatgtatatagagggtatctaatcccaaagaacacattagtctttgcaaacacgtgggcagtcttaaacgacccagaggtttatccagacccttcggtatttagacccgagcgttatcttggaccagatggtaagccagacaataccgtccgtgaccctagaaaggctgctttcggttatgggagaagaaattgccctgggattcacttagctcaatcgacagtatggatcgctggggcaacattactgtctgcttttaatatcgagaggcctgtggaccaaaacggtaagcccattgacattcctgcggactttacaacaggtttcttcaggcaccctgtcccgttccagtgcaggtttgttcctagaacagagcaggtgtctcaaagcgtgtccggcccatag

SEQ ID NO: 26 - DNA, 1089 bp, synthetic construct
  source 1..1089: mol_type = other DNA
atggccttcgacctaaagacagaagatggattgatcacatacctgactaagcacctgtctctagacgtggataccagtggagtcaaaagattaagcggtggattcgttaatgttacatggagaattaaattaaacgccccttaccaaggacatacctcgattatattgaagcacgcccagccacatatgagtacagacgaggactttaagataggcgtcgagcgtagtgtgtacgaataccaagctataaagctgatgatggccaacagagaggtattaggcggtgtcgacgggatagttagtgttccagaaggtttgaactacgacttggagaataacgctttaattatgcaggatgttggaaagatgaaaactttgttggattatgtaacagctaaaccacctttagcaactgacatcgccaggttggttggtacggagatcggtggatttgttgctcgtctacataacatcggtcgtgaaagacgtgacgatccagaattcaaattcttttcaggaaacatagtgggacgtactacaagtgaccaactgtatcagaccattataccaaacgcagcaaagtacggtgtcgacgatcccttgttgcccaccgttgttaaagaccttgttgacgatgtcatgcattctgaggagacactggtcatggctgatctttggtcaggcaacattttgctacagctagaggaaggtaatccttcgaagttgcagaagatatacatattggattgggagttatgtaagtacggcccagcttctttagatctaggatattttctaggagactgttatcttatatcgagattccaagatgaacaggtcggcaccactatgagacaagcctacttgcagtcgtatgcaaggacatcaaagcattctattaattatgccaaagttactgctggtatagcggcccacatcgtaatgtggaccgatttcatgcaatggggctccgaagaggagaggataaatttcgtgaagaagggtgtagccgcattccacgacgcaaggggaaacaacgataatggcgaaatcacgagtacattattgaaggagtctagcactgcttag

SEQ ID NO: 27 - DNA, 930 bp, synthetic construct
  source 1..930: mol_type = other DNA
atgcatattagaaatccatacaggacccctatcgactatcaggccttgtcagaggcctttccaccactaaagccgttcgtgtcggtaaacgccgatggcacttcctcggtggatttgactatacctgaagctcaaagggcattcactgctgcgcttcttcacagggattttggtttaacgatgaccatacctgaggacaggctatgtcctactgtgccgaatagattaaattacgtgctatggattgaagatatattcaactacacaaataaaacgctaggactaagcgacgatagacccataaagggagttgacatcggaaccggggcatccgcaatttatcccatgttagcttgcgctaggtttaaagcatggagcatggtcggtacagaagtagaaaggaaatgtattgacacagcaagacttaatgttgtggccaataaccttcaggaccgtttatccattctagaaactagcatagacggtcctatattggttcccattttcgaagccactgaagagtacgagtatgagttcacgatgtgtaacccaccgttctatgatggcgctgccgacatgcagacatctgatgcagcaaagggatttggcttcggagtcggtgcaccccattcgggtactgtcatagagatgtctacagagggcggcgaatccgcatttgttgctcagatggttcgtgagtcattaaaattaaggacaagatgtaggtggtacacctctaacttgggcaagttgaagtctttgaaggagatagtgggtttgctaaaagagttagagatctctaattatgctatcaatgaatacgttcagggtagtactagaagatacgctgtggcctggtctttcactgacatccagttgcccgaggaattatcaaggccctcgaatccagagcttagttcgctattttga

SEQ ID NO: 28 - DNA, 2187 bp, synthetic construct
  source 1..2187: mol_type = other DNA
atggcatcatcatcatctgacgtgttcgtcttaggcttaggcgttgtattagctgcgctttacatatttagagatcagttgttcgcagcatcaaaaccaaaggttgcaccggtttcaactacaaaaccagcgaacgggagtgctaacccaagggattttattgcaaagatgaaacaaggaaagaagagaatcgttatcttttatggtagtcaaacaggaacggctgaggagtacgccattagattagcaaaggaagcaaaacagaagtttggtctggccagtctggtctgcgatcctgaggaatatgacttcgaaaagttggatcaactaccagaggacagtatagccttcttcgttgttgcgacttatggtgagggtgagcctacggacaatgctgtgcaattattgcaaaatctgcaagatgagagtttcgaattctcttcaggagagagaaagctttctggtcttaagtacgtagtgtttggccttggcaacaaaacgtatgagcattacaacttaattggtagaactgtagacgcacagttggctaaaatgggagccattcgtataggcgagagaggcgaaggcgatgacgacaaatctatggaagaagattacctagaatggaaggatggtatgtgggaagcattcgctacggctatgggtgtagaggaaggccagggcggtgattctgccgattttgtggtatctgaactggagtcgcacccacctgagaaggtatatcaaggggaattcagcgcacgtgctttgacaaagaccaaaggtattcatgacgctaagaatcctttcgctgcacctatagctgtggcaagagaacttttccaatccgtggtggacagaaattgtgttcacgtagagtttaacattgaaggctcaggtataacgtatcagcatggtgaccatgttggtctgtggccgttaaacccggacgtggaagtcgagagattgctttgcgttttgggtttggctgagaagagagatgccgtaataagtatcgagagcctggacccagcgcttgccaaggttccatttccagttccaacgacttatggtgcagttctaagacactatatagatatttcagcagtagcaggtagacaaattttgggcactttatcaaagtttgcgccaaccccagaggccgaagcattcctacgtaatttaaacacaaataaggaagagtatcacaatgtagtcgcgaacggctgcttaaagttgggcgagatcttgcagattgccacaggtaacgatattactgtaccaccaacgactgcgaatactaccaagtggcctattccgttcgacattatcgtgtcggctataccacgtttacaaccaagatactattcaatctcttcatcccctaagattcatcccaacactatacacgctactgtggttgtcctaaaatatgagaatgtaccaactgagcctatacccagaaagtgggtgtacggtgtcggctcaaatttcttgttaaatctgaaatatgcagtgaacaaagaaccggttccatacataacgcagaacggggaacaaagagttggagtcccagaatatcttatagcaggacctagaggtagctataaaaccgaatccttttacaaggctcctattcacgttagaaggagtacatttagattacctaccaatccgaagtcgccagttatcatgataggtccagggacaggtgtggctccgtttagaggtttcgtgcaggagagagtggcgttggcccgtagaagtatcgagaagaatggtccagattcattggcagattggggtagaatctccctgttctatggctgtagaaggtctgatgaagattttctatataaagatgaatggcctcaatacgaagccgaattaaaagggaagtttaaattgcactgcgctttctcgaggcagaactataaacctgatgggagcaagatatacgtgcaagatttaatctgggaagatagagagcacatcgccgacgcaattctaaacggtaagggatatgtatacatctgtggtgaagcgaaatcgatgtccaagcaggttgaggaagttttggccaaaatcctgggagaggcaaaaggcggttcaggtcctgttgaaggagttgctgaggttaaattgctaaaagagagatcgagattaatgttagatgtgtggtcttga

SEQ ID NO: 29 - protein, 500 aa, synthetic construct
MGSIDSTNVAMSNSPVGEFKPLEAEEFRKQAHRMVDFIADYYKNVETYPVLSEVEPGYLRKRIPETAPYLPEPLDDIMKDIQKDIIPGMTNWMSPNFYAFFPATVSSAAFLGEMLSTALNSVGFTWVSSPAATELEMIVMDWLAQILKLPKSFMFSGTGGGVIQNTTSESILCTIIAARERALEKLGPDSIGKLVCYGSDQTHTMFPKTCKLAGIYPNNIRLIPTTVETDFGISPQVLRKMVEDDVAAGYVPLFLCATLGTTSTTATDPVDSLSEIANEFGIWIHVDAAYAGSACICPEFRHYLDGIERVDSLSLSPHKWLLAYLDCTCLWVKQPHLLLRALTTNPEYLKNKQSDLDKVVDFKNWQIATGRKFRSLKLWLILRSYGVVNLQSHIRSDVAMAKMFEEWVRSDSRFEIVVPRNFSLVCFRLKPDVSSLHVEEVNKKLLDMLNSTGRVYMTHTIVGGIYMLRLAVGSSLTEEHHVRRVWDLIQKLTDDLLKEA

SEQ ID NO: 30 - RNA, 1503 nt, synthetic construct
  source 1..1503: mol_type = other RNA
atgggcagcattgattcaacaaatgtagccatgtccaattctccagttggagaatttaagccacttgaagctgaggaattccgaaaacaagcccatcgtatggtagatttcatagccgattattacaaaaatgtggaaacatatccggtccttagcgaagtcgaacctggatatctccgaaaacgtatccccgaaaccgctccttacctccccgaaccacttgacgacatcatgaaagatattcagaaggatattatcccaggaatgacaaattggatgagccctaatttttatgcattttttcctgccactgttagttcagctgcctttttaggagaaatgttgtctactgccctaaattcagtaggctttacttgggtttcttcaccagccgccaccgaattagaaatgattgttatggattggttggctcagatccttaaactccccaaatctttcatgttttcaggtaccggtggcggcgtcatccaaaacaccactagcgagtccattctttgtacaatcattgccgcccgggaaagggccctggagaagctcggtcccgatagtattggaaaacttgtctgttacggatccgatcaaacccataccatgttccccaaaacttgcaaattggcgggaatttatccgaataatattaggttaatacctacgaccgtcgaaacggatttcggcatctcacctcaagttctacgaaaaatggtcgaggatgacgtggcggccggatatgtaccgctgttcttatgcgctaccctgggtaccacctcgaccacggctaccgatcctgtggactcactttctgaaatcgctaacgagtttggtatttggatccacgtggatgctgcttatgcgggaagcgcctgtatatgtcccgagtttagacattacttggatggaatcgaacgagttgactcactgagtctgagtccacacaaatggctactcgcttacttagattgcacttgcttgtgggtcaagcaaccacatttgttactaagggcactcactacgaatcctgagtatttaaaaaataaacagagtgatttagacaaagttgtggacttcaaaaattggcaaatcgcaacgggacgaaaatttcggtcgctgaaactttggctcattttacgtagctatggagttgttaatttacagagtcatattcgttctgacgtcgcaatggcgaaaatgttcgaagaatgggttagatcagactccagattcgaaattgtggtaccgagaaacttttctcttgtttgttttagattaaaacctgacgtttcgagtttacatgtagaagaagtgaataagaaacttttggacatgcttaactcgacgggacgagtttatatgactcatactattgtgggaggcatatacatgctaagactggctgttggctcatcgctaactgaagaacatcatgtacgccgtgtttgggatttgattcaaaaattaaccgatgatttgctcaaagaagcttga

SEQ ID NO: 31 - DNA, 1476 bp, synthetic construct
  source 1..1476: mol_type = other DNA
atgccgtcacacgaagaaaagtccgacgatggtctgtccgagataatgttagagacagagtcactgttaacgaggttattcacaataaagtcccccgccccgtccgaccctatagtgcagaagcttaacgcccacgagatccagaacatgcagcaccagctaatgcctgacgaccaccctcgtgacctatcatccgccttacagcaggccatggcagtattcgacaacaggatgcgtcttacgcaccccaggtgcttctcatacatacccgtatgccctaaccctgtggcgttattaggcgaccttcttacaagtgtctttaacgtaaacgcggcatcctgggacgtaggctccgggccttccgcagtagagacagccatgatagcatggttaggcgcacagctgggcctacctgacagtgtggaaggctgcttcgtatcaggtgggagtatgggaaacatgacggcaatagtagcagccagggacgagatacttcaccctcttcagcgtgcatacgcgataatatacatcagtgaccagacacacatctccatattaaaggccctacacatcacaggattcacacaccaccaggtacgtaagatcccgacagacgacgacttcaggatcgacgtggacatactaaggcgtacagtagcaagtgaccgtcactccggacttgtgcctttcttattagtagccacatgcggaaccacaaacacagggagtatagaccccttacacgccctggccgacctagcccgtgacgaggggctatggctgcacgtggacggagcgtacggcgcaagtatagcactttcggaccagcacaggtcactagtggacggaataggcagggccgactcgataagttgggacgggcacaagtggcttttccagacatacgggtgcggaatcgtgctgacaaggagaagtggaagtctgagaaggtccttcgcagtcgacgcagagtacataagggactccaccgtacccgagggcacgacgaacttctacaaccttggacccgagttaagtaggccggcaagggcaatgagcctttggttaacactaagggtcttaggacgtaggcgtatgggagagatgatcgacaggggattcctactggcaaggaccgcagaccactcgatacgtcagtacagggactggataatagcagcacctaccgtcgcgtcaatagtcgtattcaggtacgcgcccccgggattagacgacggcgcactagacgcactaaacaaggcaatatcaaggcgtttacttgcagaggacgtcgccgccatcttaaccaccaccgtcaggggcaggacggtattaaggatgtgcgccatgaacccgctagtagagcctgagacaatcgaggacgtaatcagaagggtagacggcatagcccgtagcgaggcaagccgtaggaaggagagtcacttccagttaggatcagcaatgtacttagggtag

SEQ ID NO: 32 - DNA, 32 bp, synthetic construct
  source 1..32: mol_type = other DNA
ggtctctaggatgtctgctccagctcaaaact

SEQ ID NO: 33 - DNA, 33 bp, synthetic construct
  source 1..33: mol_type = other DNA
ggtctcaaacttcaatatctgtagtggtcggcc

SEQ ID NO: 34 - DNA, 29 bp, synthetic construct
  source 1..29: mol_type = other DNA
ggtctctgcaatgaccgcaccattggtag

SEQ ID NO: 35 - DNA, 41 bp, synthetic construct
  source 1..41: mol_type = other DNA
ggtctcatgtgctatttagagtaagatattttttcggaagg

SEQ ID NO: 36 - protein, 523 aa, synthetic construct
MELTMASTMSLALLVLSAAYVLVALRRSRSSSSKPRRLPPSPPGWPVIGHLHLMSGMPHHALAELARTMRAPLFRMRLGSVPAVVISKPDLARAALTTNDAALASRPHLLSGQFLSFGCSDVTFAPAGPYHRMARRVVVSELLSARRVATYGAVRVKELRRLLAHLTKNTSPAKPVDLSECFLNLANDVLCRVAFGRRFPHGEGDKLGAVLAEAQDLFAGFTIGDFFPELEPVASTVTGLRRRLKKCLADLREACDVIVDEHISGNRQRIPGDRDEDFVDVLLRVQKSPDLEVPLTDDNLKALVLDMFVAGTDTTFATLEWVMTELVRHPRILKKAQEEVRRVVGDSGRVEESHLGELHYMRAIIKETFRLHPAVPLLVPRESVAPCTLGGYDIPARTRVFINTFAMGRDPEIWDNPLEYSPERFESAGGGGEIDLKDPDYKLLPFGGGRRGCPGYTFALATVQVSLASLLYHFEWALPAGVRAEDVNLDETFGLATRKKEPLFVAVRKSDAYEFKGEELSEV

SEQ ID NO: 37 - DNA, 1572 bp, synthetic construct
  source 1..1572: mol_type = other DNA
atggagctcaccatggcgtcgacgatgtcgctcgcgctgctcgtgctctccgcggcgtacgtgttggtcgcgttgaggaggagccggtcgtcgtcgtcaaagccacggcggctgccgccgtcgccgccggggtggccggtgatcgggcacctccacctcatgtccggcatgccgcaccacgcgctggccgagctggcgcgcaccatgcgcgcgccgctgttccggatgcggctggggagcgtgccggcggtggtgatctccaagccggacctcgcccgcgccgcgctcaccaccaacgacgccgcgctggcgtcgcggccgcacctgctctccggccagttcctgtcgttcggctgctccgacgtgacgttcgcgccggcggggccgtaccaccggatggcgcgccgcgtggtggtgtcggagctcctgtcggcgcgtcgcgtcgccacgtacggcgccgtcagggtcaaggagctccgccgcctgctcgcgcacctcaccaagaacacctcgccggcgaagcccgtcgacctcagcgagtgcttcctcaacctcgccaacgacgtgctctgccgcgtcgcgttcggccgccggttcccgcacggcgagggcgacaagctcggcgcggtgctcgccgaggcgcaggacctcttcgccgggttcaccatcggcgacttcttccccgagctcgagcccgtcgccagcaccgtcaccggactccgccgccgcctcaagaagtgcctcgccgacctccgcgaggcctgcgacgtgatcgtggacgaacacatcagcggcaaccgccagcgcatccccggcgaccgcgacgaggacttcgtcgacgtcctcctccgcgtccagaaatcccccgacctcgaggtccccctaaccgacgacaatctcaaggccctcgtcctggacatgttcgtcgccggcacggacaccacgttcgcgacgctggagtgggtgatgacggagctagtccgccacccacggatcctcaagaaggcgcaggaggaggtccggcgagtcgtcggcgacagcggccgcgtcgaggagtcccacctcggcgagctccactacatgcgcgccatcatcaaggagacgttccggctgcacccggcggtgccgttgctagtgccgcgcgagtccgtcgcgccgtgcacgctgggcggctacgacatcccggcgaggacgcgggtgttcatcaacacgttcgccatggggcgcgacccggagatctgggacaacccgctggagtactcgccggagaggttcgagagcgccggcggcggcggcgagatcgacctcaaggacccggactacaagctgctgccgttcggcggcgggcggcgagggtgccccggctacacgttcgcgctcgccaccgtgcaggtgtcgctcgccagcttgctctaccacttcgagtgggcgctgcccgccggcgtgcgcgccgaggacgtcaacctcgacgagacgttcggcctcgccacgaggaagaaggagccgctcttcgtcgccgtcaggaagagcgacgcgtacgagtttaagggagaggagcttagtgaggtttaa

SEQ ID NO: 38 - DNA, 1572 bp, synthetic construct
  source 1..1572: mol_type = other DNA
atggaattgacaatggcaagcactatgtcactggcattattagtactatcggctgcttacgtgctggttgcccttcgtaggtccaggtcatcttcatctaagcccagaaggctgcccccttccccacccggatggcctgttattggtcaccttcacttgatgtcaggtatgcctcatcatgctttagcggagttagctaggactatgagggcccctcttttcagaatgagactaggtagcgtacctgctgtggttatatcaaaacctgacttagctcgtgccgctttaacaacaaatgacgctgccttagcttctaggccacatcttctttctggacagttcttgtccttcgggtgctcagacgtcactttcgccccagctgggccatatcacaggatggcgagaagggtggtggtcagtgaattgctttctgccaggagagtagcgacttacggcgctgtgagggtaaaagagcttagaaggttgttggctcacttaactaagaatacgtcccctgctaaaccagtcgacttatctgaatgctttctaaacttagctaacgatgtactatgtagggtcgcctttggcagacgtttcccccacggagagggtgacaaattgggtgcggtattggcagaggcccaggacttgttcgcaggtttcactataggagacttcttcccggagttagagcccgtcgcaagtacggtaactgggttaagaagaagattaaagaaatgtctagctgacctaagagaggcttgtgacgtaatagtcgacgagcatatatctgggaacagacagaggataccaggtgacagagatgaggacttcgttgacgtcctattaagagttcaaaagagtcccgacttggaagttcccttaactgatgacaacttaaaggccttagtacttgacatgtttgtcgcaggtacagacactacgtttgctacattggagtgggtcatgactgaattagtgaggcatccgcgtatattgaagaaggcccaggaagaagtcagaagagtggtaggtgatagtggaagggtggaagagagccatctgggcgaattgcattatatgagggcgattataaaggaaacgttcaggctacaccctgccgtgcctttattggtgcccagggagagtgtagcaccatgcacgttgggcggatatgacatacctgctagaactagagtgtttataaacacctttgcgatgggacgtgatcccgagatatgggacaatcccttagaatacagccccgagagattcgagtctgcgggtggtggcggcgagatagacctaaaagaccctgactacaagctgttacctttcggcggcggtaggagaggatgccctggttacacatttgcattagctaccgtgcaggtatcactggcgagtttgttgtaccacttcgaatgggcgttaccagccggagtccgtgccgaggacgtcaaccttgacgagactttcggcttagctacaaggaagaaggagcccttattcgtcgctgtcagaaagagcgacgcttatgaatttaaaggggaagaattgtccgaggtataa

SEQ ID NO: 39 - protein, 695 aa, synthetic construct
MALALEAARSWAASVLPPELAAAAGGDPLAALAATAAALVAGVVILAVWFRSGGGAPPKAAAPPPRPPPVKIEADADADDGRKRVTVFFGTQTGTAEGFAKAMAEEARARYEKAVFKVVDLDDYAAEDEEYEEKLRKETIVLLFLATYGDGEPTDNAARFYKWFTEGKEKEVWLKDLKYAVFGLGNRQYEHFNKVAKVVDELLEEQGGKRLVPVGLGDDDQCIEDDFTAWKEQVWPELDQLLRDEDDTTGASTPYTAAIPEYRIVFIDKSDVSFQDKSWSLANGSGVIDIHHPVRSNVAVRKELHKPASDRSCIHLEFDISGTGLVYETGDHVGVYSENAIETVEQAEKLLDLSPDTFFSVHADAEDGSPRKGGGSLAPPFPSPCTLRTALLRYADLLNSPKKAALVALAAHASDLAEAERLRFLASPAGKDEYSQWVVASQRSLLEVMAAFPSAKPPLGVFFAAVAPRLQPRYYSISSSPKMAPSRIHVTCALVYGPTPTGRIHQGVCSTWMKNAIPSEYSEECSWAPIYVRQSNFKLPADPTTPIIMIGPGTGLAPFRGFLQERLALKQSGVELGNSVLFFGCRNRNMDYIYEDELQNFIQEGALSELIVAFSREGPAKEYVQHKMTEKATEIWNIVSQGGYIYVCGDAKGMARDVHRALHTIVQEQGSLDSSKTESYVKSLQMDGRYLRDVW

SEQ ID NO: 40 - RNA, 2088 nt, synthetic construct
  source 1..2088: mol_type = other RNA
atggcgctggcgctggaggcggcgaggtcgtgggcggcgtccgtgctgccgccggagctcgccgccgcggcggggggcgacccgctcgccgccctcgccgccacggccgcggcgctcgtcgccggggtggtcatcctcgccgtgtggttccgatccggtggaggggcgccgcccaaggccgccgcgccgccgccgcgcccgccgccggtcaagatcgaggccgacgccgacgccgacgacggccggaagcgcgtcaccgtcttcttcggcacgcagaccggcaccgccgagggcttcgctaaggcgatggcagaggaggcgagggcgcggtacgagaaggcggtgttcaaagttgtggatctggatgactatgcagctgaggatgaggagtacgaggagaagctcaggaaggaaactatcgtgttgctcttcctagcaacatatggggatggggagcctaccgacaatgctgcaagattctacaaatggttcactgaggggaaggaaaaggaagtctggcttaaggatcttaagtatgctgtttttggcctgggaaataggcagtatgagcactttaataaggttgcaaaggtggtggacgaactgctagaggagcagggtggcaagcgtcttgtcccagttggccttggagatgatgatcagtgcattgaggatgacttcacagcatggaaagaacaagtttggccagaattggaccaactgctccgtgatgaagatgatacaactggtgcatccactccttacactgctgctatacctgaatacagaattgtatttattgataaatcagatgtatcgttccaagacaaatcgtggtcacttgccaatggcagtggtgttattgatattcaccatcctgtcaggtctaatgttgctgttcgaaaggaactccataagccagcttcagatcgctcttgcattcatctggagttcgatatctcaggcactggtcttgtgtatgaaactggtgaccatgttggtgtttattcagaaaatgctatcgagacagtggagcaggctgaaaagctgctagatctttccccagatacatttttctccgtccatgcagacgcagaagatggatcacctcgtaaagggggtggttctttggccccacctttcccttctccttgcaccttacggactgcacttctaagatatgctgatctacttaattctccaaagaaggctgctttggttgctttagctgctcatgcgtctgacctagctgaagctgaaagattgagatttttggcttcccctgctggaaaggatgaatattctcaatgggtagtagctagccaaagaagccttcttgaagttatggctgcattcccttcagctaagcccccactgggagtattctttgcagctgttgcacctcgcctacaaccgcgttactactccatatcatcatctccaaagatggctccttccagaattcatgtaacatgtgcgttagtttatggacctacaccaacgggaaggattcaccaaggagtttgttcgacatggatgaagaacgcaatcccatcagaatatagtgaagagtgtagctgggcaccaatttatgtgaggcagtcaaattttaagttgcctgcagatccgaccactccaattatcatgattggtcctggtacagggttagcaccatttagaggcttcttgcaggaaagattagctctaaaacaatctggagttgaactgggcaattcagttctcttctttggatgcaggaaccgtaatatggactacatatatgaagacgagttgcaaaacttcattcaggagggtgcactttctgagctgattgttgccttctctcgtgaaggaccagccaaagaatacgtgcagcataagatgacagagaaggccacagaaatttggaacatcgtctcacaaggtggttacatatatgtgtgcggtgatgccaagggtatggcaagagacgtacacagagctcttcatacaattgttcaagagcagggatctttggatagctccaaaaccgagagctatgtaaagagcctacagatggacggcaggtacctccgtgacgtatggtga

SEQ ID NO: 41 - DNA, 2088 bp, synthetic construct
  source 1..2088: mol_type = other DNA
atggcgcttgcattagaagcagcaagaagttgggcagcctctgttttaccgccagagttggctgccgccgcaggcggcgatcctttggccgcattggcagccacagctgctgctttggtagcaggcgtggtcatcttagcagtttggtttagaagcggtggtggtgccccgcctaaggctgcagctcccccacccagacccccaccagtaaagatagaagcagatgcggacgcagacgacggacgtaaaagggtgacagtgttctttggcacccagacgggaacggcagagggtttcgcaaaagctatggctgaagaggccagggcgaggtatgagaaggctgtctttaaagtcgttgatcttgacgactatgcggccgaggacgaggagtacgaggaaaagttaagaaaggaaactatcgtattactgttcttggcgacatacggcgacggtgagcctactgacaatgcggccaggttttacaagtggtttacagagggaaaggagaaagaggtatggcttaaggacctgaagtacgcagtctttggattgggtaataggcagtacgaacacttcaacaaggttgcgaaagttgtggacgaactacttgaagaacagggtggcaagagactggttccagtaggtctgggtgacgacgaccagtgcattgaggatgactttaccgcctggaaggagcaagtttggcccgagcttgatcagctgttgcgtgacgaggacgacactactggcgcctcaactccatacactgccgcgataccggagtatcgtatagtattcattgacaaatccgatgtgtcttttcaagacaagtcgtggagcttagctaatggctccggcgtcatagacattcaccatccagttagatctaacgttgccgtacgtaaagagttacataagccggcatccgacaggtcttgcatccatttagagtttgatattagtggaaccgggcttgtttatgagacaggtgaccacgtcggtgtctactccgagaacgcaatagagactgtggaacaggccgagaagctgctagacttatcccctgacaccttcttttctgtccatgccgacgcagaggatggctcaccaaggaaaggtggcggctctctagctccaccctttccttccccatgcacgttgcgtactgctttattgaggtacgctgatttgttaaacagtcctaagaaagctgctttggttgctctagcggcccacgcttcagacctagctgaggcggagagattgcgtttcttagcttccccagcagggaaggacgaatattcacaatgggttgtggcttctcagcgttctctgcttgaagtgatggcagcttttccaagtgccaaaccacccttgggagtattcttcgcagccgtggcgccaaggttacagccaagatattattccatatcttcttcaccgaagatggcgccttccaggatacacgtcacctgcgcattagtatatggaccaacaccgacaggaagaatacaccagggtgtatgctctacctggatgaagaatgctataccttcagagtattctgaggagtgttcttgggcccctatatacgtacgtcagagtaacttcaagcttccggccgatcctaccacgccaattataatgattggaccaggaacaggactggcgccatttcgtggatttctgcaggagagactggctttgaagcaatctggagtagagctgggaaactcagttcttttcttcggttgccgtaataggaatatggactatatatacgaagatgagttacagaactttattcaggaaggagcgctttctgaattgatcgtcgccttctctagagagggtccggcaaaggagtatgtccagcataagatgacggagaaagccacggagatatggaacatcgtcagccagggcggttacatttatgtgtgtggcgacgccaagggtatggcccgtgacgtgcacagggcccttcataccatcgtccaagagcagggcagtttggactcatcaaagacggagtcttacgttaaaagccttcagatggacgggcgttacttgagggacgtttggtag

SEQ ID NO: 42 - protein, 263 aa, synthetic construct
MENSYSAQMYIDEFDPVHYYQTYYSSGKGGIAREWTDFALQNLHETFGPGGVKGDILIDFGAGPTIYQLLSACEVFNSIITSDFLEQNREQLEKWLRKDPDALDWSHFTKYVCELEGNRDNWEKKEETLRRKVTKVLKCDALAEKPFDDVPMPEADCLISCLCLENPCQDQEAYINILKKLKELLKPGGHIIIQSILNCSYYHIGNSCFSHLSLSKDDVEKSFKEAGYEIVKLKVLPRSVMSEMEISDSNGYYFIHARKPQKE

SEQ ID NO: 43 - RNA, 792 nt, synthetic construct
  source 1..792: mol_type = other RNA
atggagaactcatactctgcacagatgtacattgatgaatttgatcctgtacactactaccagacatactattcttcggggaaaggaggtatcgctagagaatggacagactttgccttacagaatctgcatgaaacatttggtccaggaggtgtgaaaggagacatactaattgactttggagctggacccacaatttatcaactgctctctgcttgtgaagtgttcaacagcattataacctctgattttcttgagcaaaaccgagagcaattggaaaaatggctgagaaaggacccagatgccttagactggtctcattttaccaaatatgtgtgtgaactggaaggtaacagagataactgggaaaaaaaggaagaaacgctgaggagaaaagtaaccaaagtgttgaagtgtgacgctcttgcggagaagccatttgacgacgtgcctatgccagaagccgactgtctgattagctgcctctgcttagaaaatccttgtcaagaccaagaagcttatatcaatatattgaaaaagcttaaggagctacttaaacccggtggtcacattataattcagagtattcttaattgttcatactaccatattggtaacagctgtttttcccatctgtccttatctaaggatgacgtggaaaaatcatttaaagaggccgggtatgaaattgttaaattaaaggtgctgccacgatctgtaatgtcagaaatggagatcagtgattctaatggctattactttatccatgctcgtaaaccacagaaagagtaa

SEQ ID NO: 44 - DNA, 792 bp, synthetic construct
  source 1..792: mol_type = other DNA
atggagaactcttactccgctcagatgtacatagatgaatttgatcctgttcactactaccagacttactattcctcgggcaaaggcggcatagctagagaatggaccgactttgcactacagaatctacatgaaacatttggaccgggtggggttaaaggcgacatattgattgactttggcgctggacctacaatctatcaactactgtcagcatgtgaagtgttcaacagcattataacatccgattttctggagcaaaacagagagcaactagaaaagtggttaagaaaggaccccgatgcccttgactggtcccattttactaaatatgtatgtgaacttgaaggtaacagagataactgggaaaagaaggaagaaaccctgcgtagaaaagtcacaaaggttctaaagtgtgacgcgttggccgagaaaccttttgacgacgtaccgatgccagaagccgactgtcttattagttgcttatgcttagaaaatccatgtcaagaccaagaggcttatatcaacatcttgaagaagttgaaagagttgctaaagccgggcggtcacatcataattcaaagcatcttaaattgtagctactaccatatcggaaattcgtgcttttctcatctatcgttgtctaaggatgacgtggaaaagtcgtttaaagaggccgggtatgaaattgtaaaactaaaggtcctaccaaggtctgtaatgtcagaaatggagatttccgattctaatggatattactttatccatgccagaaaaccacagaaagagtga

SEQ ID NO: 45 - DNA, 1320 bp, synthetic construct
  source 1..1320: mol_type = other DNA
atgcaggttataccggcatgtaactctgcggcgatacgtagcttatgcccaacaccggaatcatttcgtaacatgggatggcttagcgtgtctgacgcggtctatagtgaattcatcggtgagcttgcgactcgtgctagcaaccggaactattctaatgaattcggtttaatgcagccgatccaggaatttaaggcttttattgaaagtgatccggttgttcaccaggaattcattgacatgtttgaaggcatccaggacagtccgcgtaactatcaggaactgtgcaatatgttcaacgacatttttagaaaagccccagtctatggcgatctggggccccccgtatatatgattatggccaaattgatgaatactcgtgctggttttagtgcttttactcgtcagcgcctcaacctgcattttaaaaagctgttcgatacgtgggggctgtttttatctagtaaagatagccgcaatgttttagtagccgatcagttcgacgatagacactgtggttggcttaacgaacgtgcgttaagcgcaatggtgaaacactataacgggagagccttcgatgaggtctttctgtgtgataaaaatgcgccatattatggttttaattcttatgatgatttctttaaccgccgctttcgtaatcgcgatattgatcgcccggtggttggtggtgttaataatacgacgttaatatccgccgcgtgtgaatctttgtcttataatgtatcatatgatgtgcaatcgctcgatacccttgtctttaaaggagaaacatattctctgaaacatctgcttaacaatgatccttttaccccacaattcgaacatggatccatcttgcagggtttcttaaacgtcacggcgtaccatagatggcacgctccagttaatgggaccattgttaagatcattaacgtgccaggcacttatttcgcgcaggcaccgtccacgatcggtgacccgataccagacaatgattacgatccgcccccctatttgaagtcattagtgtacttctctaatatcgcggcacgtcagatcatgtttatagaagccgacaacaaagagatcgggttaatctttttggtattcattggtatgactgaaatttctacttgtgaggcaaccgtatcagaagggcaacatgtcaaccgtggcgacgaccttgggatgttccatttcggcggctcttcttttgctctcggcttgcgaaaagactgcagagccgaaatagtagaaaaattcactgagccaggtactgtcattcggattaatgaagtggtcgctgctcttaaggcatga

SEQ ID NO: 46 - DNA, 1476 bp, synthetic construct
  source 1..1476: mol_type = other DNA
atgccgtctcatgaggaaaaatcagatgatggcctgtcggaaatcatgctggaaaccgagtccttattaacgcggctcttcacaattaaaagtccagccccgagcgatccgattgtacaaaagctgaacgcacatgaaattcagaacatgcagcaccagctgatgccggatgatcatccacgggatttatctagtgctttgcagcaggcgatggcagtgttcgataaccgcatgagattaactcatccgcgttgttttagttatatacctgtttgccctaatccggtggccttacttggggatcttcttacatccgtcttcaacgtgaatgcagcgtcttgggacgtcggtagtgggcctagcgctgttgagaccgcgatgatagcttggctgggtgcacaactgggacttccagattctgttgaaggttgtttcgtgtcgggtggatcaatgggcaacatgaccgctattgttgcggcccgcgatgaaattctgcatccgttacagcgtgcttacgccattatttatatttcggatcagactcatattagtatcctgaaagcgctgcatattaccggtttcacccatcaccaagtacggaaaataccgactgatgacgattttcgtatcgatgttgatattctgcggcgtaccgtggcgagcgatcgccacagtggactggtgccttttctcctggttgccacatgtgggactacaaatacaggatcaatcgatcctcttcatgcattggctgacttggcacgagatgaaggtctgtggctccatgttgacggagcgtatggcgcgtcgatcgccttgtctgatcagcatcgctcattagtggatggaattggtcgcgctgatagtattagttgggatggacataaatggttattccaaacatatggatgcggaatcgttttaactcggcgtagcggctctttacgccgctccttcgccgttgatgcagagtatatccgggacagtacagttcccgaagggaccacgaacttttacaaccttggtccggagttaagccgcccagctcgtgcgatgagcctttggttgactctgcgtgtgcttggtcgtcgccgtatgggtgagatgattgatcgcggttttttgttggctcgaacggccgaccactcgatccgtcagtatcgtgattggataatcgccgctcctacagtcgcctccattgtcgtgttccggtatgctcctccagggctggacgacggagccctcgacgcactcaataaggcaattagtcgtcgtctgctggcagaagacgttgcagcgattcttactactaccgtacgtggccgtacggttctccgtatgtgtgcgatgaatccactggtggaaccggagactatcgaggatgtgattagaagagtggatggtattgcacgtagtgaggcgagccgccgtaaggagtctcatttccagttagggtctgctatgtatctgggctaa

SEQ ID NO: 47 - DNA, 1503 bp, synthetic construct
  source 1..1503: mol_type = other DNA
atgggaagcatagactcaacaaacgtagcaatgtcgaactcccccgtcggagagttcaagccgcttgaggcagaggagttcaggaagcaggcccacaggatggtagacttcatcgccgactactacaagaacgtggagacataccccgtacttagcgaggtggagcccggctacttaaggaagaggatacccgaaacggcaccttacctgcccgagcccctagacgacatcatgaaggacatacagaaggacataatccccggaatgaccaactggatgagtcccaacttctacgcattcttccctgcaacagtctcttccgcagcgttcttaggggagatgctaagtacagcgttaaactccgtcggattcacatgggtaagttcacccgccgcgaccgagttagagatgatcgtcatggactggctagcgcagatattaaagcttccgaagtccttcatgttctcagggacaggcggcggcgtaatacagaacacgacatcagagtcgatcctttgcacgatcatagcagcaagggagcgtgcactagagaagttaggacctgacagtatcggcaagttagtatgctacggctccgaccagacccacaccatgttccctaagacatgcaagttagccgggatataccctaacaacatacgtctaatacctacgacggtcgagacagacttcggcataagtccgcaggtgctgaggaagatggttgaggacgacgtcgccgcaggatacgtacctctattcttatgcgccaccttagggacgacatcaaccaccgcgacggaccccgtggactcactttcagagatcgcaaacgagttcggaatctggatacacgtagacgccgcgtacgcaggaagtgcctgcatatgcccggagttcaggcactacctggacggaatagagcgtgtggactcattatcgttatcacctcacaagtggttacttgcctacttagactgcacgtgcctatgggtaaagcagccgcacttactactaagggccctgacaaccaacccggagtacttaaagaacaagcagagtgacctagacaaggtagtagacttcaagaactggcagatagccacaggaaggaagttcaggagcttaaagctatggctgatcctgaggtcgtacggggtcgtgaacttacagtcacacatacgtagtgacgtggcaatggcaaagatgttcgaggagtgggtgcgttcggactcccgtttcgagatcgtggtccctaggaacttctcgcttgtatgcttccgtttaaagcccgatgtctcatcccttcacgtggaagaggtaaacaagaagttacttgacatgttaaactccacgggccgtgtctacatgacacacacgatagtcggtggcatctacatgcttcgtcttgccgtcgggagctcattaacggaagagcaccacgtacgtcgtgtatgggacttaatacagaagctaaccgacgacctgttgaaagaggcctga

SEQ ID NO: 48 - protein, 507 aa, synthetic construct
MGSKGPEGKPDDGLSEIMLEAESFLTKLLTINPPAPSDPIVRKLNTHEIQHMQHEVMPDDHPRDLSSVLQEAMAIFDNRMRLTHPHCFSYIPACPTSLAYLGDLLTSICNVNAASWDVSSGPSAVEKAMIAWLGAQLGLPDSVGGCFVSGGSMANMAAIIAARDEVLHPRQRGDAVIYISDQTHISILKALHITGFMDYQVHKIPTDDEFRIDVDILRRAIITDRQSGRVPFLLVANCGSTNTGSIDPLHALADLARDEGIWLHVDGAYGASIALSDRHHDLIDGIGRADSVSWDGHKWLFQTYGCGIVLTRDVHSLRRSFAVDAEYISSTLAPRDNTNFYNLGPELSRPARAMSLWLTLRTLGRQRMGEMIDQGFLLARTADGKIRQYKNWIIPAPTVASIVVFRYAPPGIDEEGLDTLNKAISQRLLSENIAAILTTKIRGRTTLRMCAMNPLVKPETIEDLIRQVDILAQSEASERHKSYPKMAKMLHAHCQERPGVDVFQHSH

SEQ ID NO: 49 - RNA, 1524 nt, synthetic construct
  source 1..1524: mol_type = other RNA
atgggttccaaggggccagaaggaaaaccagatgacggactgtccgagatcatgttagaggcggagtctttcttgacgaaactgctcacgattaatcctccggcaccgtccgaccctattgttcgaaagcttaatacccatgagatacaacacatgcaacatgaggttatgcccgatgaccatcctcgagatctttcatccgtccttcaagaagctatggcaatctttgataaccggatgcgcctcacccacccacattgcttttcctatataccagcttgcccaacttcccttgcctatctaggtgatctcctcacttctatttgcaacgtaaatgctgctagttgggatgtgagctctggtccaagtgcggttgaaaaagccatgatagcctggctgggtgcccagctgggactgccagactcagttggaggttgctttgtctcaggtggctctatggcaaacatggcggctataattgcagcccgagatgaggttcttcatcctaggcagcgaggagatgcggtcatctacatttctgaccagacacatatctctattcttaaggcgctacatataaccggcttcatggattatcaagtccacaagatacccaccgatgatgaatttcggatagatgtggacatcttacgacgcgctatcattacagaccgccaatccggacgtgtaccctttcttcttgtcgccaattgtgggagcaccaacacagggagtatcgaccctctccatgcactcgctgatcttgcacgagatgaaggcatatggctacatgtggatggtgcatatggtgcttccatcgcgctctctgacaggcatcacgatctcattgatgggatcggtcgcgcagatagtgtttcttgggatgggcataagtggctcttccagacatatggctgtggaatcgtgctcacgcgcgacgttcattctcttaggcgcagcttcgccgttgatgccgagtatatcagcagtacattggcaccacgggacaacaccaatttctacaaccttggtccagagctcagtcggccagcgcgtgccatgtctctttggctcaccctcagaaccctaggtcgtcagcgtatgggtgaaatgatcgaccaaggtttccttcttgcccgtactgcagatggtaagatccgtcagtacaagaactggattattcctgctccaacagttgctagcattgtggtttttcgatacgcaccgcctgggattgacgaagaagggctagacacattaaacaaggctatttcacagcgactcttgtcggagaatattgctgctatactcaccaccaaaattcgcggacggaccacacttcgaatgtgtgctatgaacccccttgtgaagccagaaactatcgaggatcttatacgccaggttgatatcttggcccaatctgaggcttctgagcgacacaagtcttatccaaagatggctaaaatgcttcatgcgcattgtcaagaacgccctggtgttgatgtttttcaacatagtcattga

SEQ ID NO: 50 - DNA, 1524 bp, synthetic construct
  source 1..1524: mol_type = other DNA
atgggctcaaaaggtcctgaaggtaagcctgacgatggactttcagagatcatgctagaggcagagagtttccttacgaagctgttaaccataaacccacccgcacccagcgaccccatcgtaaggaagttaaacacacacgagatacagcacatgcagcacgaggtaatgccggacgaccacccgcgtgacttatcgtccgtcttacaagaggcaatggccatattcgacaaccgtatgaggttaacgcaccctcactgcttctcatacatacctgcctgccctacaagtcttgcgtacctaggcgacttactgacctcaatatgcaacgtgaacgccgcgtcatgggatgtctcgtccggaccttcggcagtagagaaggcaatgatagcgtggctaggagcacagttaggactacccgactcagtcggtggatgctttgtctccggtggaagtatggcaaacatggccgccatcatagcggcaagggacgaggtcttacaccctaggcagaggggcgacgcagtaatatacatcagcgaccagacacacatcagcatcttaaaggcactgcacatcaccggattcatggactaccaggtacacaagatccccaccgacgacgagttccgtatagacgtagacatcttacgtcgtgcaatcataacagacaggcagagtggacgtgtaccgttcttattagtcgcgaactgcgggtcaacaaacaccggatcaatcgacccgcttcacgcacttgcagacctagcaagggacgaggggatctggttacacgtcgacggcgcctacggcgccagcatcgccttatcggacaggcaccacgacctgatagacggaataggccgtgccgactcagtcagttgggacggccacaagtggttattccagacatacggctgcggcatcgtcctaacgcgtgacgtgcacagcttaagaaggagtttcgcggtcgacgccgagtacatcagctccacactggcacctagggacaacaccaacttctacaacttagggcccgagctaagccgtcctgcacgtgccatgagcttatggttaaccctgaggaccttagggagacagcgtatgggtgagatgatagaccagggattcctgttagcaaggacagcagacgggaagatccgtcagtacaagaactggatcatccccgcccctacagtcgcaagtatcgtggtattccgttacgcgccgcctggaatcgacgaggaaggactagacacgcttaacaaggcaatctcgcagcgtctgttatcagagaacatcgcagccatactaacaacaaagatacgtggcaggacgacattaaggatgtgcgccatgaaccctcttgtgaagcctgagacaatcgaggacctaatcaggcaggtagacatcttagcacagagcgaggcaagtgagcgtcacaagagctaccctaagatggcaaagatgttacacgcccactgccaggagaggcctggagtcgacgtattccagcacagccactga

SEQ ID NO: 51 - DNA, 1458 bp, synthetic construct
  source 1..1458: mol_type = other DNA
atgactgagaaagaagaggacgtcgaccaattccacacgaaggtgtcgatcgccaccctagccgcccttacaaggctattaaagatgaacacacagacgccttcagaccccatcgtcaggacacttgacgccgcaggaaccaaggagatgcaagaggcattcctgcctggcgagagccctcacgacgtcgcaagcgtcatacaggagactatggacatattcgacaagaggatgaggcttacgcaccctcactgcttctgctacgtcccgtcctgcccttcgccgttagcctgggtgggagatcttttcacctcattatacaacgtcaacgcagtgtcctgggatgtctcatccggccctagcgccatggagcaggcaatgatcaggtggttagcacaccaggtcggactacccccgtccgcaaccggctgcttcgtaagcggtggctccatggcgaacttatccgccgtggttgccgccagggacaagttattaccccctctacgtcgtcctgaggggacaatatacgtgtccgaccagacccacatctcaataaagaagtgccttcacatcatcgggttccacgagcaccaggtgagggtcatccctacggacgaggcgttccgtatggacatcgccatccttcagaggacgatcgcagcagaccgtcagtgcggacgtttacctttcttactagtcgcaagctgcggcacgaccaacacggggagcatcgaccctctagacgccctggccgacgtagccaaggaagagggactttggttacacgtggacggagcctacggagcctcgatagcgcttagtgacaagcacggccacttagtaaacggaatcggaagggcagacagtataagctgggacggacacaagtggttattccagacatacgggtgcggcatcgtcctgaccagggactcaacctcgctagcaacatcattcgccgtggacgccgagtacataaactccacaacacctcacggagcagtgaacttctacaacttcgggcccgagttatcaaggcccgcccgtgcaatccctctttggttcacgttaagggtattaggacgtaggaagatgggagagatgatagaccacggattcttacttgcaaggacagcagaggacgagataaggcgttacgcacactggctgatacttgccccggtgaccgcaagtatagtggtattccgttactcgccgcccgagctaaaggacaacgagctagacgagctaaacctagccatatcaacacgtttacttaccgagaacgtggcaagtatattcacaaccaacatccgtgggagagtcgccttaaggttctgcgcaatgaaccctgcgacgaggacagacacaatcatggaagtagtacagcagatggacgaggtggcgcgtaccatctcgacgagtaggccggggctttgcaaggactgcaaggactga